(19) United States
(12) Patent Application Publication
(10) Pub. No.: US 2026/0183362 A1
(43) Pub. Date: Jul. 2, 2026

(54) Methods for Modulating Macrophage Activity

(71) Applicant: Riptide Bioscience, Inc., Vallejo, CA (US)

(72) Inventors: Jesse M. Jaynes, Auburn, AL (US); Henry Wilfred Lopez, Napa, CA (US); George R. Martin, Rockville, MD (US); Clayton Yates, Auburn, AL (US); Balasubramanyam Karanam, Auburn, AL (US)

(21) Appl. No.: 19/549,361

(22) Filed: Feb. 25, 2026

Related U.S. Application Data
(63) Continuation of application No. 17/478,042, filed on Sep. 17, 2021, now Pat. No. 12,564,617.
Application No. 17/478,042 is a continuation of application No. PCT/US2020/027672, filed on Apr. 10, 2020.
(60) Provisional application No. 62/833,352, filed on Apr. 12, 2019.
(60) Provisional application No. 62/966,961, filed on Jan. 28, 2020.

(51) Int. Cl. A61K 38/08; A61P 35/00; C12N 5/0786

(52) U.S. Cl. CPC A61K 38/08; A61P 35/00; C12N 5/0645

(57) ABSTRACT

Aspects of the present disclosure include methods for modulating macrophage activity. Methods according to certain embodiments include contacting a macrophage with a mannose receptor (CD206) binding agent in a manner sufficient to modulate activity of the macrophage. Methods for converting a phenotype of a macrophage from an M2 phenotype to an M1 phenotype are also provided. Methods for inhibiting growth of a CD206-expressing cell as well as methods for treating a subject for a neoplastic condition (e.g., cancer) or a condition associated with chronic inflammation are described. Immuno-modulating peptides suitable for use in the subject methods are also presented.

### CROSS-REFERENCE TO RELATED APPLICATIONS

This application is a continuation of U.S. patent application Ser. No. 17/478,042 filed Sep. 17, 2021, which application is a continuation of PCT Application No. PCT/US2020/027672 filed Apr. 10, 2020, which application, pursuant to 35 U.S.C. § 119 (e), claims priority to the filing dates of U.S. Provisional Patent Application Ser. No. 62/966,961 filed Jan. 28, 2020 and U.S. Provisional Patent Application Ser. No. 62/833,352 filed Apr. 12, 2019; the disclosures of which applications are herein incorporated by reference.

### INTRODUCTION

Cancer remains one of the leading causes of death globally, with an estimated 12.7 million annual cases around the world affecting both sexes. This number is expected to increase to 21 million by 2030.

Recent advances in immunotherapy have transformed the care of many cancer patients. Immunotherapy approaches in the form of checkpoint inhibitor monoclonal antibody (CIMA) therapy or chimeric antigen receptor (CAR) T cell therapy have become first- or second-line treatment options, and afford some patients sustained, durable treatment responses generally not observed with standard systemic chemotherapy. To date, these positive findings are limited to a small number of immunologically ‘hot’ cancers. This is in stark contrast to the majority of solid organ cancers classified as immunologically ‘cold’, where the promise of immunotherapy via T cell activation has largely evaded patients. These tumors create an immune milieu, which excludes cytotoxic T cells or induces an exhausted T cell phenotype through an abundance of immune evasive cues, frequently involving innate immune cells such as tumor-associated macrophages (TAMs) or immature myeloid-derived suppressor cells (MDSCs).

Macrophages are one of the primary innate immune cell populations and play a pivotal role in many human diseases including cancer, where tumor-associated macrophages (TAMs) are a major driver of cancer biology. Tumor cells attract and reprogram myeloid cells to support tumor growth and metastatic spread. While the dichotomous M1 versus M2 classification omits to capture the ontogeny and tissue-specific cues and stress responses in macrophages, in general terms, TAMs in the early stages of tumorigenesis are more frequently M1-like, secreting proinflammatory type I cytokines and inhibit immune evasion, whereas M2-tumor associated macrophages often become the predominant phenotype during tumor progression and further evolved tumor stages. M2-like TAMs may harness tumor growth directly via the excretion of type II cancer-promoting factors, or indirectly via promotion of angiogenesis, the nurturing of cancer stem cells affording resistance to cytotoxic chemotherapy, or the generation of an immune-evasive tumor microenvironment.

### SUMMARY

Aspects of the present disclosure include methods for modulating macrophage activity. Methods according to certain embodiments include contacting a macrophage with a mannose receptor (CD206) binding agent in a manner sufficient to modulate activity of the macrophage. Methods for converting a phenotype of a macrophage from an M2 phenotype to an M1 phenotype are also provided. Methods for inhibiting growth of a CD206-expressing cell as well as methods for treating a subject for a neoplastic condition (e.g., cancer) or a condition associated with chronic inflammation are described. As used herein “inhibiting growth of a CD206-expressing cell includes killing the cell, or reprogramming the cell. In some embodiments, methods include killing a CD206-expressing cell. In other embodiments, methods include reprogramming a CD206-expressing cell. Immuno-modulating peptides suitable for use in the subject methods are also presented. Aspects of the present disclosure also include active agents for binding to an activity modulating domain of CD206. Methods for determining whether a compound binds to an activity modulating domain of CD206 are also provided.

In certain embodiments, methods include modulating a macrophage activity. Methods according to certain embodiments include contacting a macrophage with a CD206-binding agent to modulate activity of the macrophage. In these embodiments, the CD206-binding agent binds to a site selected from the fibronectin II domain of CD206, C-type lectin carbohydrate recognition domain 3 (CRD3) of CD206, C-type lectin carbohydrate recognition domain 4 (CRD4) of CD206 and C-type lectin carbohydrate recognition domain 5 (CRD5) of CD206. In some instances, the CD206-binding agent binds to CD206 with a binding energy of at least −650 kcal/mol. In some instances, the macrophage activity that is modulated is macrophage polarization. In other instances, the macrophage viability is reduced. The macrophage according to certain embodiments is a macrophage with an M2 phenotype. In other embodiments, the macrophage is a tumor associated macrophage. In some embodiments, the CD206-binding agent inhibits macrophage activity. In other embodiments, the CD206-binding agent induces apoptosis of the macrophage. In still other embodiments, the CD206-binding agent stimulates phagocytosis. The macrophage may be contacted in vivo or in vitro.

In other embodiments, methods include inhibiting growth of a CD206-expressing cell. Methods according to certain embodiments include contacting a target CD206-expressing cell with a CD206-binding agent to inhibit growth of the cell. In some instances, the target CD206-expressing cell is a cancer cell. For example, the cancer cell may be a pancreatic cancer cell, a prostate cancer cell, a colon cancer cell, a skin cancer cell or breast cancer cell.

In certain embodiments, methods include treating a subject for a neoplastic condition. Methods according to certain embodiments include administering a therapeutically effective amount of a CD206-binding agent to a subject diagnosed as having a neoplastic condition to treat the neoplastic condition in the subject. In these embodiments, the neoplastic condition may be a solid-tumor cancer. For example, the neoplastic condition may be a cancer selected from pancreatic cancer, prostate cancer, colon cancer, breast cancer and skin cancer. In some instances, methods further include administering an effective amount of a chemotherapeutic agent, antibody agent or cell therapy to the subject. For example, the chemotherapeutic agent, antibody agent or cell therapy may be selected from steroids, anthracyclines, thyroid hormone replacement drugs, thymidylate-targeted drugs, antibodies, checkpoint inhibitor drugs, Chimeric Antigen Receptor/T cell therapies, and other cell therapies. In some embodiments, the chemotherapeutic agent is a non-peptidic compound that reduces proliferation of cancer cells. For example, the chemotherapeutic agent may be a compound selected from alkylating agents, metal complexes, nitrosoureas, antimetabolites, antitumor antibiotics, plant (vinca) alkaloids, hormone modulators, and steroid hormones. In some instances, the antibody agent is a chemotherapeutic antibody agent. For example, the antibody agent may be an antibody raised against a tumor-associated antigen selected from the group consisting of CD20, CD30, CD33, CD52, CD47, EpCAM, CEA, gpA33, Mucins, TAG-72, CAIX, PSMA, Folate-binding protein, Gangliosides (e.g., GD2, GD3, GM2, etc.), Le y, VEGF, VEGFR, Integrin alpha-V-beta-3, Integrin alpha-5-beta-1, EGFR, ERBB2, ERBB3, MET, IGF1R, EPHA3, TRAILR1, TRAILR2, RANKL, FAP and Tenascin. In certain embodiments, methods include administering a checkpoint inhibitor. For example, the checkpoint inhibitor may be an inhibitory compound that targets one or more of PD1, PD-L1, CTLA4, TIM3, LAG3, VISTA, BTLA, TIGIT, LAIR1, CD160, 2B4 and TGFRβ.

In yet other embodiments, methods include treating a subject for a condition associated with chronic inflammation. Methods according to certain embodiments include administering a therapeutically effective amount of a CD206-binding agent to a subject to treat the subject for the condition associated with chronic inflammation. In some embodiments, the condition associated with chronic inflammation is selected from scleroderma or multiple sclerosis, irritable bowel disease, ulcerative colitis, colitis, Crohn's disease, idiopathic pulmonary fibrosis, asthma, keratitis, arthritis, osteoarthritis, rheumatoid arthritis, auto-immune diseases, a feline or human immunodeficiency virus (FIV or HIV) infection, cancer, age-related inflammation and/or stem cell dysfunction, graft-versus-host disease (GVHD), keloids, obesity, diabetes, diabetic wounds, other chronic wounds, atherosclerosis, Parkinson's disease, Alzheimer's disease, macular degeneration, gout, gastric ulcers, gastritis, mucositis, toxoplasmosis, and chronic viral or microbial infections.

In still other embodiments, methods include converting a phenotype of a macrophage from an M2 phenotype to an M1 phenotype. Methods according to certain embodiments include contacting a macrophage having an M2 phenotype with a CD206-binding agent in a manner sufficient to convert the phenotype of the macrophage to an M1 phenotype. In some instances, contacting the CD206-binding agent induces a conformational change in a CD206 receptor of the macrophage sufficient to convert the phenotype of the macrophage to an M1 phenotype. In some instances, converting the phenotype of the macrophage includes inducing expression of CD86 by the macrophage. In other instances, converting the phenotype of the macrophage includes reducing expression of CD206 by the macrophage. In other instances, converting the phenotype of the macrophage includes reducing expression of CD163 by the macrophage. In still other instances, converting the phenotype of the macrophage includes converting the macrophage to a phenotype that exhibits upregulation of M1 cytokines and markers. For example, the M1 cytokine and marker is selected from the group consisting of IL-1β, IL-12, TNFα and nitric oxide synthetase. In other instances, converting the phenotype of the macrophage includes converting the macrophage to a phenotype that exhibits decreased expression of signal regulatory protein α (SIRPα).

In certain embodiments, there is provided an active agent that binds to an activity modulating domain of CD206. In these embodiments, the active agent binds to an activity modulating domain of CD206 selected from the fibronectin II domain of CD206, C-type lectin carbohydrate recognition domain 3 (CRD3) of CD206, C-type lectin carbohydrate recognition domain 4 (CRD4) of CD206 and C-type lectin carbohydrate recognition domain 5 (CRD5) of CD206. In some instances, the active agent binds to the CRD5 domain of CD206. In some instances, the active agent binds to the fibronectin II domain of CD206. In some other instances, the active agent binds to the CRD3 domain of CD206.

In certain embodiments, the methods include determining if an active agent binds to an activity modulating domain of CD206. In these embodiments, the methods include contacting a macrophage comprising CD206 with a compound, and determining whether the compound binds to an activity modulating domain of CD206. In some instances, the method includes determining the activity modulating domain of CD20 that binds the compound. In certain instances of these methods, the macrophage is a macrophage comprising one or more mutations in the activity modulating domains of CD206. In certain embodiments, the activity modulating domain of CD206 is selected from the fibronectin II domain of CD206, C-type lectin carbohydrate recognition domain 3 (CRD3) of CD206, C-type lectin carbohydrate recognition domain 4 (CRD4) of CD206 and C-type lectin carbohydrate recognition domain 5 (CRD5) of CD206. In certain cases, the activity modulating domain of CD206 is the CRD5 domain. In certain instances, the activity modulating domain of CD206 is the fibronectin II domain. In certain other cases, the activity modulating domain of CD206 is the CRD3 domain.

The CD206-binding agent according to certain embodiments of the present disclosure is an immunomodulatory peptide. In some instances, the immunomodulatory peptide is of 5 to 18 amino acid residues in length and includes a striapathic region of alternating hydrophilic and hydrophobic modules that adopts an amphipathic conformation under physiological conditions. In these instances, the striapathic region may include 3 or more hydrophobic modules; and 2 or more hydrophilic modules each comprising at least one cationic residue. In some embodiments, the immunomodulatory peptide includes a sequence defined by one of the formulae:

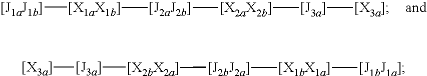

wherein:
J₁ₐ, J_{1b}, J₂ₐ, J_{2b} and J₃ₐ are each independently selected from phenylalanine, tryptophan, alanine, valine, and glycine; and
X₁ₐ, X_{1b}, X₂ₐ, X_{2b} and X₃ₐ are each independently selected from lysine, arginine, histidine, aspartic acid, glutamic acid, asparagine and glutamine. In some instances, J₁ₐ, J_{1b}, J₂ₐ, J_{2b} and J₃ₐ are each phenylalanine; and X₁ₐ, X_{1b}, X₂ₐ, X_{2b} and X₃ₐ are each independently selected from lysine and arginine. In certain embodiments, the immunomodulatory peptide includes: a) a sequence selected from: KFRKAFKRFF (RP182); FFRKFAKRFK (RP183); FFKKFFKKFK (RP185); FFKKFFKKFK (RP186); and FFKKFFKKFK (RP233); orb) a sequence having one or two amino acid substitutions relative to the sequence defined in a). In other embodiments, the immunomodulatory peptide includes: a) a peptide sequence selected from: RWKFGGFKWR (RP832C); FKWRGGRWKF (RP837C); FWKRGGRKWF (RP837A); FWKRFV (RP837N); FVRKWR (RP837C1); FAOOFAOOFO (RP850); FWKRFVRKWR (RP837); FWKKFVKKWK (RP841); WWHHWWHHWH (RP847); WWRHWWHRWR (RP848); WWKHWWHKWK (RP849); GDRGIKGHRGF (RP842); LYKKIIKKLL (RP846); FYPDFFKKFF (RP844); FFRKSKEKIG (RP853); FFRHFATHLD (RP845); and EKLSAFRNFF (RP843); orb) a sequence having one or two amino acid substitutions relative to the sequence defined in a).

CD206-binding agent according to certain embodiments of the present disclosure also includes immunomodulatory peptides including a sequence defined by one of the formulae:

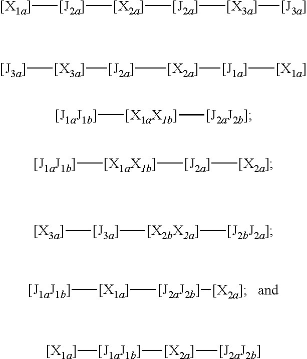

wherein:
J₁ₐ, J_{1b}, J₂ₐ, J_{2b} and J₃ₐ are each independently selected from phenylalanine, tryptophan, alanine, valine, leucine, isoleucine, methionine, tyrosine, threonine, serine, cysteine, proline, and glycine; and
X₁ₐ, X_{1b}, X₂ₐ, X_{2b} and X₃ₐ are each independently selected from lysine, arginine, histidine, aspartic acid, glutamic acid, asparagine and glutamine. In certain embodiments, the immunomodulatory peptide includes: a) a sequence selected from: AFKRFF (182-FN6); FFKKFF (185-FN6); FWKRFV (837-FN6); WVRRVV (WLUB-F1-N6); IFKKIE (CEC-F1-N6) FLRNLV (LL37F-3-N6); FLHSAK (MAG-F1-N6); FFHHIF (PISC-F-N6); FFKKAA (PLEU-F-N6); ALKKVF (PSEU-F-N6); LYKKII (CXCL4-F-N6); LFRRAF (IL24-FN6); FLKRLL (IL7-FN6); FFRRFA (ABCP-FN6); FFRHFA (E1P-FN6); AIRRIP (gP120-FN6); AFHRFF (GP2B-FN6); FFNRFA (MCPH-FN6); AFKRFF (SPRA-FN6); AFKRFF (TPRO-FN6); IVRRAD (COL18-FN6); FWRWFK (HX5/CPAP); KFWRWF (HX6/YJPA); WFRFWK (HX7/CLPB) KWFRFW (HX8/ATG1); AFHHFF (HEX16F/STPK); FFRNFA (HEXF13/SIF1); AFHRFF (HEX9FflHIF); FFRQFA (HEXF1/ATPB); AFNRFF (HEX2F/AATF); WIQRMM (CXCL13-FN6); WVQRVV (CXCL8-FN6); AFRNFF (HEX3F/FBNA); and TLRRFM (HEX18/HSHK); or b) a sequence having one or two amino acid substitutions relative to the sequence defined in a). In other embodiments, the immunomodulatory peptide includes: a) a sequence selected from: DVRMRL (MCMV-FN6); and RRAELG (TONB-FN6) or b) a sequence having one or two amino acid substitutions relative to the sequence defined in a). In some other embodiments, the immunomodulatory peptide includes: a) a sequence selected from: FWRWFA (HX1/MMPL); AFWRWF (HX2/ABCT); WFRFWA (HX3/GTRF); AWFRFW (HX4/AXES); VAVRIW (HX9/IDRF/AMIA); FFRFFA (HEXF2/AMTl); and AFFRFF (HEX13F/TGME); or b) a sequence having one or two amino acid substitutions relative to the sequence defined in a). In other embodiments, the immunomodulatory peptide includes: a sequence selected from: FFKKFF; WWKKFF; FWKKWF; FFKKWW; WWKKWW; YYKKYY; IIKKYY; YIKKIY; YYKKII; IIKKII; MMKKMM; LLKKMM; MLKKLM; MMKKLL; LLKKLL; VVKKVV; AAKKVV; VAKKAV; VVKKAA; AAKKAA; GGKKGG; TTKKGG; GTKKTG; GGKKTT; TTKKTT; SSKKSS; CCKKSS; SCKKCS; SSKKCC; and CCKKCC; or b) a sequence having one or two amino acid substitutions relative to the sequence defined in a). In certain other cases, the immunomodulatory peptide includes: a sequence selected from: FKFKFK; WKWKWK; YKYKYK: IKIKIK; MKMKMK; LKLKLK; VKVKVK; AKAKAK; GKGKGK; TKTKTK; SKSKSK; CKCKCK; KFKFKF; KWKWKW; KYKYKY; KIKIKI; KMKMKM; KLKLKL; KVKVKV; KAKAKA; KGKGKG; KTKTKT; KSKSKS; and KCKCKC; or b) a sequence having one or two amino acid substitutions relative to the sequence defined in a).

CD206-binding agent according to certain embodiments of the present disclosure also includes immunomodulatory peptides including a sequence defined by one of the formulae:

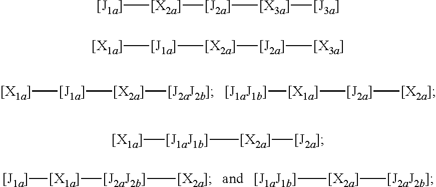

wherein:
J₁ₐ, J_{1b}, J₂ₐ, J_{2b} and J₃ₐ are each independently selected from phenylalanine, tryptophan, alanine, valine, leucine, isoleucine, methionine, tyrosine, threonine, serine, cysteine, proline, and glycine; and
X₁ₐ, X_{1b}, X₂ₐ, X_{2b} and X₃ₐ are each independently selected from lysine, arginine, histidine, aspartic acid, glutamic acid, asparagine and glutamine. In certain embodiments, the immunomodulatory peptide includes: a) a sequence selected from: AFKRF; FFKKF; FWKRF; WVRRV; IFKKI; FLRNL; FLHSA; FFHHI; FFKKA; ALKKV; LYKKI; LFRRA; FLKRL; FFRRF; FFRHF; AIRRI; AFHRF; FFNRF; IVRRA; FWRWF; KFWRW; WFRFW; KWFRF; AFHHF; FFRNF; FFRQF; AFNRF; WIQRM; WVQRV; AFRNF; TLRRF; FKRFF; FKKFF; WKRFV; VRRVV; FKKIE; LRNLV; LHSAK; FHHIF; FKKAA; LKKVF; YKKII; FRRAF; LKRLL; FRRFA; FRHFA; IRRIP; FHRFF; FNRFA; VRRAD; WRWFK; FRFWK; FHHFF; FRNFA; FRQFA; FNRFF; IQRMM; VQRVV; FRNFF; LRRFM; DVRMR; VRMRL; RRAEL; RAELG; and RWKFG; or b) a sequence having one or two amino acid substitutions relative to the sequence defined in a). In other embodiments, the immunomodulatory peptide includes: a) a peptide sequence selected from: AFWRW; AWFRF; VAVRI; FFRFF; AFFRF; WRWFA; FRFWA; AVRIW; and FRFFA; or b) a sequence having one or two amino acid substitutions relative to the sequence defined in a). In other embodiments, the immunomodulatory peptide includes: a) a peptide sequence selected from: FFKKF; WWKKF; FWKKW; FFKKW; WWKKW; YYKKY; IIKKY; YIKKI; YYKKI; IIKKI; MMKKM; LLKKM; MLKKL; MMKKL; LLKKL; VVKKV; AAKKV; VAKKA; VVKKA; AAKKA; GGKKG; TTKKG; GTKKT; GGKKT; TTKKT; SSKKS; CCKKS; SCKKC; SSKKC; and CCKKC; FKKFF; WKKFF; WKKWF; FKKWW; WKKWW; YKKYY; IKKYY; IKKIY; YKKII; IKKII; MKKMM; LKKMM; LKKLM; MKKLL; LKKLL; VKKVV; AKKVV; AKKAV; VKKAA; AKKAA; GKKGG; TKKGG; TKKTG; GKKTT; TKKTT; SKKSS; CKKSS; CKKCS; SKKCC; and CKKCC; or b) a sequence having one or two amino acid substitutions relative to the sequence defined in a). In certain other cases, the immunomodulatory peptide includes: a sequence selected from: a) a peptide sequence selected from: FKFKF; WKWKW; YKYKY: IKIKI; MKMKM; LKLKL; VKVKV; AKAKA; GKGKG; TKTKT; SKSKS; CKCKC; KFKFK; KWKWK; KYKYK; KIKIK; KMKMK; KLKLK; KVKVK; KAKAK; KGKGK; KTKTK; KSKSK; and KCKCK; or b) a sequence having one or two amino acid substitutions relative to the sequence defined in a).

CD206-binding agent according to certain embodiments of the present disclosure also includes immunomodulatory peptides including a sequence defined by one of the formulae:

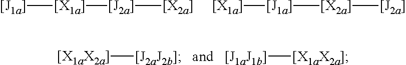

wherein:
J₁ₐ, J_{1b}, J₂ₐ, and J_{2b} are each independently selected from phenylalanine, tryptophan, alanine, valine, leucine, isoleucine, methionine, tyrosine, threonine, serine, cysteine, proline, and glycine; and
X₁ₐ, X_{1b}, X₂ₐ, and X_{2b} are each independently selected from lysine, arginine, histidine, aspartic acid, glutamic acid, asparagine and glutamine. In certain embodiments, the immunomodulatory peptide includes: a) a sequence selected from: AFKR; FFKK; FWKR; WVRR; IFKK; FLRN; FLHS; FFHH; ALKK; LYKK; LFRR; FLKR; FFRR; FFRH; AIRR AFHR; FFNR; IVRR; FWRW; KFWR; WFRF; KWFR; AFHH; FFRN; FFRQ; AFNR; WIQR; WVQR; AFRN; TLRR; KRFF; KKFF; KRFV; RRVV; KKIE; RNLV; HSAK; HHIF; KKAA; KKVF; KKII; RRAF; KRLL; RRFA; RHFA; RRIP; HRFF; NRFA; RRAD; RWFK; RFWK; HHFF; RNFA; RQFA; NRFF; QRMM; QRVV; RNFF; RRFM; VRMR; RMRL; RAEL; AELG; and WKFG; or b) a sequence having one or two amino acid substitutions relative to the sequence defined in a). In other embodiments, the immunomodulatory peptide includes: a) a peptide sequence selected from: FWRW; AFWR; WFRF; AWFR; VAVR; FFRF; AFFR; RWFA; WRWF; RFWA; FRFW; VRIW; RFFA; and FRFF; or b) a sequence having one or two amino acid substitutions relative to the sequence defined in a). In other embodiments, the immunomodulatory peptide includes: a) a peptide sequence selected from: FFKK; WWKK; FWKK; YYKK; IIKK; YIKK; MMKK; LLKK; MLKK; VVKK; AAKK; VAKK; GGKK; TTKK; GTKK; SSKK; CCKK; SCKK; KKFF; KKWF; KKWW; KKYY; KKIY; KKII; KKMM; KKLM; KKLL; KKVV; KKAV; KKAA; KKGG; KKTG; KKTT; KKSS; KKCS; and KKCC; or b) a sequence having one or two amino acid substitutions relative to the sequence defined in a). In certain other cases, the immunomodulatory peptide includes: a sequence selected from: a) a peptide sequence selected from: FKFK; WKWK; YKYK: IKIK; MKMK; LKLK; VKVK; AKAK; GKGK; TKTK; SKSK; CKCK; KFKF; KWKW; KYKY; KIKI; KMKM; KLKL; KVKV; KAKA; KGKG; KTKT; KSKS; and KCKC; or b) a sequence having one or two amino acid substitutions relative to the sequence defined in a).

CD206-binding agent according to certain embodiments of the present disclosure also includes small molecule active agents. In certain instances, the small molecule active agent is described by formula (I):

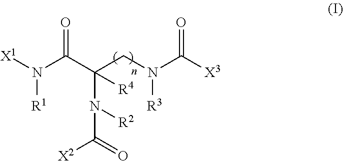

wherein:
R¹-R⁴ are each independently selected from hydrogen, alkyl and substituted alkyl;
X¹ is selected from alkyl, substituted alkyl, aryl, substituted aryl, aralkyl, substituted aralkyl, heterocycle, substituted heterocycle, heteroaryl, substituted heteroaryl;
X² is selected from alkyl, substituted alkyl, aryl, substituted aryl, amino, substituted amino, heteroaryl, substituted heteroaryl, heterocycle, substituted heteroaryl;
X³ is selected from alkyl, substituted alkyl, aryl, substituted aryl, naphthyl, substituted naphthyl, heterocycle, substituted heterocycle, heteroaryl, substituted heteroaryl, aryl heterocycle, substituted aryl heterocycle; and
n is an integer from 1 to 10,
or a pharmaceutically acceptable salt or solvate thereof.

A CD206-binding agent according to certain embodiments of the present disclosure also includes small molecule active agents described by formula (II):

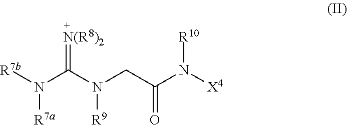

wherein:
R^{7a}, R^{7b}, R⁸, R⁹ and R¹⁰ are each independently selected from hydrogen, alkyl and substituted alkyl; and
X⁴ is selected from alkyl, aryl, aralkyl, heterocycle, and heteroaryl, acyl, wherein X⁴ is optionally further substituted with one or more groups selected from, alkyl, substituted alkyl, aryl, substituted aryl, amino, substituted amino, carboxamide, substituted carboxamide, heterocycle, substituted heterocycle, and a second compound of formula (II)
or a pharmaceutically acceptable salt or solvate thereof.

CD206-binding agent according to certain embodiments of the present disclosure also includes small molecule active agents described by formula (III):

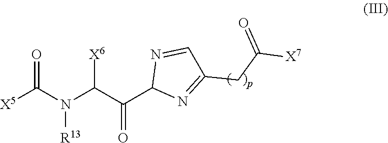

wherein:
R¹³ is selected from hydrogen, alkyl and substituted alkyl;
X⁵ is selected from alkyl, substituted alkyl, aryl, substituted aryl, amino, substituted amino, heteroaryl, substituted heteroaryl, heterocycle, substituted heteroaryl;
X⁶ is selected from alkyl, substituted alkyl, aryl, substituted aryl, aralkyl, substituted aralkyl, heterocycle, substituted heterocycle, heteroaryl, substituted heteroaryl;
X⁷ is selected from alkyl, substituted alkyl, aryl, substituted aryl, naphthyl, substituted naphthyl, heterocycle, substituted heterocycle, heteroaryl, substituted heteroaryl, aryl heterocycle, substituted aryl heterocycle; and
p is an integer from 1 to 10,
or a pharmaceutically acceptable salt or solvate thereof.

A CD206-binding agent according to certain embodiments of the present disclosure also includes specific binding members. In certain cases, the specific binding pair is an antibody, or a binding fragment thereof. In certain cases, the specific binding member targets a sequence of CD206 selected from NFGDLVSIQSESEKK, NDAQSAYFIGLLISL, SKEKETMDNARAF, and EDENCVTMYSNSGFWN.

### BRIEF DESCRIPTION OF THE DRAWINGS

FIG. 1 shows biophysical homology screening using the Molly font, which identifies conservation of 10mer structural motif across representative HDPs, outer surface virulence factors, and collagens. A. Molly font heuristic denoting the chemical nature of amino acids for biophysical homology comparisons. The size of the circle indicates the proportional steric amino acid volumes in water, and the hydrophobicity/hydrophilicity of each amino acid is indicated by a color scale in which the most hydrophobic amino acids are the most intense cyan color, and those that are less hydrophobic are proportionally less concentrated cyan. Amino acids that are most hydrophilic possess the deepest magenta color, and a graduated scale of less intense magenta color indicates amino acids of lower hydrophilic character. Mnemonic glyphs used to capture biophysical relationships and further characterize chemical properties of amino acids include thickness depicting dissociation constant of their ionizable protons and charge, with charged amino acids having a “+” or a “−” sign incorporated within their glyph. Numerical values encode the energy in kcal/mol required to move the amino acid side chain from the interior of a lipid bilayer to the outside aqueous environment.

FIG. 2 shows biophysical homology screening using the Molly font, which identifies conservation of a 10mer structural motif across representative HDPs, outer surface virulence factors, and collagens. Conservation of the 10mer structural motif with secondary a-helical structure with amphiphilic surface topology of hydrophobic and hydrophilic faces across representative HDPs, outer surface virulence factors, and collagens, is shown by Molly font. “DHDP” designates a designed host defense peptide.

FIG. 3 shows secondary α-helical structures, helical hydrophobicity wheel projections, and Molly font alignments (bottom) of RP-182 and RP-426.

FIG. 4 shows relative binding affinities (BE; in kcal/mol) of individual 10mer homology sequence ligand-lectin receptor combinations by ClusPro®, identifying MRC1/CD206 as the top binding target.

FIG. 5 shows relative binding energies to top ten C-type lectin receptors utilizing ClusPro®.

FIG. 6 shows the docking campaign of biophysical 10mer homology motifs and C-type lectin receptors. A. Relative binding energies, in kcal/mol, of top 10mer peptide motif ligand—C-type lectin receptor combinations utilizing ClusPro®. Origin of 10mer peptide sequence shown on bottom; predicted affinities to individual lectin receptors annotated on top. B. Mannose receptor CD206 shows the highest affinity to 10mer biophysical peptide sequences across all tested peptide-ligand receptor combinations. Cumulative score of the top three peptide ligand-receptor combinations is shown, with each receptor examined by 23 peptide ligand-receptor combinations. The receptor with the highest affinity (lowest −BE; in kcal/mol) was allocated three points, the second highest two, and the third highest one point. The plot shows the sum of points for each receptor across all peptides. CD206FL, full length CD206; CRD, carbohydrate recognition domain.

FIG. 7A shows a full length MRC1/CD206 model. Known functional domains of human MRC1/CD206. CRD, carbohydrate recognition domains 1-8.

FIG. 7B shows a full length MRC1/CD206 model. SAXS profiles are shown as a function of concentration of the full length MRC1/CD206 protein where I(q) is scattering intensity and q (in Å-1) is the scattering vector. Structural parameters extracted from SAXS data are listed on bottom; Rg Guinier: the radius of gyration produced from Guinier plot of SAXS data extrapolated to 0 concentration; Rest Rg and Dmax (maximum dimension of the particle) are produced from GNOM (https://www.embl-hamburg.de/biosaxs/manuals/gnom.html). MW1 and MW2 are molecular weights estimated from SAXS data using Porod volume (Vporod) and correlation volume (Vc). For Vc calculation qmax=0.15 Å-1 was used. The results suggest that MRC1/CD206 forms a dimer in the solution.

FIG. 7C shows a full length MRC1/CD206 model. C. 5 top-ranking human MRC1/CD206 models created by I-TASSER tested for best fit with experimental SAXS data. The discrepancy between models and experimental curves (χ2) are in the bottom table. In the fitting, MRC1/CD206 dimer models were used based on Model1-5 as monomer, respectively.

FIG. 7D shows a full length MRC1/CD206 model. C. 5 top-ranking human MRC1/CD206 models created by I-TASSER tested for best fit with experimental SAXS data. The discrepancy between models and experimental curves (χ2) are in the bottom table. In the fitting, MRC1/CD206 dimer models were used based on Model1-5 as monomer, respectively.

FIG. 8A shows a model of conformational bend in CRD4 and CRD5 of MRC1/CD206 induced by RP-182. Hydrophobic plane of RP-182 bound to CRD5 (cyan color).

FIG. 8B shows another model of conformational bend in CRD4 and CRD5 of MRC1/CD206 induced by RP-182. Hydrophobic plane of RP-182 bound to CRD5 (cyan color).

FIG. 9A shows negatively stained electron microscopy micrographs of full length CD206 proteins incubated with vehicle (blue squares) and RP-182 (red squares) and corresponding 2D classes (inlets), schematic of open ‘elongated’ and ‘closed’ conformations on left.

FIG. 9B shows another negatively stained electron microscopy micrographs of full length CD206 proteins incubated with vehicle (blue squares) and RP-182 (red squares) and corresponding 2D classes (inlets), schematic of open ‘elongated’ and ‘closed’ conformations on left.

FIG. 10 shows that RP-182 binds to MRC1/CD206. A. Dose-response relationship of increasing concentrations of RP-182 and the induction of the closed conformation of the CD206 receptor. Percent closed conformation of 100 examined CD206 particles on negatively stained EM micrographs of CD206 full length proteins at increasing concentrations of RP-182 (in μM).

FIG. 11 shows ratios of closed vs open conformations of MRC1/CD206 (low, CD206: peptide ratio 1:40, high, 1:500).

FIG. 12A shows that RP-182 binds to MRC1/CD206. Induction of the closed conformation of the CD206 receptor by 10mer homology motifs. Representative micrographs of full length CD206 incubated with 10mer biophysical homology peptides RP-185, RP-832C, AVP1, LL37F, and control peptide RP-426, 2D classes are shown on bottom.

FIG. 12B shows additional views of RP-182 binding to MRC1/CD206. Induction of the closed conformation of the CD206 receptor by 10mer homology motifs. Representative micrographs of full length CD206 incubated with 10mer biophysical homology peptides RP-185, RP-832C, AVP1, LL37F, and control peptide RP-426, 2D classes are shown on bottom.

FIG. 12C shows an extra view of RP-182 binding to MRC1/CD206. Induction of the closed conformation of the CD206 receptor by 10mer homology motifs. Representative micrographs of full length CD206 incubated with 10mer biophysical homology peptides RP-185, RP-832C, AVP1, LL37F, and control peptide RP-426, 2D classes are shown on bottom.

FIG. 13 shows Microscale Thermophoresis (MST) shifts of human (left) and murine (right) MRC1/CD206 at increasing concentrations of RP-182. KD model response curves are fit to the MST data of three independent experiments; control peptide RP-426 shown on bottom. Error bars represent standard deviations of the means.

FIG. 14A shows the human peripheral blood monocyte-derived macrophage polarization protocol.

FIG. 14B shows the cellular Thermal Shift Assay (CETSA) of human macrophages.

FIG. 14C shows the murine bone marrow-derived macrophage (BMDM) polarization protocol.

FIG. 14D shows Cellular Thermal Shift Assay (CETSA) of murine macrophages.

FIG. 15A shows abbreviated synthesis scheme of RP-182 analogues with diazirine-containing phenylalanine and biotin. Synthesis of diazirinyl D-phenylalanine derivative and Fmoc-diazirine-containing phenylalanine (enantiomers were resolved using a chiral column (Chiralpac IB 4.6×250 mm, 100% EOH; 1 ml/min)). Fmoc solid-phase peptide synthesis, and coupling with biotinylated linkers.

FIG. 15B shows derivatives of RP-182 harboring diazirine group and biotin via PEG (NCGC-00510433 and -35) or hydrocarbon (NCGC-00510432 and -34) linkers were tested for in vitro binding to recombinant MRC1/CD206 via microscale thermophoresis (MST) assay. Measured KD constants shown on the right. NCGC-00510434 was used for all further pulldown experiments.

FIG. 16 shows Mapping of binding region of RP-182 to MRC1/CD206 via crosslinking followed by pulldown and proteomic analysis identifies fragment NFGDLVSIQSESEKK within the carbohydrate recognition domain 5 (CRD5; CTLD5) as the binding region of RP-182 to CD206. A. Peptide fragments (highlighted in rectangles) from full length rMRC1/CD206 pulled down with biotinylated diazirine RP-182 analogue NCGC-00510434 after trypsin digestion. B. Peptide fragments of MRC1/CD206 identified after crosslink to the RP-182 analogue, digestion, and pulldown as measured by decreased MRC1/CD206 fragments in the supernatant by mass spectrometry. Carbohydrate recognition domain 5 (CRD5; CTLD5) fragment NFGDLVSIQSESEKK (highlighted in red) was identified by both approaches. C. MS/MS spectrum of MRC1/CD206 CRD5 peptide NFGDLVSIQSESEKK precipitated by biotin-coupled RP-182. Panel A, LC/MS/MS chromatography showing the retention of CRD5 peptide, Panel B, MS1 full scan showing the peptide's triply charged ion m/z=560.9519 (monoisotopic), panel C, MS2 fragment scan showing the spectrum of monoisotopic m/z: 560.95319 Da (+0.34 mmu/+0.61 ppm), RT: 27.01 min. Spectrum was obtained by nano-HPLC-MS/MS analysis.

FIG. 17 shows a volcano plot of RNASeq analysis of vehicle- vs RP-182-treated M2 macrophages.

FIG. 18 shows Gene expression changes induced by RP-182 in M1- and M2-polarized BMDMs. Volcano plots of RNASeq analysis of vehicle- vs RP-182-treated M1 and M2 macrophages, treatment times indicated on top. Differentially expressed genes identified by EdgeR analysis with false discovery rate (FDR) q<0.05, −1<Log 2[FC]>1 are shown (FC, fold change). Summary of identified DEGs shown on right.

FIG. 19 shows Cytoscape® Functional GO Enrichment and Network Analyses of DEGs of vehicle vs RP-182-treated M2 BMDMs.

FIG. 20 shows Pathway Studio® graph of GO Cell Processes of most common genes across enriched gene sets in RP-182-treated M2 BMDMs.

FIG. 21A shows protein network and related cellular processes of CD206 interactome induced by RP-182 in M2 macrophages

FIG. 21B shows RP182 vs. vehicle-treated M2 BMDM

FIG. 21C shows RP182 treated vs. untreated macrophage RNA-Seq

FIG. 21D shows RP182 treated M2 protein pull down

FIG. 21E shows RP182/MS M2 macrophage protein pulldown

FIG. 22 shows electron microscopy images (at 1,000×; zoom 2,400×) of M1- and M2-polarized BMDMs.

FIG. 23 shows immunofluorescence images of BMDMs polarized into M2 stained with anti-Rab5, Rab7, LAMP-1, and CD206 antibodies, nuclei with DAPI. Quantification of induced fluorescence on bottom. For all figures, data shown are representative of three independent experiments and normalized to corresponding vehicle treatment unless indicated otherwise.

FIG. 24 shows that RP-182 activates phagocytosis and phagolysosome formation in M2- but not M1-polarized human macrophages derived from peripheral blood mononuclear cells (PBMC)s of healthy volunteers. Immunofluorescence of human macrophages derived from CD14-positive PBMCs of healthy donors and polarized into M1 and M2 populations stained with anti-Rab5, Rab7, LAMP-1, and CD206 antibodies. Representative images at 40×. Quantification of RP-182-induced Rab5, Rab7, LAMP-1, and CD206 expression levels in M1 (blue bars) and M2 (red bars) BMDMs is shown on the left. Fluorescence (bright objects) was normalized to the number of nuclei (DAPI), and vehicle-treated signal was set to 1. At least 100 cells in ≥5 independent fields were measured. Macrophages were treated with 20 μM RP-182 for 2 hours.

FIG. 25A shows that RAB5a and RAB7 RP-182 activates phagocytosis and phagolysosome formation in M2- but not M1-polarized human macrophages derived from peripheral blood mononuclear cells (PBMC)s of healthy volunteers. Immunofluorescence of human macrophages derived from CD14-positive PBMCs of healthy donors and polarized into M1 and M2 populations stained with anti-Rab5, Rab7, LAMP-1, and CD206 antibodies. Representative images at 40×. Quantification of RP-182-induced Rab5, Rab7, LAMP-1, and CD206 expression levels in M1 (blue bars) and M2 (red bars) BMDMs is shown on the left. Fluorescence (bright objects) was normalized to the number of nuclei (DAPI), and vehicle-treated signal was set to 1. At least 100 cells in ≥5 independent fields were measured. Macrophages were treated with 20 μM RP-182 for 2 hours.

FIG. 25B shows that MAP1 and CD206 RP-182 activates phagocytosis and phagolysosome formation in M2- but not M1-polarized human macrophages derived from peripheral blood mononuclear cells (PBMC)s of healthy volunteers. Immunofluorescence of human macrophages derived from CD14-positive PBMCs of healthy donors and polarized into M1 and M2 populations stained with anti-Rab5, Rab7, LAMP-1, and CD206 antibodies. Representative images at 40×. Quantification of RP-182-induced Rab5, Rab7, LAMP-1, and CD206 expression levels in M1 (blue bars) and M2 (red bars) BMDMs is shown on the left. Fluorescence (bright objects) was normalized to the number of nuclei (DAPI), and vehicle-treated signal was set to 1. At least 100 cells in ≥5 independent fields were measured. Macrophages were treated with 20 μM RP-182 for 2 hours.

FIG. 26 shows RP-182 activates phagocytosis, autophagy, and apoptosis in BMDMs co-cultured with conditioned media from PANC1 cells. Representative images at 40× on top. Quantification of RP-182-induced Rab7, LC-3, LAMP-1, and cleaved caspase 8 expression levels (red bars) cells is shown on bottom. Fluorescence (bright objects) was normalized to the number of nuclei (DAPI), and vehicle-treated signal was set to 1. At least 100 cells in ≥5 independent fields were measured. Macrophages were treated with 20 μM RP-182 for 2 hours.

FIG. 27A shows RP-182 but not control peptide RP-426 induces phagocytosis in M2-polarized BMDMs. A. Rab7 levels by immunocytochemistry in M2-polarized macrophages treated with vehicle, 20 μM of RP-182 or RP-426. Fluorescence (bright objects) was normalized to number of nuclei (DAPI), and vehicle-treated was set at 1. At least 100 cells in ≥5 independent fields were measured.

FIG. 27B shows induction of phagosomes in RP-182-treated but not RP-426-treated M2 macrophages. Electron microscopy images (representative image at 1,000× magnification shown on left; zoom 2,400× on right), shows phagosome with internal membrane structures.

FIG. 28 shows immunofluorescence images of M2 BMDMs stained with anti-NF-kB/p65.

FIG. 29 shows that RP-182 reduces cytoplasmic expression of the negative regulator of macrophage activation and inflammation IKKα subunit of the IkappaB kinase (IKK) complex and activates autophagy and caspase 8 in M2-polarized BMDMs. A. Immunofluorescent staining of IKKα in M2 BMDMs after 2 hours treatment with vehicle and RP-182.

FIG. 30 shows quantification of activation of phagocytosis, autophagy, and apoptosis in M1 and M2 macrophages over time.

FIG. 31 shows activation of autophagy and apoptosis in M2 macrophages by RP-182 measured by immunofluorescent staining of LC3 and cleaved caspase 8. Representative immunofluorescent images of vehicle- and RP-182-treated M2 macrophages at 24 hours.

FIG. 32 shows RP-182 activates autophagy and apoptosis in human M2 macrophages. LC3 and cleaved caspase 8 levels were measured by immunocytochemistry staining of human M2 macrophages after 24 hours treatment with RP-182. Measured fluorescence (bright objects) was normalized to number of nuclei (DAPI), and fold change relative to vehicle control was plotted. Error bars are standard deviations of n≥2 independent experiments, ≥50 cells in 5 independent fields were measured.

FIG. 33 shows quantification of cleaved caspase 3 and 7 levels after 24 hours treatment.

FIG. 34 shows cell viability of human and murine M1 (blue curves) and M2 (red curves) macrophages after 48 hours treatment with RP-182 relative to vehicle treatment.

FIG. 35 shows RP-182 but not RP-426 induces cell death in M2-polarized macrophages. A. Representative images of M2 BMDMs treated with vehicle or RP-182 and stained after 48 hours using dual calcein AM (green)-ethidium homodimer (red). Viability for dose response curves shown in FIG. 2K was calculated as ratio of live cells (positive calcein AM and negative red staining) and dead cells (absent green, positive red staining).

FIG. 36 shows dose-response curves of control peptide RP-426 in human (left) and murine (right) M2-polarized macrophages. Ratios of viable cells were normalized to vehicle-treated control, error bars represent standard deviations of two independent experiments in triplicates.

FIG. 37 shows RP-182 does not inhibit cell growth of pluripotent progenitor cells, cancer cells, fibroblasts, or endothelial or dendritic cells. Dose-response curves of RP-182 in human pancreas cancer cells PANC1, murine KPC, human fibroblasts, endothelial, and DC2.4 dendritic cells. Cells were stained after 48 hours using dual calcein AM (green)-ethidium homodimer (red) and experiments were conducted in parallel with RP-182 testing in M2 BMDMs as positive control. Representative curves of N=2 independent experiments conducted are shown.

FIG. 38 shows flow cytometry plots of CD86 and CD206-positive CD11B+F4/80+Gr-1-macrophage fractions of M2 BMDMs after treatment with vehicle or RP-182 at indicated timepoints.

FIG. 39A shows gating strategy for the determination of CD86+ and CD206-positive CD11b+F4/80+Gr-1− macrophage fractions. A. Representative flow cytometry plots depicting gating strategy for determination of CD86 and CD206-positive macrophages after treatment with vehicle, RP-182 for 24 (middle) and 48 hours (bottom). Alive cells circled in black

FIG. 39B shows representative immunocytometry images of M2 BMDMs stained with CD206 (left) and quantification (right) of total anti-CD206 immunofluorescence signal intensity in M2 BMDMs after 24 hours treatment with RP-182 or vehicle.

FIG. 40A shows quantification of fractions of IL-1β, IL-12p40, and TNF-α positive CD11b+F4/80+Gr-1− cells, and of CD86+CD206−, CD86+CD206+ double positive, and CD86−CD206+ subpopulations of M2 BMDMs by flow cytometry.

FIG. 40B shows flow cytometry plots fractions of IL-1β, IL-12p40, and TNF-α positive CD11b+F4/80+Gr-1− cells, and of CD86+CD206−, CD86+CD206+ double positive, and CD86−CD206+ subpopulations of M2 BMDMs.

FIG. 40C shows flow cytometry plots fractions of IL-1β, IL-12p40, and TNF-α.

FIG. 41 shows M1 and M2 gene expression profile in CD11b+F4/80+Gr-1−CD206+M2 BMDMs isolated by fluorescence activated cell sorting (FACS) after 2 hours treatment with vehicle (black bars) and RP-182 (red bars). Relative transcript levels by qRT-PCR after normalization to internal housekeeping genes, vehicle-treated signal was set to 1. N≥3 per group, in triplicates, error bars indicate SEM.

FIG. 42 shows immune checkpoint and M1 cytokine positive macrophage populations in vehicle-treated CD86− CD206+ M2 BMDMs, and RP-182-treated CD86+ CD206− and CD86+CD206+ subpopulations. Univariate histograms with percentage positive cell fractions, quantifications of N=3 independent experiments are shown.

FIG. 43 shows representative flow cytometry plots of employed gating strategy including univariate histograms (with percentage of positive cell fractions) for determination of SIRPα checkpoint-positive as well as TNFα-positive macrophage cell fractions in vehicle-treated CD86−CD206+ (top row) and RP-182-treated (bottom) CD86+CD206+ (double positive) and CD86+CD206− positive M2 BMDMs. Quantification of N=3 independent experiments done in triplicates shown on bottom. 1: CD86−CD206+ fraction treated with vehicle; 2: CD86+CD206+ double positive fraction treated with RP-182; 3: CD86+CD206− positive cell fraction treated with RP-182.

FIG. 44 shows flow cytometry analysis and quantification of CD11b+F4/80+Gr-1− cell fractions with phagocytosed E. coli-covered latex beads, quantification of N=3 independent experiments.

FIG. 45 shows quantification of annexin V-positive (top) and cleaved caspase 3-positive cell fractions (bottom) of CD86+CD206−, CD206+CD86+, and CD86−CD206+ M2 BMDMs treated with RP-182 at indicated time points.

FIG. 46 shows quantification of vehicle- and RP-182-treated CD86+ (top) and CD206+ (bottom) CD11b+F4/80+Gr-1− BMDM-M2 cells co-incubated with NF-kB inhibitors JSH-23 and QNZ (EVP4593) and autophagy inhibitors bafilomycin (BF) and chloroquine (CQ). MEK inhibitor AZD6244 (selumetinib; AZD) shown as negative control).

FIG. 47 shows Pearson's correlation analysis of gene expression matrices between samples using global RNASeq data (left) and M1 M2 marker set derived from BMDMs (right). Values ranges from 0 to 1, a high value between samples indicates high degree of correlation between two sample sets.

FIG. 48A shows biological activity of RP-182 is CD206-dependent and initiates activation of Rac1/CDC42 signaling. A. RP-182 dose-response curves of binding to recombinant CD206 (MST assay; red curve), induction of the closed conformation of CD206 (electron microscopy; green curve), induction of phagocytosis (anti-Rab7; purple), and M2 cell viability (blue) in M2 BMDMs.

FIG. 48B shows M1 and M2 gene expression profiles of BMDMs isolated from B6.129P2-Mrc1tm1Mnz/J (CD206−/−) and wild type C57BL/6 mice (CD206+/+) after polarization into M1 and M2. Similar M1 and M2 gene expression profiles of BMDMs isolated CD206−/− and wild type C57BL/6 mice (CD206+/+) after polarization into M1 and M2. Relative gene expression levels measured by qRT-PCR of M1 markers CD86, iNOS, IL-1β, and IL-27 and M2 markers CD206, Fizz1, and YM1 in M1 (blue color) and M2 populations (red color) of CD206−/− and CD206+/+ BMDMs. For quantification of the relative expression levels see materials and methods. Mean±SEM of n≥3 per group is shown.

FIG. 49 shows immunofluorescence images of BMDMs derived from wild type and CD206−/− mice polarized into M2 stained with anti-Rab7, LC3, and cleaved caspase 8, quantification of three independent experiments on bottom.

FIG. 50 shows cell viability of M2 BMDMs derived from CD206^{wt} (red curve) and CD206^{-/-} (brown curve) mice. D. Flow cytometry plots of CD86+ and CD206+ fractions (top) and quantification of M1 marker-positive cells in CD11b+F4/80+Gr-1− M2-BMDMs isolated from CD206^{-/-} mice. Mean % positive cells of N=3 independent experiments in triplicates are shown.

FIG. 51 shows characterization of binding partners of CD206 induced by RP-182 in M2 macrophages. Proteomic analysis of CD206 pulled down from M2 BMDMs incubated with biotinylated RP-182 coupled to beads versus beads alone. PSM, peptide-spectrum match. Applied cut-off criteria included ratio of RP-182 to control was ≥5, MS identified peptides covered more than 10% of the protein sequence, and FDR<0.01 yielding 119 proteins (attached separately).

FIG. 52A shows immunoblot of lysates immunoprecipitated with biotinylated peptides (left) or with anti-GRB2 antibody (right). Proteins visualized using anti-GRB2 and anti-CD206 antibodies. Preloading control superoxide dismutase 1 (SOD1) shown on bottom

FIG. 52B shows immunoblot analysis of lysates from M2 BMDMs immunoprecipitated for active GTP-bound form of Rac1/CDC42 and visualized with anti-CDC42 or anti-Rac1 antibodies (left). Immunoblots of M2 lysates with anti-phospho-Pak1 and anti-phospho-AKT on the right. Quantification of band intensities summarizes N=3 independent experiments.

FIG. 53 shows RP-182 induces binding of IQGAP1 to CD206 and recruitment of IQGAP1 to the cell membrane of M2-polarized macrophages. A. Lysates from M2 BMDMs treated with biotinylated RP-182, biotinylated control peptide RP-426, or vehicle control for 10 minutes were precipitated with streptavidin magnetic beads, and proteins visualized with anti-IQGAP1 antibody. Preloading control superoxide dismutase 1 (SOD1) levels shown on bottom. B. Membrane recruitment of IQGAP1 by RP-182. Immunofluorescence staining of M2 BMDMs for IQGAP1, timepoints after administration of RP-182 indicated on bottom.

FIG. 54A shows immunofluorescence images after anti-IQGAP1 staining of M2 BMDMs treated with RP-182 and pre-incubated with indicated inhibitors.

FIG. 54B shows quantification of Rab7 immunofluorescence levels of M2 BMDMs treated with RP-182 and preincubated with ZCL278 (left), NSC23766 (middle), and negative control selumetinib (right). Data shown are representative of two independent experiments and normalized to corresponding vehicle treatment unless indicated otherwise.

FIG. 55A shows LC3 and cleaved caspase 8 expression in M2 macrophages pre-incubated with chloroquine (CQ) and bafilomycin (BF). N=2, in triplicates.

FIG. 55B shows quantification of phagocytosis (Rab7) and cleaved caspase 8 levels in M2 BMDMs in presence of NF-kB inhibitors JSH23 and QNZ. N=2, in triplicates. K. Schematic diagram of RP-182 functions in M2 macrophages.

FIG. 56 shows induction of cleaved caspase 8 expression in M2 macrophages treated with RP-182 is not dependent on induction of autophagy. Representative images of LC3 and cleaved caspase 8 immunofluorescence levels in M2 BMDMs treated for 2 hours with 20 μM RP-182 and pre-incubated with autophagy inhibitors chloroquine (CQ) and bafilomycin (BF).

FIG. 57 shows blockade of RP-182-induced TNF signaling abrogates autocrine activation of apoptosis in M2-polarized macrophages. A. Cartoon depicting employed interference with RP-182-induced TNF signaling.

FIG. 58 shows immunohistochemical staining of CD206 in CD206^{high} and CD206^{low} PDAC (at 10×, inlets at 40× at right).

FIG. 59 shows immunofluorescence images of M2 BMDMs stained with anti-cleaved caspase 3 and anti-cleaved caspase 8 antibodies. Lack of caspase 8 and lack of, or reduced (with anti-TNFα antibody), caspase 3 activation upon addition of neutralizing anti-TNFα antibodies, small molecule-mediated blockade of the TNF receptor (TNFR1), or for caspase 3 activation blockade of caspase 8 to M2 BMDMs treated with RP-182 (panel on left). Controls with anti-TNFα antibody, TNFR1 inhibitor R-7050, and caspase 8 inhibitor alone are shown on top. Cleaved caspase 8 and 3 staining of M2 BMDMs upon addition of conditioned media (panel on right). The addition of conditioned media from M2 BMDMs treated with RP-182 or recombinant TNFα to M2 BMDMs strongly activated caspase 8 and 3, an effect which was reduced through the addition of neutralizing TNFα to the conditioned media (bottom). TNFα, recombinant TNFα ligand; α-TNF, anti-TNFα antibody; CM, conditioned media; TNFR1i, TNF receptor inhibitor R-7050.

FIG. 60 shows CD206 expression in human pancreas cancer. A. CD206 is overexpressed in two out of three clinical pancreas cancer sample sets compared to matched uninvolved normal glandular tissue. MRC1/CD206 gene expression levels from gene sets GSE15471, GSE16515, and GSE28735. B. Tissue microarray (TMA) of 80 cases of adenocarcinoma of the pancreas, representative images at ×10 magnification of original cores shown on top, waterfall plot indicates percent CD206 positive cells of total cells ranked from highest (left) to lowest (right) for each case. Bottom shows representative images at ×40 magnification of CD206high (left) and CD206low (right) cases.

FIG. 61 shows Kaplan-Meier plots of overall survival of 125 patients afflicted with PDAC stratified by CD206 expression. Log-rank test, 2-tailed.

FIG. 62 shows Kaplan-Meier analysis of patients with CD206high TAMs stratified by CD8high vs CD8low.

FIG. 63A shows correlation of CD8A, INFG expression, 8 and 6 gene CD8 T cell activation signatures (bottom) and M2 marker expression levels in clinical specimens from TCGA cancer dataset. Samples across all cancers were divided into CD206high and CD86low using median of all specimens.

FIG. 63B shows correlation of CD8A, INFG expression, 8 and 6 gene CD8 T cell activation signatures (bottom) and M2 marker expression levels in clinical specimens from TCGA cancer dataset. Samples across all cancers were divided into CD206high and CD86low using median of all specimens. Correlation of macrophage subtypes and CD8+ T cell function in pancreas cancer TCGA dataset.

FIG. 64 shows Kaplan-Meier analysis of KPC tumors allografted in CD206−/− B6.129P2-Mrc1tm1Mnz/J mice (red curve) and in C57B/L wild type mice (black curve). Log-rank, 2-tailed.

FIG. 65 shows immunohistochemical staining of KPC wild type (KPC) and KPC tumors generated in CD206−/− B6.129P2-Mrc1tm1Mnz/J mice (KPC CD206 KO−/−), quantification on right (N≥4 per group).

FIG. 66 shows quantification of flow cytometry of TAM subpopulations in KPC tumors grown in C57B/L6 wild type and CD206−/− mice (N≥5 per group).

FIG. 67A shows Kaplan Meier analysis of KP16 mice. Log-rank test, 2-tailed. B. Waterfall plot of best objective response.

FIG. 67B shows survival and anti-tumor activity in KPC mice.

FIG. 68 shows images of immunohistochemical stains of tumors from KP16 mice treated with vehicle or RP-182, quantification depict mean percent positive cells by computer-based tissue analysis of N=4/group. Arrows indicate membranous staining of CD206-positive cells or CD8+ T cells.

FIG. 69 shows immunohistochemical staining of E-cadherin and vimentin, quantification of N≥4 per group on right.

FIG. 70 shows treatment with RP-182 reduces induction of expression of EMT markers vimentin and SNAIL in KPC cancer cells upon co-culture of M2-polarized macrophages. Flow cytometry analysis of Ecadherin, vimentin, and SNAIL2-positive cancer cell fractions after co-culture with no cells (blue bars), M2 BMDMs treated with vehicle (black), and M2 BMDMs pre-treated with 20 μM RP-182 for 2 hours. Percent positive cells were gated on live CD11b-CK19-9+ cells; results from two independent experiments conducted in triplicates are shown.

FIG. 71 shows percentage of CD206-positive M2 macrophage, MDSCs, dendritic, CD8- and CD4-positive cell fractions of total cells in KP16 tumors treated for 7 days. Fraction of FoxP3-positive Tregs of CD4+ T cells shown on the right. Representative flow cytometry plots shown on bottom.

FIG. 72 shows gating strategy for the identification of tumor infiltrating CD8+ T cell (top) and CD206high M2-like TAM fraction in pancreatic tumor digests. Representative flow cytometry plots depicting gating strategy for determination of tumor infiltrating CD8a T cells and CD206-positive TAMs from KP16 animals after treatment with vehicle and RP-182 for 1 week. For final percentages shown in FIG. 6G percentages were normalized to total live cells.

FIG. 73 shows RP-182 in combination with gemcitabine reduces CD206high monocytic MDSCs in autochthonous KPC tumors. Quantification of MDSC cell fractions by flow cytometry of tumor digests of KPC mice treated with vehicle and RP-182 in combination with gemcitabine for 7 days, % cells for each examined individual tumor are shown. Monocytic MDSCs, M-MDSC determined by CD11b+Gr-1+Ly6ChighLy6G− cells; polymorphonuclear (PMN) MDSCs determined by CD11b+Gr-1+Ly6ClowLy6G+ cells.

FIG. 74 shows INFγ-positive T cells after addition of TAMs isolated from KP16 and KPC tumors added to co-cultured cancer and splenic CD8+ T cells from tumor bearing mice.

FIG. 75 shows quantification of qRT-PCR analysis of M1 and M2 gene expression levels of TAMs isolated from KPC and KP16 tumors (N≥3 per group, in triplicate). Quantification of percentage of CD206- and CD86-positive macrophages of CD11b+F4/80+/Gr-1− TAMs, analysis of cell fractions of TAMs expressing M1 cytokines and immune checkpoints shown on the right.

FIG. 76 shows RP-182-induced M1-like CD86+ TAM populations in KPC mice have increased M1 cytokine and decreased PD-1 and SIRPα-positive cell fractions. Flow cytometry analysis of KPC treated with vehicle (top) and RP-182 (bottom) for 7 days, percent positive cells for PD-1, SIRPα, and TNFα of CD11b+F4/80+Gr-1− TAMs are shown (N≥5 per group).

FIG. 77 shows RP-182-induced M1-like CD86+ TAM populations in KPC mice have increased M1 cytokine positive cell fractions. Increased cell fractions staining positive for M1 cytokines IL-1β, IL-12, TNFα and M1 marker iNOS in RP-182-induced double positive CD86+CD206+ and CD86+CD206− TAMs but not in the CD86−CD206+ TAM population. Flow cytometry analysis of KPC treated with vehicle and RP-182 for 7 days, percent positive cell fractions of CD86+CD206−, CD86+CD206+, and CD86−CD206+ macrophage populations are shown (N≥5 per group).

FIG. 78 shows flow cytometry plots including employed gating strategy to determine cleaved caspase 3, Rab7, and LAMP1-positive CD11b+F4/80+Gr-1− TAM fractions in KPC tumors of vehicle and RP-182-treated mice. Quantification of % positive TAM fractions in N≥7 mice per group shown on bottom.

FIG. 79 shows analysis of apoptosis, phagocytosis, and phagolysosome formation by flow cytometry in CD11b-CK19+ cancer cells of tumor digests of KPC mice treated for 7 days with vehicle (top) and RP-182 (bottom). Flow cytometry plots including employed gating strategy for the determination of cleaved caspase 3, Rab7, and LAMP1-positive CD11b-CK19+ cell fractions of vehicle-treated and RP-182-treated mice. Quantification shown on bottom, bar graph indicates mean of percent fraction of N≥7 animals per group.

FIG. 80 shows T-distributed Stochastic Neighbor Embedding (t-SNE) plots after tumor single cell sequencing of CD11b+ and filtering out KRT19+, CD11c+, Ly6G+ cells (N=4/group) of vehicle- (light blue) vs RP-182-treated (dark blue) tumors, color bar indicates log 10[molecules/cell]. Increased expression levels of RP-182-induced DEGs identified in M2 BMDMs in vitro (‘in vitro gene M2 signature’) project onto CD11b+KRT19−CD11c−Ly6G− cluster from RP-182-treated tumors (right), color bar indicates log 10[sum of in vitro gene expression changes].

FIG. 81 shows images of immunofluorescence of KP16 tumors treated with RP-182 (top) and vehicle (bottom) co-stained with anti-CD206 (red) and anti-LC3 (green), computer-based quantification of co-staining cells (of total cells, N=4/group).

FIG. 82 shows tumor growth after intratumoral cell transfer of M2 BMDMs into KPC allografts (N=5).

FIG. 83 shows quantification of EliSpots (INFγ-secreting CD8+ T cells of added total CD8+ T cells) after co-culture of KP16 (left) and KPC cancer cells (right) and CD8+ T cells isolated from tumors and spleens.

FIG. 84 shows Kaplan-Meier analysis of KP16 mice treated with anti-CD8 neutralizing antibody, anti-IgG2 isotype control, and indicated combinations.

FIG. 85 shows quantification of flow cytometry analysis of CD45−CK19+ cancer cells expressing PD-L1 (left) and PD-1 expression on CD45+CD3e+CD8+ T cells (right) in KP16 tumors.

FIG. 86 shows best objective response (BOR) of KP16 tumors treated with vehicle (grey bars), anti-PD-L1 injections (purple), RP-182 (red), and anti-PD-L1 in combination with RP-182. N≥7 animals/group.

FIG. 87 shows tumor growth of CT-26 allografts, tumor weights at study endpoint shown on right. Tumor growth of murine B16 melanoma tumors.

FIG. 88A shows pancreas cancer patient-derived xenotransplantation models with CD206high (#133R, #328R) and CD206low (#295R, #057R) expression levels treated with RP-182, RP-426, and vehicle-control. N≥7/group. H. RP-182 administration rescues mice with chemically induced pulmonary fibrosis. Quantification of total body weights and Kaplan-Meier survival analysis is shown. N=6/group, error bars indicate standard deviations.

FIG. 88B shows pulmonary inflammatory infiltrates after bleomycin installation. Images of H&E stains, quantification of measured weights of excised lungs on right. N=4/group.

FIG. 88C shows images of Masson's trichrome staining, quantification of surface area (in μm2) of 100 mm2 examined lung surface on right.

FIG. 89 shows reduction of CD206-positive alveolar cell infiltrate in RP-182-treated mice. Immunofluorescence lung field images of non-instilled and bleomycin-instilled mice treated with vehicle or RP-182 stained with DAPI and anti-CD206 (green). Quantification of number of CD206-positive cells shown on right, N=4/group.

FIG. 90 shows immunofluorescence images of KPC tumors treated with biotinylated RP-182 and co-stained with anti-CD206 (green), streptavidin (red), and DAPI (blue). Laser intensity profiles (A 1) of linear scanning of random tissue section measuring intensity (fluorescence intensity, y-axis) across distance (in μm; x-axis). Co-localization of CD206 expressing cells (green) and bio-RP-182 (red channel) (B1) generating yellow/purple emission.

FIG. 91 shows tissue distribution of RP-182 after imaging of indicated organs including subcutaneous grown CT-26 tumor in BALB/c mice that were excised at 10 min after administration of Alexa-488 labelled peptide RP-182 at 20 mg/kg via intraperitoneal injection. Fluorescence in manually set regions of interest was quantified by Living Image, color scale of intensity of emitted photons shown on right.

FIG. 92 shows Kaplan-Meier analysis of KPC wild type (left) and KPC CD206 KO−/− allografts (right) treated with vehicle (black curve) or RP-182 (red). Phase-contrast images of M2 BMDMs incubated with carboxyfluorescein succinimidyl ester (CFSE)-labelled cancer cells (green). Inlet (at 100×) shows representative macrophage with engulfed green-labelled cancer cell and induced cytoplasmatic vacuoles and vesicles

FIG. 93 shows toxicity measures after daily 14-day administration of increasing concentrations of RP-182. Body weights after 14-day daily administration of RP-182 (left) and red and white blood cell counts (RBC, WBC) of vehicle and RP-182-treated mice at 30 mg/kg (right). C. Individual organ weights (in grams) of mice treated for 14 days with indicated daily doses of RP-182, n≥7/group.

FIG. 94A shows Phase-contrast images of M2 BMDMs incubated with carboxyfluorescein succinimidyl ester (CFSE)-labelled cancer cells (green). Inlet (at 100×) shows representative macrophage with engulfed green-labelled cancer cell and induced cytoplasmatic vacuoles and vesicles.

FIG. 94B shows Quantification of number of CFSE-positive macrophages of total number of M2 BMDMs (%, phagocytic index).

FIG. 95 shows Flow cytometry plots of CFSE-positive CD11b+F4/80+Gr-1−CD86+ (top) and CD206+ BMDMs after addition of CFSE-labelled KPC cancer cells and treatment with vehicle or RP-182.

FIG. 96 shows tumor growth of KPC xenografts (N≥7/group).

FIG. 97 shows RP-182 restricts tumor growth of MDA-MB231 breast cancer and C4-2 prostate cancer xenografts grown in T cell deficient nu/J mice. A. MDA-MB231 tumors (N≥7/group) grown in nu/J mice treated with vehicle (red), RP-182 (purple), gemcitabine (green), and the combination (brown) shown on left, impact of RP-182 treatment onto lymph node metastasis in MDA-MB231 mice after 42 days of treatment shown right. Analysis shows fraction of lymph nodes involved by cancer per examined total number of lymph nodes in draining basin. Representative H&E staining of metastatic cancer in involved lymph node shown on right, green arrows highlight cancer cells with abnormal mitotic figures. B. Tumor growth of C4-2 prostate cancer xenografts grown in T cell deficient nu/J mice.

FIG. 98 shows transmission electron microscopy of KP16 tumors treated with vehicle and RP-182. TAMs with intracellular vesicles indicated by arrow, clasping of cancer cell and partial or complete cancer cell phagocytosis events highlighted in red.

### DETAILED DESCRIPTION OF THE INVENTION

Aspects of the present disclosure include methods for modulating macrophage activity. Methods according to certain embodiments include contacting a macrophage with a mannose receptor (CD206) binding agent in a manner sufficient to modulate activity of the macrophage. Methods for converting a phenotype of a macrophage from an M2 phenotype to an M1 phenotype are also provided. Methods for inhibiting growth of a CD206-expressing cell as well as methods for treating a subject for a neoplastic condition (e.g., cancer) or a condition associated with chronic inflammation are described. Immuno-modulating peptides suitable for use in the subject methods are also presented. Aspects of the present disclosure also include active agents for binding to an activity modulating domain of CD206. Methods for determining if a compound binds to an activity modulating domain of CD206 are also provided.

Before the present invention is described in greater detail, it is to be understood that this invention is not limited to particular embodiments described, which as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods for Modulating Macrophage Activity

Methods for modulating macrophage activity and for converting a phenotype of a macrophage from an M2 phenotype to an M1 phenotype, methods of inhibiting growth of a CD206-expressing cell, methods for treating a neoplastic condition or a condition associated with chronic inflammation as well as combination therapy methods are described in greater detail, along with examples, as set forth below.

In some embodiments, methods include modulating macrophage activity: the method including contacting a macrophage with a CD206-binding agent (e.g., as described herein) to modulate activity of the macrophage.

In certain cases, the CD206-binding agent binds to a site selected from fibronectin II domain of CD206, C-type lectin carbohydrate recognition domain 3 (CRD3) of CD206, C-type lectin carbohydrate recognition domain 4 (CRD4) of CD206 and C-type lectin carbohydrate recognition domain 5 (CRD5) of CD206. In certain cases, the site is fibronectin II domain of CD206. In certain cases, the site is C-type lectin carbohydrate recognition domain 3 (CRD3) of CD206. In certain other cases, the site is C-type lectin carbohydrate recognition domain 5 (CRD5) of CD206.

In certain embodiments, the CD206-binding agent binds to CD206 with a binding energy of at least −650 kcal/mol, such as at least −700 kcal/mol, and in certain embodiments at least −750, −800, −900, −1000, −1100, −1200, −1250, −1300, −1350, −1400, −1425, −1450, −1475, −1500, −1525, −1550, −1575, −1600 kcal/mol, or greater. The energy of binding can be determined, e.g., in silico, in vitro, or in vivo, using methods well-known in the art (e.g., using the ClusPro™ algorithm).

In certain embodiments, of the methods of modulating macrophage activity, the macrophage activity that is modulated is macrophage polarization. In certain embodiments of the method, the viability of the macrophage is reduced. In certain embodiments, of the methods of modulating macrophage activity, the macrophage is an M2 macrophage or a tumor associated macrophage (TAM). In certain embodiments of the methods of modulating macrophage activity, the CD206-binding agent (e.g., as described herein) inhibits macrophage activity. In certain embodiments of the method, the CD206-binding agent induces apoptosis of the macrophage. In certain embodiments of the method, the CD206-binding agent stimulates phagocytosis.

In certain embodiments of the methods of modulating macrophage activity, the macrophage is in vitro. In certain other embodiments, the macrophage is in vivo. In still other embodiments, methods include converting a phenotype of a macrophage from an M2 phenotype to an M1 phenotype. Methods according to certain embodiments include contacting a macrophage having an M2 phenotype with a CD206-binding agent in a manner sufficient to convert the phenotype of the macrophage to an M1 phenotype. In some instances, contacting the CD206-binding agent induces a conformational change in a CD206 receptor of the macrophage sufficient to convert the phenotype of the macrophage to an M1 phenotype. In some instances, converting the phenotype of the macrophage includes inducing expression of CD86 by the macrophage. In other instances, converting the phenotype of the macrophage includes reducing expression of CD206 by the macrophage. In still other instances, converting the phenotype of the macrophage includes converting the macrophage to a phenotype that exhibits upregulation of M1 cytokines and markers. For example, the M1 cytokine and marker may be selected from the group consisting of IL-1β, IL-12, TNFα and nitric oxide synthetase. In other instances, converting the phenotype of the macrophage includes converting the macrophage to a phenotype that exhibits decreased expression of signal regulatory protein α (SIRPα).

In certain embodiments of the methods of converting a phenotype of a macrophage from an M2 phenotype to an M1 phenotype, the CD206-binding agent binds to a site selected from fibronectin II domain of CD206, C-type lectin carbohydrate recognition domain 3 (CRD3) of CD206, C-type lectin carbohydrate recognition domain 4 (CRD4) of CD206 and C-type lectin carbohydrate recognition domain 5 (CRD5) of CD206. In certain cases, the site is fibronectin II domain of CD206. In certain cases, the site is C-type lectin carbohydrate recognition domain 3 (CRD3) of CD206. In certain other cases, the site is C-type lectin carbohydrate recognition domain 5 (CRD5) of CD206.

In certain embodiments of the methods of converting a phenotype of a macrophage from an M2 phenotype to an M1 phenotype, the macrophage is contacted with the CD206-binding agent in vitro. In certain other embodiments, the macrophage is contacted with the CD206-binding agent in vivo.

In some embodiments, methods include inhibiting growth of a CD206-expressing cell: the method including contacting a target CD206-expressing cell with a CD-206-binding agent (e.g., as described herein) to inhibit growth of the cell. In certain instances, the cell is a cancer cell. Cancer cells include, without limitation, a pancreatic cancer cell, a prostate cancer cell, a colon cancer cell, a skin cancer cell or a breast cancer cell. In certain cases, the cancer is a solid tumor cancer. Solid tumor cancers include, without limitation, pancreatic, prostate, colon, breast, or skin tumors. In certain embodiments, of the methods of inhibiting growth of a CD206-expressing cell, the contacting of the target CD206-expressing cell comprises administering to a subject in need thereof a therapeutically effective amount of the CD206-binding agent (e.g., as described herein), to treat the subject for a cancer. In some embodiments, methods include treating a subject for a condition associated with chronic inflammation. Methods according to certain embodiments include administering a therapeutically effective amount of a CD206-binding agent to a subject to treat the subject for the condition associated with chronic inflammation. In some embodiments, the condition associated with chronic inflammation is selected from scleroderma or multiple sclerosis, irritable bowel disease, ulcerative colitis, colitis, Crohn's disease, idiopathic pulmonary fibrosis, asthma, keratitis, arthritis, osteoarthritis, rheumatoid arthritis, auto-immune diseases, a feline or human immunodeficiency virus (FIV or HIV) infection, cancer, age-related inflammation and/or stem cell dysfunction, graft-versus-host disease (GVHD), keloids, obesity, diabetes, diabetic wounds, other chronic wounds, atherosclerosis, Parkinson's disease, Alzheimer's disease, macular degeneration, gout, gastric ulcers, gastritis, mucositis, toxoplasmosis, an ophthalmic inflammatory condition (e.g., keratitis), a skin disease (e.g., atopic dermatitis, or psoriasis), an inflammatory condition such as sinusitis or otitis media, a parasitic infection (e.g., malaria), and chronic viral or microbial infections.

In certain embodiments of methods for treating chronic inflammation, the CD206-binding agent (e.g., as described herein) is administered in conjunction with another drug known to be effective in treating the condition. In some instances, the condition is cancer. In some instances, the cancer includes, without limitation, pancreatic, prostate, colon, breast or skin cancer. In certain cases, the methods further include administering an effective amount of a chemotherapeutic agent, antibody agent, or cell therapy to the subject. In certain cases, the chemotherapeutic agent, antibody agent or cell therapy is selected from steroids, anthracyclines, thyroid hormone replacement drugs, thymidylate-targeted drugs, antibodies, checkpoint inhibitor drugs, Chimeric Antigen Receptor/T cell therapies, and other cell therapies.

In some embodiments of the methods of treating chronic inflammation, the condition associated with chronic inflammation is a fibrosis. In some instances, the condition associated with chronic inflammation is scleroderma.

In some embodiments, methods include treating a subject for a neoplastic condition, such as cancer (e.g., a solid tumor cancer). The methods of the present disclosure may be employed to target and treat a variety of cancers, including e.g., primary cancer, secondary cancers, re-growing cancers, recurrent cancers, refractory cancers and the like. For example, in some instances, the methods of the present disclosure may be employed as an initial treatment of a primary cancer identified in a subject. In some instances, the methods of the present disclosure may be employed as a non-primary (e.g., secondary or later) treatment, e.g., in a subject with a cancer that is refractory to a prior treatment, in a subject with a cancer that is re-growing following a prior treatment, in a subject with a mixed response to a prior treatment (e.g., a positive response to at least one tumor in the subject and a negative or neutral response to at least a second tumor in the subject), and the like.

In some instances, the method of the present disclosure may be employed to target, treat or clear a subject for minimal residual disease (MRD) remaining after a prior cancer therapy. Targeting, treating and/or clearance of MRD may be pursued using the instant methods whether the MRD is or has been determined to be refractory to the prior treatment or not. In some instances, a method of the present disclosure may be employed to target, treat and/or clear a subject of MRD following a determination that the MRD is refractory to a prior treatment or one or more available treatment options.

Cancers of interest associated with commonly mutated genes include e.g., ABI1, ABL1, ABL2, ACKR3, ACSL3, ACSL6, AFF1, AFF3, AFF4, AKAP9, AKT1, AKT2, ALDH2, ALK, AMER1, APC, ARHGAP26, ARHGEF12, ARID1A, ARID2, ARNT, ASPSCR1, ASXL1, ATF1, ATIC, ATM, ATP1A1, ATP2B3, ATRX, AXIN1, BAP1, BCL10, BCL11A, BCL11B, BCL2, BCL3, BCL6, BCL7A, BCL9, BCOR, BCR, BIRC3, BLM, BMPR1A, BRAF, BRCA1, BRCA2, BRD3, BRD4, BRIP1, BTG1, BUB1B, C15orf65, C2orf44, CACNA1D, CALR, CAMTA1, CANT1, CARD11, CARS, CASC5, CASP8, CBFA2T3, CBFB, CBL, CBLB, CBLC, CCDC6, CCNB1IP1, CCND1, CCND2, CCND3, CCNE1, CD274, CD74, CD79A, CD79B, CDC73, CDH1, CDH11, CDK12, CDK4, CDK6, CDKN2A, CDKN2C, CDX2, CEBPA, CEP89, CHCHD7, CHEK2, CHIC2, CHN1, CIC, CIITA, CLIP1, CLP1, CLTC, CLTCL1, CNBP, CNOT3, CNTRL, COL1A1, COL2A1, COX6C, CREB1, CREB3L1, CREB3L2, CREBBP, CRLF2, CRTC1, CRTC3, CSF3R, CTNNB1, CUX1, CYLD, DAXX, DCTN1, DDB2, DDIT3, DDX10, DDX5, DDX6, DEK, DICER1, DNM2, DNMT3A, EBF1, ECT2L, EGFR, EIF3E, EIF4A2, ELF4, ELK4, ELL, ELN, EML4, EP300, EPS15, ERBB2, ERC1, ERCC2, ERCC3, ERCC4, ERCC5, ERG, ETV1, ETV4, ETV5, ETV6, EWSR1, EXT1, EXT2, EZH2, EZR, FAM46C, FANCA, FANCC, FANCD2, FANCE, FANCF, FANCG, FAS, FBXO11, FBXW7, FCGR2B, FCRL4, FEV, FGFR1, FGFR1 OP, FGFR2, FGFR3, FH, FHIT, FIP1L1, FLCN, FLI1, FLT3, FNBP1, FOXA1, FOXL2, FOXO1, FOXO3, FOXO4, FOXP1, FSTL3, FUBP1, FUS, GAS7, GATA1, GATA2, GATA3, GMPS, GNA11, GNAQ, GNAS, GOLGA5, GOPC, GPC3, GPHN, H3F3A, H3F3B, HERPUD1, HEY1, HIP1, HISTIH4I, HLA-A, HLF, HMGA1, HMGA2, HNF1A, HNRNPA2B1, HOOK3, HOXA11, HOXA13, HOXA9, HOXC11, HOXCl3, HOXD11, HOXD13, HRAS, HSP90AA1, HSP90AB1, IDH1, IDH2, IKZF1, IL2, IL21R, IL6ST, IL7R, IRF4, ITK, JAK1, JAK2, JAK3, JAZF1, JUN, KAT6A, KAT6B, KCNJ5, KDM5A, KDM5C, KDM6A, KDR, KDSR, KIAA1549, KIAA1598, KIF5B, KIT, KLF4, KLF6, KLK2, KMT2A, KMT2C, KMT2D, KRAS, KTN1, LASP1, LCK, LCP1, LHFP, LIFR, LMNA, LMO1, LMO2, LPP, LRIG3, LSM14A, LYL1, MAF, MAFB, MALT1, MAML2, MAP2K1, MAP2K2, MAP2K4, MAX, MDM2, MDM4, MECOM, MED12, MEN1, MET, MITF, MKL1, MLF1, MLH1, MLLT1, MLLT10, MLLT11, MLLT3, MLLT4, MLLT6, MN1, MNX1, MPL, MSH2, MSH6, MSI2, MSN, MTCP1, MUC1, MUTYH, MYB, MYC, MYCL, MYCN, MYD88, MYH11, MYH9, MYO5A, NAB2, NACA, NBN, NCKIPSD, NCOA1, NCOA2, NCOA4, NDRG1, NF1, NF2, NFATC2, NFE2L2, NFIB, NFKB2, NIN, NKX2-1, NONO, NOTCHI, NOTCH2, NPM1, NR4A3, NRAS, NRG1, NSD1, NT5C2, NTRK1, NTRK3, NUMA1, NUP214, NUP98, NUTM1, NUTM2A, NUTM2B, OLIG2, OMD, P2RY8, PAFAH1B2, PALB2, PATZ1, PAX3, PAX5, PAX7, PAX8, PBRM1, PBX1, PCM1, PCSK7, PDCD1LG2, PDE4DIP, PDGFB, PDGFRA, PDGFRB, PER1, PHF6, PHOX2B, PICALM, PIK3CA, PIK3R1, PIM1, PLAG1, PLCG1, PML, PMS1, PMS2, POT1, POU2AF1, POU5F1, PPARG, PPFIBP1, PPP2R1A, PRCC, PRDM1, PRDM16, PRF1, PRKAR1A, PRRX1, PSIP1, PTCH1, PTEN, PTPN11, PTPRB, PTPRC, PTPRK, PWWP2A, RABEPI, RAC1, RAD21, RAD51B, RAF1, RALGDS, RANBP17, RAP1GDS1, RARA, RB1, RBM15, RECQL4, REL, RET, RHOH, RMI2, RNF213, RNF43, ROS1, RPL10, RPL22, RPL5, RPN1, RSPO2, RSPO3, RUNX1, RUNX1T1, SBDS, SDC4, SDHAF2, SDHB, SDHC, SDHD, SEPT5, SEPT6, SEPT9, SET, SETBP1, SETD2, SF3B1, SFPQ, SH2B3, SH3GL1, SLC34A2, SLC45A3, SMAD4, SMARCA4, SMARCB1, SMARCE1, SMO, SOCS1, SOX2, SPECCI, SRGAP3, SRSF2, SRSF3, SS18, SS18L1, SSX1, SSX2, SSX2B, SSX4, SSX4B, STAG2, STAT3, STAT5B, STAT6, STIL, STK11, SUFU, SUZ12, SYK, TAF15, TAL1, TAL2, TBLIXR1, TCEA1, TCF12, TCF3, TCF7L2, TCL1A, TERT, TET1, TET2, TFE3, TFEB, TFG, TFPT, TFRC, THRAP3, TLX1, TLX3, TMPRSS2, TNFAIP3, TNFRSF14, TNFRSF17, TOP1, TP53, TPM3, TPM4, TPR, TRAF7, TRIM24, TRIM27, TRIM33, TRIP11, TRRAP, TSC1, TSC2, TSHR, TTL, U2AF1, UBR5, USP6, VHL, VTI1A, WAS, WHSC1, WHSC1L1, WIF1, WRN, WT1, WWTR1, XPA, XPC, XPO1, YWHAE, ZBTB16, ZCCHC8, ZMYM2, ZNF331, ZNF384, ZNF521 and ZRSR2.

Cancers of interest for treatment according to embodiments of the present disclosure include but are not limited to, e.g., Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, etc.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenström, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sézary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like.

The methods of treating described herein may, in some instances, be performed in a subject that has previously undergone one or more conventional treatments. For example, in the case of oncology, the methods described herein may, in some instances, be performed following a conventional cancer therapy including but not limited to e.g., conventional chemotherapy, conventional radiation therapy, conventional immunotherapy, surgery, etc. In some instances, the methods described herein may be used when a subject has not responded to or is refractory to a conventional therapy.

With respect to the cancer as a whole, desired effects of the described treatments may result in a reduction in the number of cells in the cancer, a reduction in the size of a tumor, a reduction in the overall proliferation of the cancer, a reduction in the overall growth rate of a tumor, etc. For example, an effective treatment is in some cases a treatment that, when administered in one or more doses to an individual in need thereof, reduces the number of cancer cells in the individual and/or reduces tumor mass in the individual, by at least about 10%, at least about 15%, at least about 20%, at least about 25%, at least about 30%, at least about 40%, at least about 50%, at least about 75%, or more than 75%, compared to the number of cancer cells and/or tumor mass in the absence of the treatment.

In some embodiments, an effective treatment is a treatment that, when administered alone (e.g., in monotherapy) or in combination (e.g., in combination therapy) with one or more additional therapeutic agents, in one or more doses, is effective to reduce one or more of tumor growth rate, cancer cell number, and tumor mass, by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the tumor growth rate, cancer cell number, or tumor mass in the absence of the treatment.

In some instances, treatment may involve modulation, including induction, of the expression and/or secretion of a cytokine by an immune cell. Non-limiting examples of cytokines, the expression/secretion of which may be modulated, include but are not limited to e.g., Interleukins and related (e.g., IL-1-like, IL-1α, IL-1β, IL-1RA, IL-18, IL-2, IL-4, IL-7, IL-9, IL-13, IL-15, IL-3, IL-5, GM-CSF, IL-6-like, IL-6, IL-11, G-CSF, IL-12, LIF, OSM, IL-10-like, IL-10, IL-20, IL-14, IL-16, IL-17, etc.), Interferons (e.g., IFN-α, IFN-β, IFN-γ, etc.), TNF family (e.g., CD154, LT-β, TNF-α, TNF-β, 4-1BBL, APRIL, CD70, CD153, CD178, GITRL, LIGHT, OX40L, TALL-1, TRAIL, TWEAK, TRANCE, etc.), TGF-β family (e.g., TGF-β1, TGF-β2, TGF-β3, etc.) and the like. The amount of the increase may vary and may range from a 10% or greater increase, including but not limited to e.g., 10% or greater, 25% or greater, 50% or greater, 75% or greater, 100% or greater, 150% or greater, 200% or greater, 250% or greater, 300% or greater, 350% or greater 400% or greater, etc.

### Chemotherapeutic Agents and Combination Therapy

As will be readily understood, the methods of treating by administering a therapeutically effective amount of a CD206-binding agent to a subject described herein may, in some instances, be combined with one or more conventional treatments. For example, in the case of oncology, the methods described herein may, in some instances, be combined with a conventional cancer therapy including but not limited to e.g., conventional chemotherapy, conventional radiation therapy, conventional immunotherapy, surgery, etc.

In conjunction with any of the subject methods, the CD206-binding agent (e.g., as described herein) (or pharmaceutical compositions comprising such compounds) can be administered in combination with another drug designed to reduce or prevent inflammation, treat or prevent chronic inflammation, or treat cancer. In some embodiments, the condition associated with chronic inflammation is a fibrosis. In some instances, the condition associated with chronic inflammation is scleroderma. For example, the CD206-binding agent may be combined with a conventional agent or therapy for treating chronic inflammation or fibrosis, including but limited to e.g., pirfenidone, nintedanib, a nonsteroidal anti-inflammatory drug (NSADs), a steroidal agent, standard scleroderma treatments. In each case, the CD206-binding agent can be administered prior to, at the same time as, or after the administration of the other drug.

In some instances, the methods described herein may be used before or after a conventional therapy. For example, the methods described herein may be used as an adjuvant therapy, e.g., after a subject has seen improvement from a conventional therapy, or may be used when a subject has not responded to a conventional therapy. In some instances, the methods described herein may be used prior to an additional therapy, e.g., to prepare a subject for an additional therapy, e.g., a conventional therapy as described herein.

Standard cancer therapies include surgery (e.g., surgical removal of cancerous tissue), radiation therapy, bone marrow transplantation, chemotherapeutic treatment, antibody treatment, biological response modifier treatment, and certain combinations of the foregoing.

Radiation therapy includes, but is not limited to, x-rays or gamma rays that are delivered from either an externally applied source such as a beam, or by implantation of small radioactive sources.

Suitable antibodies for use in cancer treatment include, but are not limited to, naked antibodies, e.g., trastuzumab (Herceptin), bevacizumab (Avastin™), cetuximab (Erbitux™), panitumumab (Vectibix™), Ipilimumab (Yervoy™), rituximab (Rituxan), alemtuzumab (Lemtrada™), Ofatumumab (Arzerra™), Oregovomab (OvaRex™), Lambrolizumab (MK-3475), pertuzumab (Perjeta™), ranibizumab (Lucentis™) etc., and conjugated antibodies, e.g., gemtuzumab ozogamicin (Mylortarg™), Brentuximab vedotin (Adcetris™), 90Y-labelled ibritumomab tiuxetan (Zevalin™), 131I-labelled tositumoma (Bexxar™), etc. Suitable antibodies for use in cancer treatment include, but are not limited to, antibodies raised against tumor-associated antigens. Such antigens include, but are not limited to, CD20, CD30, CD33, CD52, EpCAM, CEA, gpA33, Mucins, TAG-72, CAIX, PSMA, Folate-binding protein, Gangliosides (e.g., GD2, GD3, GM2, etc.), Le y, VEGF, VEGFR, Integrin alpha-V-beta-3, Integrin alpha-5-beta-1, EGFR, ERBB2, ERBB3, MET, IGF1R, EPHA3, TRAILR1, TRAILR2, RANKL, FAP, Tenascin, etc.

Conventional cancer therapies also include targeted therapies for cancer including but not limited to e.g., Ado-trastuzumab emtansine (Kadcyla) targeting HER2 (ERBB2/neu) (approved for use in Breast cancer); Afatinib (Gilotrif) targeting EGFR (HER1/ERBB1), HER2 (ERBB2/neu) (approved for use in Non-small cell lung cancer); Aldesleukin (Proleukin) targeting (approved for use in Renal cell carcinoma, Melanoma); Alectinib (Alecensa) targeting ALK (approved for use in Non-small cell lung cancer); Alemtuzumab (Campath) targeting CD52 (approved for use in B-cell chronic lymphocytic leukemia); Atezolizumab (Tecentriq) targeting PD-L1 (approved for use in Urothelial carcinoma, Non-small cell lung cancer); Avelumab (Bavencio) targeting PD-L1 (approved for use in Merkel cell carcinoma); Axitinib (Inlyta) targeting KIT, PDGFRβ, VEGFR1/2/3 (approved for use in Renal cell carcinoma); Belimumab (Benlysta) targeting BAFF (approved for use in Lupus erythematosus); Belinostat (Beleodaq) targeting HDAC (approved for use in Peripheral T-cell lymphoma); Bevacizumab (Avastin) targeting VEGF ligand (approved for use in Cervical cancer, Colorectal cancer, Fallopian tube cancer, Glioblastoma, Non-small cell lung cancer, Ovarian cancer, Peritoneal cancer, Renal cell carcinoma); Blinatumomab (Blincyto) targeting CD19/CD3 (approved for use in Acute lymphoblastic leukemia (precursor B-cell)); Bortezomib (Velcade) targeting Proteasome (approved for use in Multiple myeloma, Mantle cell lymphoma); Bosutinib (Bosulif) targeting ABL (approved for use in Chronic myelogenous leukemia); Brentuximab vedotin (Adcetris) targeting CD30 (approved for use in Hodgkin lymphoma, Anaplastic large cell lymphoma); Brigatinib (Alunbrig) targeting ALK (approved for use in Non-small cell lung cancer (ALK+)); Cabozantinib (Cabometyx, Cometriq) targeting FLT3, KIT, MET, RET, VEGFR2 (approved for use in Medullary thyroid cancer, Renal cell carcinoma); Carfilzomib (Kyprolis) targeting Proteasome (approved for use in Multiple myeloma); Ceritinib (Zykadia) targeting ALK (approved for use in Non-small cell lung cancer); Cetuximab (Erbitux) targeting EGFR (HER1/ERBB1) (approved for use in Colorectal cancer, Squamous cell cancer of the head and neck); Cobimetinib (Cotellic) targeting MEK (approved for use in Melanoma); Crizotinib (Xalkori) targeting ALK, MET, ROS1 (approved for use in Non-small cell lung cancer); Dabrafenib (Tafinlar) targeting BRAF (approved for use in Melanoma, Non-small cell lung cancer); Daratumumab (Darzalex) targeting CD38 (approved for use in Multiple myeloma); Dasatinib (Sprycel) targeting ABL (approved for use in Chronic myelogenous leukemia, Acute lymphoblastic leukemia); Denosumab (Xgeva) targeting RANKL (approved for use in Giant cell tumor of the bone); Dinutuximab (Unituxin) targeting B4GALNT1 (GD2) (approved for use in Pediatric neuroblastoma); Durvalumab (Imfinzi) targeting PD-L1 (approved for use in Urothelial carcinoma); Elotuzumab (Empliciti) targeting SLAMF7 (CS1/CD319/CRACC) (approved for use in Multiple myeloma); Enasidenib (Idhifa) targeting IDH2 (approved for use in Acute myeloid leukemia); Erlotinib (Tarceva) targeting EGFR (HER1/ERBB1) (approved for use in Non-small cell lung cancer, Pancreatic cancer); Everolimus (Afinitor) targeting mTOR (approved for use in Pancreatic, gastrointestinal, or lung origin neuroendocrine tumor, Renal cell carcinoma, Nonresectable subependymal giant cell astrocytoma, Breast cancer); Gefitinib (Iressa) targeting EGFR (HER1/ERBB1) (approved for use in Non-small cell lung cancer); Ibritumomab tiuxetan (Zevalin) targeting CD20 (approved for use in Non-Hodgkin's lymphoma); Ibrutinib (Imbruvica) targeting BTK (approved for use in Mantle cell lymphoma, Chronic lymphocytic leukemia, Waldenstrom's macroglobulinemia); Idelalisib (Zydelig) targeting PI3Kδ (approved for use in Chronic lymphocytic leukemia, Follicular B-cell non-Hodgkin lymphoma, Small lymphocytic lymphoma); Imatinib (Gleevec) targeting KIT, PDGFR, ABL (approved for use in GI stromal tumor (KIT+), Dermatofibrosarcoma protuberans, Multiple hematologic malignancies); Ipilimumab (Yervoy) targeting CTLA-4 (approved for use in Melanoma); Ixazomib (Ninlaro) targeting Proteasome (approved for use in Multiple Myeloma); Lapatinib (Tykerb) targeting HER2 (ERBB2/neu), EGFR (HER1/ERBB1) (approved for use in Breast cancer (HER2+)); Lenvatinib (Lenvima) targeting VEGFR2 (approved for use in Renal cell carcinoma, Thyroid cancer); Midostaurin (Rydapt) targeting FLT3 (approved for use in acute myeloid leukemia (FLT3+)); Necitumumab (Portrazza) targeting EGFR (HER1/ERBB1) (approved for use in Squamous non-small cell lung cancer); Neratinib (Nerlynx) targeting HER2 (ERBB2/neu) (approved for use in Breast cancer); Nilotinib (Tasigna) targeting ABL (approved for use in Chronic myelogenous leukemia); Niraparib (Zejula) targeting PARP (approved for use in Ovarian cancer, Fallopian tube cancer, Peritoneal cancer); Nivolumab (Opdivo) targeting PD-1 (approved for use in Colorectal cancer, Head and neck squamous cell carcinoma, Hodgkin lymphoma, Melanoma, Non-small cell lung cancer, Renal cell carcinoma, Urothelial carcinoma); Obinutuzumab (Gazyva) targeting CD20 (approved for use in Chronic lymphocytic leukemia, Follicular lymphoma); Ofatumumab (Arzerra, HuMax-CD20) targeting CD20 (approved for use in Chronic lymphocytic leukemia); Olaparib (Lynparza) targeting PARP (approved for use in Ovarian cancer); Olaratumab (Lartruvo) targeting PDGFRα (approved for use in Soft tissue sarcoma); Osimertinib (Tagrisso) targeting EGFR (approved for use in Non-small cell lung cancer); Palbociclib (Ibrance) targeting CDK4, CDK6 (approved for use in Breast cancer); Panitumumab (Vectibix) targeting EGFR (HER1/ERBB1) (approved for use in Colorectal cancer); Panobinostat (Farydak) targeting HDAC (approved for use in Multiple myeloma); Pazopanib (Votrient) targeting VEGFR, PDGFR, KIT (approved for use in Renal cell carcinoma); Pembrolizumab (Keytruda) targeting PD-1 (approved for use in Classical Hodgkin lymphoma, Melanoma, Non-small cell lung cancer (PD-L1+), Head and neck squamous cell carcinoma, Solid tumors (MSI-H)); Pertuzumab (Perjeta) targeting HER2 (ERBB2/neu) (approved for use in Breast cancer (HER2+)); Ponatinib (Iclusig) targeting ABL, FGFR1-3, FLT3, VEGFR2 (approved for use in Chronic myelogenous leukemia, Acute lymphoblastic leukemia); Ramucirumab (Cyramza) targeting VEGFR2 (approved for use in Colorectal cancer, Gastric cancer or Gastroesophageal junction (GEJ) adenocarcinoma, Non-small cell lung cancer); Regorafenib (Stivarga) targeting KIT, PDGFRβ, RAF, RET, VEGFR1I/2/3 (approved for use in Colorectal cancer, Gastrointestinal stromal tumors, Hepatocellular carcinoma); Ribociclib (Kisqali) targeting CDK4, CDK6 (approved for use in Breast cancer (HR+, HER2−)); Rituximab (Rituxan, Mabthera) targeting CD20 (approved for use in Non-Hodgkin's lymphoma, Chronic lymphocytic leukemia, Rheumatoid arthritis, Granulomatosis with polyangiitis); Rituximab/hyaluronidase human (Rituxan Hycela) targeting CD20 (approved for use in Chronic lymphocytic leukemia, Diffuse large B-cell lymphoma, Follicular lymphoma); Romidepsin (Istodax) targeting HDAC (approved for use in Cutaneous T-cell lymphoma, Peripheral T-cell lymphoma); Rucaparib (Rubraca) targeting PARP (approved for use in Ovarian cancer); Ruxolitinib (Jakafi) targeting JAK1/2 (approved for use in Myelofibrosis); Siltuximab (Sylvant) targeting IL-6 (approved for use in Multicentric Castleman's disease); Sipuleucel-T (Provenge) targeting (approved for use in Prostate cancer); Sonidegib (Odomzo) targeting Smoothened (approved for use in Basal cell carcinoma); Sorafenib (Nexavar) targeting VEGFR, PDGFR, KIT, RAF (approved for use in Hepatocellular carcinoma, Renal cell carcinoma, Thyroid carcinoma); Temsirolimus (Torisel) targeting mTOR (approved for use in Renal cell carcinoma); Tositumomab (Bexxar) targeting CD20 (approved for use in Non-Hodgkin's lymphoma); Trametinib (Mekinist) targeting MEK (approved for use in Melanoma, Non-small cell lung cancer); Trastuzumab (Herceptin) targeting HER2 (ERBB2/neu) (approved for use in Breast cancer (HER2+), Gastric cancer (HER2+)); Vandetanib (Caprelsa) targeting EGFR (HER1/ERBB1), RET, VEGFR2 (approved for use in Medullary thyroid cancer); Vemurafenib (Zelboraf) targeting BRAF (approved for use in Melanoma); Venetoclax (Venclexta) targeting BCL2 (approved for use in Chronic lymphocytic leukemia); Vismodegib (Erivedge) targeting PTCH, Smoothened (approved for use in Basal cell carcinoma); Vorinostat (Zolinza) targeting HDAC (approved for use in Cutaneous T-cell lymphoma); Ziv-aflibercept (Zaltrap) targeting PIGF, VEGFA/B (approved for use in Colorectal cancer); and the like.

Biological response modifiers suitable for use in connection with the methods of the present disclosure include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) interferon-α.; (7) interferon-γ; (8) colony-stimulating factors; (9) inhibitors of angiogenesis; and (10) antagonists of tumor necrosis factor.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used.

Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, deoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3N-desbenzoyl-3N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

In some instances, methods of treating a subject for cancer may further include administering an agent which enhances the activity of the treatment. Such agents that enhance the activity of the treatment will vary widely and may include but are not limited to e.g., agents that inhibit an inhibitor molecule. Suitable inhibitory molecules that may be targeted include but are not limited to e.g., PD1, PD-L1, CTLA4, TIM3, LAG3, VISTA, BTLA, TIGIT, LAIR1, CD160, 2B4 and TGFR beta.

Inhibiting of inhibitory molecules may be achieved by any convenient method including but not limited to e.g., the administration of a direct inhibitor of the inhibitory molecule (e.g., an antibody that binds the inhibitory molecule, a small molecule antagonist of the inhibitory molecule, etc.), administration of an agent that inhibits expression of the inhibitory molecule (e.g., an inhibitory nucleic acid, e.g., a dsRNA, e.g., an siRNA or shRNA targeting a nucleic acid encoding the inhibitory molecule), an indirect inhibitor of the inhibitory signaling, and the like. In some instances, an agent that may be administered may be an antibody or antibody fragment that binds to an inhibitory molecule. For example, the agent can be an antibody or antibody fragment that binds to PD1, PD-L1, PD-L2 or CTLA4 (e.g., ipilimumab (also referred to as MDX-010 and MDX-101, and marketed as Yervoy (Bristol-Myers Squibb)), Tremelimumab (Pfizer, formerly known as ticilimumab, CP-675,206)), TIM3, LAG3, or the like.

In some embodiments, methods include administering to the subject an immune checkpoint inhibitors like anti-CTLA4 or anti-PD-1 and anti-PD-1L agents. The immune system depends on multiple checkpoints to avoid over-activation of the immune system on healthy cells, and tumor cells often take advantage of these checkpoints in order to escape detection by the immune system. CTLA-4, shown to be aberrantly upregulated and present on the surface of T cells in certain cancers, and PD-1, also upregulated in certain tumors and found to inhibit T-cell function, are checkpoints that have been studied as targets for cancer therapy (Pardoll, D. M. 2012 Nat Rev Cancer 12(4):252-264; Sharma, et al. 2011 Nat Rev Cancer 11(11):805-812).

In some instances, the methods of the instant disclosure may be used without any additional conventional therapy including e.g., where the method described herein is the sole method used to treat the subject. For example, in the case of oncology, the methods described herein may, in some instances, be the sole method used to treat the subject for a cancer.

Determining when combination therapies, e.g., involving the administration of one or more agents that ameliorates one or more side effects of a therapy described herein or involving the administration of one or more agents that enhances a therapy described herein, are indicated and the specifics of the administration of such combination therapies are within the skill of the relevant medical practitioner. In some instances, dosage regimens and treatment schedules of combination therapies may be determined through clinical trials.

In some instances, a subject may be evaluated, in certain contexts, through one or more of the following diagnostics procedures: 3D CT angiography, Angiography, Anoscopy, Autofluorescence bronchoscopy/fluorescence bronchoscopy, Barium swallow or enema, Biopsy, Bone Marrow Aspiration and Biopsy, Bone Scan, Bronchoscopy, CA-125 test, CAD for mammography, CTC Test, Chest x-ray, Colonoscopy, Complete Blood Count Test, Computed Tomography Scan, CT-guided biopsy, DEXA scan, Digital Breast Tomosynthesis, Electrocardiogram, Endobronchial ultrasound, Endoscopic ultrasound, ERCP, Flow cytometry, Full-field digital mammography, Genetic testing, Large bore CT scanner/RT with simulation, Lumbar puncture, Magnetic Resonance Imaging, Mammography, Miraluma breast imaging, MRI-Guided Breast Biopsy, Multi-detector CT scanner, Multiple-gated acquisition (MUGA) scan, Navigational Bronchoscopy, Nuclear Medicine Imaging, Oncotype DX Test, Pap test, Pelvic exam, PET Scan, PET-CT Scan, Radiofrequency ablation, Sentinel lymph node biopsy, Spiral CT, Tumor marker testing, Tumor molecular profiling, Ultrasound, Video Capsule Endoscopy, X-ray, and the like.

Diagnostic procedures may be performed for a variety of reasons including but not limited to e.g., to screen for cancer or precancerous conditions before a person has any symptoms of disease; to help diagnose cancer; to provide information about the stage of a cancer; to provide information about the malignancy of a tumor; to provide information about the size and/or extent of a primary tumor; to provide information about whether or not a tumor has metastasized; to plan treatment; to monitor a patient's general health during treatment; to check for potential side effects of the treatment; to determine whether a cancer is responding to treatment; to find out whether a cancer has recurred; etc.

### Active Agents

The active agent for binding to an activity modulating domain of CD206, also referred to herein as a CD206-binding agent, can include any convenient compound. According to certain embodiments disclosed herein, the active agent can be an immunomodulatory peptide, a small molecule active agent, or a specific binding member.

### Immunomodulatory Peptides

In certain embodiments of the present disclosure the CD206-binding agent is an immunomodulatory peptide. The terms “immune-modulatory” and “immunomodulatory” are used interchangeably herein. In some cases, an immunomodulatory peptide described herein can be referred to as an anti-inflammatory peptide and vice versa. In certain instances, the immunomodulatory peptide (e.g., as described herein) is an anti-inflammatory peptide, e.g., the peptide has at least one anti-inflammatory property.

Certain aspects of immunomodulatory polypeptides of interest which may be applied to, or adapted for use with, the peptides of the present disclosure are described by Jaynes et al. in WO2016/061133, the disclosure of which is herein incorporated by reference in its entirety.

The terms “peptide” and “polypeptide” are used synonymously herein to refer to polymers constructed from amino acid residues. The term “amino acid residue,” as used herein, refers to any naturally occurring amino acid, non-naturally occurring amino acid, or amino acid mimetic (such as a peptoid monomer). An amino acid residue can be in an L- or D-form.

This disclosure includes immunomodulatory peptides having a striapathic region that comprises at least 25% of the length of the polypeptide and at least one immunomodulatory property. The term “striapathic region,” refers to a region or portion of a peptide sequence that is composed of a sequence of alternating hydrophobic and hydrophilic modules. A “hydrophobic module” is a peptide sequence consisting of one to five (e.g., 1 to 3 or 1 to 2) hydrophobic amino acid residues, e.g., 1, 2, 3, 4 or 5 hydrophobic amino acid residues. A “hydrophilic module” is a peptide sequence consisting of one to five (e.g., 1 to 3 or 1 to 2) hydrophilic amino acid residues, e.g., 1, 2, 3, 4 or 5 hydrophilic amino acid residues.

A striapathic region can thus be represented by the formulae (X₁₋₅Y₁₋₅)ₙ or (J₁₋₅J₁₋₅)ₙ, where each X signifies a hydrophilic amino acid residue, each J signifies a hydrophobic amino acid residue, and each n is an integer from 1 to 10, such as 2 to 10, 2 to 8, 3 to 8, 4 to 8, or 5 to 10. As described in further detail below, aspects of the present disclosure include immunomodulatory peptides having a striapathic region having a specific degree of cationic charge. Immunomodulatory peptides of this disclosure can include a striapathic region having a cationic surface. In certain embodiments, the striapathic region has a cationic charge (i.e., charge >0, e.g., +1, +2, +3, +4, +5, +6 or more). In certain embodiments, the immunomodulatory peptide includes a tail region (e.g., a hydrophobic tail sequence). In certain embodiments, an immunomodulatory peptide includes two or more striapathic regions. In such embodiments, two amphipathic regions of the peptide are in the form of a dimer, where the two amphipathic regions can have the same or different amino acid sequences (i.e., be a homodimer or a heterodimer). In certain embodiments, the two (or more) striapathic regions are connected via a linker or linking region. The linker can be a contiguous (or in-line) amino acid sequence or a non-amino acid moiety as desired.

Hydrophobic amino acid residues are characterized by a sidechain group that has predominantly non-polar chemical or physical properties, e.g., in an environment in which a peptide finds use, e.g., physiological conditions. Such hydrophobic amino acid residues can be naturally occurring or non-naturally occurring. A hydrophobic amino acid residue can be a mimetic of a naturally occurring amino acid that is characterized by a sidechain group that has predominantly non-polar chemical or physical properties. Conversely, hydrophilic amino acid residues are characterized by a sidechain group that is predominantly polar (e.g., charged or neutral hydrophilic), e.g., in an environment in which a peptide finds use, e.g., physiological conditions. Such hydrophilic amino acid residues can be naturally occurring or non-naturally occurring. A hydrophilic amino acid residues can be a mimetic of a naturally occurring amino acid characterized by a sidechain group that is predominantly hydrophilic (charged or neutral polar). Examples of hydrophilic and hydrophobic amino acid residues are shown in Table 1, below. Suitable non-naturally occurring amino acid residues and amino acid mimetics are known in the art. See, e.g., Liang et al. (2013), “An Index for Characterization of Natural and Non-Natural Amino Acids for Peptidomimetics,” PLoS ONE 8(7):e67844.

Although most amino acid residues can be considered as either hydrophobic or hydrophilic, a few, depending on their context, can behave as either hydrophobic or hydrophilic. For example, due to their relatively weak non-polar characteristics, glycine, proline, serine and/or cysteine can sometimes function as hydrophilic amino acid residues. Conversely, due to their bulky, slightly hydrophobic side chains, histidine and arginine can sometimes function as hydrophobic amino acid residues.

TABLE 1
Hydrophobic and Hydrophilic Amino Acid Residues

| Hydrophilic Residues | Hydrophobic Residues |
|---|---|
| (X) | (J) |
| Arginine | Tryptophan |
| Histidine | Phenylalanine |
| Lysine | Tyrosine |
| Aspartic Acid | Isoleucine |
| Glutamic Acid | Leucine |
| Asparagine | Valine |
| Glutamine | Methionine |
| Pyrrolysine | Cysteine |
| Ornithine | Threonine |
| | Serine |
| | Alanine |
| | Proline |
| | Glycine |
| | Selenocysteine |
| | N-formylmethionine |
| | Norleucine |
| | Norvaline |

In some instances, the immunomodulatory peptide is of 5 to 18 amino acid residues in length and includes a striapathic region of alternating hydrophilic and hydrophobic modules that adopts an amphipathic conformation under physiological conditions. In these instances, the striapathic region may include 3 or more hydrophobic modules; and 2 or more hydrophilic modules each comprising at least one cationic residue. In some embodiments, the immunomodulatory peptide includes a sequence defined by one of the formulae:

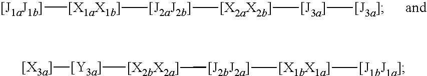

wherein:
J₁ₐ, J_{1b}, J₂ₐ, J_{2b} and J₃ₐ are each independently selected from phenylalanine, tryptophan, alanine, valine, and glycine; and
X₁ₐ, X_{1b}, X₂ₐ, X_{2b} and X₃ₐ are each independently selected from lysine, arginine, histidine, aspartic acid, glutamic acid, asparagine and glutamine. In some instances, J₁ₐ, J_{1b}, J₂ₐ, J_{2b} and J₃ₐ are each phenylalanine; and X₁ₐ, X_{1b}, X₂ₐ, X_{2b} and X₃ₐ are each independently selected from lysine and arginine.

In certain embodiments, the immunomodulatory peptide includes: a) a sequence selected from: KFRKAFKRFF (RP182); FFRKFAKRFK (RP183); FFKKFFKKFK (RP185); FFKKFFKKFK (RP186); and FFKKFFKKFK (RP233); or b) a sequence having one or two amino acid substitutions relative to the sequence defined in a). In certain embodiments, the immunomodulatory peptide includes the amino acid sequence KFRKAFKRFF (RP182). In certain instances, the immunomodulatory peptide includes the amino acid sequence FFRKFAKRFK (RP183). In certain instances, the immunomodulatory peptide includes the amino acid sequence FFKKFFKKFK (RP185).

In other embodiments, the immunomodulatory peptide includes: a) a peptide sequence selected from: RWKFGGFKWR (RP832C); FKWRGGRWKF (RP837C); FWKRGGRKWF (RP837A); FWKRFV (RP837N); FVRKWR (RP837C1); FAOOFAOOFO (RP850); FWKRFVRKWR (RP837); FWKKFVKKWK (RP841); WWHHWWHHWH (RP847); WWRHWWHRWR (RP848); WWKHWWHKWK (RP849); GDRGIKGHRGF (RP842); LYKKIIKKLL (RP846); FYPDFFKKFF (RP844); FFRKSKEKIG (RP853); FFRHFATHLD (RP845); and EKLSAFRNFF (RP843); orb) a sequence having one or two amino acid substitutions relative to the sequence defined in a). In certain embodiments, the immunomodulatory peptide includes the amino acid sequence selected from: RWKFGGFKWR (RP832C), FKWRGGRWKF (RP837C) and FWKRGGRKWF (RP837A). In certain instances, the immunomodulatory peptide includes the amino acid sequence selected from FWKRFV (RP837N) and FVRKWR (RP837C1). In certain instances, the immunomodulatory peptide includes the amino acid sequence selected from FAOOFAOOFO (RP850), FWKRFVRKWR (RP837) and FWKKFVKKWK (RP841). In certain instances, the immunomodulatory peptide includes the amino acid sequence selected from WWHHWWHHWH, WWRHWWHRWR and WWKHWWHKWK (RP847-849).

In certain embodiments, the immunomodulatory peptide includes the amino acid sequence LYKKIIKKLL (RP846). In certain instances, the immunomodulatory peptide includes the amino acid sequence FYPDFFKKFF (RP844). In certain instances, the immunomodulatory peptide includes the amino acid sequence FFRKSKEKIG (RP853). In certain instances, the immunomodulatory peptide includes the amino acid sequence FFRHFATHLD (RP845). In certain instances, the immunomodulatory peptide includes the amino acid sequence FFRKSKEKIG (RP853).

In some embodiments, the immunomodulatory peptide includes a sequence defined by one of the formulae:

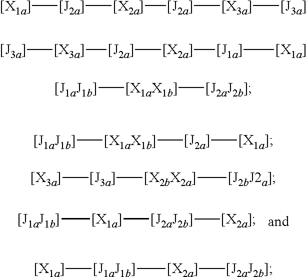

wherein:
J₁ₐ, J_{1b}, J₂ₐ, J_{2b} and J₃ₐ are each independently selected from phenylalanine, tryptophan, alanine, valine, leucine, isoleucine, methionine, tyrosine, threonine, serine, cysteine, proline, and glycine; and
X₁ₐ, X_{1b}, X₂ₐ, X_{2b} and X₃ₐ are each independently selected from lysine, arginine, histidine, aspartic acid, glutamic acid, asparagine and glutamine. In certain embodiments, the immunomodulatory peptide includes: a) a sequence selected from: AFKRFF (182-FN6); FFKKFF (185-FN6); FWKRFV (837-FN6); WVRRVV (WLUB-F1-N6); IFKKIE (CEC-F1-N6) FLRNLV (LL37F-3-N6); FLHSAK (MAG-F1-N6); FFHHIF (PISC-F-N6); FFKKAA (PLEU-F-N6); ALKKVF (PSEU-F-N6); LYKKII (CXCL4-F-N6); LFRRAF (IL24-FN6); FLKRLL (IL7-FN6); FFRRFA (ABCP-FN6); FFRHFA (E1P-FN6); AIRRIP (gP120-FN6); AFHRFF (GP2B-FN6); FFNRFA (MCPH-FN6); AFKRFF (SPRA-FN6); AFKRFF (TPRO-FN6); IVRRAD (COL18-FN6); FWRWFK (HX5/CPAP); KFWRWF (HX6/YJPA); WFRFWK (HX7/CLPB) KWFRFW (HX8/ATG1); AFHHFF (HEX16F/STPK); FFRNFA (HEXF13/SIF1); AFHRFF (HEX9F/THIF); FFRQFA (HEXF1/ATPB); AFNRFF (HEX2F/AATF); WIQRMM (CXCL13-FN6); WVQRVV (CXCL8-FN6); AFRNFF (HEX3F/FBNA); and TLRRFM (HEX18/HSHK); or b) a sequence having one or two amino acid substitutions relative to the sequence defined in a). In other embodiments, the immunomodulatory peptide includes: a) a sequence selected from: DVRMRL (MCMV-FN6); and RRAELG (TONB-FN6) or b) a sequence having one or two amino acid substitutions relative to the sequence defined in a). In some other embodiments, the immunomodulatory peptide includes: a) a sequence selected from: FWRWFA (HX1/MMPL); AFWRWF (HX2/ABCT); WFRFWA (HX3/GTRF); AWFRFW (HX4/AXES); VAVRIW (HX9/IDRF/AMIA); FFRFFA (HEXF2/AMT1); and AFFRFF (HEX13F/TGME); or b) a sequence having one or two amino acid substitutions relative to the sequence defined in a). In other embodiments, the immunomodulatory peptide includes: a sequence selected from: FFKKFF; WWKKFF; FWKKWF; FFKKWW; WWKKWW; YYKKYY; IIKKYY; YIKKIY; YYKKII; IIKKII; MMKKMM; LLKKMM; MLKKLM; MMKKLL; LLKKLL; VVKKVV; AAKKVV; VAKKAV; VVKKAA; AAKKAA; GGKKGG; TTKKGG; GTKKTG; GGKKTT; TTKKTT; SSKKSS; CCKKSS; SCKKCS; SSKKCC; and CCKKCC; or b) a sequence having one or two amino acid substitutions relative to the sequence defined in a). In certain other cases, the immunomodulatory peptide includes: a sequence selected from: FKFKFK; WKWKWK; YKYKYK: IKIKIK; MKMKMK; LKLKLK; VKVKVK; AKAKAK; GKGKGK; TKTKTK; SKSKSK; CKCKCK; KFKFKF; KWKWKW; KYKYKY; KIKIKI; KMKMKM; KLKLKL; KVKVKV; KAKAKA; KGKGKG; KTKTKT; KSKSKS; and KCKCKC; or b) a sequence having one or two amino acid substitutions relative to the sequence defined in a).

In certain embodiments, the immunomodulatory peptide comprises a peptide sequence as described in Tables 2A-2C.

Tables 2A-2C: Exemplary immunomodulatory peptides sequences including 6 or 10 amino acids.

TABLE 2A

| Name | Sequence |
|---|---|
| FF13FF | FFHHFF |
| FF3FF | FFRHFF |
| FF11FF | FFKNFF |
| FLAG23 | LWHHWP |
| E1P-FN6 | FFRHFA |
| FF4FF | FFRNFF |
| PISC-FN6 | FFHHIF |
| PFMB6 | FFNGYP |
| FF14FF | FFHNFF |
| FF23FF | FFQHFF |
| FF5FF | FFRQFF |
| PREV17 | WFRDVF |
| HX3/GTRF | WFRFWA |
| FF18FF | FFNHFF |
| TRAC10 | LFHTLF |
| HX4/AXES | AWFRFW |
| FIBR6 | FLHHMV |
| FF15FF | FFHQFF |
| HX1/MMPL | FWRWFA |
| GP2B-FN6 | AFHRFF |
| SEQ1 | AFHRFF |
| FF34FF | FFTGFF |
| FF30FF | FFTSFF |
| FF8FF | FFKHFF |
| FF24FF | FFQNFF |
| FLHB28 | YFTRLF |
| FF12FF | FFKQFF |
| FF29FF | FFSTFF |
| LL37-3FN6 | FLRNLV |
| PREV9 | FFQNIF |
| FF19FF | FFNNFF |
| FF33FF | FFGTFF |
| TRAC52 | SWHRLF |
| TRAC24 | IMNHMI |
| FF22FF | FFQKFF |
| FF31FF | FFGSFF |
| TRAG33 | LFRNYA |
| HX8/ATG1 | KWFRFW |
| FF26FF | FFGGFF |
| TRAG20 | IFSDMF |
| ADHE16 | YLKSFF |
| PREV16 | VFDSIF |
| FIMH19 | LWQTLL |
| TRAC70 | YYTHAA |
| HX7/CLPB | WFRFWK |
| HX2/ABCT | AFWRWF |
| FLAG39 | VYTSVV |
| TRAG81 | YVQQIF |
| ABCP-FN6 | FFRRFA |
| SPRA17 | YLDHYF |
| PORPH2 | FMQSFI |
| HIV6 | LLSGIV |
| FIBR2 | AFRNFF |
| VIRB59 | VYGGWF |
| 837-FN6 | FWKRFV |
| ADHE53 | YVTNFI |
| FLHB2 | FFGEMF |
| ADHE23 | IINRII |
| VIRB58 | TLRSYL |
| RHSP17 | AVHHWA |
| HIV5 | LFRRAF |
| FIMH27 | TYRSYV |
| ADHE47 | VTRRFI |
| FIBR9 | FYDDIV |
| TRAG46 | LYTQLF |
| SPRA5 | FLSQYF |
| ADHE12 | FLKDLV |
| TRAC28 | ITQNIV |
| FIMH16 | LLRNTP |
| SPRA8 | IIRNYY |
| PORPH1 | FMQNFV |
| FIMH18 | LSTQIF |
| ADHE11 | FFSSLA |
| FIMH34 | YFQEYI |
| FIMH6 | AYGGVL |
| VIRB56 | LYQQMA |
| VIRB57 | MLSSAM |
| TRAG10 | FFGDFV |
| TRAC11 | LINTMP |
| FIBR15 | LLDRFY |
| FIMH13 | LFSQAG |
| YOPB15 | MATRGL |
| FIMH11 | IFRNTA |
| PREV12 | MLRNYY |
| FLAG28 | MLTGAF |
| TRAG43 | LMNGMM |
| RHSP13 | VAHHYA |
| RHSP9 | FFSKLP |
| YOPB8 | LARQVV |
| SEQ2 | AFNNFF |
| PREV7 | AYQNFL |
| TRAC17 | FLNNLI |
| PFMB4 | AWHDVA |
| SPRA14 | VFNELY |
| PREV14 | VFEQLY |
| FIMH7 | FVSGVY |
| FLID3 | ALQSFM |
| 182-FN6 | AFKRFF |
| FLAG27 | MASQMV |
| SIPB3 | ALSKMM |
| SPRA6 | FTDNFY |
| SIPD4 | PLQKMV |
| ADHE31 | LLKNIL |
| FLAG38 | VVDRAL |
| FLIC33 | VASEMV |
| FLAG19 | IYQRYS |
| TRAC14 | FAGDMM |
| FLAG11 | FGQNIY |
| FIBR3 | ALDGIF |
| FIMH37 | YVSELP |
| PFMB19 | WWGNSY |
| YOPB6 | ILGKIA |
| YOPB27 | WLKQSV |
| FIMH35 | YFQEYL |
| FLHB14 | GLKRMF |
| FLHB23 | MFDRAT |
| FLAG31 | TAQRYL |
| TRAG72 | WLNEFG |
| TRAG18 | IADRVF |
| FIBR16 | LLDRYY |
| FIBR7 | FSKEIF |
| PORPH3 | AFNQFL |
| HIV1 | AVERYL |
| FLAG10 | AVRGIF |
| FLID7 | LTQQFT |
| SIPD3 | AFSDIL |
| RHSP3 | YLDNVS |
| VIRB510 | YISDFT |
| ADHE43 | SYNNWY |
| TRAG48 | MFNNIW |
| TRAC19 | SWQEVV |
| FLAG2 | ALSEII |
| RHSP8 | GLKRVV |
| ADHE34 | LYRKIV |
| TRAC7 | ALKQVL |
| FLHB15 | GTKRLF |
| FIMH22 | PPRNYI |
| RHSP15 | LLRKAM |
| ADHE22 | IASKLL |
| FLHB13 | GLKRIF |
| FLIC12 | AITRLS |
| FLIC22 | ILQQAG |
| TRAC3 | AASKWL |
| FLAG29 | MMSEVP |
| FLHB19 | LIPRFA |
| FIMH1 | AFGGAL |
| ADHE1 | AAQEVI |
| FLIC29 | TLDKAL |
| ADHE32 | LLQQVG |
| FIMH2 | AFNDYL |
| FLIC5 | AAKQMV |
| FLHB12 | GLKNMF |
| FLIC8 | AIERLS |
| SPRA13 | VAKEFL |
| TRAC33 | LINNYL |
| FLIC6 | AANEII |
| TRAC6 | FLSSAT |
| FLIC36 | VPQQVL |
| PFMB17 | TLDGFF |
| FIMH3 | AFNDYS |
| YOPB25 | VVEGFL |
| ADHE5 | AISNIW |
| 185-FN6 | FFKKFF |
| FLIC31 | TTQNVL |
| FLHB7 | GFKRIF |
| FIMH12 | ILGQAM |
| TRAC39 | LMNDYI |
| TRAC22 | ILDEAW |
| FLHB11 | GIKRMF |
| FIBR10 | IAKNIF |
| SIPB6 | LIGKAI |
| YOPB10 | LIGKAI |
| FLIC27 | SLGTAI |
| FIMH31 | VPKHVY |
| FLAG15 | HIQQAG |
| FLID9 | TLKSAL |
| TRAG21 | IIDKIF |
| ADHE14 | FMDKYI |
| FLAG18 | ITNKAA |
| VIRB55 | IVSTAA |
| TRAG3 | AFSEAW |
| TRAC16 | MSGGYL |
| FLHB8 | GIAKMF |
| SPRA10 | MIKTAF |
| SIPB7 | VLKQLA |
| FIBR1 | AFDEVM |
| FLIC35 | VIDEAI |
| HIV3 | IVGGLV |
| FLAG25 | MAKEMS |
| FLAG30 | SFDNYI |
| FLIC19 | GAQSAV |
| FIBR8 | FTKKMV |
| FIMH26 | TITDYV |
| FLHB17 | ISKDFF |
| FLAG34 | VAEELY |
| FLIC11 | AINNAI |
| FLIC32 | VADELL |
| HIV2 | IVGGLI |
| YOPB1 | AFKDVV |
| PFMB13 | MKNLF |
| FLIC7 | AFTDGA |
| FIMH30 | VGSKLY |
| TRAG51 | MIRKYY |
| YOPB21 | SVQQAA |
| TRAC14 | MAEKAY |
| FLIC17 | ASDKLF |
| ADHE38 | PPQDYV |
| ADHE26 | TYKKFY |
| PREV4 | MMKNVY |
| YOPB14 | LVKRGV |
| FIMH8 | GSANVY |
| FLAG6 | AIDNLF |
| TRAC49 | SINDLY |
| CXCL4-F | LYKKII |
| FLIC30 | TTGKYY |
| TRAC54 | TYKKYY |
| FLIC3 | AAGKMG |
| YOPB24 | VMEKLG |
| YOPB13 | LMSKFG |
| FLIC13 | ALDEAI |
| TONB-FN6 | RRAELG |
| FLIC10 | AINKVS |
| FLIC4 | AAGQAI |
| YOPB16 | MFKKIL |
| ADHE39 | PPRKYI |
| FLAG4 | SLKGAM |
| FLHB1 | AMGNAM |
| FLAG20 | LANQSA |
| PFMB15 | TADSWF |
| FLIC16 | AMEKLS |
| CEC1-FN6 | IFKKIE |
| FIBR18 | LLKKFS |
| FLIC9 | AIKDTY |
| PLEU-FN6 | FFKKAA |
| 832c-FN6 | WKFKWR |
| FF21FF | FFQRFF |
| SPRA2 | FFESIF |
| SPRA16 | WLTNMI |
| FF32FF | FFSGFF |
| TRAC8 | GWRTYV |
| FF28FF | FFSSFF |
| FF27FF | FFTTFF |
| FF16FF | FFNRFF |
| FF1FF | FFRRFF |
| SPRA3 | FFPSLF |
| TRAG59 | PYHHII |
| TRAG63 | TLHRMM |
| RHSP10 | VVRHWT |
| SEQ3 | AFRHFA |
| FIMH14 | LFTTLL |
| TRAC67 | YLRDVY |
| SEQ4 | AYRHFA |
| CXCL13-F | WIQRMM |
| SEQ5 | AFHRYA |
| FF25FF | FFQQFF |
| TRAC8 | VLSRLW |
| HX9/IDRF/AMIA | VAVRIW |
| TRAC41 | LWNNLL |
| TRAG71 | WLGHLM |
| PFMB10 | ILRRVF |
| PFMB18 | VWRGIT |
| FLHB25 | MYRHVL |
| PREV2 | FFEQFF |
| HX5/CPAP | FWRWFK |
| FF20FF | FFNQFF |
| SPRA18 | YLTEII |
| FIBR14 | LLDRFF |
| HIV4 | IWNNMT |
| FF2FF | FFRKFF |
| TRAC24 | WPERVF |
| FIMH33 | WSHGIY |
| PREV8 | AFNRFV |
| TRAC11 | ATRHII |
| YOPB26 | VVERMV |
| SPRA4 | FLHKTL |
| HX6/YJPA | KFWRWF |
| TRAC1 | ATRNFV |
| FLHB3 | FVRESF |
| TRAC63 | WYSEIM |
| TRAG79 | YLGRYM |
| TRAC31 | LAQHIV |
| FLAG17 | ISDRML |
| FLHB26 | VMRNFV |
| SPRA15 | VFREMY |
| FLID4 | AYNSLI |
| YOPB2 | IMERIF |
| ADHE20 | GYTRIL |
| FF6FF | FFKRFF |
| RHSP4 | AWRQAA |
| CXCL8-F | WVQRVV |
| RHSP5 | SGHHLL |
| YOPB19 | MVERMV |
| TRAC25 | IMRDMY |
| FF17FF | FFNKFF |
| FIMH38 | YVTDLP |
| RHSP6 | YFHQPL |
| TRAC2 | AARNIW |
| MAG1-FN | FLHSAK |
| FLAG33 | TYHEFA |
| SIPD2 | VYTDFY |
| TRAC18 | PVRQLL |
| TRAG35 | LFRRFG |
| TRAG9 | FFETAL |
| TRAG8 | FFESAL |
| SIPD5 | LYDNLV |
| TRAG55 | MYQQLF |
| YOPB7 | IMEHVL |
| COL18-FN6 | IVRRAD |
| PREV1 | SYQNFL |
| TRAG82 | YYKQLY |
| TRAG1 | AARDMM |
| FIMH24 | TARGYL |
| PREV10 | MLNKIP |
| TRAC20 | TLNTLF |
| FIBR12 | ILENYY |
| ADHE15 | FPDNML |
| TRAC5 | FLSQLA |
| RHSP19 | FGRRIA |
| TRAG7 | FFDQLI |
| TRAC2 | AYDRVM |
| FIMH4 | ALSTPV |
| WLUB-F1-N6 | WVRRVV |
| ADHE8 | ATEHFF |
| SPRA7 | FYDQLM |
| PREV15 | YTKELF |
| PREV3 | MVRNYY |
| FLHB20 | LIREAM |
| TRAC3 | AYDSMT |
| FLIC21 | IANRFT |
| YOPB12 | LLGKLM |
| FLHB21 | LLKSLI |
| RHSP16 | YFDGFV |
| FLAG26 | MASEIV |
| FIMH36 | YTQRLA |
| FIMH9 | GWDSII |
| PFMB3 | AVSHAA |
| FLIC34 | VGDDYY |
| TRAG24 | IINNFI |
| TRAC17 | MYESMA |
| FIBR11 | ILDNYY |
| YOPB3 | ALQTAV |
| PFMB7 | FITNYL |
| FIMH10 | GWDSIV |
| YOPB18 | MMTSLI |
| RHSP18 | YFDGLV |
| FIMH21 | MMTDYI |
| PREV13 | MTRDYY |
| SPRA12 | TYQKYL |
| FLAG16 | ILQQAA |
| YOPB11 | LISNYV |
| TRAC7 | FYEKLI |
| VIRB54 | FYSSAG |
| TRAC16 | FIQKGY |
| FLAG8 | AMERIS |
| TRAC36 | LLKDVL |
| YOPB17 | MLHNLA |
| FLHB27 | YFKRVF |
| PFMB14 | MKRVI |
| PREV6 | FSEQFL |
| TRAC46 | PFRKWL |
| FLAG12 | GGNKLL |
| FLID5 | GIRDAI |
| FIMH39 | YWSEYF |
| TRAC23 | ILRKAI |
| TRAC4 | FIDDYI |
| IL7-F | FLKRLL |
| TRAC25 | YFRRTG |
| SIPD1 | VYENVV |
| PFMB16 | TAQNWI |
| RHSP14 | ILEDVA |
| PFMB11 | LIKSWV |
| FIBR13 | LFGELL |
| FLHB10 | GIKRLF |
| FLIC24 | MAKEMM |
| FLAG32 | TATNYM |
| FLHB6 | GFKNMF |
| YOPB20 | SLNDLW |
| SPRA1 | AGQKLM |
| HIV7 | SLDDIW |
| FLAG37 | VLQQAG |
| PFMB9 | HIRDAG |
| FLID6 | ILDSLT |
| FLIC28 | STQNVL |
| FLID8 | PLDQLL |
| FLHB16 | GVKNLF |
| FLID2 | AIKSWV |
| ADHE7 | APKNML |
| FLAG22 | LISTLL |
| FIMH5 | AVKRIV |
| YOPB23 | VLNKLF |
| FLIC23 | LPQQVL |
| TRAC23 | VYSDAM |
| FLIC37 | YATEVS |
| YOPB5 | FIQQAL |
| PFMB8 | IIKNLY |
| FLID10 | TVNNVA |
| FIBR20 | VLRKYL |
| FLIC20 | GGRKLL |
| FLIC14 | ALGTAI |
| FIMH17 | LMKTFP |
| FIMH29 | VGQNLV |
| FLAG13 | GGRKLL |
| YOPB9 | LASKVA |
| FIBR5 | ATKNIF |
| TRAC12 | LPEDVW |
| FLHB18 | LARKLA |
| FIMH25 | TATDIF |
| FLIC25 | MIQTAS |
| FIBR4 | ALKKLI |
| ADHE17 | GIKNLY |
| TRAC21 | IIEEAW |
| TRAC9 | IAQNLA |
| FLIC15 | ALNKLG |
| RHSP12 | MTRKGL |
| FLIC26 | PADEIL |
| FLID1 | AIKDWV |
| TRAG29 | LASKYF |
| TRAC20 | IIDEAW |
| FLHB24 | MMDDVP |
| FLAG36 | VLKKLI |
| FIMH28 | VAKDIS |
| RP426-FN6 | AAKRAF |
| FLHB9 | GIKRIF |
| RHSP7 | VYDEMG |
| FLAG1 | TADKAM |
| ADHE49 | WFKRGL |
| SPRA11 | MMKNIY |
| FIRB19 | MLKKYF |
| FLIC18 | AYNDAP |
| FIMH15 | LLDEAL |
| RHSP1 | WIDKGG |
| TRAG23 | IIKGYI |
| SPRA9 | MIKNIG |
| FLHB5 | FYKKIV |
| RHSP2/107 | FAKKFA |
| TRAC48 | SADRYY |
| CFC5/185B2 | FFKFKF |
| FIMH20 | MMKKII |
| PSEU-FN6. | ALKKVF |
| FLIC2 | AADNAI |
| PFMB5 | AYKKVF |
| CFC4/ADB2 | FFRFHF |
| PFMB12 | MFKKTL |
| FIBR17 | LLKKFG |
| CFC3/ADB1 | FRFHFF |
| CFC5/185B1 | FKFKFF |
| TRAG31 | YAKKYG |
| PFMB1 | AIDDFG |
| ADHE3 | AIKKII |
| CFC1 | GGKKGG |
| CFC2 | KGGGGK |
| PFMB2 | AMKKVV |
| FIMH23 | SAGGVA |
| FLAG9 | AMKKLA |
| FLIC1 | AADDAA |

TABLE 2B

| Origin | Sequence | Origin | Sequence |
|---|---|---|---|
| CCL1 | RKMLRH | XCL2 | wvrdvv |
| MCP-1-A | SYRRIT | CXCL3 | mvqkiiekilnkgs |
| MCP-1-B | WVQDSM | CXCL4 | lYkkiikkll |
| (CCL4) | WVQEYV | CXCL5 | flkkviqkil |
| CCL11-A | SYRRIT | CXCL6 | flkkviqkil |
| CCL11-B | WVQDSM | CXCL13 | rrfidr |
| CCL18 | WVQKYI | CXCL14 | stkrfi |
| CX3CL1 | WVKDAM | LIG15-MSE | liknim |
| CXCL1-A | MVKKII | CXCL16 | wvqelm |
| CXCL1-B | IIEKML | IL-1F10 | Yfeqsw |
| CXCL7 | KKIVQK | IL-1F9 | felnin |
| CXCL8-A | WVQRVV | IL-1F8 | tldqwg |
| CXCL8-B | VVEKFL | IL-3 | mideii |
| CXCL9 | KELIKK | IL-5 | lvketl |
| CXCL10 | AIKNLL | IL-7 | flkrll |
| IL-2 | FLNRWI | IL-11 | wlrrag |
| IL-4-A | TLQEII | IL-12 | asrkts |
| IL-4-B | TLENFL | IL-13 | kklfre |
| IL-6-A | WLQDMT | TAXILLIN | llkeav |
| IL-6-B | SFKEFL | IL-16 | kkliee |
| IL-9 | FSERLS | IL-17A | ivhhva |
| IL-10-A | YLEEVM | IL-19 | Yvdrvf |
| IL-10-B | LRLRLR | IL-21 | kefler |
| IL-12-A | ASRKTS | IL-22 | itnrtf |
| IL-12-B | TIDRVM | IL-23 | fYekll |
| IL-15 | TVENLI | IL-25 | tseell |
| IL-18 | LFEDMT | IL-26 | fmedvf |
| IL-20-A | LLRHLL | IL-27 | larkll |
| IL-20-B | TLRKIS | IL-29 | aledvl |
| IL-24 | WMQKFY | IL-31 | qkivee |
| IL-28 | KRLLEK | IL-32 | erfYdk |
| IL-34 | YMKHYF | IL-33 | rettkr |
| IFN-gamma | AIHELI | TNF-BETA | ssqkmv |
| CRP-A | MSRKAF | IFN-ALPHA | vlheliqqif |
| CRP-B | WSKDIG | SCF | aYkels |
| CRP-C | SLKKGY | NGF | lskqmv |
| LIF-A | NNLMNQ | HGCSF | tvqeat |
| LIF-B | SGKDVF | TachYkinin | mgkral |
| ONCSTN-A | LGRRGF | CCL22 | vvkhfY |
| ONCSTN-B | ALRKGV | CCL23 | lkldtriktr |
| (CCL3) | wvqkYv | CCL24 | wvqrYm |
| CCL5 | wvreYi | CCL25 | vlrraw |
| CCL6 | isrrgt | CCL26 | wvqkYi |
| CCL7 | sYrrtt | CCL27 | mirkmg |
| CCL8 | wvrdsm | CCL28 | vshhisrrller |
| LIG-9 MSE | iskrgf | XCL1 | wvrdvv |
| CCL12 | sYrrit | CCL19 | wverii |
| CCL13 | wvqnYm | CCL20 | Ytdril |
| (CCL14) | wvqdYi | CCL21 | wvqqlm |

TABLE 2C

| Name | Sequence | Name | Sequence |
|---|---|---|---|
| AP00143 | KLLKWLKKLL | AP02253 | FIKKFYKQIM |
| AP00163 | LWKDILKNVG | AP02569 | RLLKSVRRAV |
| AP00325 | KVVNVLKNLF | AP02664 | KWRRWVRWI |
| AP00366 | KFKKLFKKLS | AP02671 | KLFKKILKYL |
| AP00371 | LSDRGRRLGE | AP02899 | KFWKKVLNGA |
| AP00385 | KGKEMLKDYA | AP02924 | LWKEVLKNAG |
| AP00484 | KLVKKVKHTI | AP02964 | KIKKGFRKIF |
| AP00496 | KVFKRLEKLF | AP02981 | KGKELLRDYA |
| AP01010 | RKLKKLRNAL | AP00012 | GLFDIIKKIA |
| AP00680 | SLQRGGQKIL | AP00016 | GLFDIVKKVV |
| AP00541 | KLLDAAKQIL | AP00082 | GLKNVGKEVG |
| AP00624 | RIKDFLRNLV | AP00155 | GLRRLGRKIA |
| AP00629 | RIVQRIKDFL | AP00376 | GWKDWAKKAG |
| AP00691 | KVKHAGRRVL | AP00555 | GKVRAKAKTR |
| AP00722 | LGKRALKKII | AP01239 | KFFRKLKKSV |
| AP00774 | KVRKGFKEAS | AP01456 | GKSKIKWQ |
| AP01012 | SMAKKLKEYM | AP01540 | AFQDTIRKFL |
| AP01295 | TAKNVAKNVA | AP02318 | ILKKVGKEAV |
| AP01377 | RQGWRAHKVV | AP02424 | KFFKKVKKSV |
| AP01633 | RVVRVVRRWV | AP02749 | AMEKIAEKVG |
| AP01634 | KIFEKVKNLV | AP02776 | IRWRIRV |
| AP01768 | KIGQRIRDFF | | |

In some embodiments, the immunomodulatory peptide includes a sequence defined by one of the formulae:

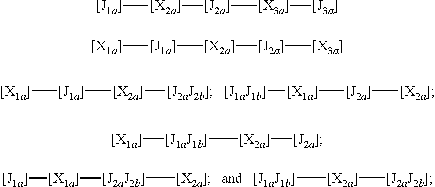

wherein:
J₁ₐ, J_{1b}, J₂ₐ, J_{2b} and J₃ₐ are each independently selected from phenylalanine, tryptophan, alanine, valine, leucine, isoleucine, methionine, tyrosine, threonine, serine, cysteine, proline, and glycine; and
X₁ₐ, X_{1b}, X₂ₐ, X_{2b} and X₃ₐ are each independently selected from lysine, arginine, histidine, aspartic acid, glutamic acid, asparagine and glutamine. In certain embodiments, the immunomodulatory peptide includes: a) a sequence selected from: AFKRF; FFKKF; FWKRF; WVRRV; IFKKI; FLRNL; FLHSA; FFHHI; FFKKA; ALKKV; LYKKI; LFRRA; FLKRL; FFRRF; FFRHF; AIRRI; AFHRF; FFNRF; IVRRA; FWRWF; KFWRW; WFRFW; KWFRF; AFHHF; FFRNF; FFRQF; AFNRF; WIQRM; WVQRV; AFRNF; TLRRF; FKRFF; FKKFF; WKRFV; VRRVV; FKKIE; LRNLV; LHSAK; FHHIF; FKKAA; LKKVF; YKKII; FRRAF; LKRLL; FRRFA; FRHFA; IRRIP; FHRFF; FNRFA; VRRAD; WRWFK; FRFWK; FHHFF; FRNFA; FRQFA; FNRFF; IQRMM; VQRVV; FRNFF; LRRFM; DVRMR; VRMRL; RRAEL; RAELG; and RWKFG; or b) a sequence having one or two amino acid substitutions relative to the sequence defined in a). In other embodiments, the immunomodulatory peptide includes: a) a peptide sequence selected from: AFWRW; AWFRF; VAVRI; FFRFF; AFFRF; WRWFA; FRFWA; AVRIW; and FRFFA; or b) a sequence having one or two amino acid substitutions relative to the sequence defined in a). In other embodiments, the immunomodulatory peptide includes: a) a peptide sequence selected from: FFKKF; WWKKF; FWKKW; FFKKW; WWKKW; YYKKY; IIKKY; YIKKI; YYKKI; IIKKI; MMKKM; LLKKM; MLKKL; MMKKL; LLKKL; VVKKV; AAKKV; VAKKA; VVKKA; AAKKA; GGKKG; TTKKG; GTKKT; GGKKT; TTKKT; SSKKS; CCKKS; SCKKC; SSKKC; and CCKKC; FKKFF; WKKFF; WKKWF; FKKWW; WKKWW; YKKYY; IKKYY; IKKIY; YKKII; IKKII; MKKMM; LKKMM; LKKLM; MKKLL; LKKLL; VKKVV; AKKVV; AKKAV; VKKAA; AKKAA; GKKGG; TKKGG; TKKTG; GKKTT; TKKTT; SKKSS; CKKSS; CKKCS; SKKCC; and CKKCC; or b) a sequence having one or two amino acid substitutions relative to the sequence defined in a). In certain other cases, the immunomodulatory peptide includes: a sequence selected from: a) a peptide sequence selected from: FKFKF; WKWKW; YKYKY: IKIKI; MKMKM; LKLKL; VKVKV; AKAKA; GKGKG; TKTKT; SKSKS; CKCKC; KFKFK; KWKWK; KYKYK; KIKIK; KMKMK; KLKLK; KVKVK; KAKAK; KGKGK; KTKTK; KSKSK; and KCKCK; or b) a sequence having one or two amino acid substitutions relative to the sequence defined in a).

In certain embodiments, the immunomodulatory peptide comprises a peptide sequence as described in Table 2, truncated by 1 amino acid at the N-terminus. In some other instances, the immunomodulatory peptide comprises a peptide sequence as described in Table 2, truncated by 1 amino acid at the C-terminus.

In some embodiments, the immunomodulatory peptide includes a sequence defined by one of the formulae:

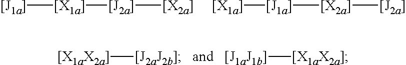

wherein:
J₁ₐ, J_{1b}, J₂ₐ, and J_{2b} are each independently selected from phenylalanine, tryptophan, alanine, valine, leucine, isoleucine, methionine, tyrosine, threonine, serine, cysteine, proline, and glycine; and
X₁ₐ, X_{1b}, X₂ₐ, and X_{2b} are each independently selected from lysine, arginine, histidine, aspartic acid, glutamic acid, asparagine and glutamine. In certain embodiments, the immunomodulatory peptide includes: a) a sequence selected from: AFKR; FFKK; FWKR; WVRR; IFKK; FLRN; FLHS; FFHH; ALKK; LYKK; LFRR; FLKR; FFRR; FFRH; AIRR AFHR; FFNR; IVRR; FWRW; KFWR; WFRF; KWFR; AFHH; FFRN; FFRQ; AFNR; WIQR; WVQR; AFRN; TLRR; KRFF; KKFF; KRFV; RRVV; KKIE; RNLV; HSAK; HHIF; KKAA; KKVF; KKII; RRAF; KRLL; RRFA; RHFA; RRIP; HRFF; NRFA; RRAD; RWFK; RFWK; HHFF; RNFA; RQFA; NRFF; QRMM; QRVV; RNFF; RRFM; VRMR; RMRL; RAEL; AELG; and WKFG; or b) a sequence having one or two amino acid substitutions relative to the sequence defined in a). In other embodiments, the immunomodulatory peptide includes: a) a peptide sequence selected from: FWRW; AFWR; WFRF; AWFR; VAVR; FFRF; AFFR; RWFA; WRWF; RFWA; FRFW; VRIW; RFFA; and FRFF; or b) a sequence having one or two amino acid substitutions relative to the sequence defined in a). In other embodiments, the immunomodulatory peptide includes: a) a peptide sequence selected from: FFKK; WWKK; FWKK; YYKK; IIKK; YIKK; MMKK; LLKK; MLKK; VVKK; AAKK; VAKK; GGKK; TTKK; GTKK; SSKK; CCKK; SCKK; KKFF; KKWF; KKWW; KKYY; KKIY; KKII; KKMM; KKLM; KKLL; KKVV; KKAV; KKAA; KKGG; KKTG; KKTT; KKSS; KKCS; and KKCC; or b) a sequence having one or two amino acid substitutions relative to the sequence defined in a). In certain other cases, the immunomodulatory peptide includes: a sequence selected from: a) a peptide sequence selected from: FKFK; WKWK; YKYK: IKIK; MKMK; LKLK; VKVK; AKAK; GKGK; TKTK; SKSK; CKCK; KFKF; KWKW; KYKY; KIKI; KMKM; KLKL; KVKV; KAKA; KGKG; KTKT; KSKS; and KCKC; or b) a sequence having one or two amino acid substitutions relative to the sequence defined in a).

In certain embodiments, the immunomodulatory peptide comprises a peptide sequence as described in Table 2, truncated by 2 amino acid at the N-terminus. In some other instances, the immunomodulatory peptide comprises a peptide sequence as described in Table 2, truncated by 2 amino acid at the C-terminus.

The exemplary immunomodulatory peptide sequences described herein are merely examples and are not the only immunomodulatory polypeptides provided herein. Indeed, fragments and variants of the sequences of the disclosed peptides are also within the scope of the present disclosure.

The present disclosure provides immunomodulatory polypeptides, sometimes referred to as “RP peptides,” that satisfy one or more of the structural formulae described above. The present disclosure also provides immunomodulatory polypeptides that share a minimum degree of homology with any of the exemplary RP peptides disclosed herein, or variant thereof, or a fragment thereof. Thus, a peptide or polypeptide of the present disclosure is an immunomodulatory peptide that satisfies one of the formulae described herein or shares a minimum degree of homology with any of the exemplary RP peptides disclosed herein.

A “fragment” of the invention includes at least 4, 5, 6, 7, 8, 9, 10, 11, 12, 13, 14, 15, 16, 17, 18, 19, 20, 21, 22, or 23 contiguous amino acid residues of a peptide disclosed herein (or up to one less than the number of amino acid residues in the subject peptide) and retains at least one immunomodulatory property of the subject peptide. Thus, fragments of the invention include peptides that are missing one, two, three, four, or more amino acids from the N-terminus and/or the C-terminus relative to a parent immunomodulatory peptide disclosed herein.

A “variant” of the invention is a polypeptide that is substantially similar to a polypeptide disclosed herein and retains at least one immunomodulatory property of the subject polypeptide. Variants can include deletions (i.e., truncations) of one or more amino acid residues at the N-terminus or the C-terminus of a subject polypeptide disclosed herein; deletion and/or addition of one or more amino acid residues at one or more internal sites in the subject polypeptide disclosed herein; and/or substitution of one or more amino acid residues (e.g., one, two, three, or even more) at one or more positions in the subject polypeptide disclosed herein. For subject polypeptides that are 12 amino acid residues in length or shorter, variant polypeptides can include three or fewer (e.g., three, two, one, or none) deleted amino acid residues, whether located internally, at the N-terminal end, and/or at the C-terminal end.

Accordingly, the invention further provides immunomodulatory polypeptides that are at least 50% identical (i.e., at least 50% sequence identity) (e.g., at least 60%, at least 70%, at least 80%, at least 85%, at least 90%, at least 95% or more) to any one of the immunomodulatory polypeptides disclosed herein (e.g., Table 2) and still retain at least one immunomodulatory property. Sequence identity is based on a comparison of two peptide sequences or fragments thereof of the same or similar length.

As such, in certain embodiments, this disclosure provides polypeptides that include an amino acid sequence having from 1 to 10 amino acid differences (e.g., 10 or fewer, 9 or fewer, 8 or fewer, 7 or fewer, 6 or fewer, 5 or fewer, 4 or fewer, 3 or fewer, 2 or fewer, or 1 amino acid difference) to any one of the polypeptides disclosed herein and still retain at least one immunomodulatory property. An “amino acid difference” as used herein includes: an amino acid substitution, an amino acid insertion, a terminal amino acid addition, an amino acid deletion, a terminal amino acid truncation, or any combination thereof.

In some embodiments, any of the peptides disclosed herein can be 1 or 2 amino acids shorter at the N-terminus. In some embodiments, any of the peptides disclosed herein can be 1 or 2 amino acids shorter at the C-terminus. In some instances, any of the peptides disclosed in table 2 may be 1 or 2 amino acids shorter at the N-terminus. In some other instances, any of the peptides disclosed in table 2 may be 1 or 2 amino acids shorter at the C-terminus.

In some embodiments the peptides disclosed herein can include deletions, additions, and/or substitutions of amino acid residues, as discussed herein. Substituted amino acid residues can be unrelated to the amino acid residue being replaced (e.g., unrelated in terms or hydrophobicity/hydrophilicity, size, charge, polarity, etc.), or the substituted amino acid residues can constitute similar, conservative, or highly conservative amino acid substitutions. As used herein, “similar,” “conservative,” and “highly conservative” amino acid substitutions are defined as shown in Table 3, below. The determination of whether an amino acid residue substitution is similar, conservative, or highly conservative is based exclusively on the side chain of the amino acid residue and not the peptide backbone, which may be modified to increase peptide stability, as discussed below.

TABLE 3
Classification of Amino Acid Substitutions

| | | | Highly |
|---|---|---|---|
| | | | Conservative |
| Amino Acid | Similar | Conservative | Amino |
| in Subject | Amino Acid | Amino Acid | Acid |
| Polypeptide | Substitutions | Substitutions | Substitutions |
| Glycine (G) | A, S, N | A | n/a |
| Alanine (A) | S, G, T, V, C, P, Q | S, G, T | S |
| Serine (S) | T, A, N, G, Q | T, A, N | T, A |
| Threonine (T) | S, A, V, N, M | S, A, V, N | S |
| Cysteine (C) | A, S, T, V, I | A | n/a |
| Proline (P) | A, S, T, K | A | n/a |
| Methionine (M) | L, I, V, F | L, I, V | L, I |
| Valine (V) | I, L, M, T, A | I, L, M | I |
| Leucine (L) | M, I, V, F, T, A | M, I, V, F | M, I |
| Isoleucine (I) | V, L, M, F, T, C | V, L, M, F | V, L, M |
| Phenylalanine (F) | W, Y, L, M, I, V | W, L | n/a |
| Tyrosine (Y) | F, W, H, L, I | F, W | F |
| Tryptophan (W) | F, L, V | F | n/a |
| Asparagine (N) | Q | Q | Q |
| Glutamine (Q) | N | N | N |
| Aspartic Acid (D) | E | E | E |
| Glutamic Acid (E) | D | D | D |
| Histidine (H) | R, K | R, K | R, K |
| Lysine (K) | R, H, O | R, H, O | R, O |
| Arginine (R) | K, H, O | K, H, O | K, O |
| Ornithine (O) | R, H, K | R, H, K | K, R |

The “length” of a subject peptide or polypeptide is the number of amino acid residues linked end-to-end that constitute the peptide or polypeptide, excluding any non-peptide linkers and/or modifications that the peptide or polypeptide may contain. In some embodiments, the peptide is of 5 to 30 amino acid residues (e.g., 5 to 25, 10 to 20 or 5 to 18, 5 to 12 or 5 to 10, or 6 to 30, 6 to 25, 6 to 20, 6 to 18, 6 to 12, 6 to 10 or 7 to 12, or 7 to 10 amino acid residues) in length, and comprises a striapathic region of alternating hydrophilic and hydrophobic modules that adopts an amphipathic conformation under physiological conditions (e.g., as described herein). In some embodiments, the peptide is of 4 to 12 amino acid residues (e.g., 4, 5, 6, 7, 8, 9 or 10 amino acid residues) in length, and comprises a striapathic region of alternating hydrophilic and hydrophobic modules that adopts an amphipathic conformation under physiological conditions. In certain instances, a striapathic region of the peptide is of 5 to 18 amino acid residues in length (e.g., 6 to 18, 6 to 14, 6 to 12, 7 to 12, or 5, 6, 7, 8, 9, 10, 11 or 12 amino acids in length), wherein the peptide is optionally further modified (e.g., as described herein). The striapathic region can comprise: 2 or more (e.g., 3 or more or 4 or more) hydrophobic modules; and one or more (e.g., 2 or more, 3 or more, or 4 or more) hydrophilic modules (e.g., each comprising at least one cationic residue). In some instances, the striapathic region of the peptide has a length of 4 to 10 amino acid residues, such as 4 to 6. In some instances, the striapathic region of the peptide has a length of 2 to 3 amino acid residues.

The hydrophobic modules can consist of any convenient residues. In certain instances, the hydrophobic modules include amino acid residues selected from phenylalanine, tryptophan, alanine, valine, and glycine. The striapathic region can include 1, 2 or more cationic amino acid residues in total, such as 3 or more, 4 or more, 5 or more, 6 or more, or even more. The immunomodulatory peptide can comprise 2, 3 or more hydrophilic modules that consist of any convenient residues. In some instances, the hydrophilic modules include amino acid residues selected from lysine, arginine, histidine, aspartic acid, glutamic acid, asparagine and glutamine.

In the formula described herein, J(N) is used to refer to a particular hydrophobic module, where N is indicates a position within the linear formula. Similarly, X(N) is used to refer to a particular hydrophilic module, where N is indicates a position within the linear formula.

In the formula described herein, J₍ₙₓ₎ is used to refer to a particular hydrophobic amino acid residue, where n indicates which module the residue is located in and x indicates its position within the module. Similarly, X₍ₙₓ₎ is used to refer to a particular hydrophilic amino acid residue, where n indicates which module the residue is located in and x indicates its position within the module.

### Small Molecules

In certain embodiments of the present disclosure the CD206-binding agent is a small molecule. Small molecules of interest include, but are not limited to, small organic or inorganic compounds having a molecular weight (MW) of more than 50 and less than about 2,500 daltons (Da), such as more than 50 and less than about 1000 Da, or more than 50 and less than about 500 Da. “Small molecules” encompasses numerous biological and chemical classes, including synthetic, semi-synthetic, or naturally-occurring inorganic or organic molecules, including synthetic, recombinant or naturally-occurring nucleic acids. Small molecules of interest can comprise functional groups necessary for structural interaction with proteins, particularly hydrogen bonding, and can include at least an amine, carbonyl, hydroxyl or carboxyl group, and can contain at least two of the functional chemical groups. The small molecules can comprise cyclical carbon or heterocyclic structures and/or aromatic or polyaromatic structures substituted with one or more of the above functional groups. Small molecules are also found among biomolecules including saccharides, fatty acids, steroids, purines, pyrimidines, derivatives, structural analogs or combinations thereof.

Some molecules of interest can comprise a backbone comprising one or more carboxamide functional groups. In some instances, the small molecule of interest comprises a backbone comprising one or more urea functional groups. In some instances, the small molecule of interest comprises one or more carboxamide functional groups and one or more urea functional groups. In certain instances, the small molecule of interest includes one or more optionally substituted aryl groups. In certain instances, the small molecule of interest includes one or more optionally substituted naphthyl groups. In certain instances, the small molecule of interest includes one or more optionally substituted heterocyclic groups. In certain cases, the small molecule of interest includes one or more optionally substituted carbazole groups.

In some embodiments, the small molecule active agent is described by formula (I):

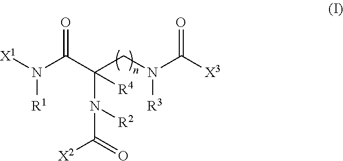

wherein:
R¹-R⁴ are each independently selected from hydrogen, alkyl and substituted alkyl;
X¹ is selected from alkyl, substituted alkyl, aryl, substituted aryl, aralkyl, substituted aralkyl, heterocycle, substituted heterocycle, heteroaryl, substituted heteroaryl;
X² is selected from alkyl, substituted alkyl, aryl, substituted aryl, amino, substituted amino, heteroaryl, substituted heteroaryl, heterocycle, substituted heterocycle;
X³ is selected from alkyl, substituted alkyl, aryl, substituted aryl, naphthyl, substituted naphthyl, heterocycle, substituted heterocycle, heteroaryl, substituted heteroaryl, aryl heterocycle, substituted aryl heterocycle; and
n is an integer from 1 to 10,
or a pharmaceutically acceptable salt or solvate thereof.

In certain embodiments of a compound of formula (I), X¹ is alkyl or substituted alkyl. In certain cases, X¹ is aryl or substituted aryl. In certain cases X¹ is selected from heterocycle, substituted heterocycle, heteroaryl, and substituted heteroaryl. In certain cases, X¹ is an optionally substituted carbazole. In certain cases, X¹ is an optionally substituted naphthyl. In certain cases, X¹ is phenol. In some cases, X¹ is phenyl. In certain cases, X¹ is aralkyl or substituted aralkyl. In certain cases, X¹ is an aralkyl including one or more aryl groups. In certain cases, the aralkyl includes a C₁-C₁₀ alkyl chain including one or more optionally substituted phenyl groups. In certain cases, X¹ is a C₁-C₁₀ alkyl chain including at least two optionally substituted phenyl groups. In certain cases, the alkyl chain terminates in at least two optionally substituted phenyl groups. In certain cases, the phenyl groups are unsubstituted. In certain cases the phenyl group is substituted with one or more groups selected from hydroxyl, amino, carboxamide, guanidine, acyl, halogen, alkyl, substituted alkyl, aryl, substituted aryl, heterocycle, substituted heterocycle, heteroaryl, substituted heteroaryl, nitrile and nitro. In certain cases X¹ is a C₁-C₆ alkyl chain substituted with two optionally substituted phenyl groups. In certain cases, the alkyl chain terminates in the two optionally substituted phenyl groups.

In certain embodiments of a compound of formula (I), X² is alkyl or substituted alkyl. In certain cases X² is C₁-C₆ alkyl. In certain cases, X² is methyl. In certain cases, X² is a C₁-C₆ alkyl group substituted by one or more groups. In certain cases the alkyl group is substituted with one or more groups selected from hydroxyl, amino, carboxamide, guanidine, acyl, halogen, alkyl, substituted alkyl, aryl, substituted aryl, heterocycle, substituted heterocycle, heteroaryl, substituted heteroaryl, nitrile and nitro. In certain cases, the alkyl group is substituted with a guanidine group. In certain cases, X² is aryl or substituted aryl. In certain cases X² is selected from heterocycle, substituted heterocycle, heteroaryl, and substituted heteroaryl. In certain cases, X² is an optionally substituted carbazole. In certain cases, X² is an optionally substituted naphthyl. In certain cases, X² is phenol. In some cases, X² is phenyl. In certain cases, X² is amino or substituted amino. In certain cases, X² is an amino group substituted by one or more aryl groups. In certain cases, the amino group is substituted by one or more optionally substituted phenyl groups. In certain cases, X² is an amino group substituted by one on more phenol groups.

In certain embodiments of a compound of formula (I), X³ is alkyl or substituted alkyl. In certain cases, X³ is aryl or substituted aryl. In certain cases, X³ is selected from heterocycle, substituted heterocycle, heteroaryl, and substituted heteroaryl. In certain cases, X³ is aryl heterocycle or substituted aryl heterocycle. In certain cases, X³ is an optionally substituted carbazole. In certain cases, X³ is an optionally substituted naphthyl. In certain cases, X³ is phenol. In some cases, X³ is phenyl. In certain cases the X³ group is substituted with one or more groups selected from hydroxyl, amino, carboxamide, guanidine, acyl, halogen, alkyl, substituted alkyl, aryl, substituted aryl, heterocycle, substituted heterocycle, heteroaryl, substituted heteroaryl, nitrile and nitro. In certain cases X³ is a carbazole substituted with one or two hydroxyl groups. In certain cases, X³ is an unsubstituted carbazole. In certain cases X³ is a naphthyl substituted with one or two hydroxyl groups. In certain cases, X³ is an unsubstituted naphthyl.

In certain embodiments of a compound of formula (I), n is an integer less than 10, such as 9 or less, 8 or less, 7 or less, 6 or less, 5 or less, 4 or less, 3 or less, or even less. In some instances, n is an integer from 1 to 6, such as 1-3 or 1-2. In certain cases, n is 1.

In some embodiments, the compound of formula (I), is described by the formula (Ia):

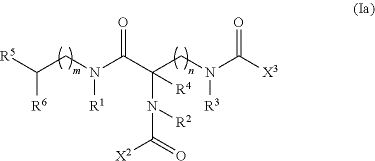

wherein:
R¹-R⁴ are each independently selected from hydrogen, and alkyl;
R⁵-R⁶ are each independently selected from aryl and substituted aryl;
X² is selected from alkyl, substituted alkyl, and NR^{2a}R^{2b}, where R^{2a} and R^{2b} are independently selected from hydrogen, aryl, and substituted aryl;
X³ is selected from aryl, substituted aryl, naphthyl, substituted naphthyl, carbazole, and substituted carbazole;
n is an integer from 1 to 6; and
m is an integer from 1 to 6.

In certain embodiments of formula (I) or (Ia), R¹-R⁴ are each hydrogen. In certain cases, at least one of R¹-R⁴ is alkyl. In certain cases, R¹ is alkyl and each of R¹-R³ is hydrogen. In certain cases, R² is alkyl and each of R¹, R³ and R⁴ are hydrogen. In certain cases, R³ is alkyl, and each of R¹, R² and R⁴ are hydrogen. In certain cases, R⁴ is alkyl, and each of R¹-R³ are hydrogen. In certain cases, R¹-R² are alkyl and R³-R⁴ are hydrogen. In certain cases, R¹ and R³ are alkyl and R² and R⁴ are hydrogen. In certain cases, R²-R³ are alkyl and R¹ and R⁴ are hydrogen. In certain cases, R³-R⁴ are alkyl and R¹-R² are hydrogen. In certain cases, R¹ is hydrogen and each of R¹-R³ is alkyl. In certain cases, R² is hydrogen and each of R¹, R³ and R⁴ are alkyl. In certain cases, R³ is hydrogen, and each of R¹, R² and R⁴ are alkyl. In certain cases, R⁴ is hydrogen, and each of R¹-R³ are alkyl. In certain cases, R¹-R⁴ are each alkyl. In certain cases, where at least one of R¹-R⁴ is alkyl, the alkyl is C₁-C₆ alkyl (e.g., methyl, ethyl, propyl, butyl, pentyl, or hexyl). In certain cases, where at least one of R¹-R⁴ is alkyl, the alkyl is methyl. In certain cases, where R⁴ is alkyl, the compound is enantiomerically pure, and carbon to which R⁴ is attached is of R-configuration. In certain cases, where R⁴ is alkyl, the compound is enantiomerically pure, and the carbon to which R⁴ is attached is of S-configuration. In certain cases, where R⁴ is alkyl, the compound is a racemic mixture.

In certain embodiments of formula (Ia), each of R⁵ and R⁶ are aryl. In certain cases each of R⁵ and R⁶ are phenyl. In certain cases, at least one of R⁵ or R^{e} is substituted aryl. In certain cases at least one of R⁵ or R⁶ is an aryl group substituted with one or more groups selected from hydroxyl, amino, carboxamide, guanidine, acyl, halogen, alkyl, substituted alkyl, aryl, substituted aryl, heterocycle, substituted heterocycle, heteroaryl, substituted heteroaryl, nitrile and nitro. In certain cases, both R⁵ and R⁶ are substituted aryl. In certain cases, both R⁵ and R⁶ are substituted phenyl. In some cases, both R⁵ and R⁶ are phenol.

In certain embodiments of a compound of formula (Ia), m is an integer less than 6, such as 5 or less, 4 or less, 3 or less, or even less. In some instances, n is an integer from 1 to 4, such as 1-3 or 1-2. In certain cases, n is 1.

In certain embodiments of formula (Ia), X² is alkyl or substituted alkyl. In certain cases X² is C₁-C₆ alkyl. In certain cases, X² is methyl. In certain cases, X² is a C₁-C₆ alkyl group substituted by one or more groups. In certain cases the alkyl group is substituted with one or more groups selected from hydroxyl, amino, carboxamide, guanidine, acyl, halogen, alkyl, substituted alkyl, aryl, substituted aryl, heterocycle, substituted heterocycle, heteroaryl, substituted heteroaryl, nitrile and nitro. In certain cases, the alkyl group is substituted with a guanidine group. In certain cases, X² is NR^{2a}R^{2b}, where R^{2a} and R^{2b}are independently selected from hydrogen, aryl, and substituted aryl. In certain cases both of R^{2a} and R^{2b} are hydrogen. In certain cases, both of R^{2a} and R^{2b} are aryl or substituted aryl. In certain cases, R^{2a} is an optionally substituted aryl group and R^{2b} is H. In certain cases the aryl group is substituted with one or more groups selected from hydroxyl, amino, carboxamide, guanidine, acyl, halogen, alkyl, substituted alkyl, aryl, substituted aryl, heterocycle, substituted heterocycle, heteroaryl, substituted heteroaryl, nitrile and nitro. In certain cases, R^{2a} phenyl and R^{2b} is H. In certain cases, R^{2a} is phenol and R^{2b} is H.

In certain embodiments of a compound of formula (Ia), X³ is aryl or substituted aryl. In certain cases, X³ is an optionally substituted carbazole. In certain cases, X³ is an optionally substituted naphthyl. In certain cases, X³ is phenol. In some cases, X³ is phenyl. In certain cases the X³ group is substituted with one or more groups selected from hydroxyl, amino, carboxamide, guanidine, acyl, halogen, alkyl, substituted alkyl, aryl, substituted aryl, heterocycle, substituted heterocycle, heteroaryl, substituted heteroaryl, nitrile and nitro. In certain cases X³ is a carbazole substituted with one or two hydroxyl groups. In certain cases, X³ is an unsubstituted carbazole. In certain cases X³ is a naphthyl substituted with one or two hydroxyl groups. In certain cases, X³ is an unsubstituted naphthyl.

In certain embodiments of a compound of formula (Ia), n is an integer less than 6, such as 5 or less, 4 or less, 3 or less, or even less. In some instances, n is an integer from 1 to 4, such as 1-3 or 1-2. In certain cases, n is 1.

In certain embodiments, the small molecule active agent is a compound selected from the group consisting of:

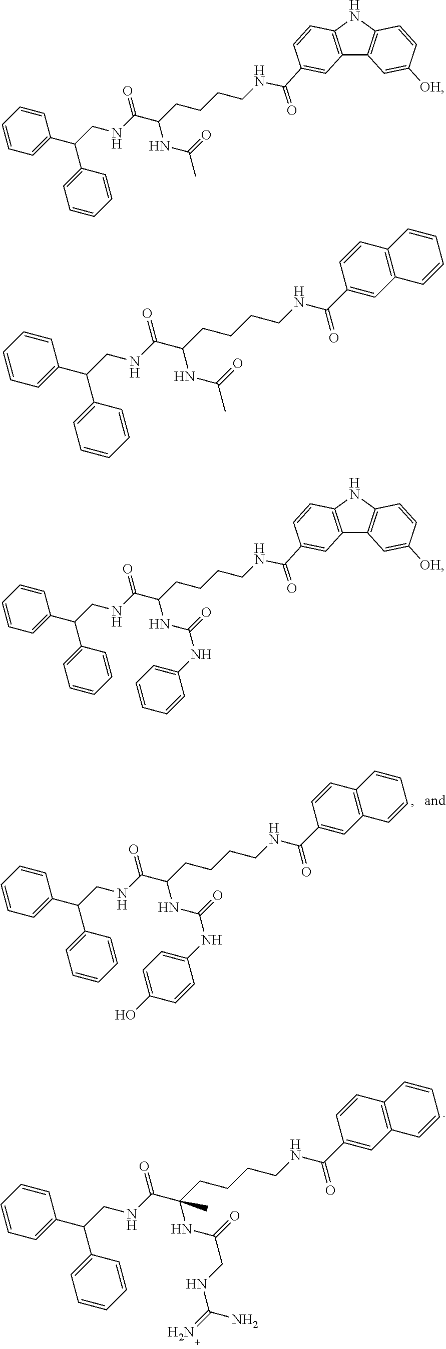 [SMILES: CC(=O)NC(CCCCNC(=O)c1ccc2[nH]c3ccc(O)cc3c2c1)C(=O)NCC(c1ccccc1)c1ccccc1 ; CC(=O)NC(CCCCNC(=O)c1ccc2ccccc2c1)C(=O)NCC(c1ccccc1)c1ccccc1 ; O=C(Nc1ccccc1)NC(CCCCNC(=O)c1ccc2[nH]c3ccc(O)cc3c2c1)C(=O)NCC(c1ccccc1)c1ccccc1 ; O=C(Nc1ccc(O)cc1)NC(CCCCNC(=O)c1ccc2ccccc2c1)C(=O)NCC(c1ccccc1)c1ccccc1 ; C[C@@](CCCCNC(=O)c1ccc2ccccc2c1)(NC(=O)CNC(=N)N)C(=O)NCC(c1ccccc1)c1ccccc1]

In certain other embodiments, the small molecule active agent is described by the formula (II):

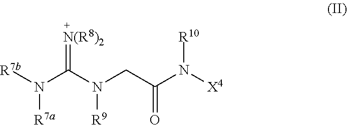

wherein:
R^{7a}, R^{7b}, R⁸, R⁹ and R¹⁰ are each independently selected from hydrogen, alkyl and substituted alkyl; and
X⁴ is selected from alkyl, aryl, aralkyl, heterocycle, and heteroaryl, acyl, wherein X⁴ is optionally further substituted with one or more groups selected from, alkyl, substituted alkyl, aryl, substituted aryl, amino, substituted amino, carboxamide, substituted carboxamide, heterocycle, substituted heterocycle, and a second compound of formula (II)
or a pharmaceutically acceptable salt or solvate thereof.

In certain embodiments of a compound of formula (II), X⁴ is optionally substituted alkyl. In certain cases, X⁴ is optionally substituted aryl. In certain cases X⁴ is selected from heterocycle, and heteroaryl, wherein either are optionally substituted. In certain cases, X⁴ is an optionally substituted aralkyl. In certain cases, X⁴ is an optionally substituted acyl. In certain cases, X⁴ is aralkyl or alkyl including one or more aryl groups. In certain cases, X⁴ is an aralkyl includes a C₁-C₂₀ alkyl chain including one or more optionally substituted phenyl groups. In certain cases, X⁴ is a C₁-C₂₀ alkyl chain including at least two optionally substituted phenyl groups. In certain cases, X⁴ is an alkyl chain that attaches to the N atom at a central point and terminates at each end of the alkyl chain in at least two optionally substituted phenyl groups. In certain cases, the phenyl groups are unsubstituted. In certain cases the phenyl group is substituted with one or more groups selected from hydroxyl, amino, carboxamide, guanidine, acyl, halogen, alkyl, substituted alkyl, aryl, substituted aryl, heterocycle, substituted heterocycle, heteroaryl, substituted heteroaryl, nitrile and nitro. In certain cases X⁴ is a C₁-C₂₀ alkyl chain substituted with two optionally substituted carboxamide groups. In certain cases, the alkyl chain attaches to the N atom at a central point and terminates at each end of the alkyl chain in the two optionally substituted carboxamide groups. In certain cases, the carboxamide groups are substituted with an aryl group. In certain cases, X⁴ is a C₁-C₂₀ alkyl chain substituted with two optionally substituted acyl groups. In certain cases, the alkyl chain attaches to the N atom at a central point and terminates at each end of the alkyl chain in the two optionally substituted acyl groups. In certain cases, the acyl groups are substituted with an aryl group. In certain cases, X⁴ is a C₁-C₂₀ alkyl chain substituted with at least one additional compound of formula (II). In some cases, the C₁₋₂₀ alkyl chain is further substituted with optionally substituted aryl groups. In some cases, X⁴ is an optionally substituted acyl group. In some cases, the acyl group is substituted with a substituent that includes at least one additional compound of formula (II). In some instances the acyl group is substituted with a substituent including a heterocyclic group.

In certain embodiments of the compound of formula (II), X⁴ comprises a chelating group. In certain cases, the chelating group is a heterocyclic compound capable of coordinating a metal (e.g., iron) via at least two heteroatoms in the chelator. In certain cases the chelating group may be selected from any of 1,4,7,10-tetraazacyclododecane-1,4,7,10-tetraacetic acid (DOTA), triethylenetetramine (TETA), 1,4,7-triazacyclononane-1,4,7-triacetic acid (NOTA), 1,4,7-triazacyclononane-1,4-diacetic acid (NODA), (tert-Butyl)₂NODA, NETA, C-NETA, L-NETA, S-NETA, NODA-MPAA, and NODA-MPAEM. In certain cases, X⁴ includes a chelator derived by 1,4,7,10-tetraazacyclododecane-1,4,7,10-tetraacetic acid (also known as DOTA, or tetraxetan) and can be attached to the compound of formula (II) via adaption of one of the pending acetic acid groups. In certain cases, X⁴ includes a chelator derived from DOTA and is attached to 1,2, 3 or 4 compounds of formula (II) via adaption of the pending acetic acid groups.

In certain embodiments, the compound of formula (II) is described by the formula (IIa):

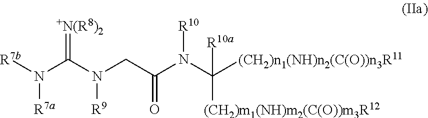

wherein:
R^{7a}, R^{7b}, R⁸, R⁹, R¹⁰ and R^{10a} are each independently selected from hydrogen, and alkyl;
R¹¹ and R¹² are each independently selected from aryl, substituted aryl, heteroaryl, substituted heteroaryl, naphthyl, substituted naphthyl, carbazole, and substituted carbazole;
n₁ and m₁ are each independently an integer from 1 to 10;
n₂ and m₂ are each independently 0 or 1; and
n₃ and m₃ are each independently 0 or 1.

In certain embodiments of the compound of formula (II) or (IIa), R^{7a}, R^{7b}, R⁸, R⁹, R¹⁰ and R^{10a} are each hydrogen. In certain cases, at least one of R^{7a}, R^{7b}, R⁸, R⁹, R¹⁰ and R^{10a} is alkyl. In certain cases, R¹⁰ is alkyl and each of R^{7a}, R^{7b}, R⁸, R⁹ and R^{10a} are hydrogen. In certain cases, R⁹ is alkyl and each of R^{7a}, R^{7b}, R⁸, R¹⁰ and R^{10a} are hydrogen. In certain cases, R^{7a} is alkyl, and each of R⁹, R^{7b}, R⁸, R¹⁰ and R^{10a} are hydrogen. In certain cases, at least one of R⁸ is alkyl, and each of R^{7a}, R^{7b}, R⁹, R¹⁰ and R^{10a} are hydrogen. In certain cases, at least one of R^{10a} is alkyl, and each of R^{7a}, R^{7b}, R⁹, R¹⁰ and R^{10a} are hydrogen. In certain cases, where at least one of R^{7a}, R^{7b}, R⁸, R⁹, R¹⁰ and R^{10a} is alkyl, the alkyl is methyl. In certain cases, where R^{10a} is alkyl, the compound is enantiomerically pure, and carbon to which R^{10a} is attached is of R-configuration. In certain cases, where R^{10a} is alkyl, the compound is enantiomerically pure, and the carbon to which R^{10a} is attached is of S-configuration. In certain cases, where R^{10a} is alkyl, the compound is a racemic mixture.

In certain embodiments of the compound of formula (IIa), n₁ and m₁ are each independently at integer from 1 to 8, such as 1-7, 1-6 or 1-5. In some cases each of n₁ and m₁ are 10 or less, such as 9, 8, 7, 6, 5, 4, or less. In some cases each of n₁ and m₁ are 4-8, such as 5-7, such as 5-6. In some cases, n₁ and m₁ are both 5. In certain cases of formula (IIa), n₂ and m₂ are each 0. In some cases, at least one of n₂ and m₂ is 1. In some cases, each of n₂ and m₂ are 1. In some cases of formula (IIa), n₃ and m₃ are each 0. In some cases, at least one of n₃ and m₃ is 1. In some cases each of n₃ and m₃ are 1. In some cases, each of n₁ and m₁ is an integer from 1 to 10, each of n₂ and m₂ is 0; and each of n₃ and m₃ is 0. In some cases, each of n₁ and m₁ is an integer from 1 to 10, each of n₂ and m₂ is 1; and each of n₃ and m₃ is 0. In some cases, each of n₁ and m₁ is an integer from 1 to 10, each of n₂ and m₂ is 0; and each of n₃ and m₃ is 1. In some cases, each of n₁ and m₁ is an integer from 1 to 10, each of n₂ and m₂ is 1; and each of n₃ and m₃ is 1.

In certain embodiments of the compound formula (IIa), R¹¹ and R¹² are each independently selected from aryl, or substituted aryl, In certain cases, R¹¹ and R¹² are each independently selected from heteroaryl, and substituted heteroaryl. In certain cases, at least one of R¹¹ and R¹² is an optionally substituted carbazole. In certain cases, at least one of R¹¹ and R¹² is an optionally substituted naphthyl. In certain cases, at least one of R¹¹ and R¹² is phenol. In some cases, at least one of R¹¹ and R¹² is phenyl. In some cases, each of R¹¹ and R¹² are phenyl. In certain cases R¹¹ and R¹² are each independently substituted with one or more groups selected from hydroxyl, amino, carboxamide, guanidine, acyl, halogen, alkyl, substituted alkyl, aryl, substituted aryl, heterocycle, substituted heterocycle, heteroaryl, substituted heteroaryl, nitrile and nitro. In certain cases at least one of R¹¹ and R¹² is a carbazole substituted with one or two hydroxyl groups. In certain cases, at least one of R¹¹ and R¹² is an unsubstituted carbazole. In certain cases at least one of R¹¹ and R¹² is a naphthyl substituted with one or two hydroxyl groups. In certain cases, at least one of R¹¹ and R¹² is an unsubstituted naphthyl.

In certain embodiments, the small molecule active agent is a compound selected from the group consisting of:

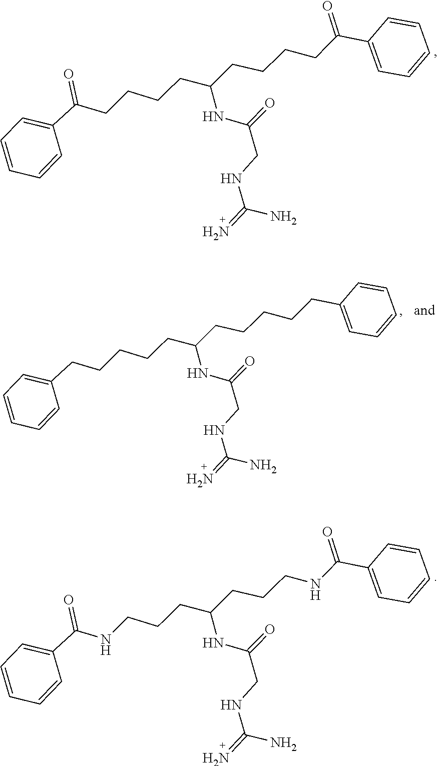 [SMILES: N=C(N)NCC(=O)NC(CCCCC(=O)c1ccccc1)CCCCC(=O)c1ccccc1 ; N=C(N)NCC(=O)NC(CCCCCc1ccccc1)CCCCCc1ccccc1 ; N=C(N)NCC(=O)NC(CCCNC(=O)c1ccccc1)CCCNC(=O)c1ccccc1]

In certain embodiments, the small molecule active agent is a compound selected from the group consisting of

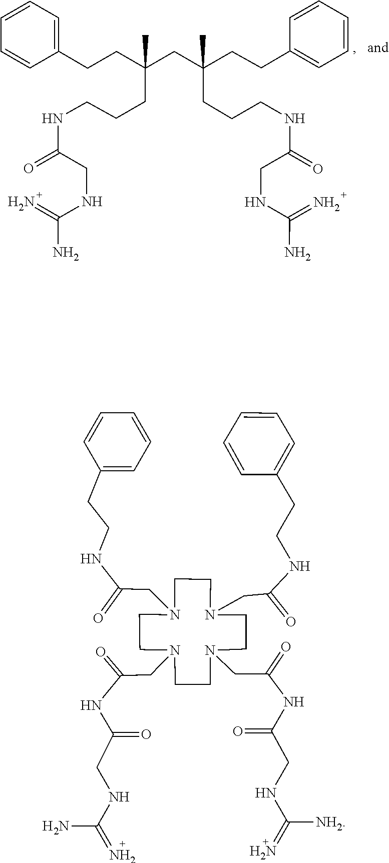 [SMILES: C[C@](CCCNC(=O)CNC(=N)N)(CCc1ccccc1)C[C@](C)(CCCNC(=O)CNC(=N)N)CCc1ccccc1 ; N=C(N)NCC(=O)NC(=O)CN1CCN(CC(=O)NCCCC2=CC=CCC=C2)CCN(CC(=O)NCCc2ccccc2)CCN(CC(=O)NC(=O)CNC(=N)N)CC1]

In certain other embodiments, the small molecule active agent is described by the formula (III):

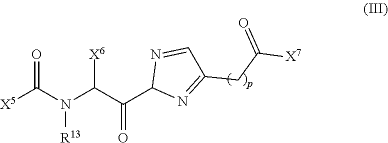

In certain embodiments of the compound of formula (III), R¹³ is hydrogen. In other instances, R¹³ is alkyl or substituted alkyl, such as C₁-C₆ alkyl. In some instances, R¹³ is methyl.

In certain embodiments of a compound of formula (III), X⁵ is alkyl or substituted alkyl. In certain cases, X⁵ is aryl or substituted aryl. In certain cases, X⁵ is selected from heterocycle, substituted heterocycle, heteroaryl, and substituted heteroaryl. In certain cases, X⁵ is an optionally substituted carbazole. In certain cases, X⁵ is an optionally substituted naphthyl. In certain cases, X⁵ is phenol. In some cases, X⁵ is phenyl. In certain cases, X⁵ is amino or substituted amino. In certain cases, X⁵ is an amino group substituted by one or more aryl groups. In certain cases, the amino group is substituted by one or more optionally substituted phenyl groups. In certain cases, X⁵ is an amino group substituted by one on more phenol groups. In certain cases the X⁵ group is substituted with one or more groups selected from hydroxyl, amino, carboxamide, guanidine, acyl, halogen, alkyl, substituted alkyl, aryl, substituted aryl, heterocycle, substituted heterocycle, heteroaryl, substituted heteroaryl, nitrile and nitro.

In certain embodiments of a compound of formula (III), X⁶ is alkyl or substituted alkyl. In certain cases X⁶ is C₁-C₆ alkyl. In certain cases, X⁶ is methyl. In certain cases, X⁶ is a C₁-C₆ alkyl group substituted by one or more groups. In certain cases the alkyl group is substituted with one or more groups selected from hydroxyl, amino, carboxamide, guanidine, acyl, halogen, alkyl, substituted alkyl, aryl, substituted aryl, heterocycle, substituted heterocycle, heteroaryl, substituted heteroaryl, nitrile and nitro. In certain cases, the alkyl group is substituted with a guanidine group. In certain cases, X⁶ is aryl or substituted aryl. In certain cases X⁶ is selected from heterocycle, substituted heterocycle, heteroaryl, and substituted heteroaryl. In certain cases, X⁶ is an optionally substituted carbazole. In certain cases, X⁶ is an optionally substituted naphthyl. In certain cases, X⁶ is phenol. In some cases, X⁶ is phenyl. In certain cases, X⁶ is aralkyl or substituted aralkyl. In certain cases, X⁶ is an aralkyl including one or more aryl groups. In certain cases, the aralkyl includes a C₁-C₁₀ alkyl chain including one or more optionally substituted phenyl groups. In certain cases, X⁶ is a C₁-C₁₀ alkyl chain including at least one optionally substituted phenyl groups. In certain cases, the alkyl chain terminates in at least one optionally substituted phenyl groups. In certain cases, the phenyl group is unsubstituted. In certain cases the phenyl group is substituted with one or more groups selected from hydroxyl, amino, carboxamide, guanidine, acyl, halogen, alkyl, substituted alkyl, aryl, substituted aryl, heterocycle, substituted heterocycle, heteroaryl, substituted heteroaryl, nitrile and nitro.

In certain embodiments of a compound of formula (I), X⁷ is alkyl or substituted alkyl. In certain cases, X⁷ is aryl or substituted aryl. In certain cases, X⁷ is selected from heterocycle, substituted heterocycle, heteroaryl, and substituted heteroaryl. In certain cases, X⁷ is aryl heterocycle or substituted aryl heterocycle. In certain cases, X⁷ is an optionally substituted carbazole. In certain cases, X⁷ is an optionally substituted naphthyl. In certain cases, X⁷ is phenol. In some cases, X⁷ is phenyl. In certain cases the X⁷ group is substituted with one or more groups selected from hydroxyl, amino, carboxamide, guanidine, acyl, halogen, alkyl, substituted alkyl, aryl, substituted aryl, heterocycle, substituted heterocycle, heteroaryl, substituted heteroaryl, nitrile and nitro. In certain cases X⁷ is a carbazole substituted with one or two hydroxyl groups. In certain cases, X⁷ is an unsubstituted carbazole. In certain cases X⁷ is a naphthyl substituted with one or two hydroxyl groups. In certain cases, X⁷ is an unsubstituted naphthyl.

In certain embodiments of a compound of formula (III), p is an integer less than 10, such as 9 or less, 8 or less, 7 or less, 6 or less, 5 or less, 4 or less, 3 or less, or even less. In some instances, p is an integer from 1 to 6, such as 1-3 or 1-2. In certain cases, p is 1.

In certain embodiments, the small molecule active agent is the following compound:

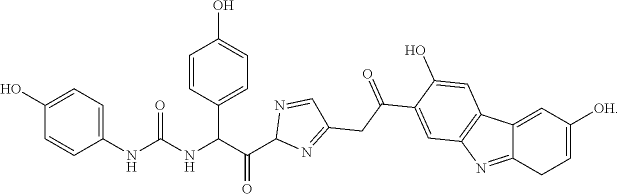 [SMILES: O=C(Nc1ccc(O)cc1)NC(C(=O)C1N=CC(CC(=O)c2cc3c(cc2O)C2=CC(O)=CCC2=N3)=N1)c1ccc(O)cc1]

It is understood that any of the compounds disclosed herein may be present in a salt form. In some cases, the salt form of the compound is a pharmaceutically acceptable salt. It is understood that any of the compounds disclosed herein may be present in a prodrug form.

Aspects of the present disclosure include small molecule active agents (e.g., as described herein), salts thereof (e.g., pharmaceutically acceptable salts), and/or solvate, hydrate and/or prodrug forms thereof. In addition, it is understood that, in any compound described herein having one or more chiral centers, if an absolute stereochemistry is not expressly indicated, then each center may independently be of R-configuration or S-configuration or a mixture thereof. It will be appreciated that all permutations of salts, solvates, hydrates, prodrugs and stereoisomers are meant to be encompassed by the present disclosure.

In some embodiments, the subject small molecule active agent, or a prodrug form thereof, are provided in the form of pharmaceutically acceptable salts. Compounds containing an amine or nitrogen containing heteroaryl group may be basic in nature and accordingly may react with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

In some embodiments, the subject compounds are provided in a prodrug form. “Prodrug” refers to a derivative of an active agent that requires a transformation within the body to release the active agent. In certain embodiments, the transformation is an enzymatic transformation. Prodrugs are frequently, although not necessarily, pharmacologically inactive until converted to the active agent. “Promoiety” refers to a form of protecting group that, when used to mask a functional group within an active agent, converts the active agent into a prodrug. In some cases, the promoiety will be attached to the drug via bond(s) that are cleaved by enzymatic or non-enzymatic means in vivo. Any convenient prodrug forms of the subject compounds can be prepared, e.g., according to the strategies and methods described by Rautio et al. (“Prodrugs: design and clinical applications”, Nature Reviews Drug Discovery 7, 255-270 (February 2008)). In some cases, the promoiety is attached to a hydroxy group of the subject compounds. In certain cases, the promoiety is an acyl or substituted acyl group. In certain cases, the promoiety is an alkyl or substituted alkyl group, e.g., that forms an ester functional group when attached to a hydroxyl functional group of the subject compounds.

In some embodiments, the subject small molecule active agents, prodrugs, stereoisomers or salts thereof are provided in the form of a solvate (e.g., a hydrate). The term “solvate” as used herein refers to a complex or aggregate formed by one or more molecules of a solute, e.g. a prodrug or a pharmaceutically-acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

In some embodiments, the small molecule active agents are provided by oral dosing and absorbed into the bloodstream. In some embodiments, the oral bioavailability of the subject compounds is 30% or more. Modifications may be made to the subject compounds or their formulations using any convenient methods to increase absorption across the gut lumen or their bioavailability.

In some embodiments, the subject compounds are metabolically stable (e.g., remain substantially intact in vivo during the half-life of the compound). In certain embodiments, the compounds have a half-life (e.g., an in vivo half-life) of 5 minutes or more, such as 10 minutes or more, 12 minutes or more, 15 minutes or more, 20 minutes or more, 30 minutes or more, 60 minutes or more, 2 hours or more, 6 hours or more, 12 hours or more, 24 hours or more, or even more.

### Specific Binding Member

In certain embodiments of the present disclosure the CD206-binding agent is a specific binding member. The term “specific binding member” refers to one member of a pair of molecules which have binding specificity for one another. One member of the pair of molecules may have an area on its surface, or a cavity, which specifically binds to an area on the surface of, or a cavity in, the other member of the pair of molecules. Thus the members of the pair have the property of binding specifically to each other to produce a binding complex. In some embodiments, the affinity between specific binding members in a binding complex is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less. In some embodiments, the specific binding members specifically bind with high avidity. By high avidity is meant that the binding member specifically binds with an apparent affinity characterized by an apparent K_{d} of 10×10⁻⁹ M or less, such as 1×10⁻⁹ M or less, 3×10⁻¹⁰ M or less, 1×10⁻¹⁰ M or less, 3×10⁻¹¹ M or less, 1×10⁻¹¹ M or less, 3×10⁻¹² M or less or 1×10⁻¹² M or less.

In some embodiments, the specific binding member is proteinaceous (e.g., composed of amino acid residues). In certain cases, the proteinaceous specific binding member is an antibody. In certain embodiments, the proteinaceous specific binding member is an antibody fragment, e.g., a binding fragment of an antibody that specific binds to a an activity modulating domain of CD206. As used herein, the terms “antibody” and “antibody molecule” are used interchangeably and refer to a protein consisting of one or more polypeptides substantially encoded by all or part of the recognized immunoglobulin genes. The recognized immunoglobulin genes, for example in humans, include the kappa (k), lambda (I), and heavy chain genetic loci, which together comprise the myriad variable region genes, and the constant region genes mu (u), delta (d), gamma (g), sigma (e), and alpha (a) which encode the IgM, IgD, IgG, IgE, and IgA isotypes respectively. An immunoglobulin light or heavy chain variable region consists of a “framework” region (FR) interrupted by three hypervariable regions, also called “complementarity determining regions” or “CDRs”. The extent of the framework region and CDRs have been precisely defined (see, “Sequences of Proteins of Immunological Interest,” E. Kabat et al., U.S. Department of Health and Human Services, (1991)). The numbering of all antibody amino acid sequences discussed herein conforms to the Kabat system. The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

The term antibody is meant to include full length antibodies and may refer to a natural antibody from any organism, an engineered antibody, or an antibody generated recombinantly for experimental, therapeutic, or other purposes as further defined herein. Antibody fragments of interest include, but are not limited to, Fab, Fab′, F(ab′)2, Fv, scFv, or other antigen-binding subsequences of antibodies, either produced by the modification of whole antibodies or those synthesized de novo using recombinant DNA technologies. Antibodies may be monoclonal or polyclonal and may have other specific activities on cells (e.g., antagonists, agonists, neutralizing, inhibitory, or stimulatory antibodies). It is understood that the antibodies may have additional conservative amino acid substitutions which have substantially no effect on antigen binding or other antibody functions.

In certain embodiments, the specific binding member is an antibody. In certain embodiments, the specific binding member is a Fab fragment, a F(ab′)2 fragment, a scFv, a diabody or a triabody. In some cases, the specific binding member is a murine antibody or binding fragment thereof. In certain instances, the specific binding member is a recombinant antibody or binding fragment thereof.

In certain embodiments the specific binding member is an antibody, or a binding fragment thereof, that targets a sequence of CD206. In certain cases, the specific binding member targets a sequence of CD206 selected from the group consisting of NFGDLVSIQSESEKK, NDAQSAYFIGLLISL, SKEKETMDNARAF, and EDENCVTMYSNSGFWN. In some cases, the antibody or fragment thereof, targets a NFGDLVSIQSESEKK sequence of CD206. In some cases, the antibody, or binding fragment thereof, targets a NDAQSAYFIGLLISL sequence of CD206. In some cases, the antibody, or binding fragment thereof, targets a SKEKETMDNARAF sequence of CD206. In some cases, the antibody, or binding fragment thereof targets a EDENCVTMYSNSGFWN sequence of CD206.

Antibodies that can bind to an activity modulating domain of CD206 in connection with the present disclosure can encompass, but are not limited to, monoclonal antibodies, polyclonal antibodies, bispecific antibodies, Fab antibody fragments, F(ab)₂ antibody fragments, Fv antibody fragments (e.g., V_{H} or V_{L}), single chain Fv antibody fragments and dsFv antibody fragments. Furthermore, the antibody molecules can be fully human antibodies, humanized antibodies, or chimeric antibodies. The antibodies that can be used in connection with the present disclosure can include any antibody variable region, mature or unprocessed, linked to any immunoglobulin constant region. Minor variations in the amino acid sequences of antibodies or immunoglobulin molecules are encompassed by the present disclosure, providing that the variations in the amino acid sequence maintain 75% or more, e.g., 80% or more, 90% or more, 95% or more, or 99% or more of the sequence.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CHI) of the heavy chain. Fab fragments differ from Fab′ fragments by the addition of a few residues at the carboxyl terminus of the heavy chain CHI domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

Antibodies that can be used in connection with the present disclosure thus can encompass monoclonal antibodies, polyclonal antibodies, bispecific antibodies, Fab antibody fragments, F(ab)2 antibody fragments, Fv antibody fragments (e.g., VH or VL), single chain Fv antibody fragments and dsFv antibody fragments. Furthermore, the antibody molecules can be fully human antibodies, humanized antibodies, or chimeric antibodies. In some embodiments, the antibody molecules are monoclonal, fully human antibodies.

The antibodies that can be used in connection with the present disclosure can include any antibody variable region, mature or unprocessed, linked to any immunoglobulin constant region. If a light chain variable region is linked to a constant region, it can be a kappa chain constant region. If a heavy chain variable region is linked to a constant region, it can be a human gamma 1, gamma 2, gamma 3 or gamma 4 constant region, more preferably, gamma 1, gamma 2 or gamma 4 and even more preferably gamma 1 or gamma 4.

Minor variations in the amino acid sequences of antibodies or immunoglobulin molecules are encompassed by the present invention, providing that the variations in the amino acid sequence maintain at least 75%, e.g., at least 80%, 90%, 95%, or 99% of the sequence. In particular, conservative amino acid replacements are contemplated (e.g., as described herein). Whether an amino acid change results in a functional peptide can readily be determined by assaying the specific activity of the polypeptide derivative. Fragments (or analogs) of antibodies or immunoglobulin molecules, can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Sequence motifs and structural conformations can be used to define structural and functional domains in accordance with the invention.

Non-limiting examples of antibodies which may find use in the present disclosure include Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Namatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sofituzumab, Catumaxomab, Ertumaxomab, Depatuxizumab, Ontuxizumab, Blontuvetmab, Tamtuvetmab, or an antigen-binding variant thereof. As used herein, the term “variant” refers to an antibody that binds to a particular cognate antigen but has fewer or more amino acids than the parental antibody, has one or more amino acid substitutions relative to the parental antibody, is a single-chain variant (such as an scFv variant) of the parental antibody, or any combination thereof.

In certain embodiments, the specific binding member is an aptamer, a nucleic acid (e.g., a DNA, a RNA, or a nucleic acid analog).

In certain embodiments the specific binding member is an aptamer or a nucleic acid (e.g., a DNA, a RNA, or a nucleic acid analog), that targets a sequence of CD206. In certain cases, the specific binding member targets a sequence of CD206 selected from the group consisting of NFGDLVSIQSESEKK, NDAQSAYFIGLLISL, SKEKETMDNARAF, and EDENCVTMYSNSGFWN.

In some cases, the an aptamer or a nucleic acid (e.g., a DNA, a RNA, or a nucleic acid analog), targets a NFGDLVSIQSESEKK sequence of CD206. In some cases, the an aptamer or a nucleic acid (e.g., a DNA, a RNA, or a nucleic acid analog), targets a NDAQSAYFIGLLISL sequence of CD206. In some cases, the an aptamer or a nucleic acid (e.g., a DNA, a RNA, or a nucleic acid analog), targets a SKEKETMDNARAF sequence of CD206. In some cases, the an aptamer or a nucleic acid (e.g., a DNA, a RNA, or a nucleic acid analog) targets a EDENCVTMYSNSGFWN sequence of CD206.

### Conjugate Compounds

In certain embodiments of the present disclosure, the CD206-binding agent is conjugated to one or more other active agent compounds (such as for example one or more active agents described above for combination therapy). In some instances, the CD206-binding agent may be conjugated to two or more other active agent compounds, such as 3 or more and including 5 or more. The CD206-binding agent may be conjugated to the one or more active agents such as by hydrogen bonding or ionic interactions. In other embodiments, the CD206-binding agent is conjugated to the one or more active agents with one or more covalent bonds. The CD206-binding agent may be directly bonded to the active agent or may be bonded to the active agent through one or more linkers, where in certain instances, the CD206-binding agent and the active agent are bonded by linking chemistry that includes but is not limited to, maleimide/thiol, succimidylester (NHS ester)/amine, azide chemistry, carboxy/EDC (1-Ethyl-3-[3-dimethylaminopropyl]carbodiimide Hydrochloride)/amine, amine/Sulfo-SMCC (Sulfosuccinimidyl 4-[N-maleimidomethyl]cyclohexane-1-carboxylate)/thiol, and amine/BMPH (N-[ß-Maleimidopropionic acid]hydrazide·TFA)/thiol.

### Screening Methods

Aspects of the present disclosure also include assays configured to identify agents that find use in methods of the invention, e.g., as reviewed above. Aspects of the present disclosure include methods for identifying a candidate agent for the ability to bind to an activity modulating domain of CD206. In some instances, the method comprises: contacting a macrophage comprising CD206 with a compound; and determining if the compound binds to an activity modulating domain of CD206. In some cases, the method further includes determining the activity modulating domain of CD206 that binds to the compound. By assessing or determining is meant at least predicting that a given test compound will have a desirable binding, such that further testing of the compound in additional assays, such as animal model and/or clinical assays, may be desired.

In certain cases, the macrophage is a macrophage comprising one or more mutations in the activity modulating domains of CD206. In certain cases, the activity modulating domain of CD206 is selected from fibronectin II domain of CD206, C-type lectin carbohydrate recognition domain 3 (CRD3) of CD206, C-type lectin carbohydrate recognition domain 4 (CRD4) of CD206 and C-type lectin carbohydrate recognition domain 5 (CRD5) of CD206. In certain cases, the compound binds to the CRD5 activity modulating domain of CD206. In some cases, the compound binds to the fibronectin II activity modulating domain of CD206. In some cases, the compound binds to the CRD3 activity modulating domain of CD206.

The candidate compound can be: an immunomodulatory peptide, a small molecule, or a specific binding member (e.g., an antibody) as described herein. In some instances, the determining step comprises detecting a cellular parameter, wherein a change in the parameter in the cell as compared to in a cell not contacted with candidate compound indicates that the candidate compound specifically binds the activity modulating domain of CD206.

Compound screening may be performed using an in vitro model, a genetically altered cell a microorganism, or purified CD206 protein. One can identify ligands that compete with, modulate or mimic the action of a lead agent. Screening identifies compounds that bind to particular domains of a CD206 motif. A wide variety of assays may be used for this purpose, including labeled in vitro binding assays, electrophoretic mobility shift assays, immunoassays for protein binding, and the like. Knowledge of the 3-dimensional structure of CD206, and experimental data provided herein, can also lead to the rational design of compounds that specifically bind to an activity modulating domain of CD206.

The term “compound” as used herein describes any molecule, e.g., immunomodulatory peptide, small molecule, specific binding member (e.g., antibody or fragment thereof), with the capability of binding an activity modulating domain of CD206. Generally, a plurality of assay mixtures are run in parallel with different agent concentrations to obtain a differential response to the various concentrations. Typically one of these concentrations serves as a negative control, i.e., at zero concentration or below the level of detection.

Candidate compounds encompass numerous chemical classes, such as oligonucleotides, antibodies, peptides, polypeptides, and organic molecules, e.g., small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate small molecule compounds often comprise cyclical carbon or heterocyclic structures and/or aromatic or polyaromatic structures substituted with one or more of the above functional groups. Candidate compounds are also found among biomolecules including peptides, saccharides, fatty acids, steroids, purines, pyrimidines, derivatives, structural analogs or combinations thereof.

Candidate compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Of interest in certain embodiments are compounds that pass the blood-brain barrier.

Where the screening assay is a binding assay, one or more of the molecules may be joined to a member of a signal producing system, e.g., a label, where the label can directly or indirectly provide a detectable signal. Various labels include, but are not limited to: radioisotopes, fluorescers, chemiluminescers, enzymes, specific binding molecules, particles, e.g., magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc. that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components is added in any order that provides for the requisite binding. Incubations are performed at any suitable temperature, typically between 4 and 40° C. Incubation periods are selected for optimum activity, but may also be optimized to facilitate rapid high-throughput screening. In some cases, between 1 and 48 hours will be sufficient.

In some embodiments, the screening step is performed at about 1 to about 1000 micromolar concentration of the compounds, such as about 10 to about 500 micromolar or about 10 to about 100 micromolar concentration. In some cases, a dose response curve is assessed for each of the compounds. In certain cases, the compounds are assessed for binding at a single concentration.

### Compositions

In embodiments, compositions for use in treating a subject according to the present disclosure can be formulated according to any of the conventional methods known in the art and widely described in the literature. Thus, the active ingredient (e.g., CD206-binding agent as described herein) may be incorporated, optionally together with other active substances, with one or more conventional pharmaceutically acceptable carriers, diluents and/or excipients, etc., appropriate for the particular use of the composition, to produce conventional preparations that are suitable or can be made suitable for administration. They may be formulated as liquids, as semi-solids or solids, liquid solutions, dispersions, suspensions, and the like, depending on the intended mode of administration and therapeutic application. In some embodiments, the inventive composition is prepared in a form of an injectable or infusible solution.

In certain embodiments, the CD206-binding agent composition may include a carrier protein, such as serum albumin (e.g., HSA, BSA, and the like). The serum albumin can be purified or recombinantly produced. By mixing the CD206-binding agent in the pharmaceutical composition with serum albumin, the CD206-binding agent can be effectively “loaded” onto the serum albumin, allowing a greater amount of CD206-binding agent to be successfully.

In certain embodiments of the inventive treatment methods, administration is via any one of a variety of routes, including intravenous (IV), intramuscular (IM), intra-arterial, intramedullary, intrathecal, subcutaneous (SQ), intraventricular, transdermal, interdermal, intradermal, intratumoral, by intratracheal instillation, bronchial instillation, and/or inhalation; as a nasal spray, and/or aerosol, and/or through a portal vein catheter. In certain embodiments, intravenous injection, or infusion may be used. Any appropriate site of administration may be used. For example, the inventive composition may be administered locally and directly at the site where action is required or may be attached or otherwise associated, e.g. conjugated, with entities which will facilitate the targeting to an appropriate location in the body.

In certain embodiments, any physiologically compatible carrier, excipient, diluent, buffer or stabilizer can be used in the compositions of the invention. Examples of suitable carriers, excipients, diluents, buffers and stabilizers include one or more of water, saline, phosphate buffered saline, dextrose, glycerol, ethanol and the like, as well as combinations thereof. In some cases isotonic agents, e.g., sugars, polyalcohols (e.g., mannitol, sorbitol), or sodium chloride may be included. In certain embodiments, the compositions of the invention may be formulated so as to provide quick, sustained, or delayed release of the active ingredient (peptide A, peptide B, or variants thereof and/or additional drug(s)) after administration to the subject by employing procedures well known in the art. As described above, in certain embodiments, the composition is in a form suitable for injection and suitable carriers may be present at any appropriate concentration, but exemplary concentrations are from 1% to 20% or from 5% to 10%.

Therapeutic compositions typically must be sterile and stable under conditions of manufacture and storage. Appropriate ways of achieving such sterility and stability are well known and described in the art.

Pharmaceutical compositions are typically formulated in unit dosage form for ease of administration and uniformity of dosage. It will be understood, however, that the total daily (or other) usage of the compositions of the present invention will be decided by the attending physician within the scope of sound medical judgment. The specific therapeutically effective dosage level for any particular subject will depend upon a variety of factors including the activity of the composition employed; the half-life of the composition after administration; the age, body weight, general health, sex, and diet of the subject; the time of administration, route of administration, and rate of excretion of peptide A and (if used) the additional therapeutic agent employed; the duration of the treatment; drugs used in combination or coincidental with the specific compound employed; and like factors, well known in the medical arts. Furthermore, effective doses may be extrapolated from dose-response curves derived from in vitro and/or in vivo animal models.

Thus, suitable doses of the CD206-binding agent and other active ingredients (if included) will vary from patient to patient. In some embodiments, said dosages constitute a therapeutically effective amount or a prophylactically effective amount, depending on the nature of the treatment involved. The ability of the CD206-binding agent to elicit a desired response in the individual will also be a factor. Exemplary daily doses are: 0.1 to 250 mg/kg, or 0.1 to 200 or 100 mg/kg, or 0.5 to 100 mg/kg, or 1 to 50 or 1 to 10 mg/kg, of the active ingredient. This can be administered as a single unit dose or as multiple unit doses administered more than once a day, for example, subcutaneously, intraperitoneally, or intravenously. It is to be noted, however, that appropriate dosages may vary depending on the patient, and that for any particular subject, specific dosage regimes should be adjusted over time according to the individual needs of the patient. Thus, the dosage ranges set forth herein are to be regarded as exemplary and are not intended to limit the scope or practice of the claimed compositions or methods.

### Kits

In one aspect, the present disclosure further provides kits the CD206-binding agent, or a composition formulated with the CD206-binding agent. Kits can include one or more other elements including, but not limited to, instructions for use; other therapeutic agents (for combination therapy); other reagents, e.g., a diluent, devices or other materials for preparing composition for administration; pharmaceutically acceptable carriers; and devices or other materials for administration to a subject. Instructions for use can include instructions for therapeutic application, including suggested dosages and/or modes of administration, e.g., in a human subject, as described herein. In some embodiments, said kits are for use in the methods and uses as described herein, e.g. therapeutic, diagnostic, or imaging methods, or are for use in in vitro assays or methods. The peptide(s) or variant in such kits may, in some embodiments, be a conjugate, e.g., may be conjugated to a detectable moiety.

### EXAMPLES

### Example 1: Biophysical Homology Screening

Native host defense peptides (HDPs) exist as short α-helices or β-sheets of 10 to 40 amino acids and frequently have a dichotomous amphipathic charge distribution with clusters of amino acids having polar charges arranged along an opposing plane of amino acids with hydrophobic residues A database of 431 α-helical antimicrobial peptides (AMPs) and HDPs (http://aps.unmc.edu/AP/main.php) were screened using Molly font under the hypothesis that phylogenetically conserved structural domains within naturally occurring HDPs harbor important innate immune functions, and that such select structure/function paradigm domains can be isolated and optimized for the design of novel therapeutics.

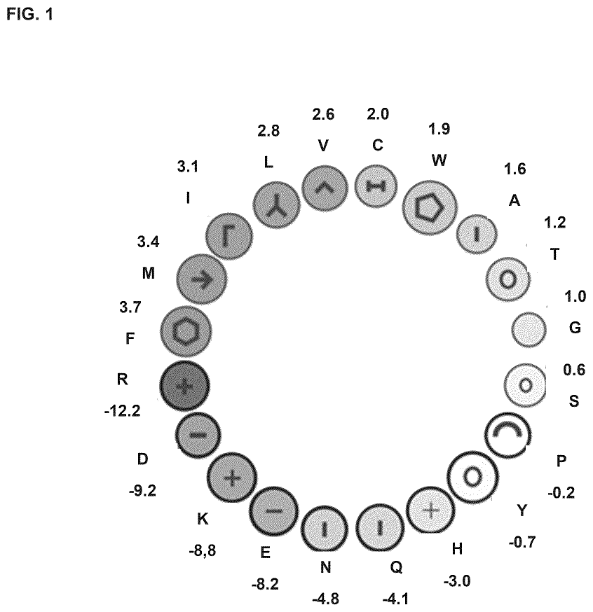

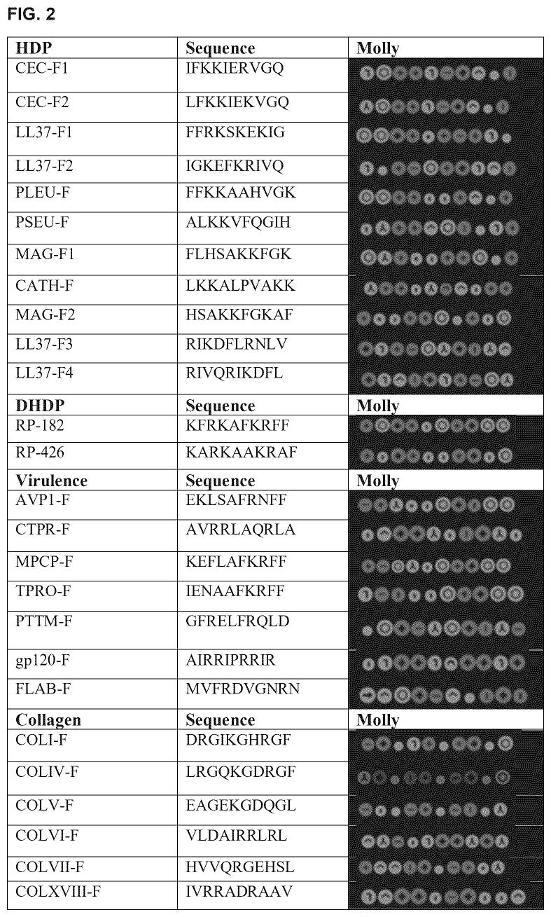

Instead of homology comparisons using primary amino acid alignments, Molly font (Molly Hydrophobicity Wheel) assesses the three key biophysical features: hydrophobicity, electrostatic charge of amino acids, and steric amino acid volume to detect structural homologies via their unique conserved biophysical nature (FIG. 1A). Out of the screened 431 peptides that were identified, 129 peptides, or 30%, were found to possess a preserved 10 amino acid domain consistent with the structural determinants of a secondary a-helical structure with amphiphilic surface topology (Table 4). Biophysically similar sequences were also identified in human collagens and various microbial virulence factors, possibly indicating peptide-structure involved in conserved, shared innate immune functionalities (FIG. 2 and Table 4).

TABLE 4
A

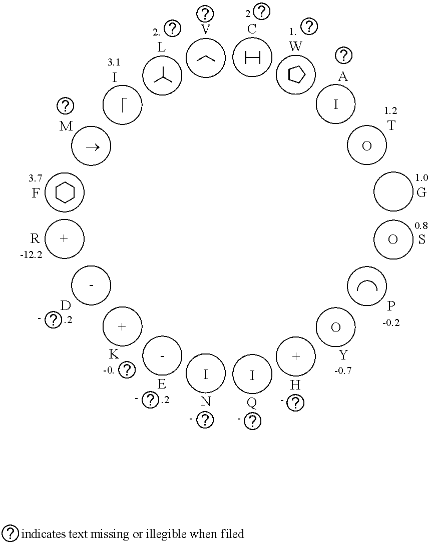

B

| | | | |
|---|---|---|---|
| HDP | Sequence | Virulence | Sequence |
| CEC-F1 | IFKKIERVGQ | AVP1-F | EKLSAFRNFF |
| CEC-F2 | LFKKIEKVGQ | CTPR-F | AVRRLAORLA |
| LL37-F1 | FFRKSKEKIG | MPCP-F | KEFLAFKRFF |
| LL37-F2 | IGKEFKRIVQ | TPRO-F | IENAAFKRFF |
| PLEU-F | FFKKAAHVGK | PTTM-F | GFRELFRQLD |
| PSEU-F | ALKKVFQGIH | gp120-F | AIRRIPRRIR |
| MAG-F1 | FLHSAKKFGK | FLAB-F | MVFRDVGNRN |
| CATH-F | LKKALPVAKK | Collagen | Sequence |
| MAG-F2 | HSAKKFGKAF | COLI-F | DRGIKGHRGF |
| LL37-F3 | RIKDFLRNLV | COLIV-F | LRGQKGDRGF |
| LL37-F4 | RIVQRIKDFL | COLV-F | EAGEKGDQGL |
| DHDP | Sequence | COLVI-F | VLDAIRRLRL |
| RP-182 | KFRKAFKRFF | COLVII-F | HVVQRGEHSL |
| RP-426 | KARKAAKRAF | COLXVIII-F | IVRRADRAAV |

TABLE 5

| HDP | Sequence | Origin |
|---|---|---|
| CATHF1 | LKKALPVAKK | ceratotoxin-A [Ceratitis capitata] Sequence ID: XP_004523341.1 |
| CECF1 | IFKKIERVGQ | cecropin A [Hyphantria cunea] Sequence ID: AID51414.1 |
| CECF2 | LFKKIEKVGQ | cecropin A [Hyalophora cecropia] Sequence ID: AAA29185.1 |
| LL37F1 | FFRKSKEKIG | cathelicidin antimicrobial peptide preproprotein [Homo sapiens] Sequence ID: NP_004336.3 |
| LL37F2 | IGKEFKRIVQ | cathelicidin antimicrobial peptide preproprotein [Homo sapiens] Sequence ID: NP_004336.3 |
| LL37F3 | RIKDFLRNLV | cathelicidin antimicrobial peptide preproprotein [Homo sapiens] Sequence ID: NP_004336.3 |
| LL37F4 | RIVQRIKDFL | catholicidin antimicrobial peptide preproprotein [Homo sapiens] Sequence ID: NP_004336.3 |
| MAGF1 | FLHSAKKFGK | magainins preproprotein [Xenopus laevis] Sequence ID: NP_001081306.1 |
| MAGF2 | HSAKKFGKAF | magainins preproprotein [Xenopus laevis] Sequence ID: NP_001081306.1 |
| PLEUF1 | FFKKAAHVGK | pleurocidin [Pleuronectes americanus] Sequence ID: Q90ZY0.1 |
| PSEUF1 | ALKKVFQGIH | pseudin-2 [Pseudis paradoxa] Sequence ID: P83189.1 |
| DHDP | Sequence | Origin |
| 182 | KFRKAFKRFF | designed |
| 426 | KARKAAKRAF | designed |
| Virulence | Sequence | Origin |
| AVP1 | EKLSAFRNFF | fibronectin-binding protein PavA [Streptococcus pneumoniae] Sequence ID: WP_079111036.1 |
| CTPR | AVRRLAQRLA | secretion protein [Streptomyces] Sequence ID: WP_079021188.1 MULTISPECIES |
| FLAB | MVFRDVGNRN | polar flagellin FlaB [Vibrio vulnificus YJ016] Sequence ID: BAC95256.1 |
| MPCP | KEFLAFKRFF | putative inner membrane protein [Chlamydia psittaci 06-1683] Sequence ID: EPJ33273.1 |
| PTTM | GFRELFRQLD | phage tail tape measure protein [Mycobacterium obuense] Sequence ID: WP_046363070.1 |
| TPRO | IENAAFKRFF | tail protein [Acinetobacter baumannii] Sequence ID: WP_031953720.1 |
| Collagen | Sequence | Origin |
| COLI | DRGIKGHRGF | pro alpha 1(I) collagen [Homo sapiens] Sequence ID: AAB94054.3 |
| COLIV | LRGQKGDRGF | collagen type IV a6 chain [Homo sapiens] Sequence ID: AAB19038.1 |
| COLV | EAGEKGDQGL | collagen type V alpha 3 chain [Homo sapiens] Sequence ID: AAF59902.1 |
| COLVII | HVVQRGEHSL | collagen alpha-1(VII) chain isoform X2 [Homo sapiens] Sequence ID: XP_016861177.1 |
| COLVII | VLDAIRRLRL | collagen alpha-3(VI) chain isoform X3 [Homo sapiens] Sequence ID: XP_005246122.1 |
| COLXVI | IVRRADRAAV | human type XVIII collagen [Homo sapiens] Sequence ID: CAB90482.1 |

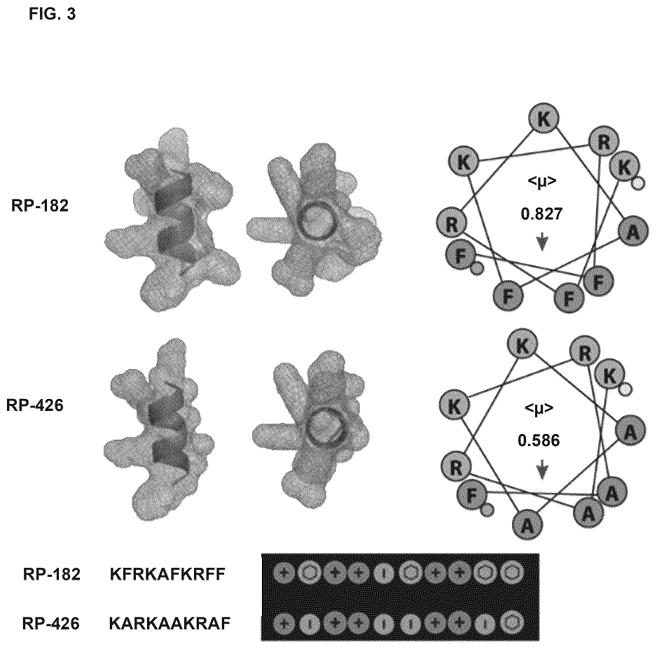

The synthetic design RP-182 was optimized for maximum amphipathy of the original conserved 10mer sequence by increasing hydrophobicity (hydrophobic moment vector <μ>) and positive charge density as visualized in Molly font (FIG. 3). RP-426 was designed as a control to test impact of hydrophobicity onto activity.

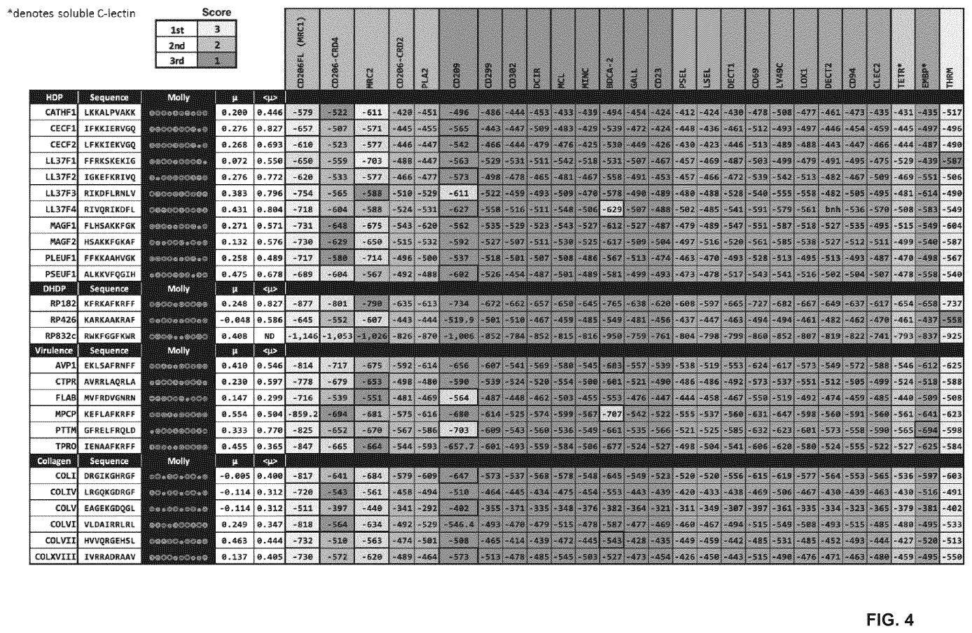

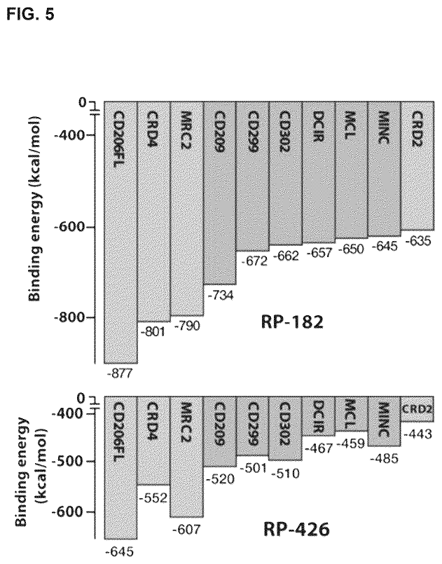

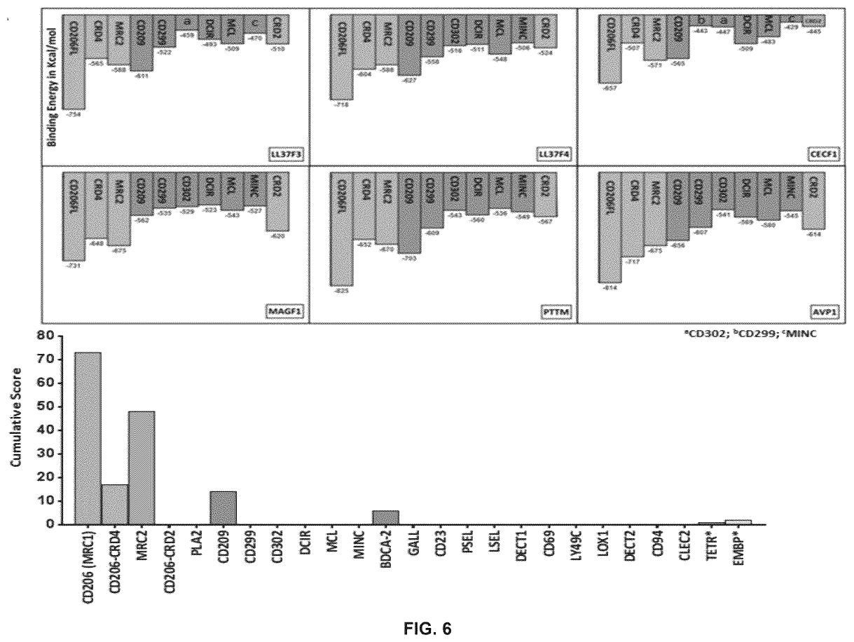

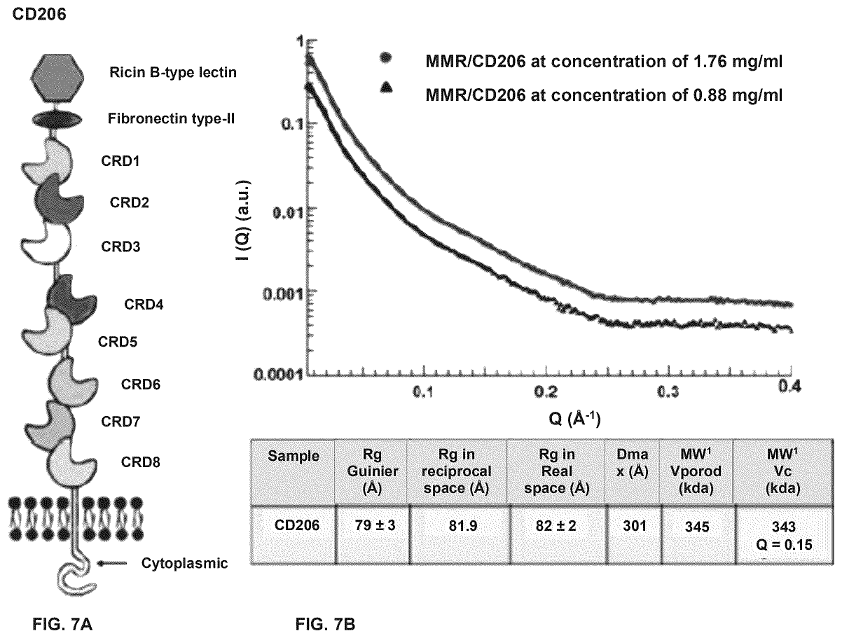

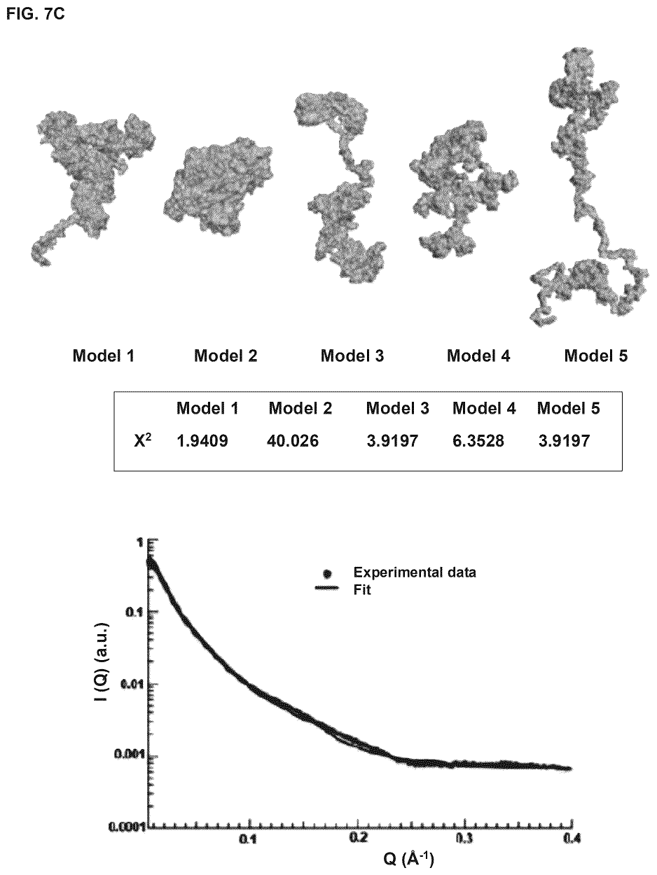

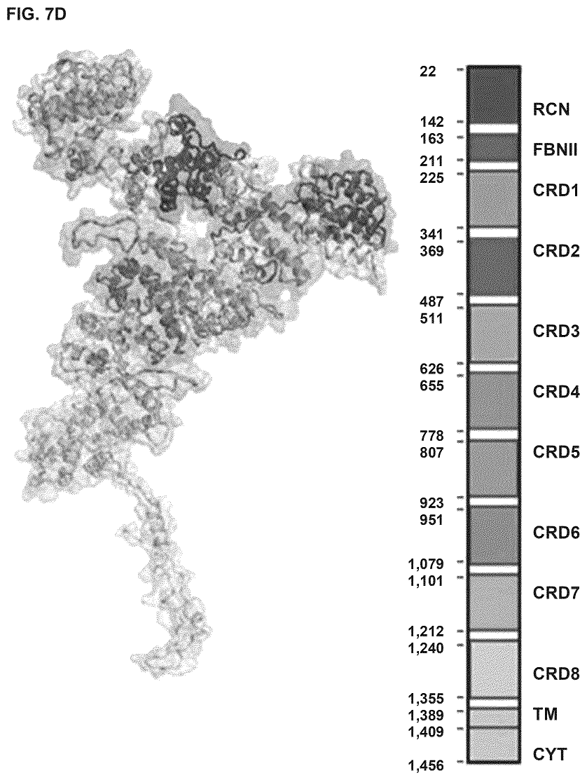

Next, to identify possible leads of putative binding partners of RP-182 and to examine whether RP-182 and the original 10mer homology sequences share common innate defense regulator function, we conducted in silico docking studies on human C-type lectin receptors which are target receptors of HDPs and major regulators of innate immune processes in higher organisms. The animal lectin database (http://ww.imperial.ac.ukresearch/animallectinstctld/mammals/bumanvmousedata.httml) contained 86 membrane-associated human C-type lectin-like domain (CTLD) containing proteins, of these had 24 crystal structures available. Using ClusPro®, the crystal structures were interrogated for binding to RP-182 and biophysically similar 10mer peptide fragments from 23 representative HDPs, virulence factors, and internal collagen sequences (FIG. 4). FIG. 5 shows the ten CTLD containing proteins with the highest predicted binding affinity to RP-182, and FIG. 6 binding affinities of top receptor/ligand combinations of other 10mer homology motifs, identifying the mannose receptor 1 (MRC1/CD206) as the target with the highest in silico affinity. MRC1/CD206 is a member of the group 6, C-type lectin receptor family and undergoes a conformational change from open, ‘elongated’ to closed state upon ligand binding or as pH in the surrounding environment decreases (FIG. 7A).

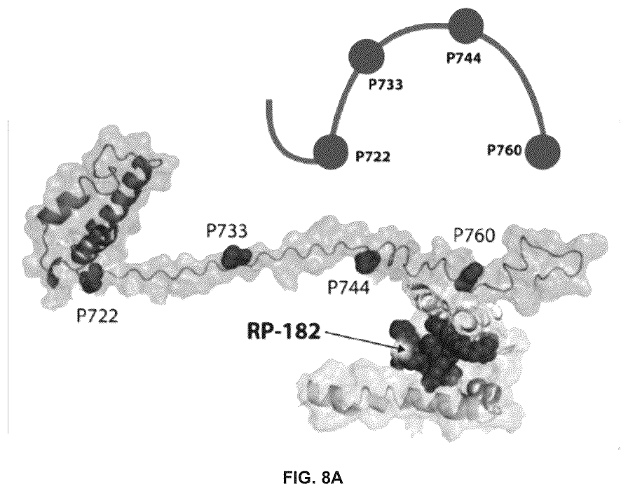

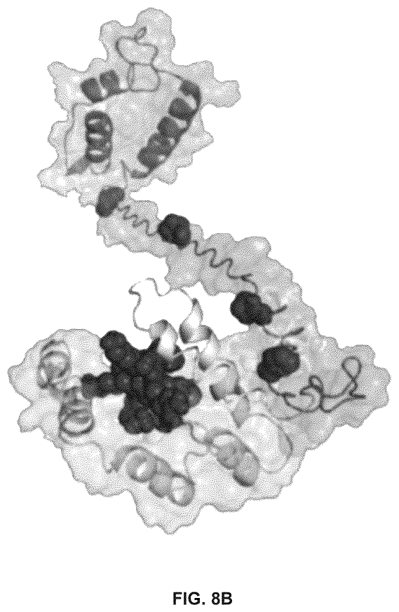

Next, in silico models were created of full length human CD206 derived from I-TASSER which we aligned with small angle X-ray scattering (SAXS) data (FIGS. 7B-D). The molecular weight estimated by SAXS data revealed that CD206 forms a dimer in solution (FIG. 7B). The dimer of CD206 based on Model1 as monomer achieved the best fit (minimum χ2) to SAXS experimental data (FIGS. 7C-D). Therefore, Model1 was selected and used to repeat docking studies which confirmed full-length CD206 being the top binding partner of RP-182 and all except two of the 10mer peptide sequences with biophysical homology (FIG. 4). Based on Model1 RP-182 was predicted by ClusPro® to nestle into a cavity of CRD5 and to engage via three equidistantly spaced prolines (P722, P733 and P744) within CRD4. P760 acts as a fulcrum enabling bending of the CRD4 ‘handle’ and rolling in of the receptor inducing the closed, ‘globular’ state of the receptor (FIGS. 8A-B).

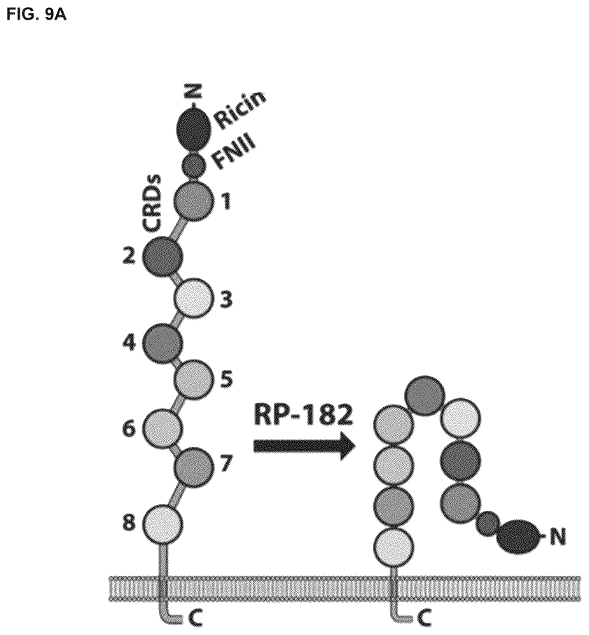

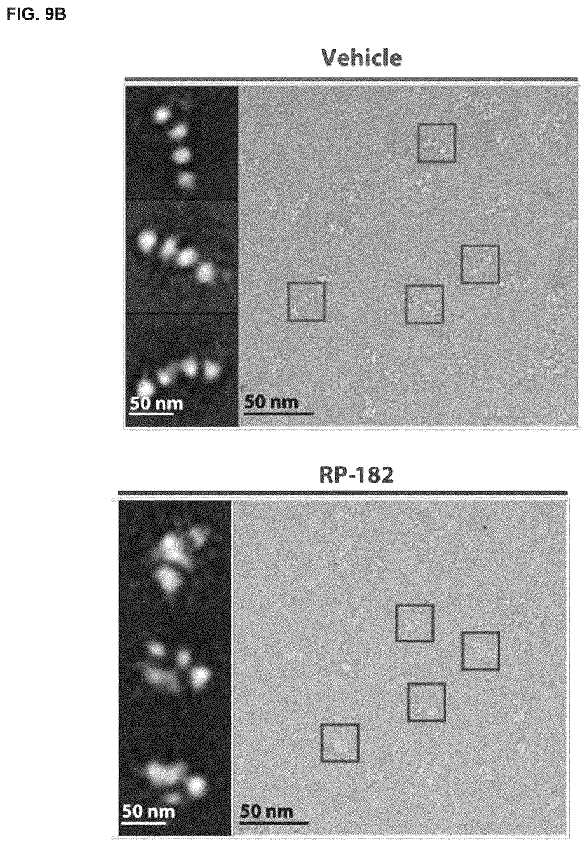

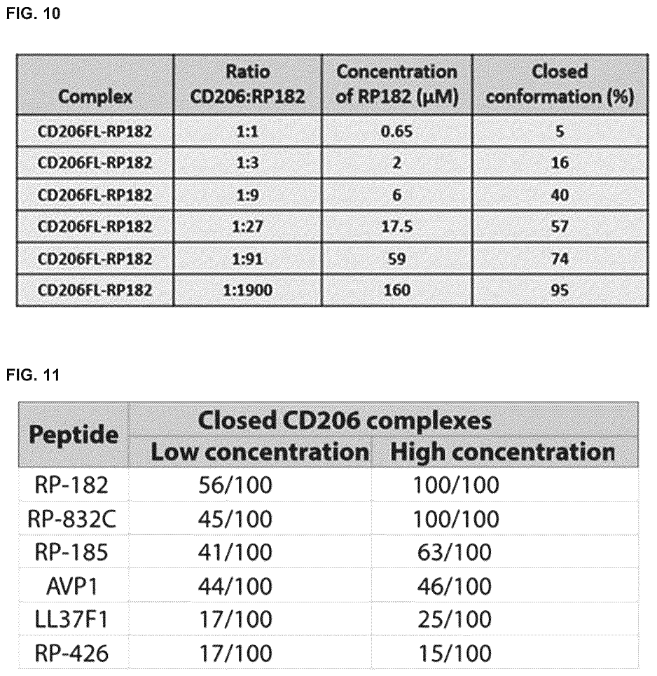

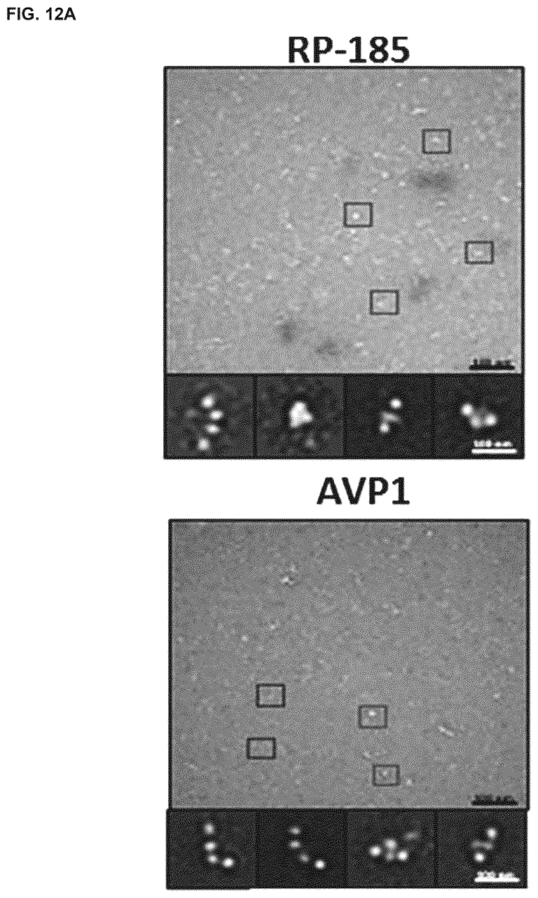

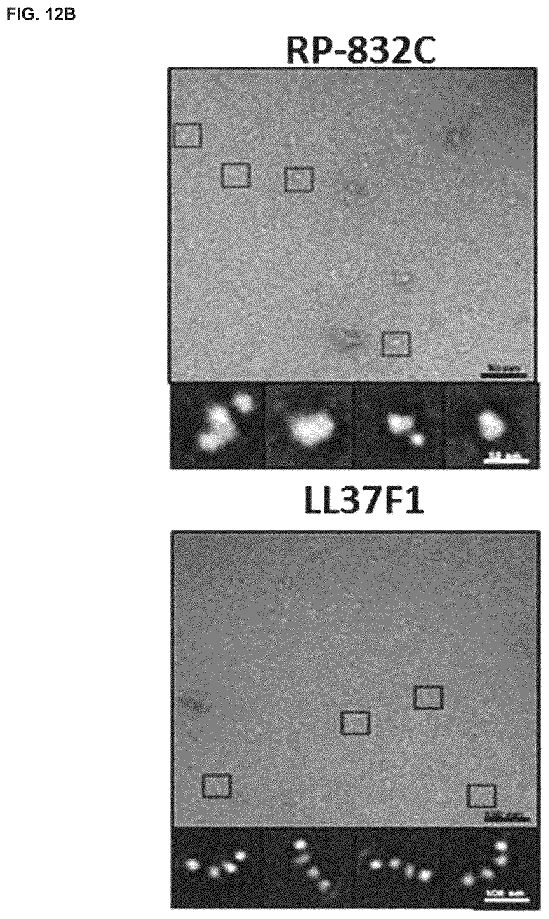

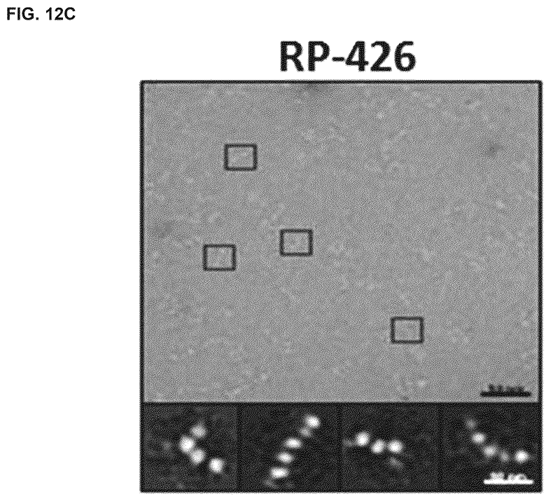

To confirm the above binding studies, first ratios of open versus closed CD206 particles were determined by visualizing by electron microscopy incubated with RP-182 and controls. Upon incubation with RP-182 the open, ‘elongated’ conformation of CD206 switched to the closed, ‘globular’ conformation (FIGS. 9A-B). The half maximal effective concentration (EC50) of RP-182 to induce the closed conformation of CD206 measured ˜11 μM, (FIG. 10). 10mer homology motifs from peptides LL37F1 or AVP1, predicted to bind less to CD206, had lower ratios of closed-to-open conformations compared to peptides RP-832C and RP-182, which were predicted to bind with higher affinity (−1,146 and −877 kcal/mol, respectively) (FIGS. 11 and 12A-C).

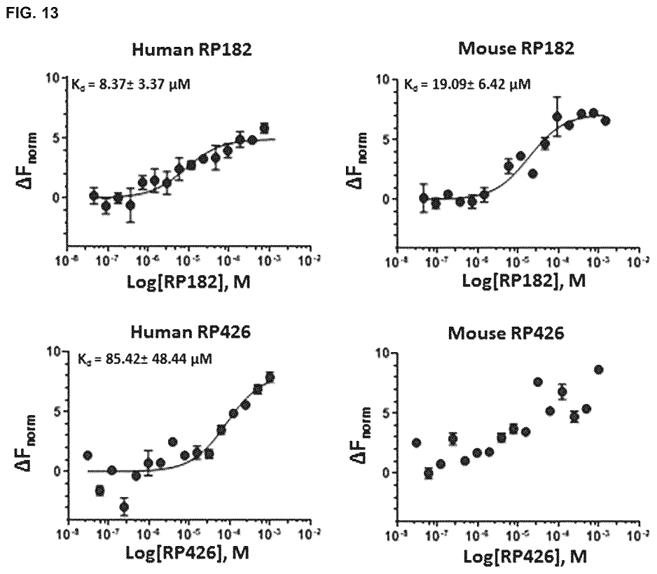

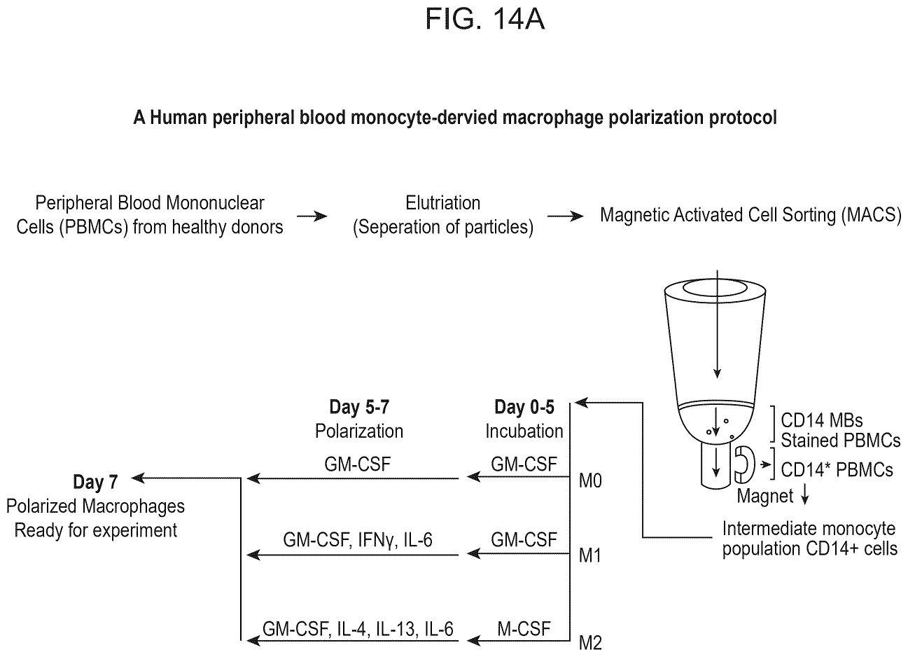

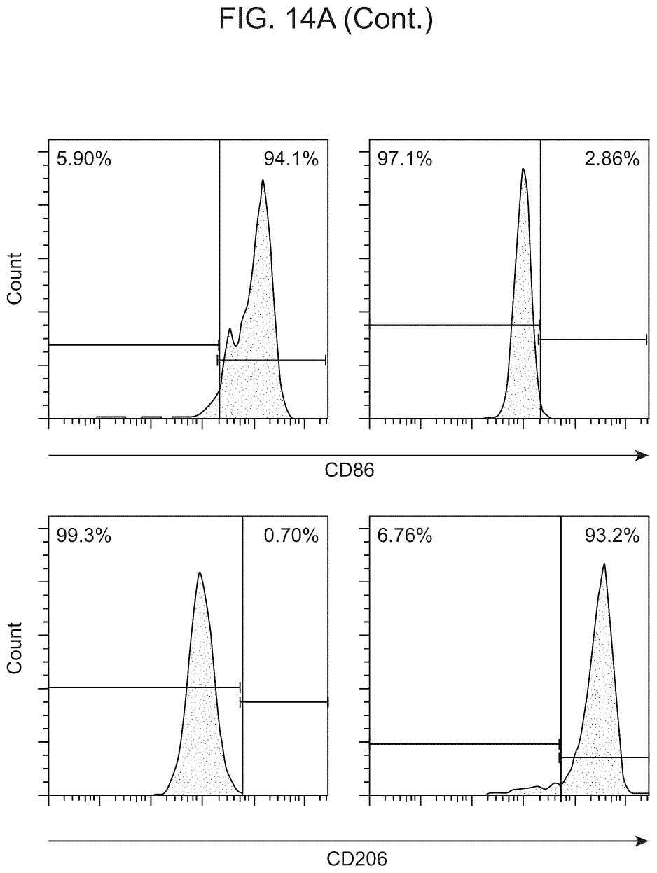

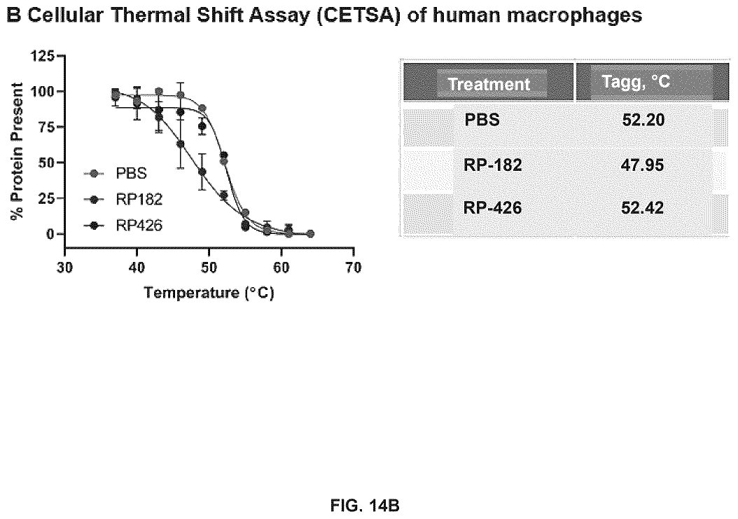

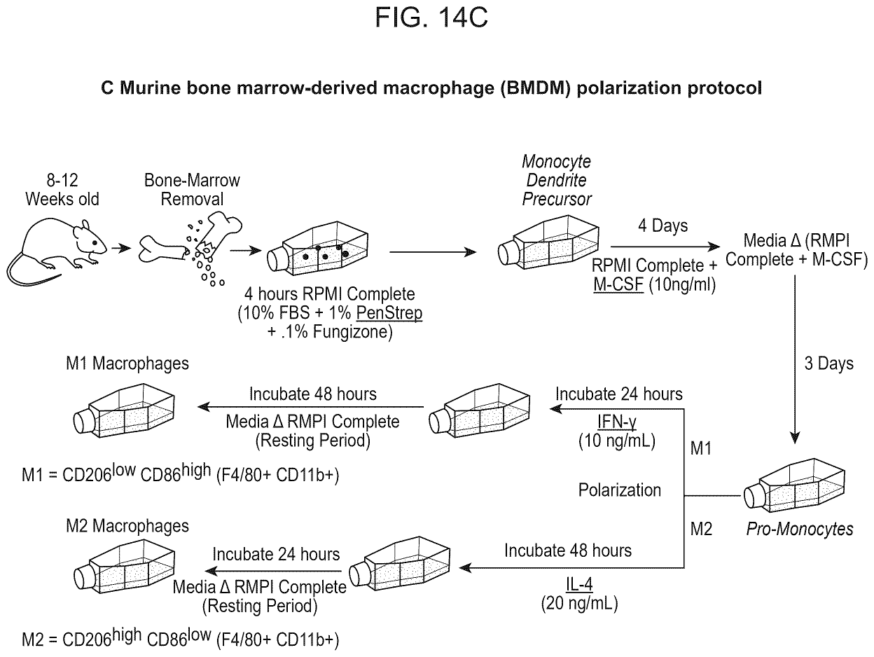

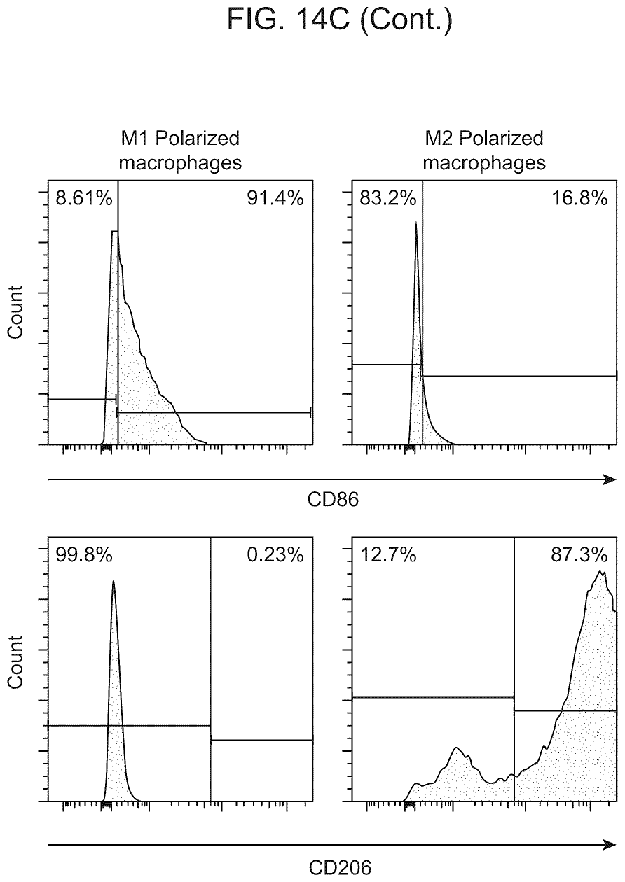

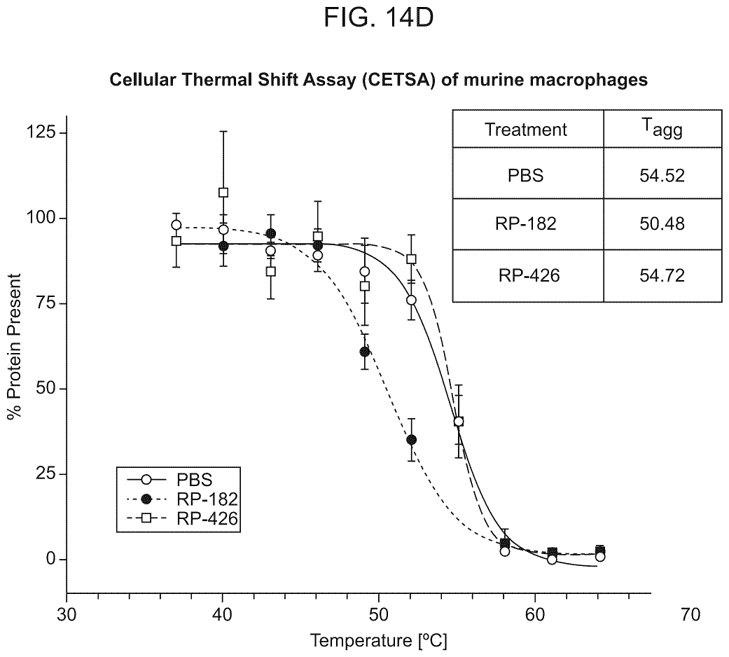

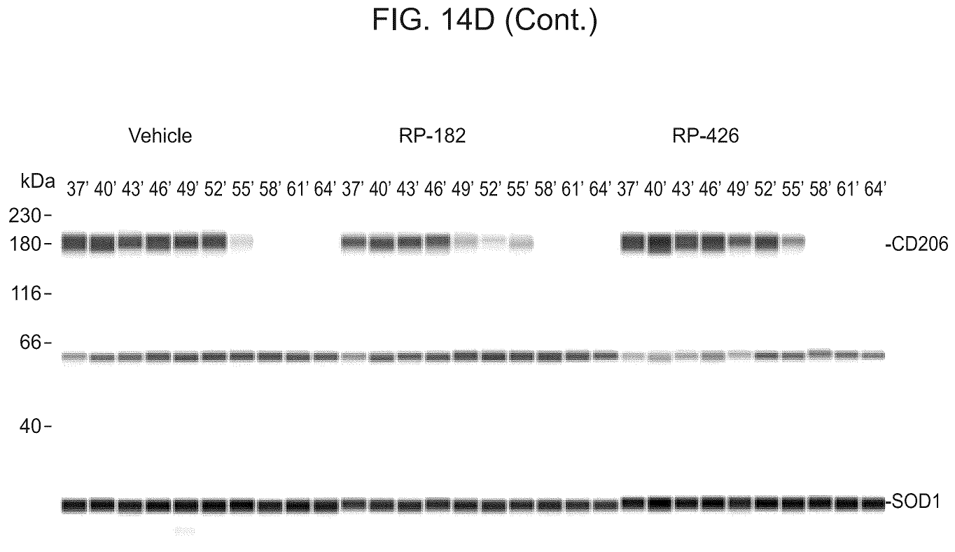

Next, using microscale thermophoresis (MST), binding of RP-182 to human CD206 was measured and determined a KD of ˜8 μM. The binding affinity of RP-426 to CD206 was about ten times lower (KD=85 μM) (FIG. 13). A KD of ˜19 μM was measured for binding of RP-182 to murine CD206. Binding of RP-182, but not control peptide RP-426, to endogenous CD206 in human and murine macrophages was confirmed on bone marrow derived macrophages polarized into the M2 phenotype expressing CD206 by cellular thermal shift assay (CETSA) (FIGS. 14A-D). CETSA assesses target engagement of ligands via thermostability shifting of target protein(s) in a cell-based context. Human and murine M2-polarized macrophages incubated with RP-182, but not with RP-426, showed changed thermostability (>4 degrees) of CD206 compared to vehicle control, indicating interaction of RP-182 and the CD206 receptor in the natural environment (FIGS. 14A-D).

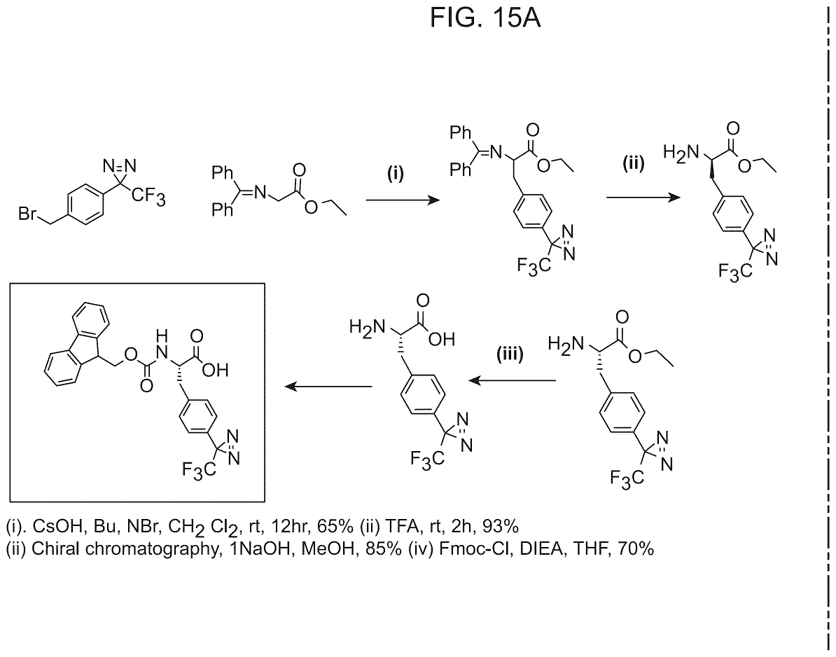

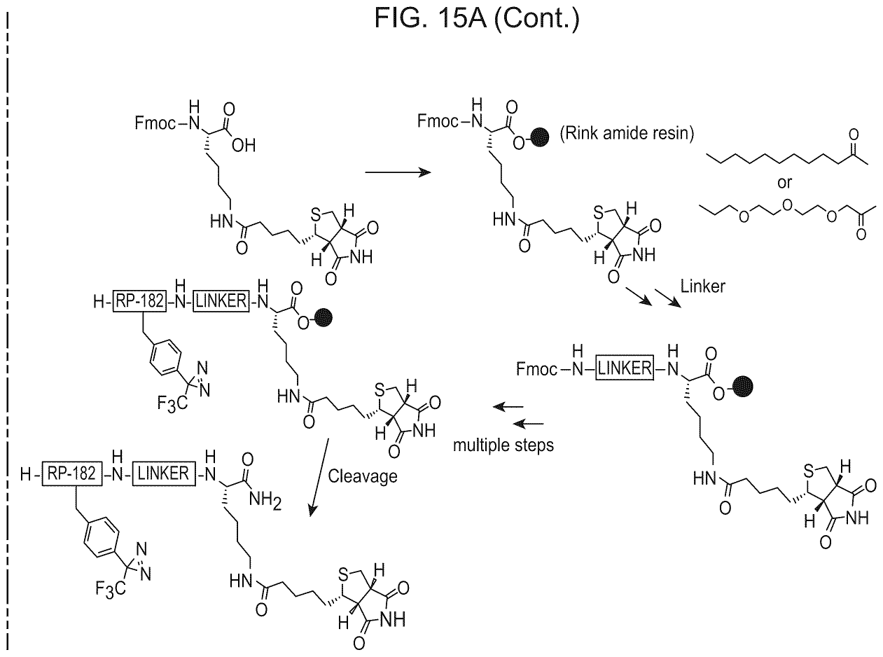

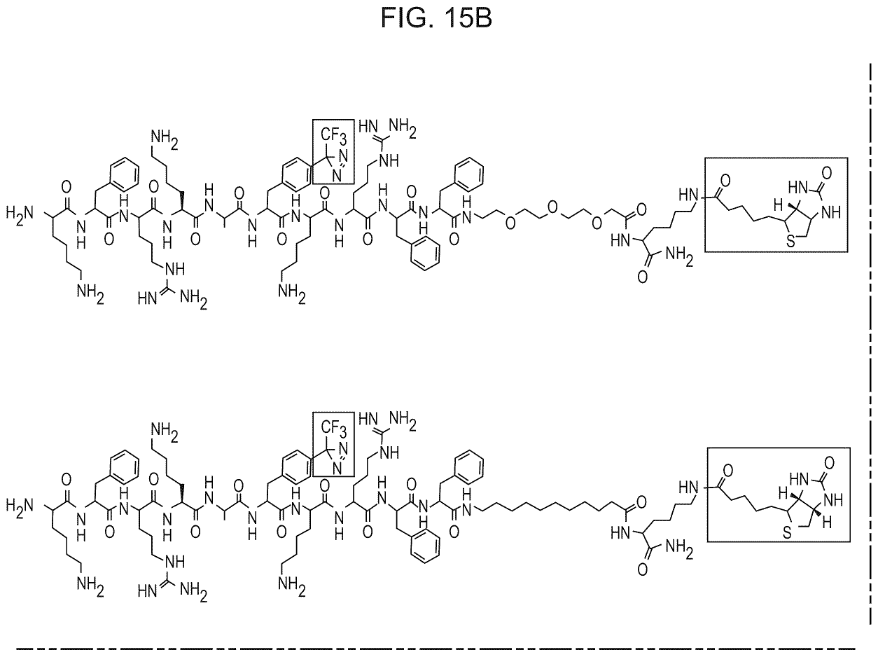

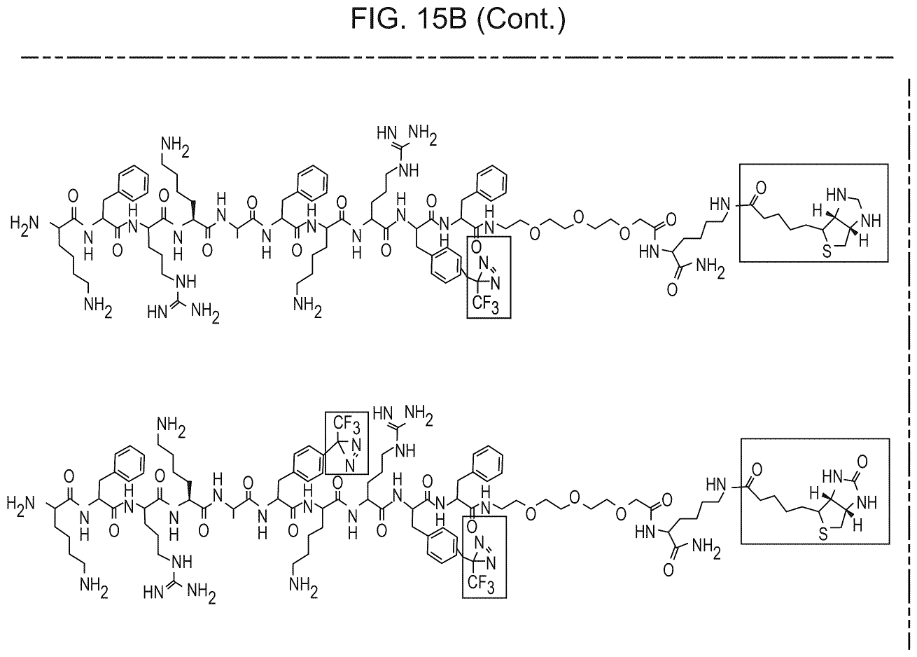

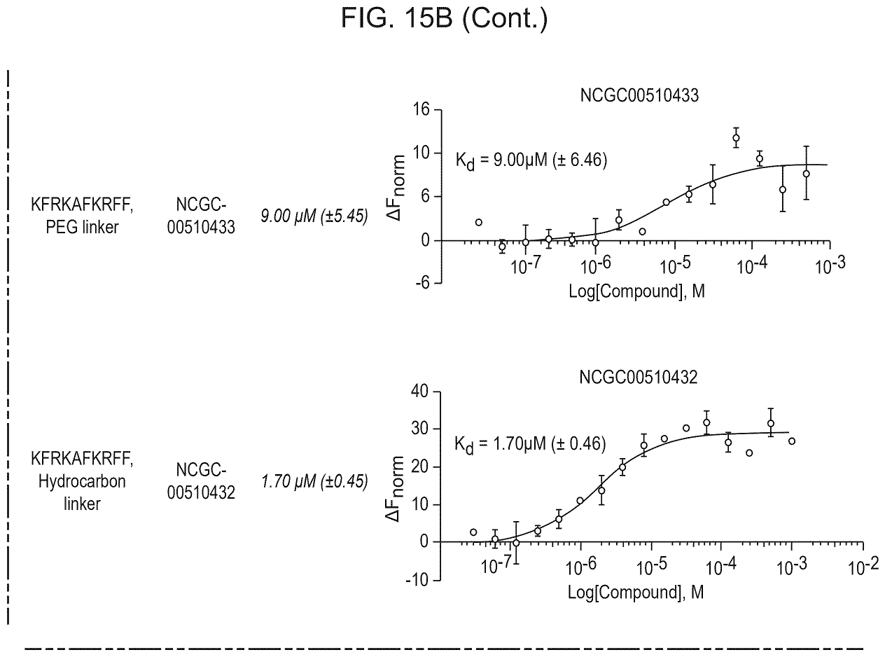

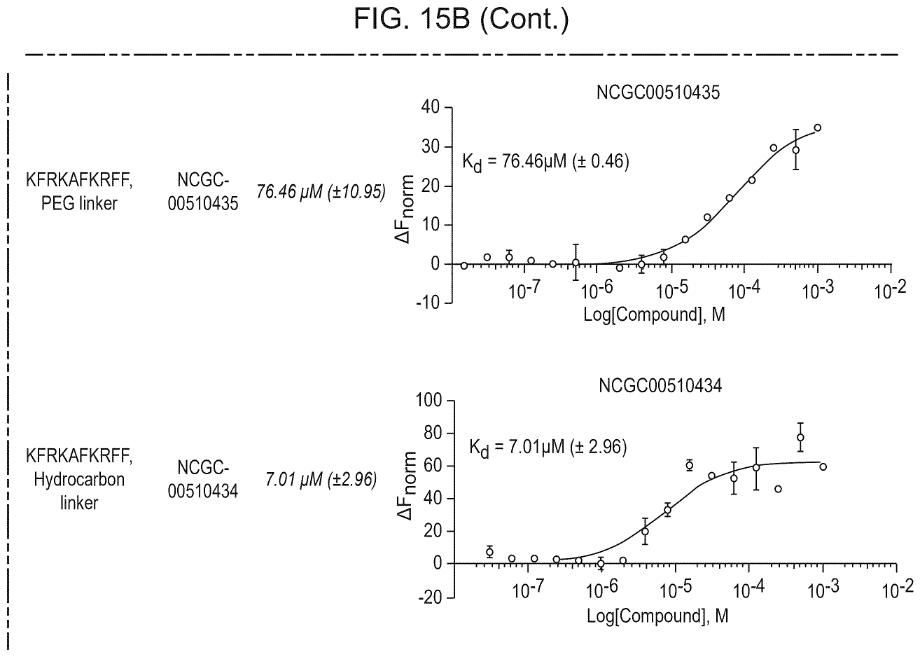

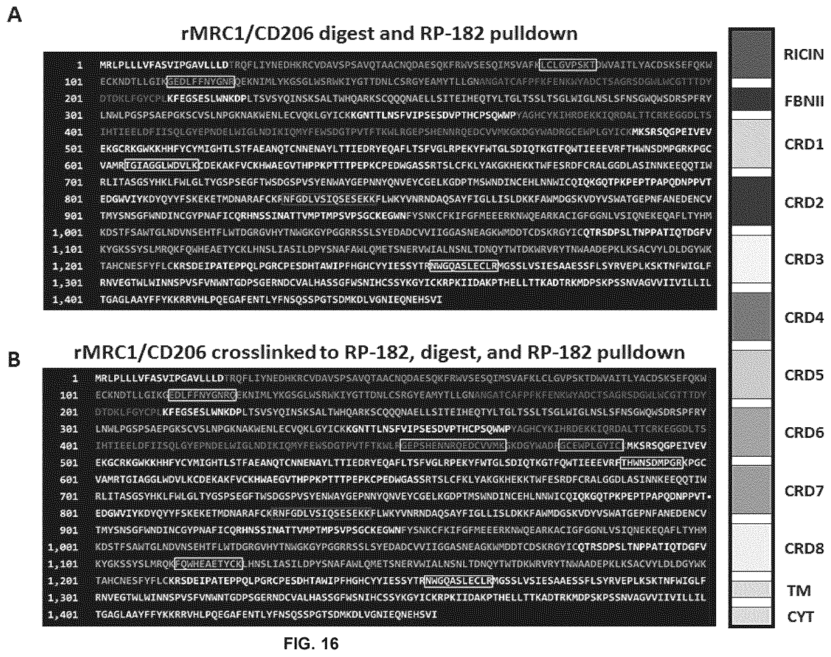

To further map the binding region of RP-182, mass spectrum studies were performed of recombinant CD206 cross-linked to RP-182 derivative NCGC-00510434. NCGC-00510434, which displays similar KD binding to recombinant CD206 as wild type RP-182, contained a diazirine-substituted phenylalanine and a C-terminally attached biotin (FIGS. 15A-B). Fragment analysis of trypsin-digested CD206 pulled-down with NCGC-00510434 identified CRD5 sequence NFGDLVSIQSESEKK, which aligned with peptide analysis of CD206 covalently crosslinked to NCGC-00510434, followed by digestion, pulldown and sequencing of peptide fragments, as well as the CRD5 motif previously predicted by in silico studies using the CD206 SAXS structure to be the binding region of RP-182 (FIG. 16).

In summary, RP-182 is a synthetic HDP derived from a conserved homology sequence found across a diverse range of peptide and protein regulators involved in innate immune processes. It selectively induces a conformational switch from the open to the closed state in the mannose receptor MRC1/CD206 which is different from the conformational change of CRD3 associated with lower pH or the binding of collagen to the fibronectin II domain.

### Example 2: Effect of RP-182 on Cell Function

It was found that RP-182 induces a program of phagocytosis, autophagy, and apoptosis in human and murine M2 macrophages.

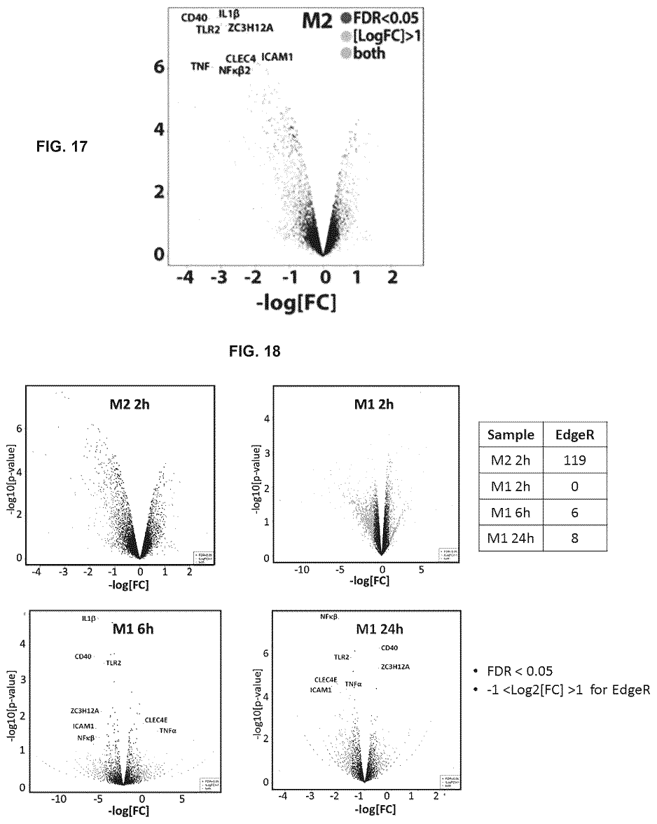

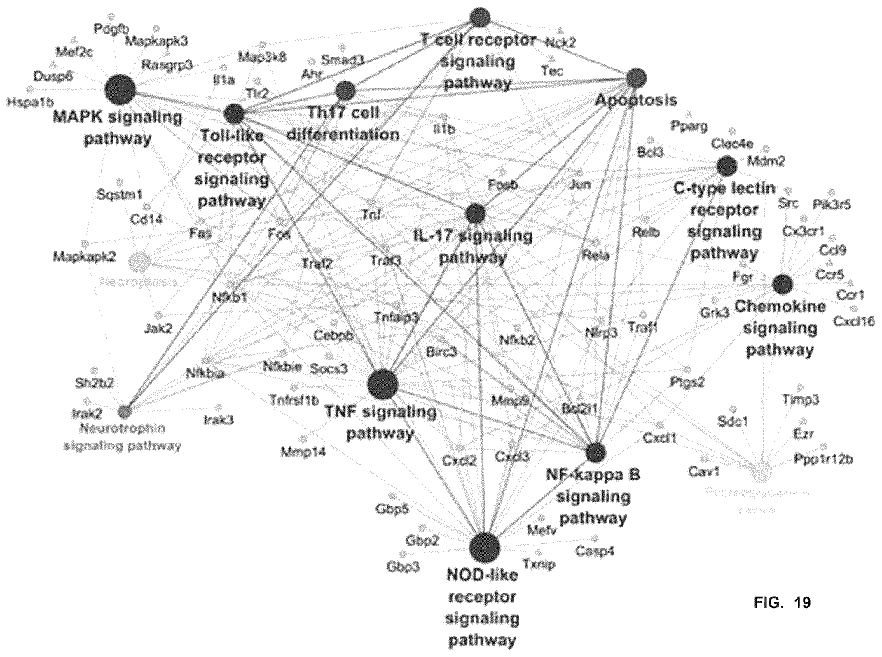

To investigate the impact of RP-182 binding and the induced conformational change of CD206 on cell function, first the effects of RP-182 were examined by global RNASeq analysis. Volcano blot analysis of gene expression changes between vehicle- and RP-182-treated M2 BMDMs showed differentially expressed genes (DEGs) skewed towards upregulation. Seven among the eight top DEGs were cytokines or regulators of the classical pro-inflammatory M1 phenotype showing ≥10- to 100-fold increased expression levels after 2 hours treatment (FIG. 17). Transcriptomic changes following RP-182 treatment in myeloid progenitors of murine bone marrow-derived macrophages (BMDM) polarized into M1 and M2 phenotypes occurred selectively in M2-polarized macrophages with no genes differentially expressed in M1 macrophages after 2 hours, six DEGs after 6 hours, and 8 after 24 hours of treatment with RP-182 (FIG. 18). Cytoscape® Functional GO Enrichment and Network Analyses identified pathways of inflammation and macrophage activation upregulated in RP-182-treated M2 BMDMs including C-type lectin receptor, NF-kB, TNF, or Toll-like receptor (TLR) signaling (FIG. 19).

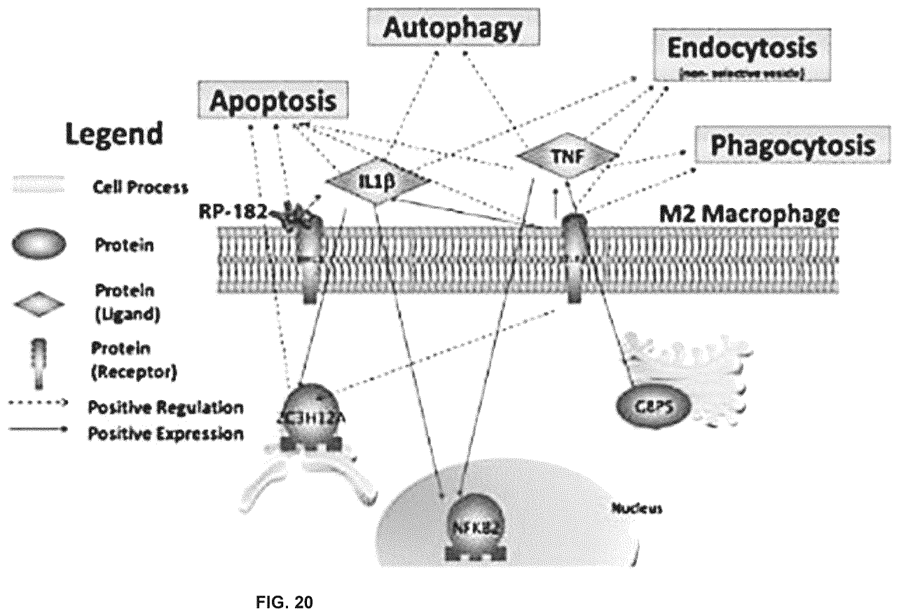

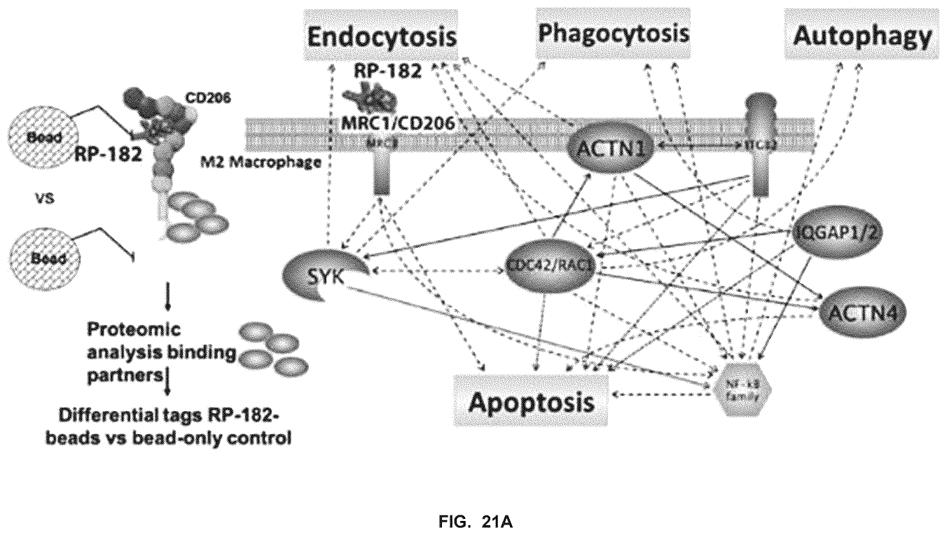

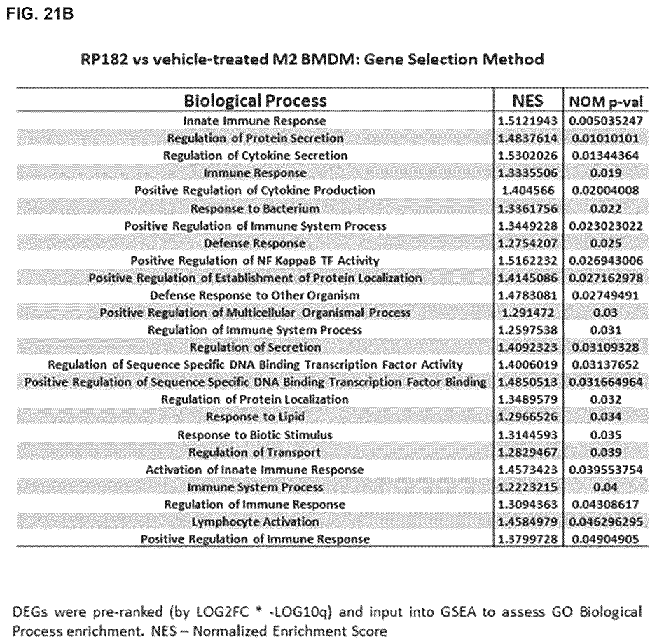

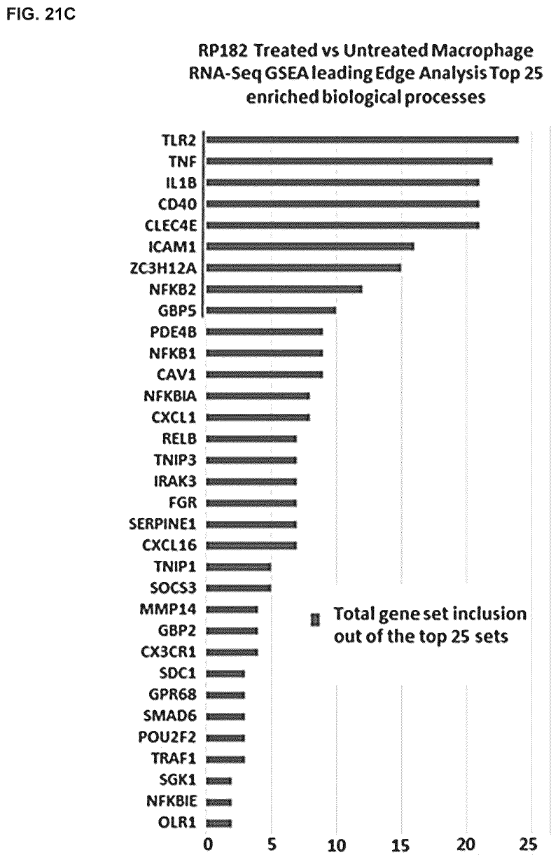

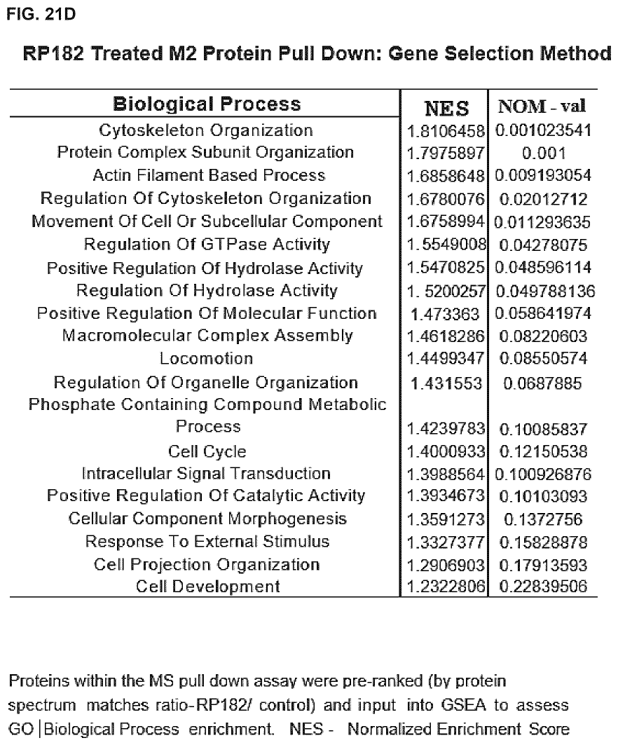

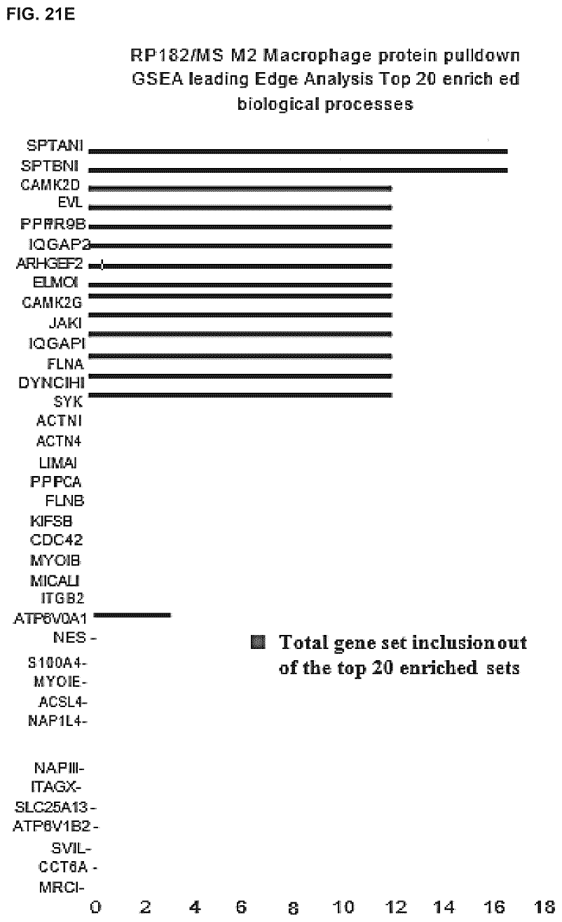

The most commonly represented genes identified by Leading Edge Analysis after Gene Set Enrichment Analysis (GSEA) were imputed into Pathway Studio® which identified processes of endocytosis, phagocytosis, autophagy, and apoptosis as top biological pathways affected by RP-182 in M2 macrophages (FIG. 20). Proteomic analysis of binding partners in CD206 complexes pulled down after 10 minutes of treatment with RP-182 compared to bead-only control showed enrichment of proteins involved in similar cell processes (FIG. 21A, FIG. 4, and FIGS. 21B-E).

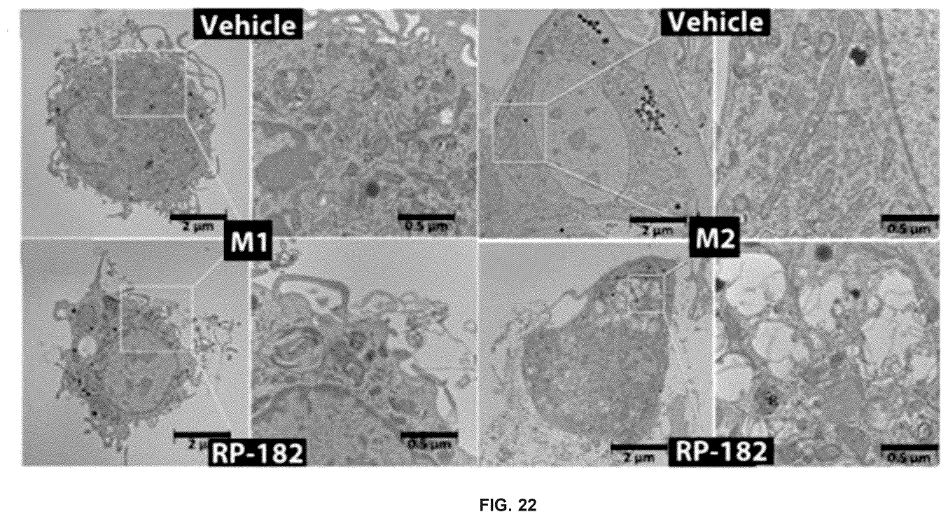

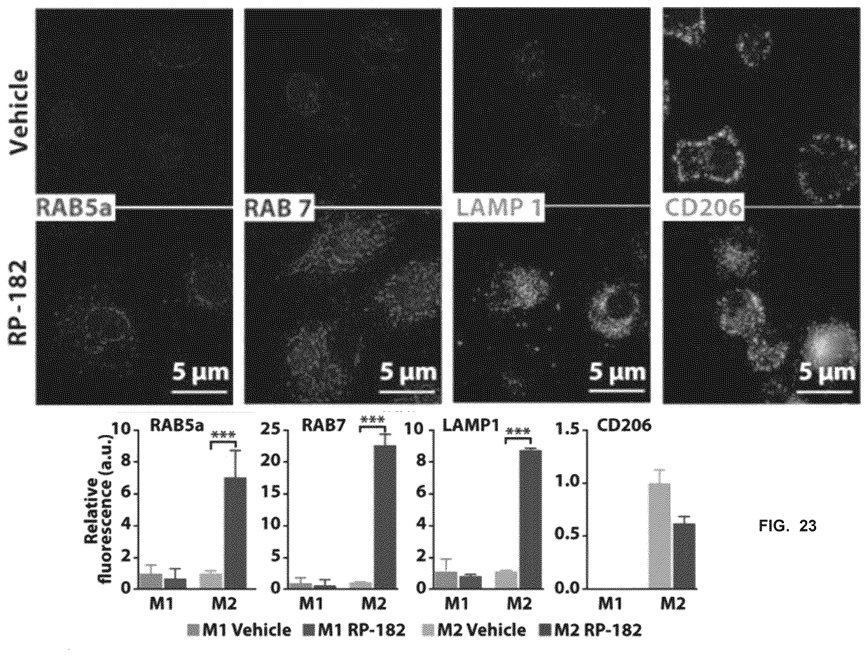

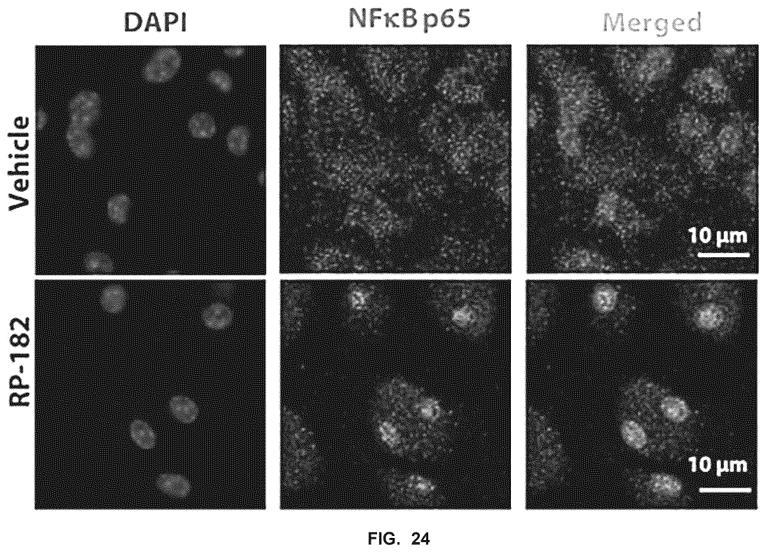

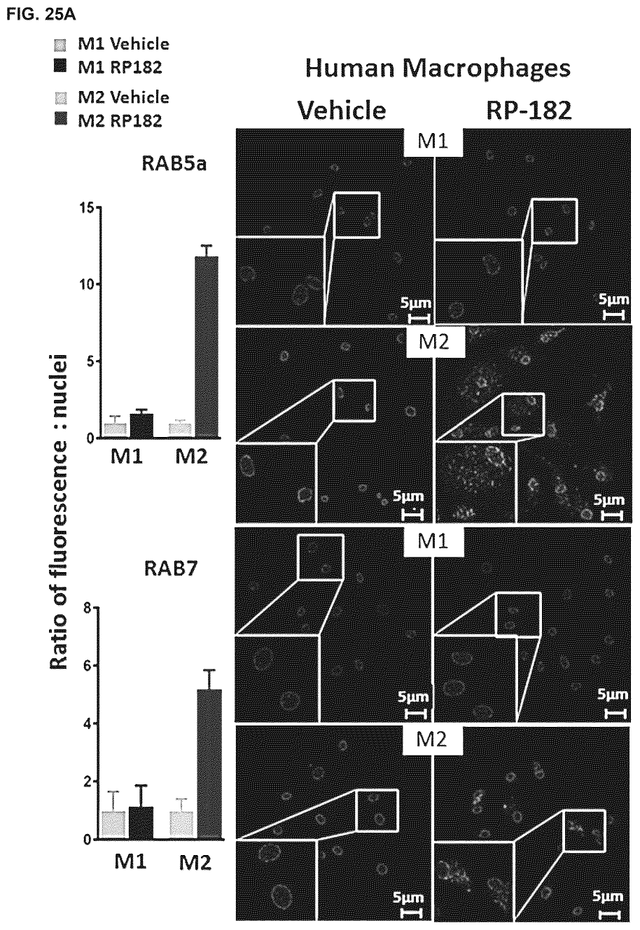

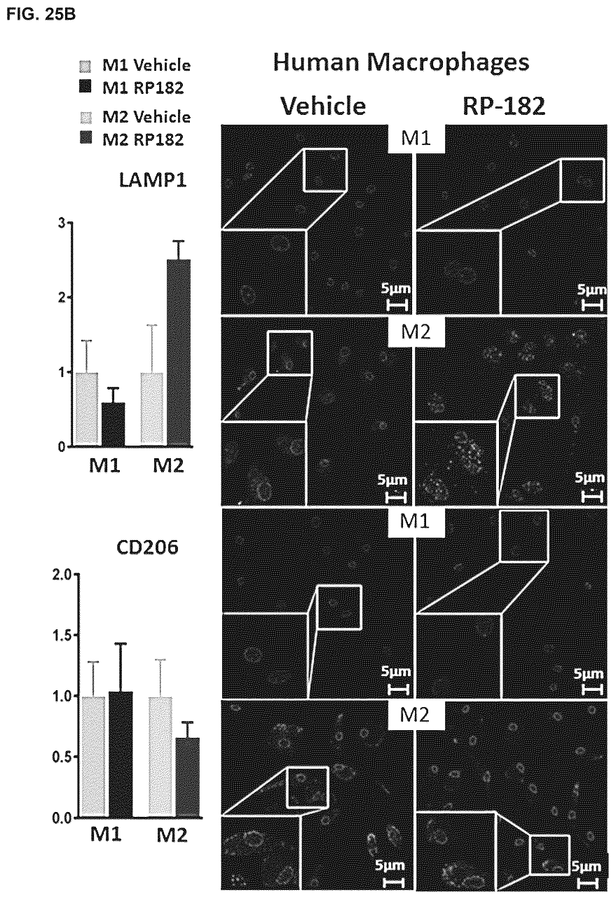

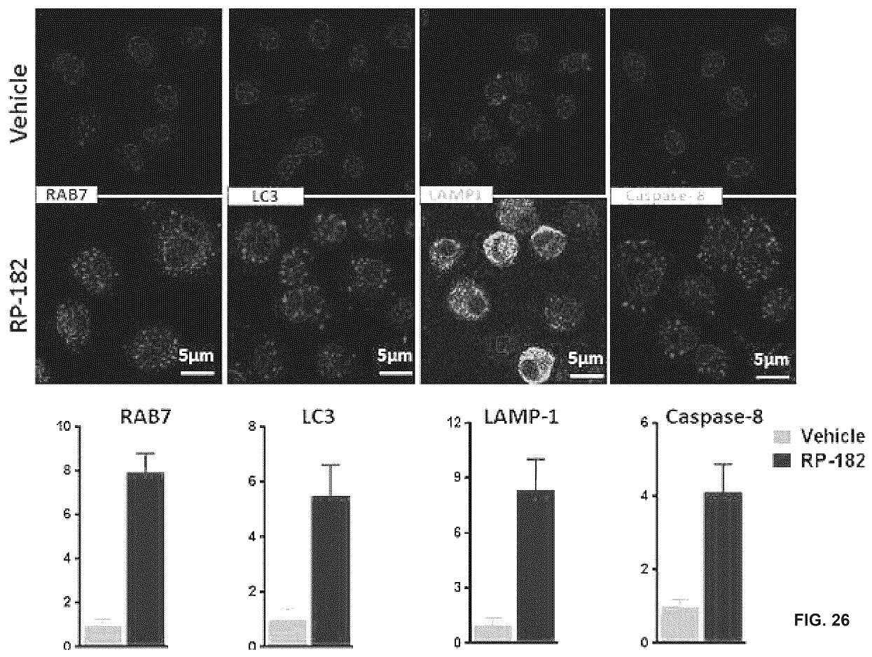

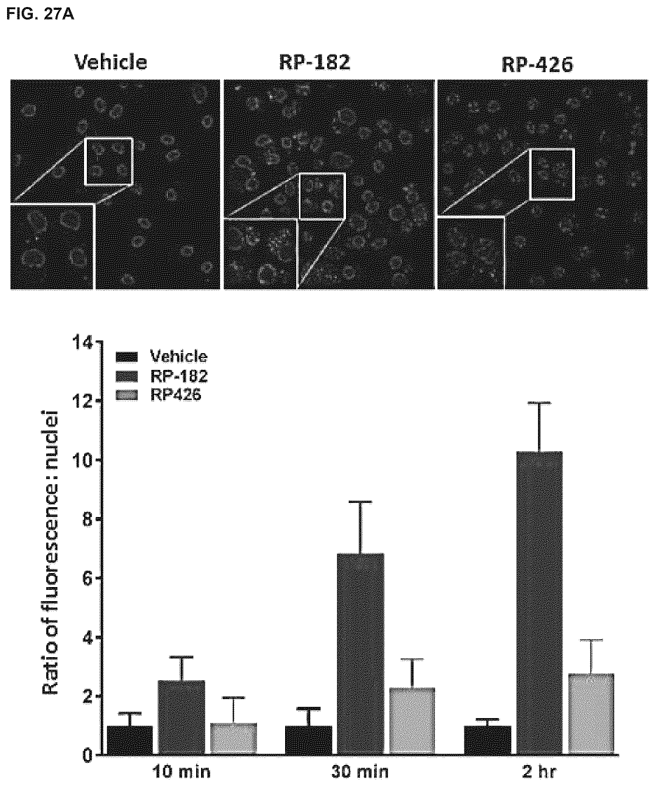

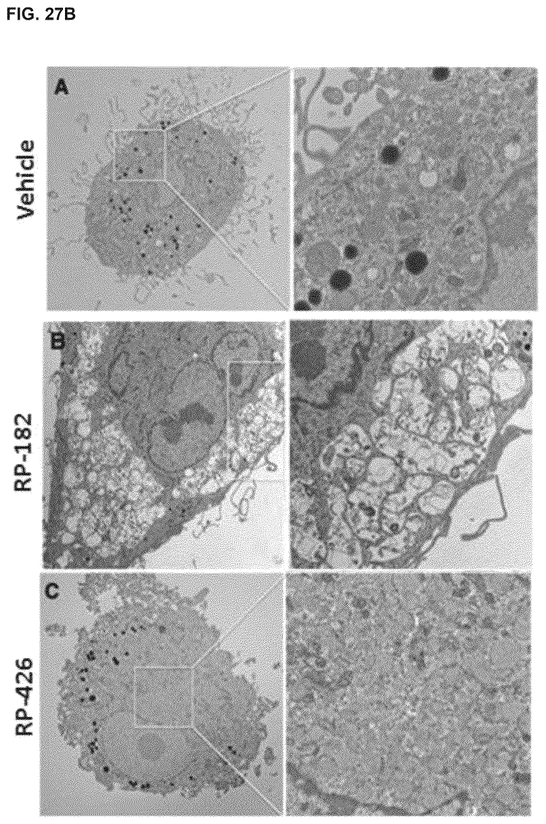

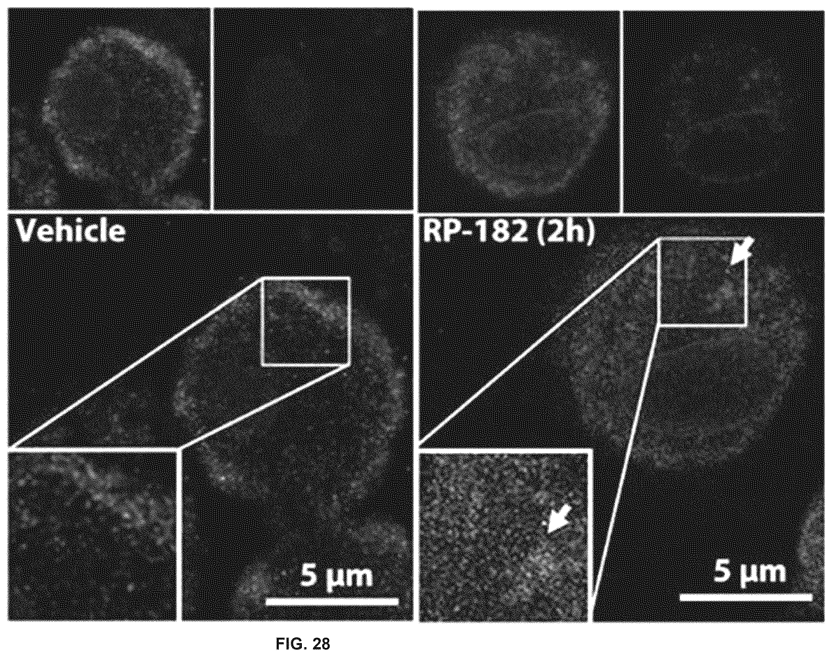

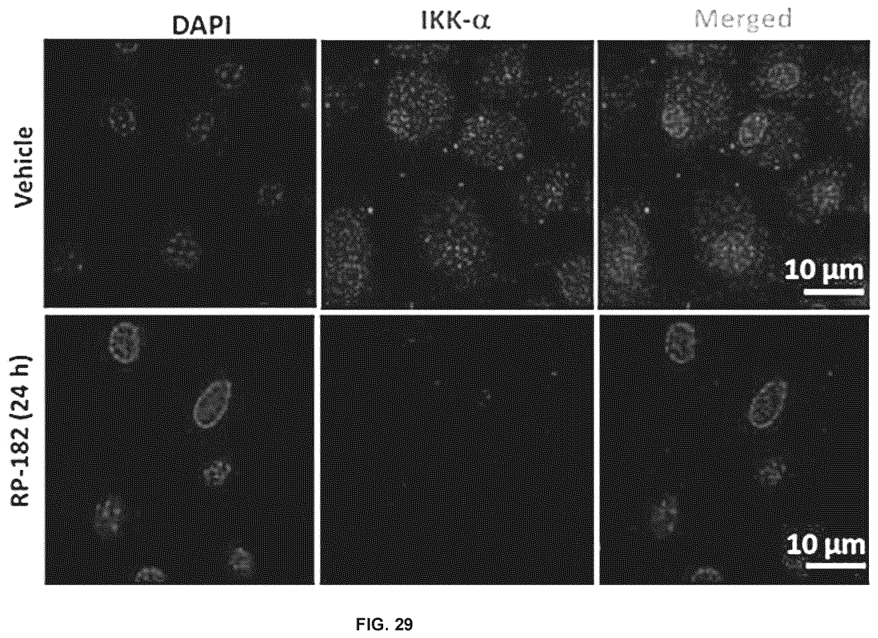

To confirm results of above analyses, BMDMs were evaluated and polarized into M1 and M2 by electron microscopy. RP-182 induced phagosomes in M2- but not M1-polarized BMDMs (FIG. 22). M2-selective induction of phagocytosis was confirmed by upregulation of the early and late endosomal markers Rab5a and Rab7, and the lysosomal-associated membrane protein 1 (LAMP-1) (FIG. 23). Upon treatment with RP-182, CD206 was increasingly detected in the cytoplasm and induced Rab7-positive phagosomes co-stained with CD206, findings in line with the known internalization of the mannose receptor (FIG. 24). Selective induction of phagosomes in the M2 phenotype upon treatment with RP-182 was equally observed in M2-polarized CD14+ peripheral monocytes isolated from healthy volunteers (FIG. 25A-B) and in BMDMs polarized into an ‘in vitro TAM-like’ phenotype after co-culture with conditioned media from cancer cells (FIG. 26). Control peptide RP-426 did not induce phagocytosis (FIG. 27A-B). RP-182 activated NF-kB signaling (FIG. 28 and FIG. 29).

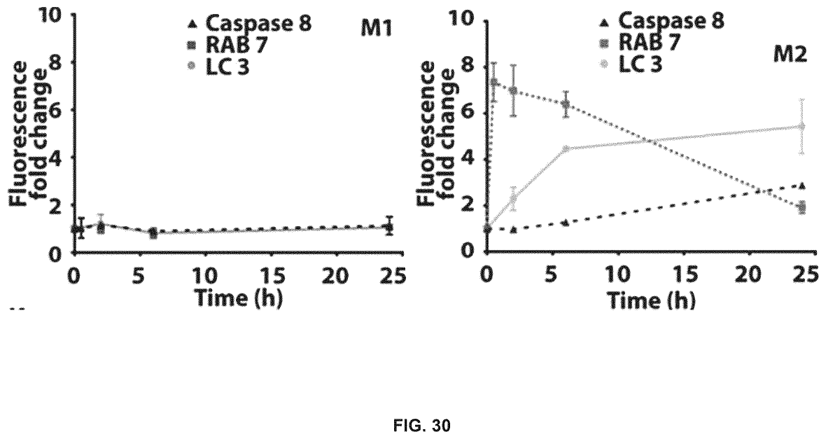

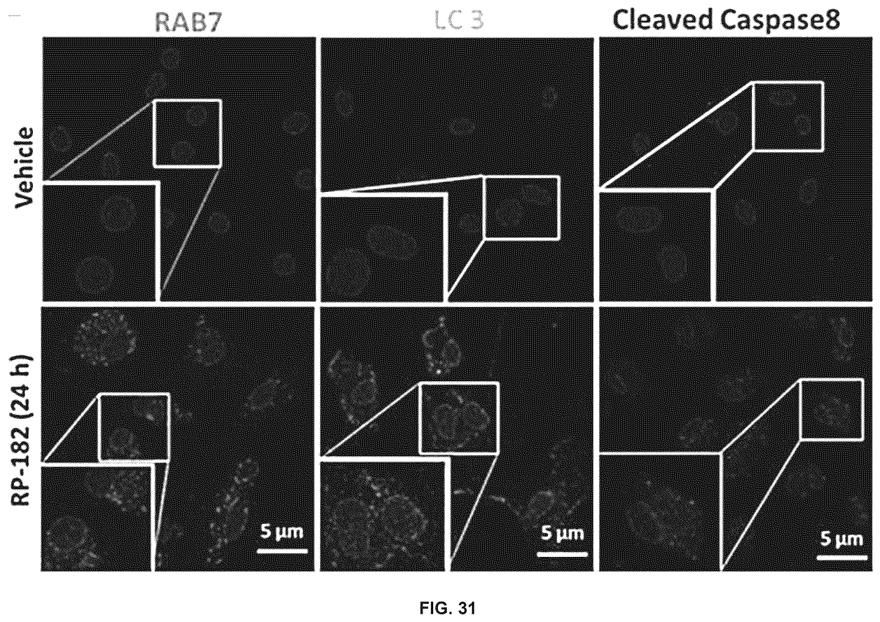

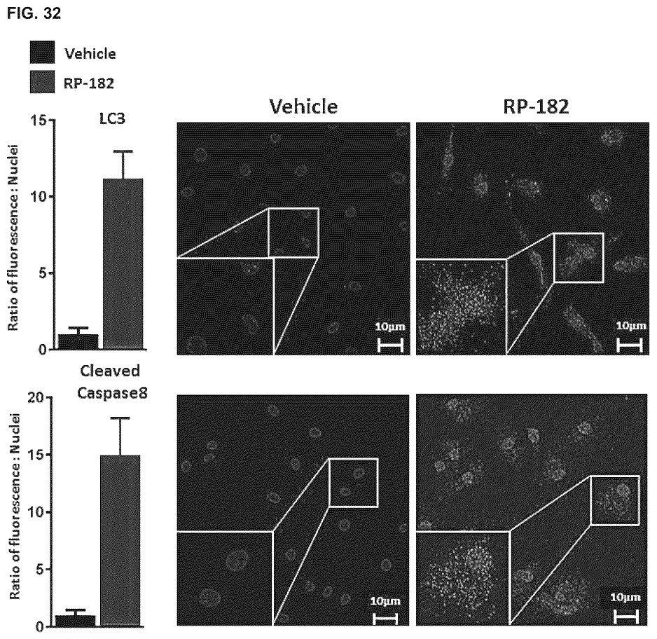

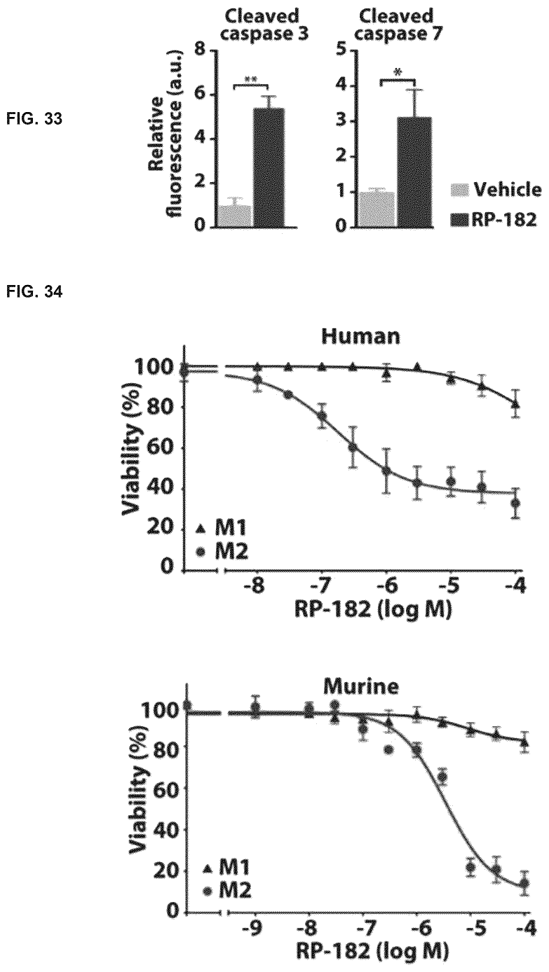

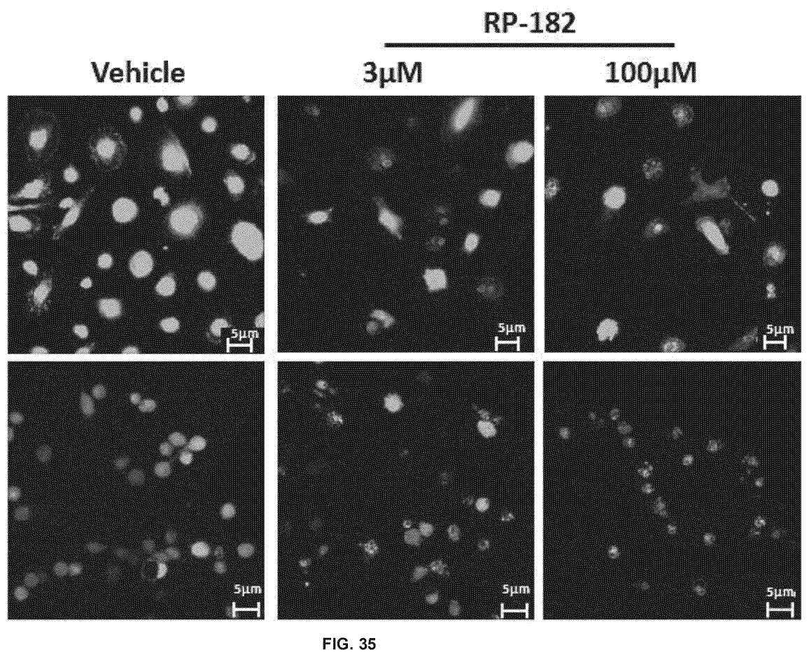

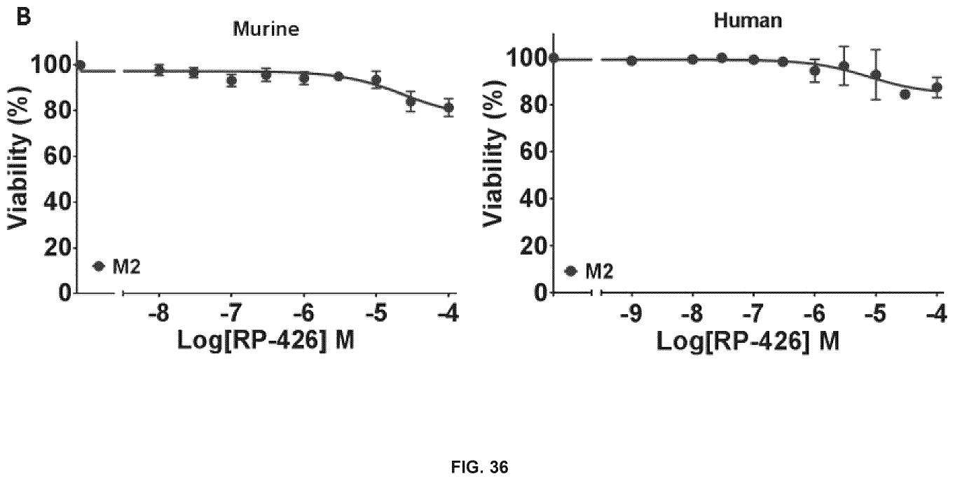

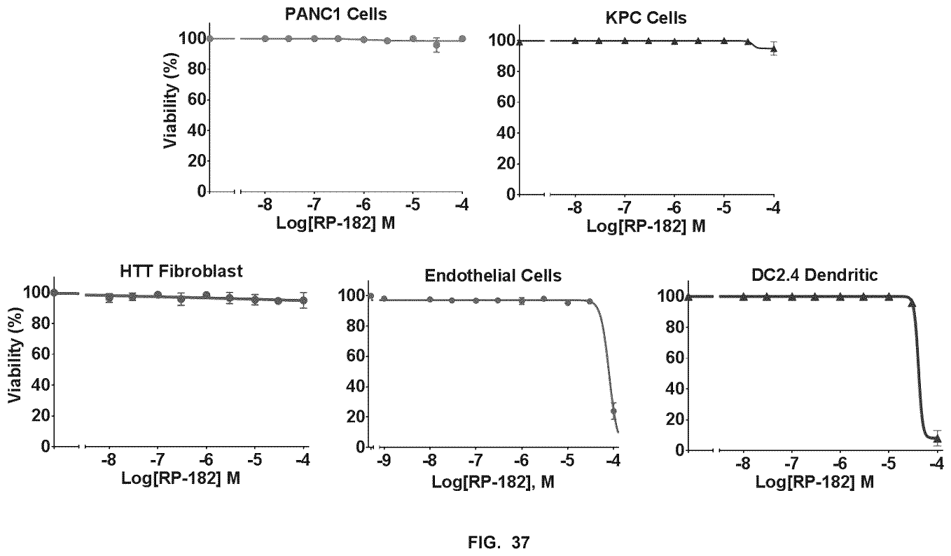

Next, treatment time was extended to 24 hours and measured induction of autophagy and apoptosis across several timepoints. RP-182 sequentially induced phagocytosis, autophagy, and apoptosis in M2 macrophages with no effect on M1 cells (FIG. 30 and FIGS. 31 and 32). RP-182 also induced cleaved caspase 3 and 7, known downstream substrates of activated caspase 8 (FIG. 33). Upon 48 hours exposure to increasing concentrations of RP-182 using a double staining cell viability assay, RP-182 led to a reduction in M2- but not M1-polarized macrophages with calculated potency (IC50) for human M2 and murine M2 of 1.1 and 3.4 μM, respectively (FIG. 34 and FIG. 35). Control peptide RP-426 did not show any activity (FIG. 36), and RP-182 did not affect growth of mesenchymal stem cells, murine and human cancer cells, fibroblasts, or endothelial and D2.4 dendritic cells (FIG. 37).

### Example 3: Effect of RP-182 on M2 Macrophages

It was found that RP-182 reprograms M2 macrophages towards a M1-like phenotype. The observation that viable cell fractions after 48 hours of treatment with RP-182 at highest concentrations were greater than the initial fraction of CD206 negative cells (31% viable cells after max response vs 6.8% CD206-negative cells in human M2 macrophages; 17.2% viable cells vs 12.7% CD206-negative cells in M2 BMDMs) led us to examine a possible second mechanism of action of RP-182. It was speculated that M2 macrophages reprogrammed by RP-182 towards a M1-like phenotype may lose CD206 expression and might not be subject to the cell killing function of RP-182.

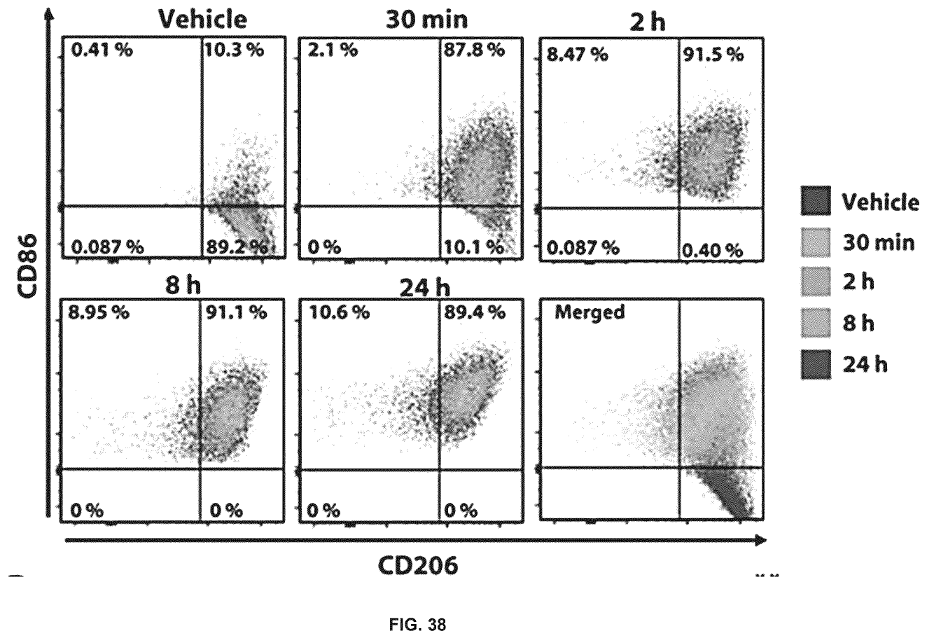

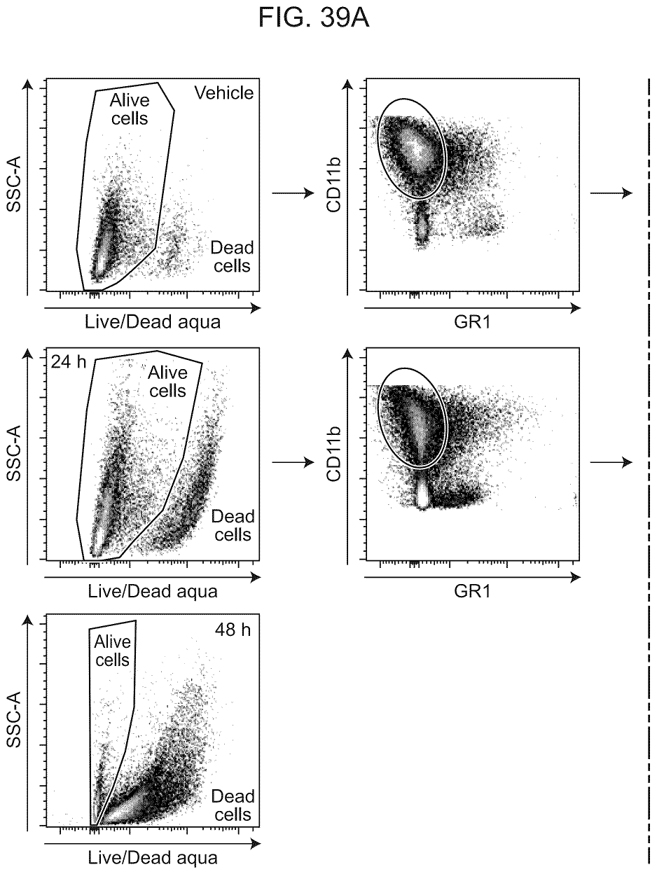

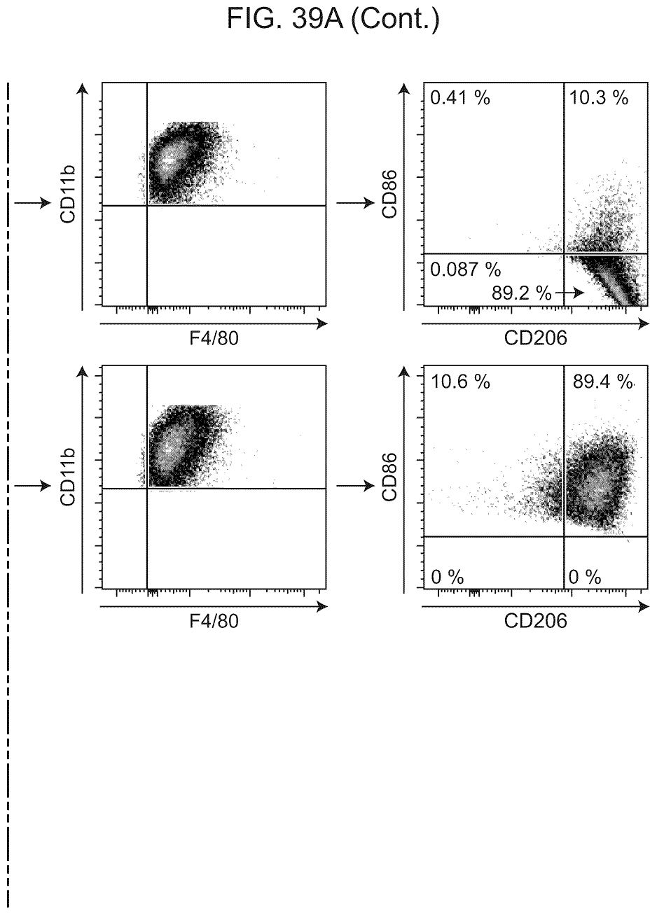

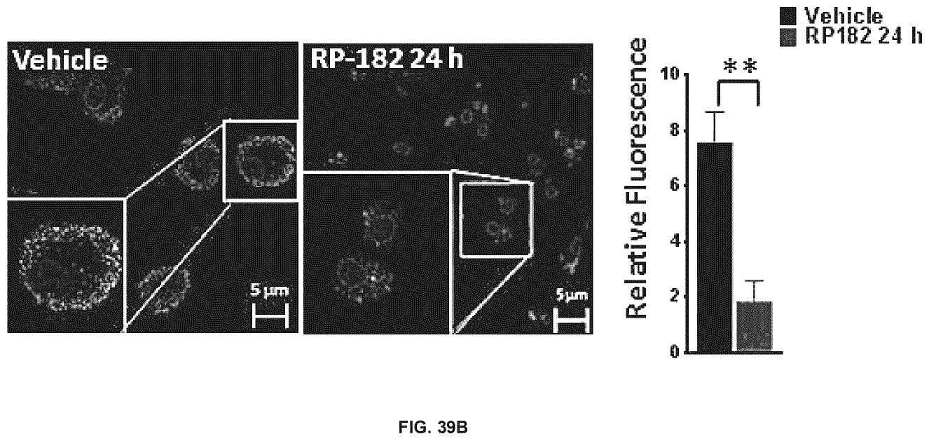

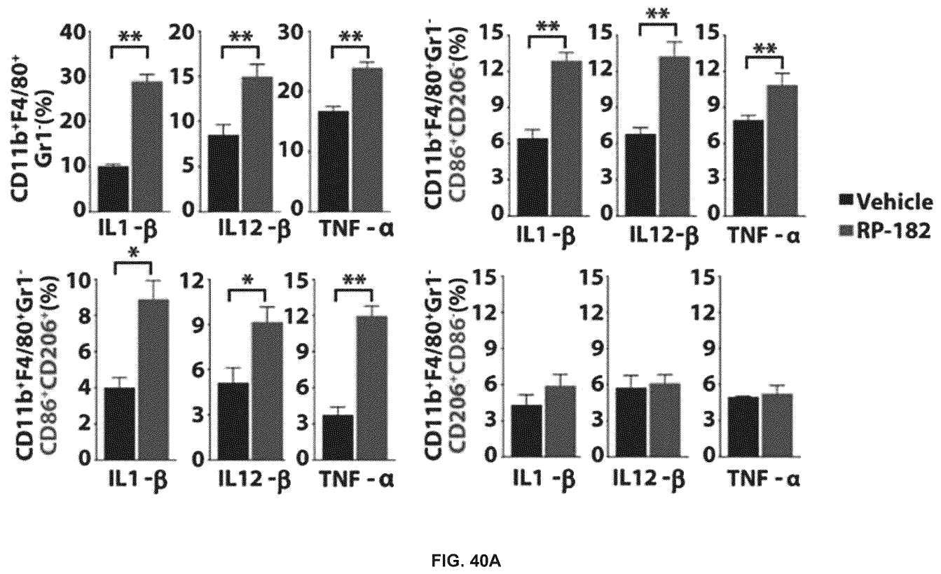

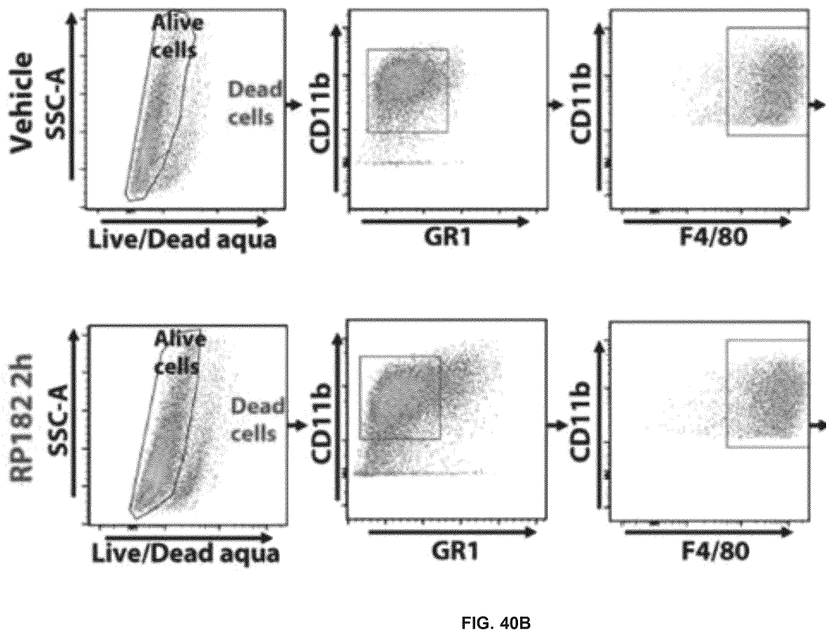

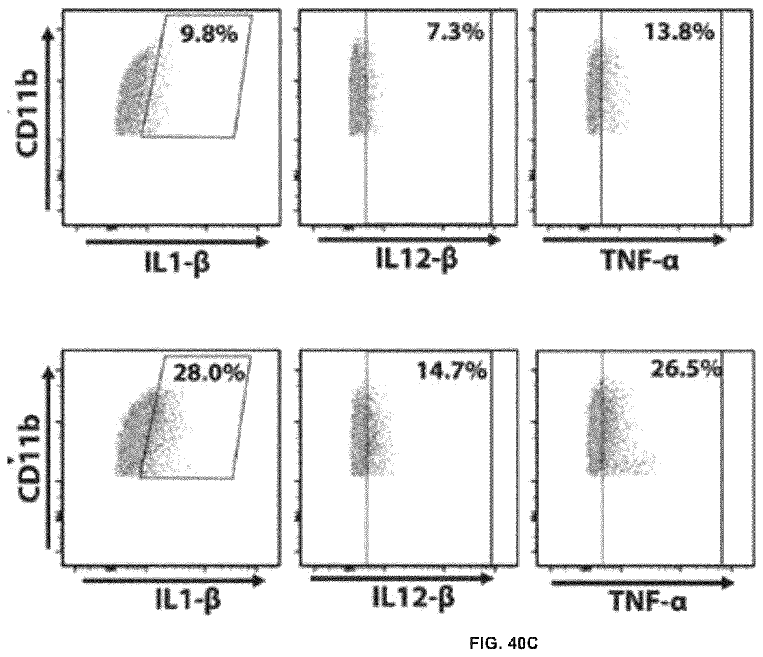

Flow cytometry experiments of CD11b+F4/80+Gr-1− macrophages gated on alive cells using the M1 marker CD86 and M2 marker CD206 showed rapid induction of CD86 expression with an increase in the CD86+CD206+ double-positive macrophages fraction (87.8% vs 10.3% in vehicle-treated control) within 30 min upon treatment with RP-182 (FIG. 38 and FIGS. 39A-B). Expression of CD86 was followed by loss of CD206 expression resulting in a CD86+M1-like fraction not expressing the M2 marker CD206 of 10.6% after 24 hours of treatment (FIG. 38). Increased CD86 expression in M2 BMDMs treated with RP-182 was accompanied by upregulation of M1 cytokines and markers, including IL-1β, IL-12, TNFα, and inducible nitric oxide synthase (iNOS) expressed by M1 macrophages (FIG. 40A-C).

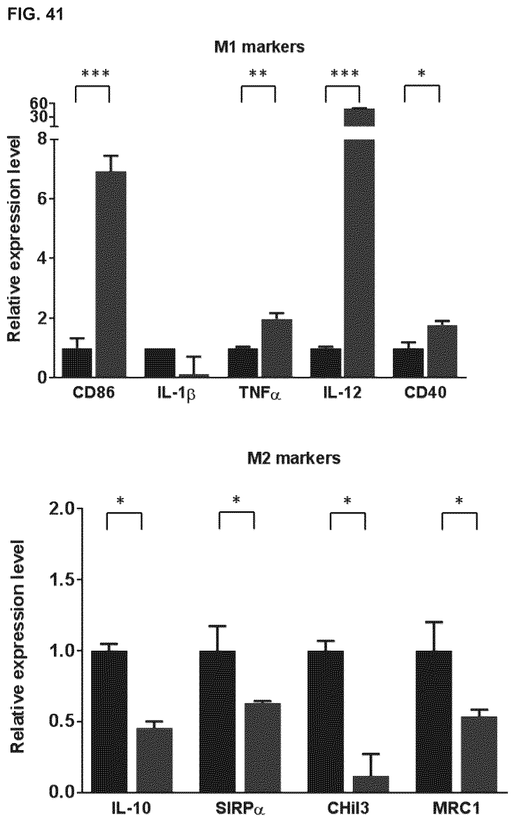

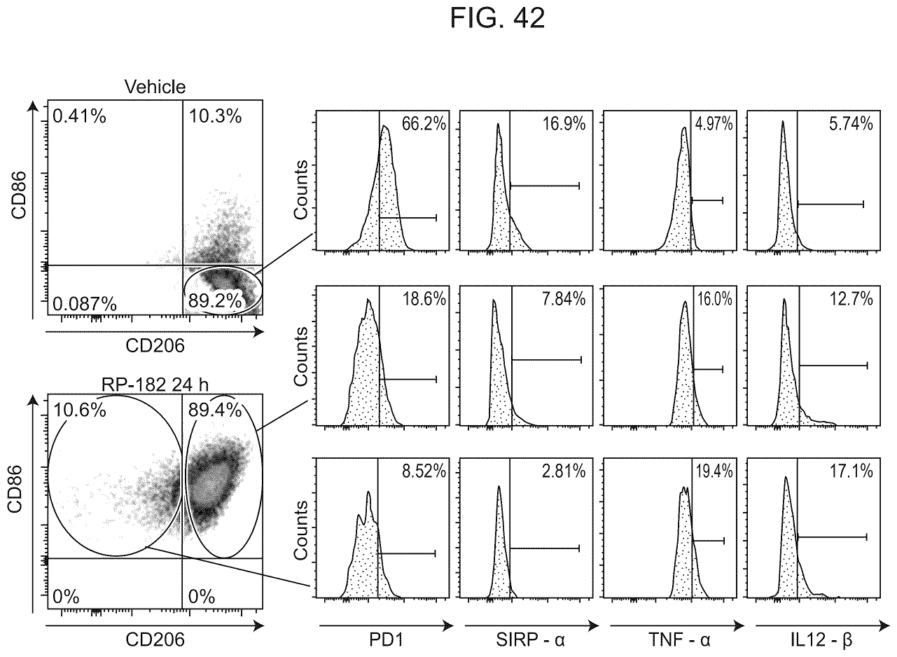

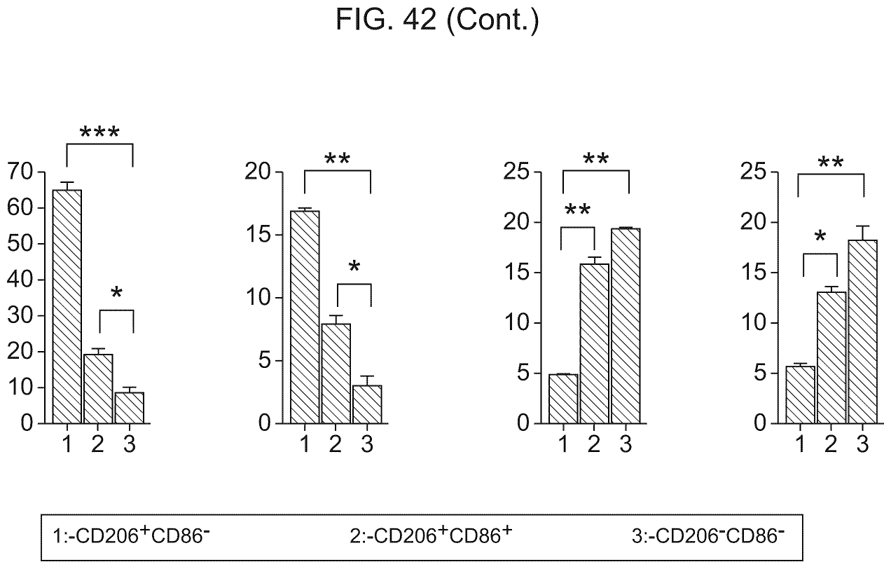

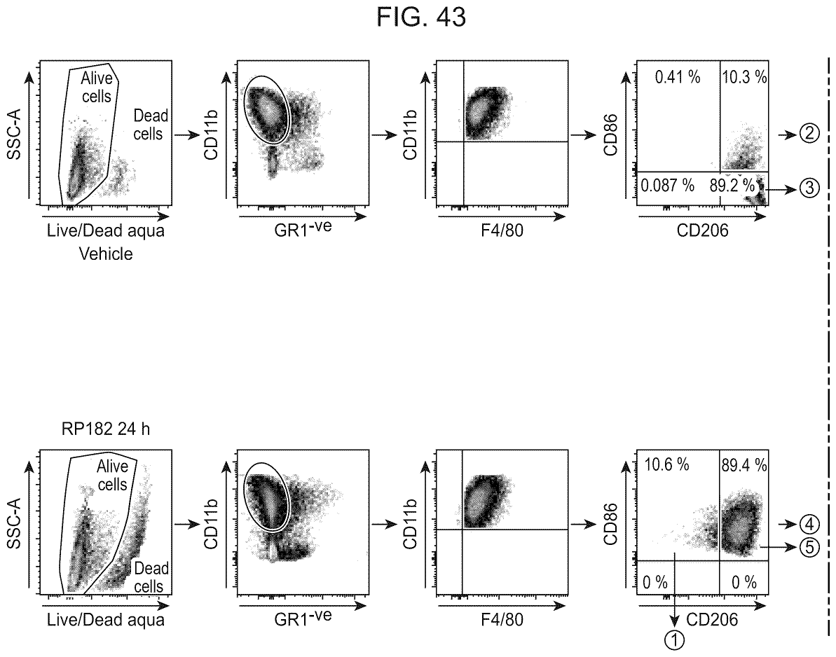

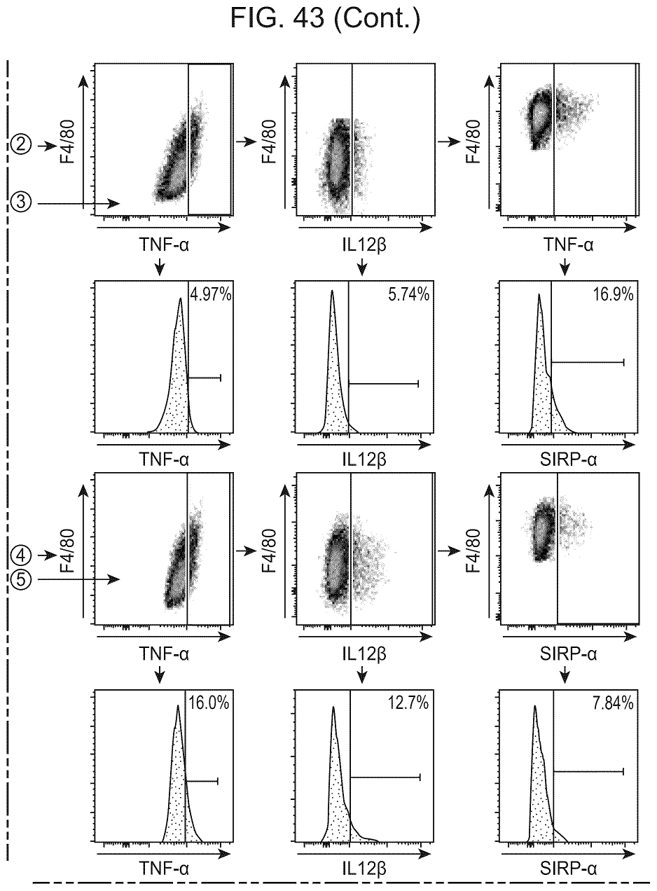

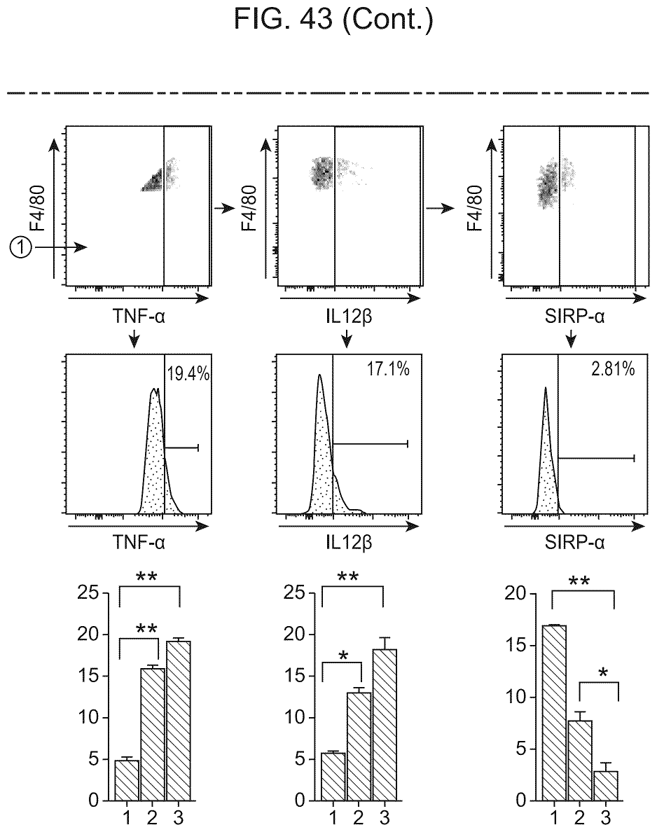

Induction of M1 and loss of M2 markers was also observed in M2 BMDMs treated with RP-182 isolated by fluorescence-activated cell sorting (FACS) (FIG. 41). The increase in M1 cytokine expression was selective for CD86+ macrophages and not observed on CD206+CD86− cells (FIG. 40A-C). The induced M1-like CD86+CD206− and double positive CD206+CD86+ macrophage cell populations showed decreased number of cells staining positive for the PD-1 (8.52% and 18.6% vs 66.2%) and inhibitory regulatory membrane glycoprotein signal regulatory protein α (SIRPα) (2.81% and 7.84% vs 16.9%) immune checkpoints compared to vehicle-treated CD86−CD206+ M2 cells (FIG. 42 and FIG. 43). The phenotypic switch induced by RP-182 towards M1 was accompanied by elevated M1 function as the fraction of macrophages involved in bacterial phagocytosis, a function more commonly associated with the M1 phenotype, increased from 20.4 to 81.3 percent after treatment with RP-Macrophage fractions staining positive for the M1 cytokines TNFα and IL-12 were increased (19.4% and 16.0% vs 4.97/a, and 17.1% and 12.7% vs 5.74/, respectively) compared to vehicle control.

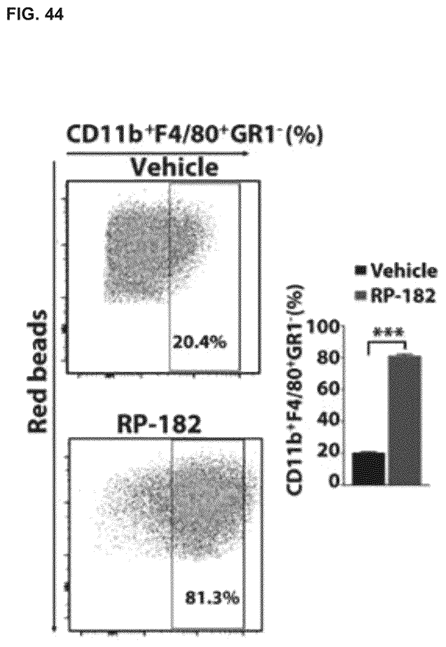

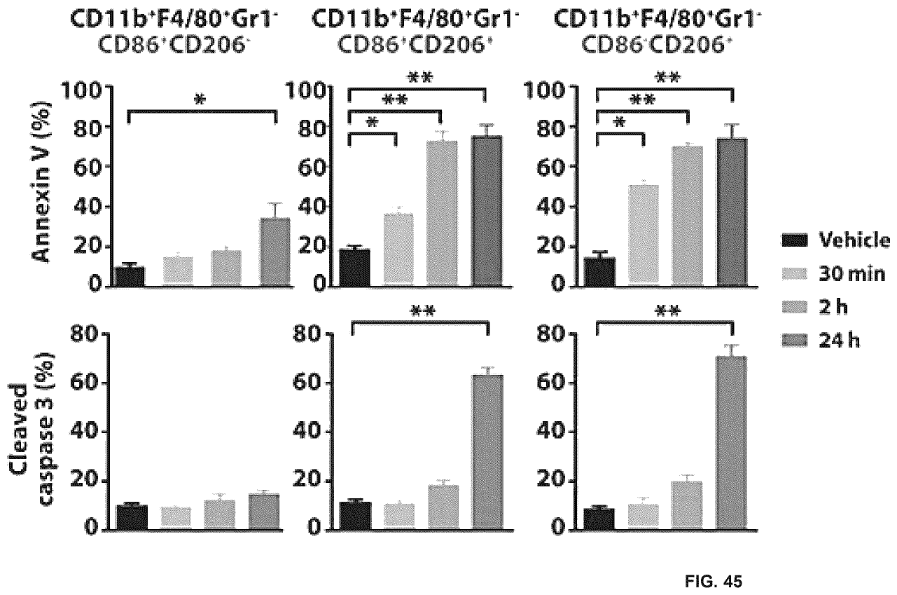

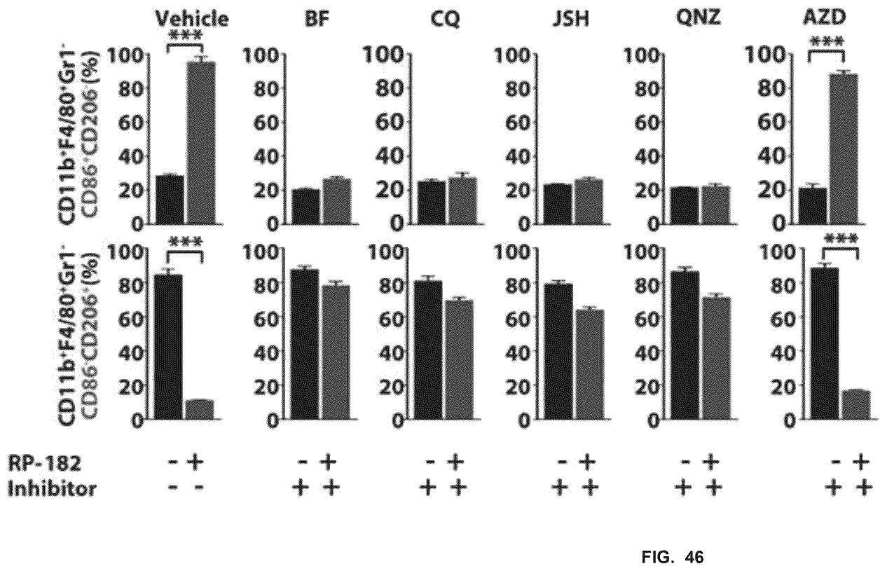

The phenotypic switch induced by RP-182 towards M1 was accompanied by increased bacterial phagocytosis, a function more commonly associated with the M1 phenotype (FIG. 44). Of note, the rate of apoptosis in the reprogrammed M1-like CD86+CD206− cell macrophages after treatment with RP-182 was significantly lower than in the CD206+CD86− positive and CD206+CD86+ double-positive cells possibly indicating that (1) CD206 negative cells escape the direct cell killing of RP-182 and (2) RP-182 reprograms M2 macrophages towards a M1-like phenotype (FIG. 45). Both, pharmacological blockade of RP-182-induced NF-kB signaling and autophagy, previously shown to be induced by RP-182, suppressed reprogramming effect of RP-182 towards the M1 phenotype (FIG. 46).

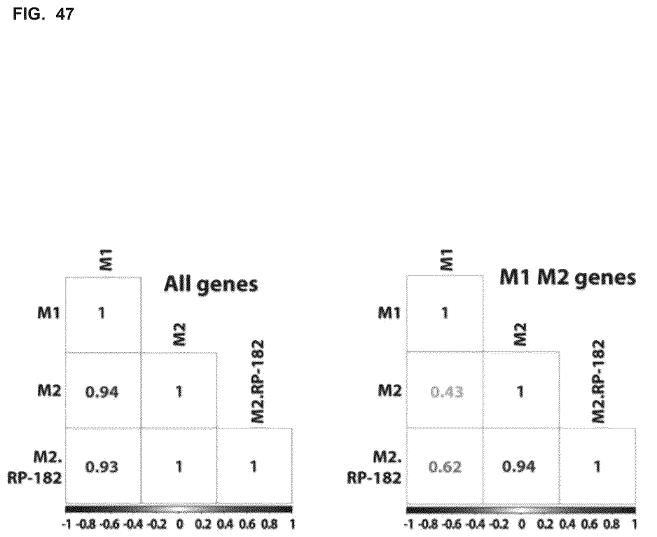

To examine whether gene expression changes induced by RP-182 support reprogramming of M2 BMDMs towards a M1-like phenotype, gene expression matrices were analyzed from RNASeq data. Pearson's correlation analysis of gene expression matrices derived from global RNASeq data of RNA isolated from M1, M2, and M2 BMDMs treated with RP-182 showed a high degree of similarity between the three datasets. Using a M1 M2 marker set previously described for characterization of macrophage phenotypes in BMDMs, M2 macrophages displayed greater similarity after RP-182 treatment to untreated M1 than to untreated M2 cells (FIG. 47). In summary, in addition to induction of phagocytosis, autophagy, and apoptosis in M2 macrophages, the synthetic HDP RP-182 induces a shift towards a M1-like phenotype.

### Example 4: Mechanism of Action of RP-182

The mechanism of action of RP-182 was investigated, showing that the action is dependent on CD206 and initiates Rac1/Cdc42 activation and IQGAP1 recruitment.

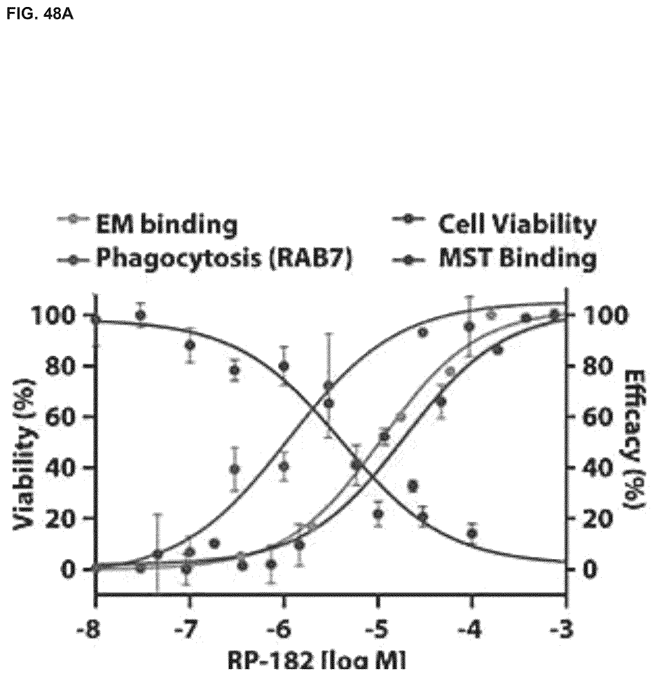

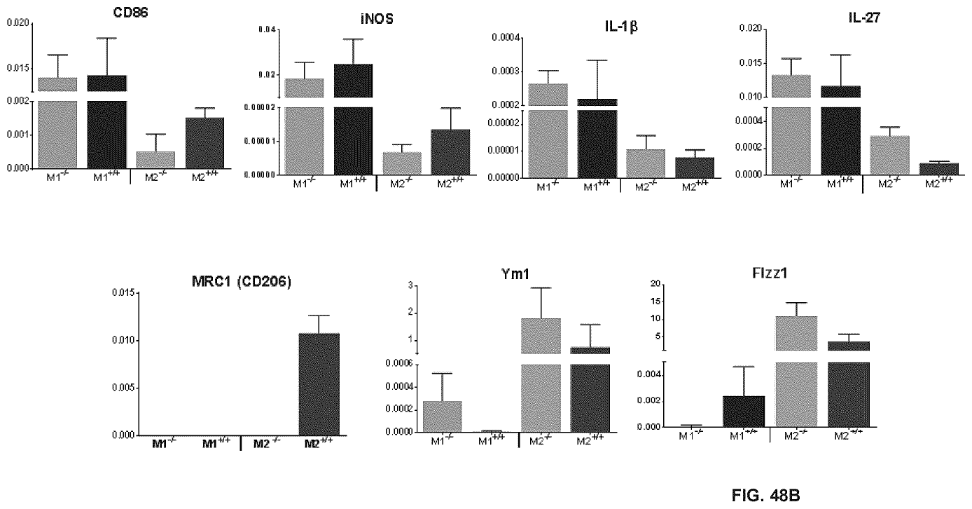

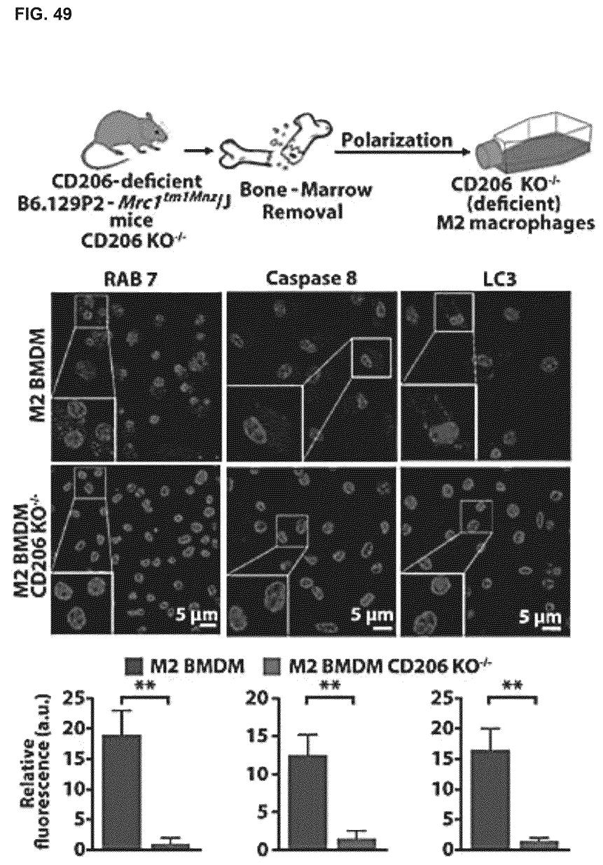

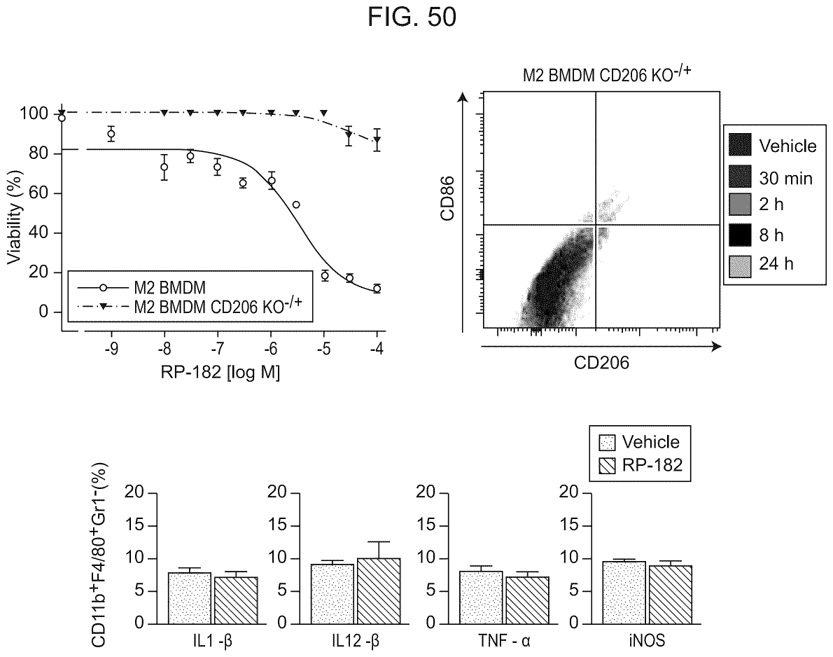

When overlaying pharmacodynamic readouts of RP-182 treatment, the close EC50 and IC50 activities of RP-182 suggest a shared mechanism of action mediated by a common CD20 target (FIG. 48A). To show that the M2-selective action of RP-182 is indeed MRC1/CD206− dependent, BMDMs were isolated from B6.129P2-Mrc1tm1Mnz/J mice, which are deficient of CD206 (24). It was first confirmed that with the exception of CD206, there was upon polarization into M1 and M2 populations no difference in the expression profile of M1 and M2 markers between CD206 wt and CD206−/− BMDMs (FIG. 48B). In contrast to M2-polarized macrophages from wild type mice, M2-polarized macrophages isolated from B6.129P2-Mrc1tm1Mnz/J mice failed to show induction of phagocytosis, autophagy, or apoptosis (FIG. 49), did not respond to RP-182 (FIG. 50), and showed no induction of M1 cytokines upon treatment with RP-182 (FIG. 50).

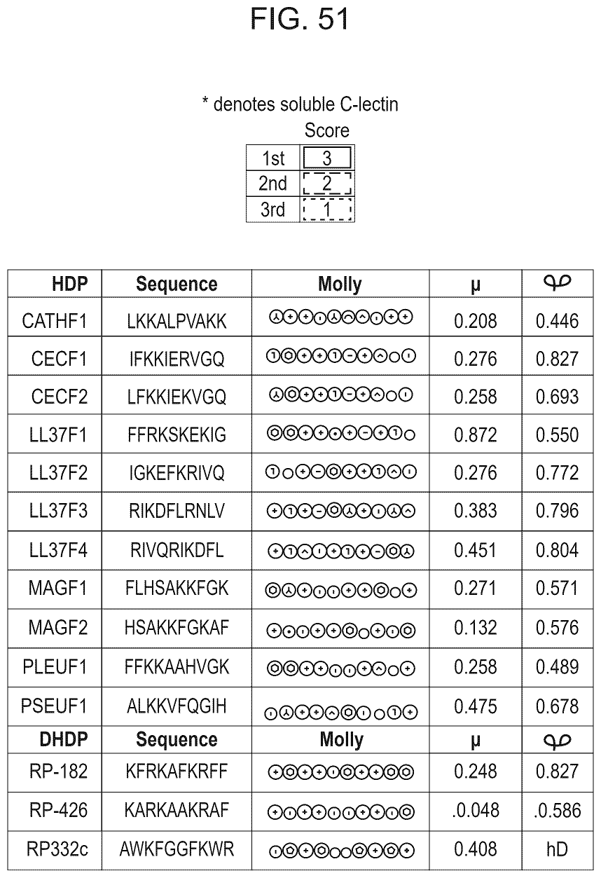

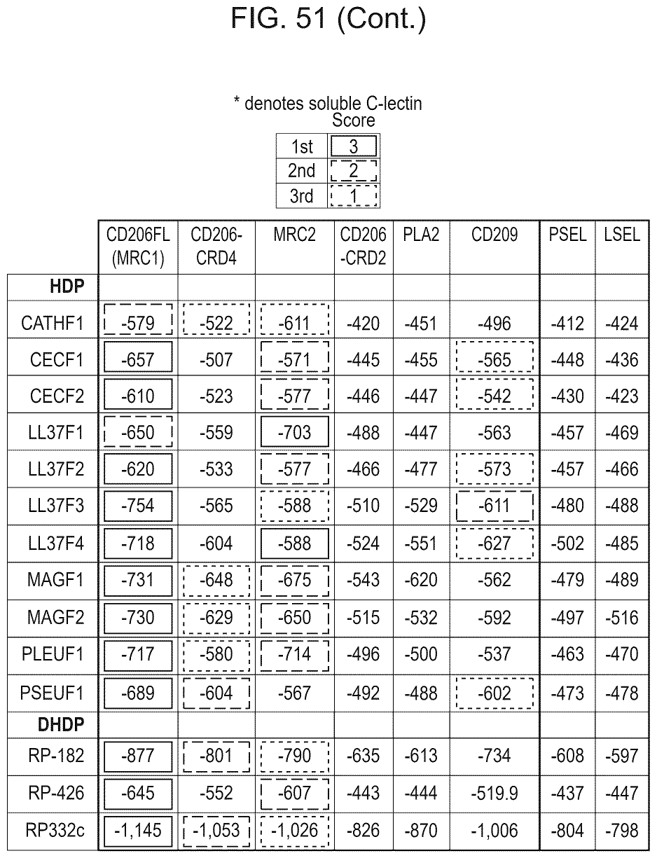

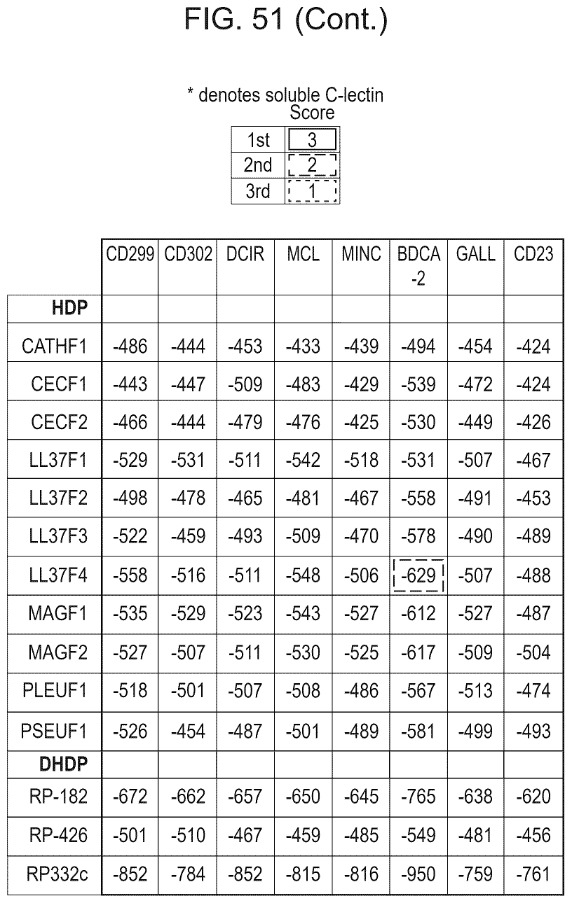

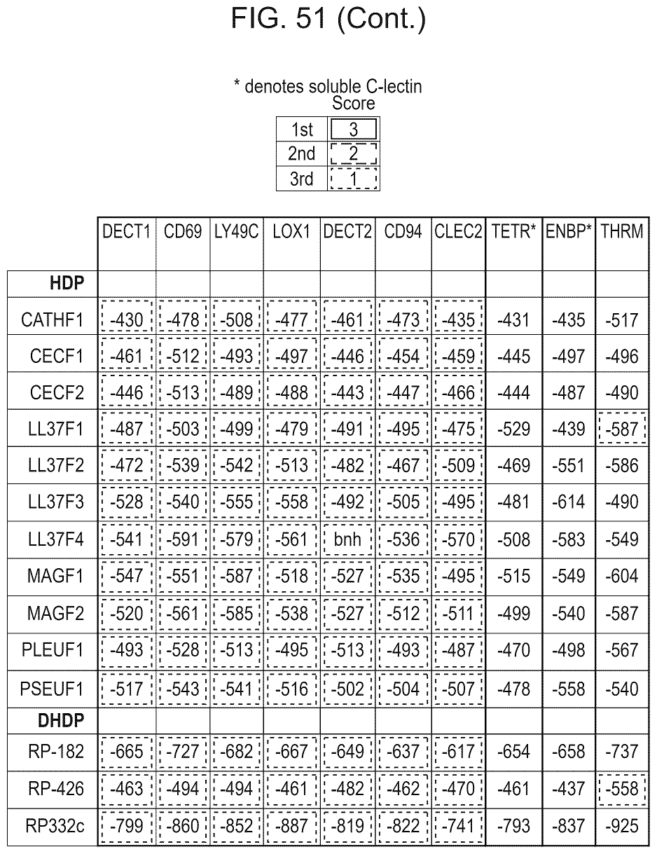

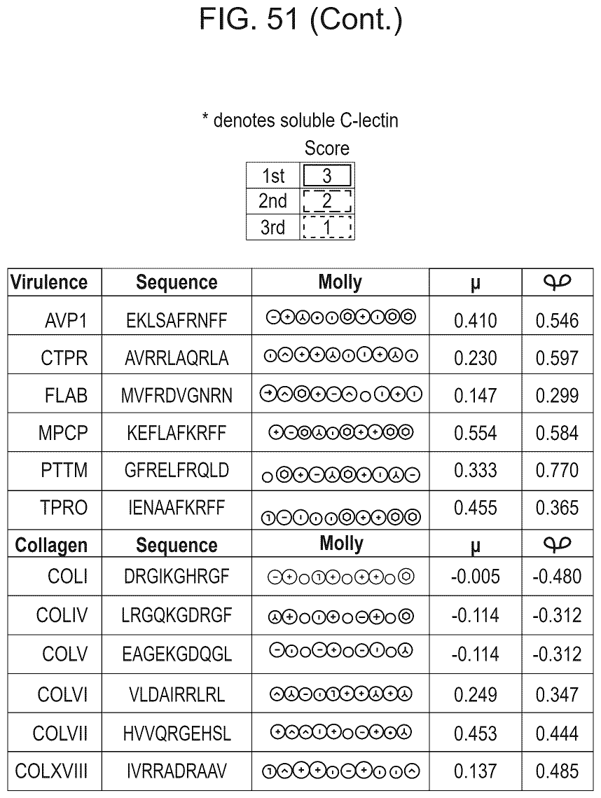

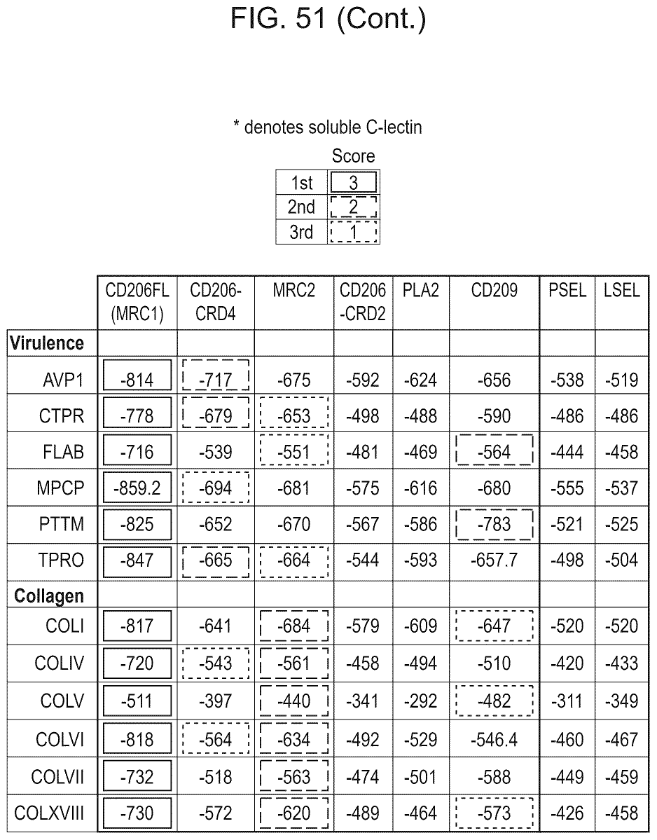

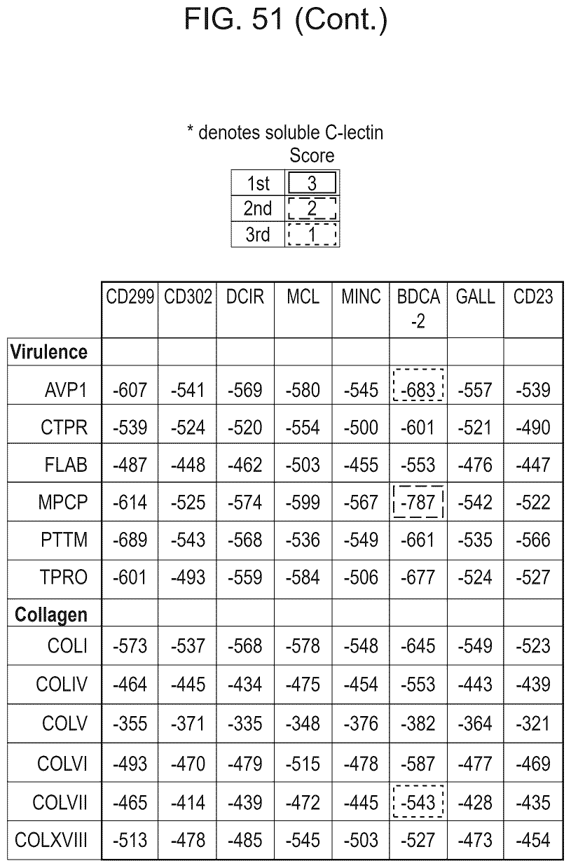

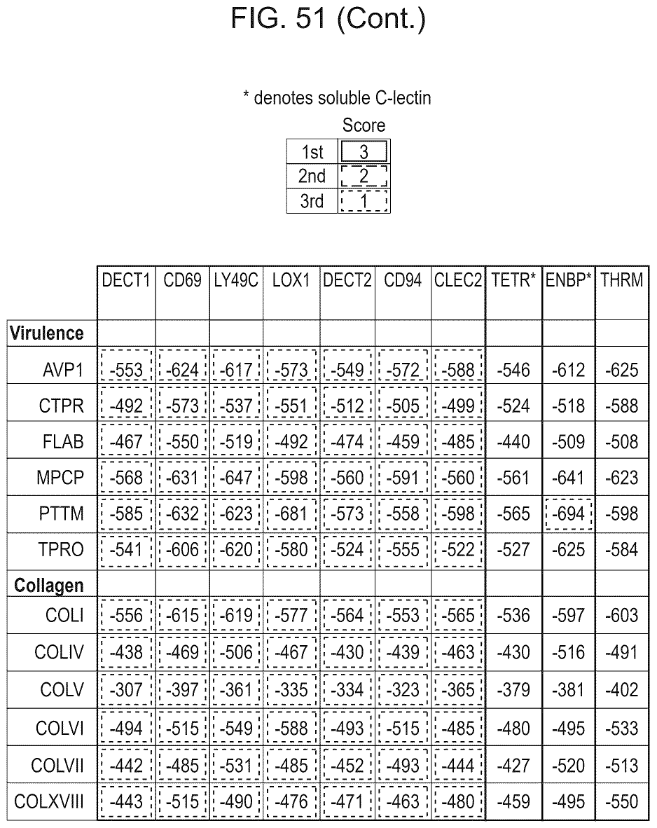

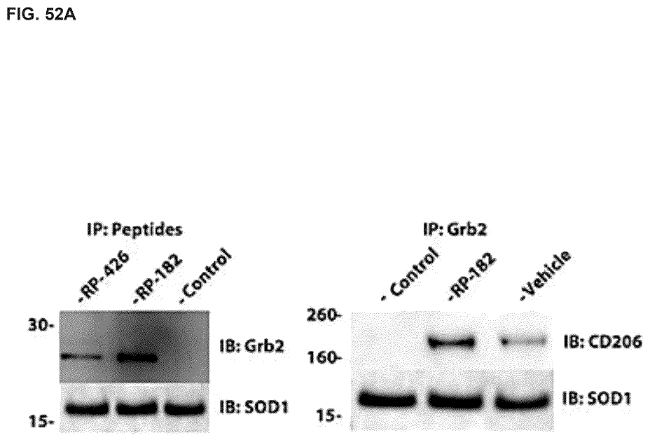

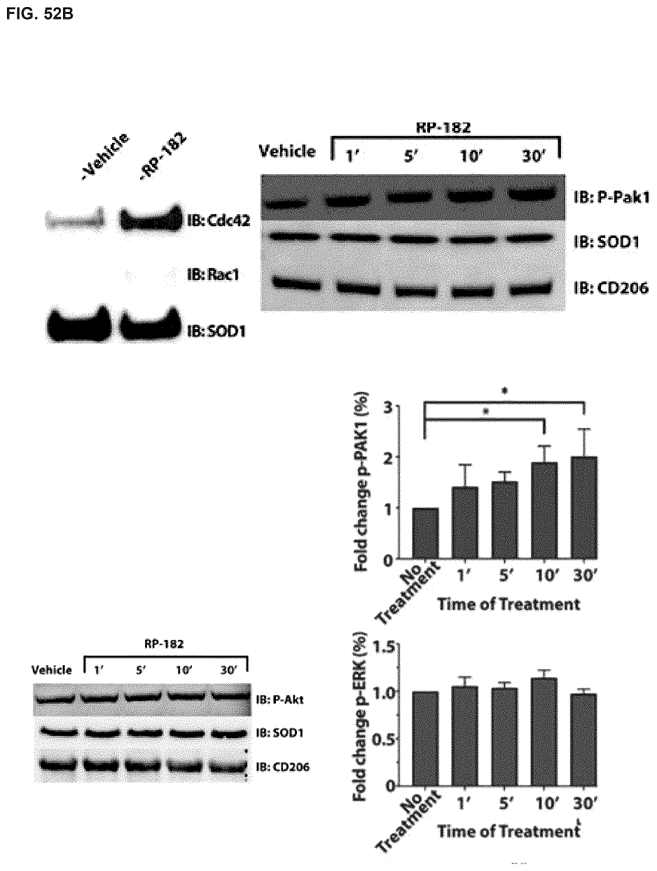

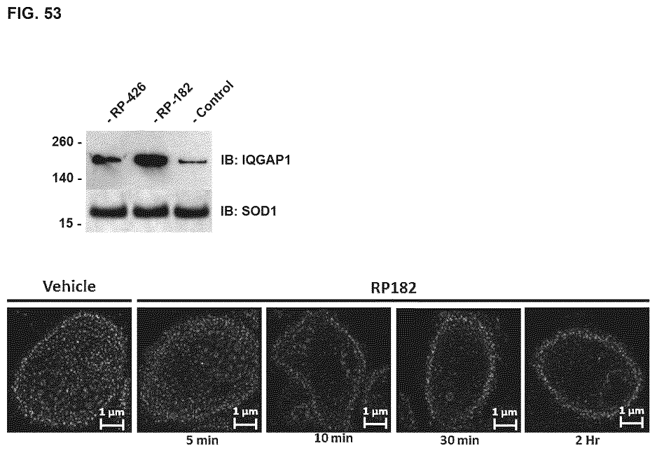

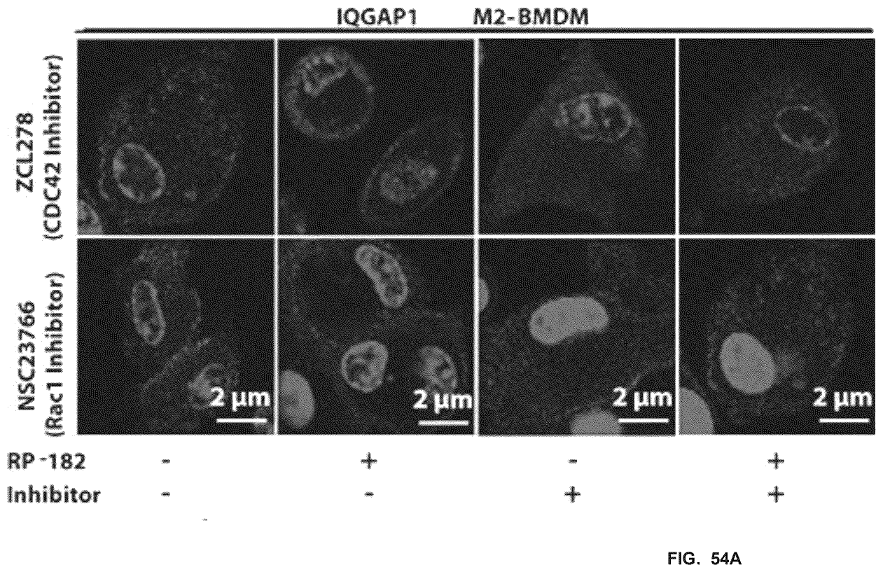

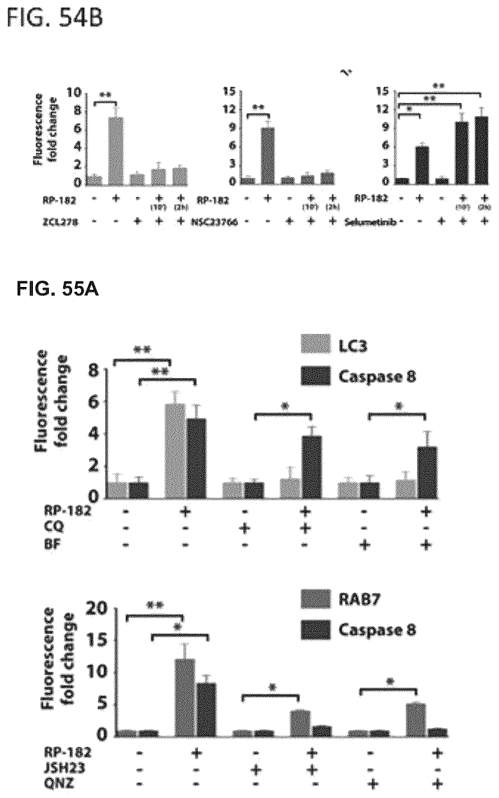

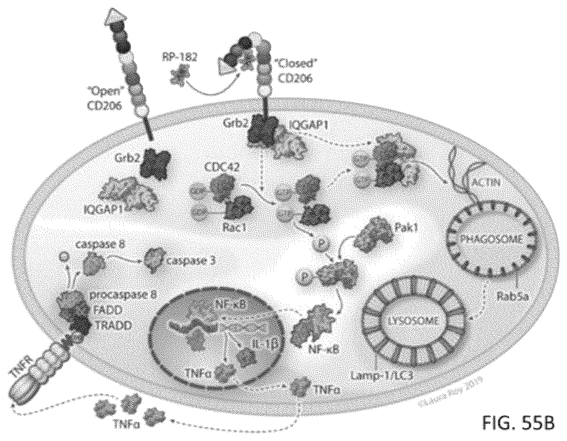

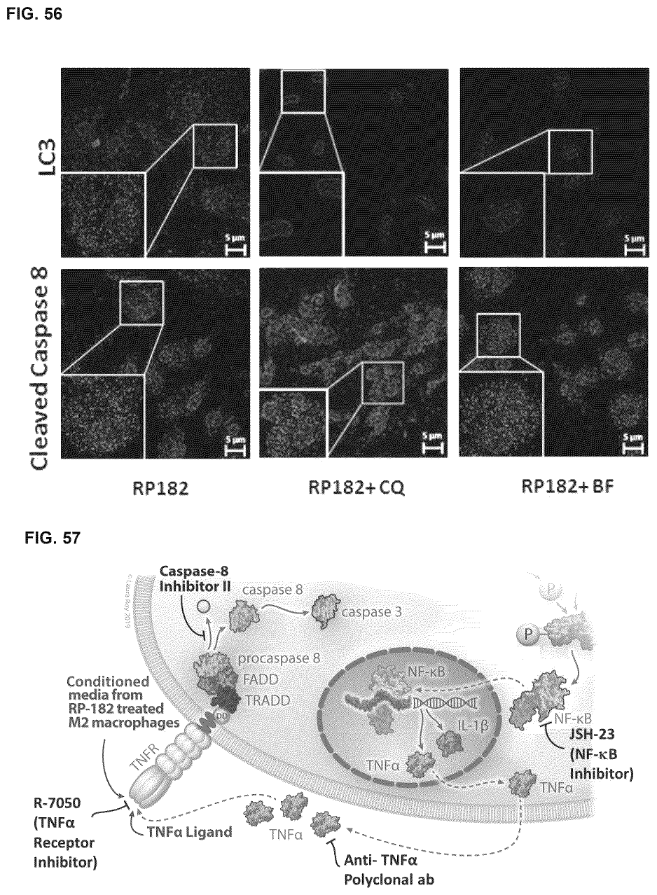

To better understand the downstream MRC1/CD206 signaling mechanism induced by RP-182, the proteomic analysis of MRC1/CD206 complexes was revisited from M2-polarized BMDMs treated with vehicle or biotinylated RP-182 (FIG. 50; FIG. 51). Previous studies identified growth factor receptor-bound protein 2 (GRB2) as an intracellular signaling adaptor molecule of mannose CD206 receptor activation of the CD206 receptor by mycobacteria induces phagocytosis via GRB2 recruitment as well as activation of the Rac1/CDC42/Pak1 signaling cascade, regulators which were GRB2 was highly enriched in the CD206 pulldown from RP-182-treated BMDMs. Co-immunoprecipitation and immunoblotting studies in M2 macrophages showed that binding of RP-182 to CD206 recruits GRB2 and activates Rac1/CDC42/Pak1 signaling (FIGS. 52A-B). As control we tested the phospho-AKT levels in RP-182 treated M2 macrophages and no activation of this pathway was identified. IQ motif containing GTPase activating proteins 1 and 2 (IQGAP1 and 2), which were 9- and 76-fold enriched in CD206 complexes pulled down from RP-182-treated cells compared to control, have previously been shown to be effectors of small GTPases Rac1/CDC42 in cytoskeletal dynamics stabilizing the GTP-bound, active state of Rac1/CDC42 and to be involved in endocytosis/phagocytosis. In M2 BMDMs, RP-182 increased the binding of IQGAP1 to the CD206 complex and induced membranous recruitment within 10 minutes (FIG. 53). Blockade of Rac1/CDC42 signaling abrogated RP-182-induced IQGAP1 membrane translocation and induction of phagocytosis (FIGS. 54A-B). Treatment with autophagy inhibitors prevented induction of RP-182-induced LC3 expression, but did not affect caspase 8 induction, suggesting that caspase 8 activation, which did not occur in the presence of NF-kB inhibitors, is not part of an autophagolysosomal cascade but driven by NF-kB activation mediated by Rac1/CDC42-Pak1 signaling activation (FIGS. 55A-B and FIG. 56). Indeed, induction of apoptosis was mediated by RP-182-induced autocrine TNFα signaling triggered by NF-kB activation. Blockade of TNFα signaling abrogated induction of caspase 8 and 3 activation, while conditioned media from RP-182-treated M2 BMDMs activated apoptosis which was not observed in the presence of anti-TNFα antibodies (FIG. 57).

These data suggest that RP-182 binding to CD206 recruits GRB2 and the Rac1/CDC42 effector IQGAP1 and activates Rac1/CDC42/Pak1 signaling promoting phagocytosis and autophagy and co-stimulates NF-kB signaling which is associated with induction of apoptosis via autocrine TNFα signaling (FIGS. 55A-B).

### Example 5: Association of CD206 Expression Status with Intratumoral Immunity

The association of CD206 expression status with intratumoral immunity was assessed, and it was found that CD206^{high} expression status is associated with decreased intratumoral immunity in human and murine pancreatic cancers.

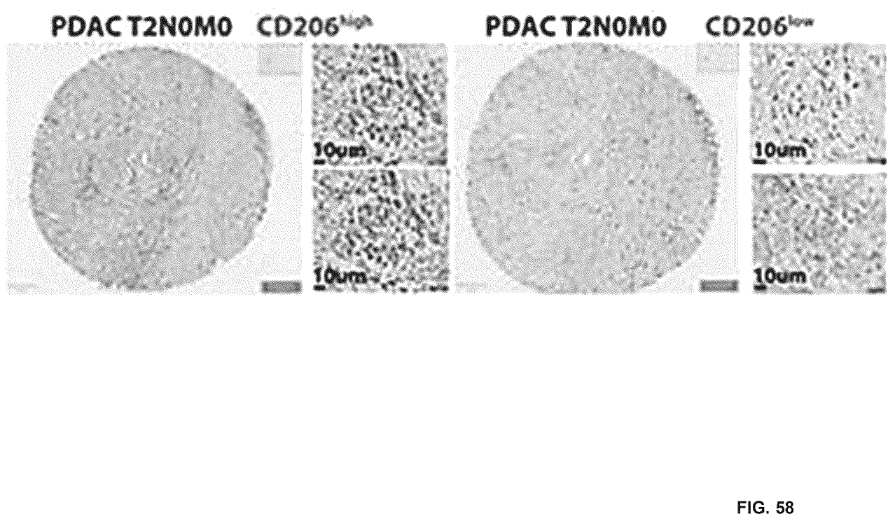

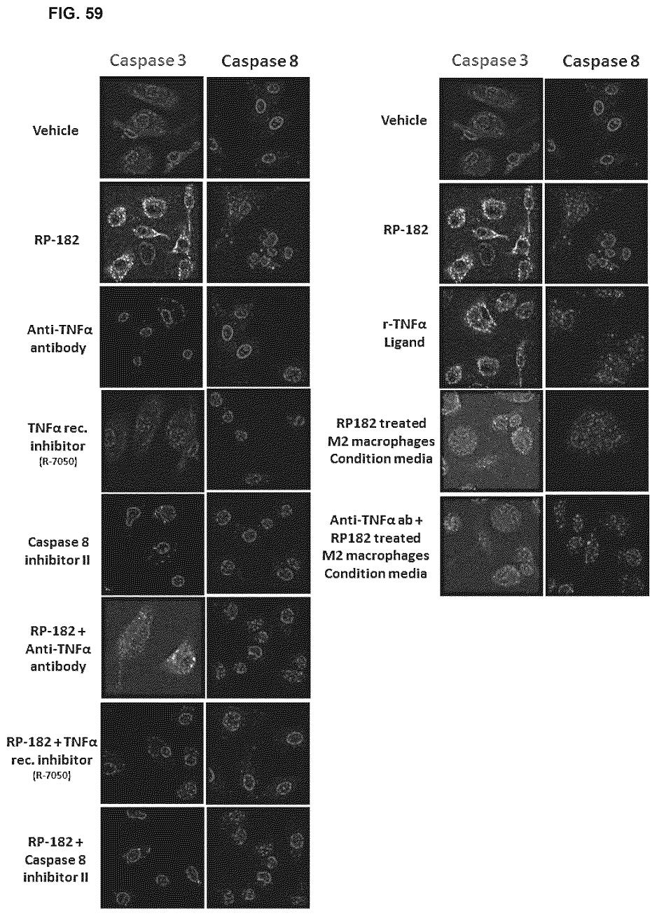

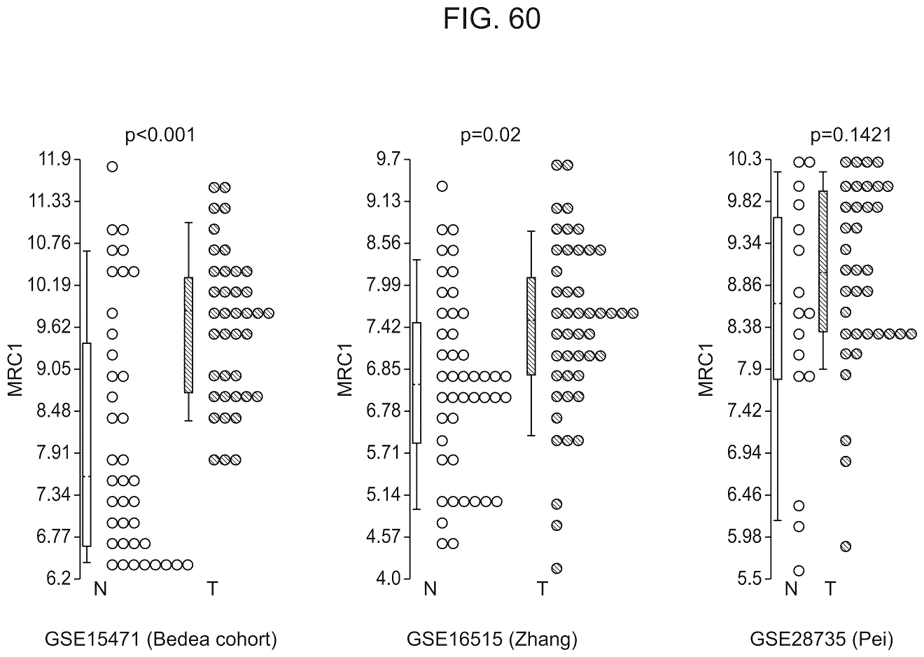

CD206 expression status as a surrogate for M2 macrophage population varied considerably across clinical pancreas cancer resection specimens (FIG. 58 and FIG. 59). CD206 was found to be overexpressed in two of three available independent gene expression sets of clinical pancreatic cancer specimens compared to matched uninvolved normal pancreas with gene set GSE28735 showing a trend towards higher CD206 expression in the tumors (gene sets GSE15471, GSE16515, and GSE28735; FIG. 60).

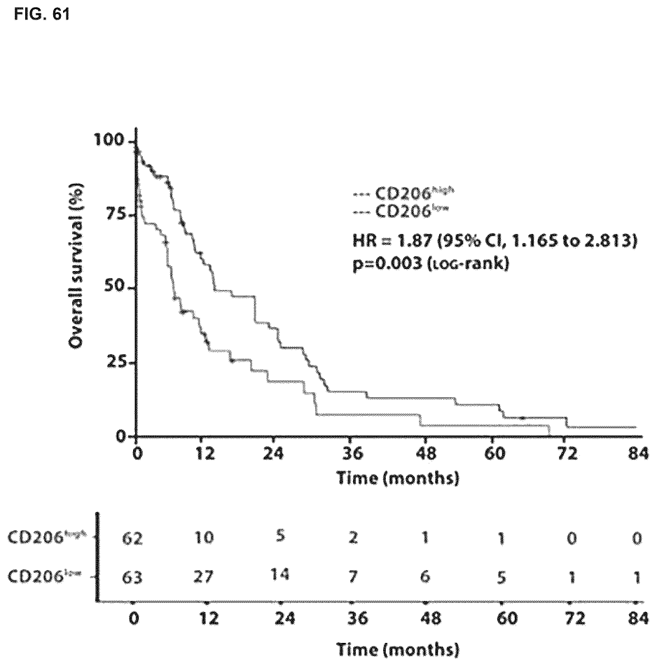

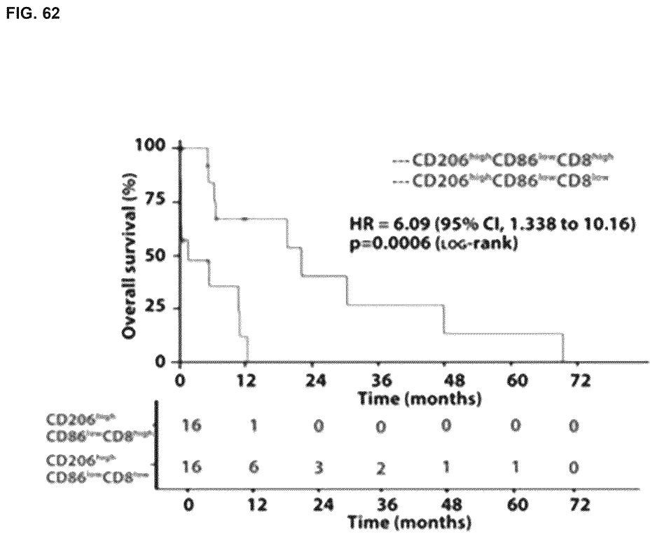

Overall survival (OS) of patients with pancreatic cancers was more unfavorable in the CD206high clinical cases (HR 1.87, 95% confidence interval (CI) 1.165 to 2.813; log-rank test; p=0.003) (FIG. 61). Infiltrating CD8+ T cells measured by CD8 transcript levels separated outcome of clinical cases with high M2-like populations further (HR 6.09, 95% CI, 1.338 to 10.16; log-rank test; p=0.0006) (FIG. 62).

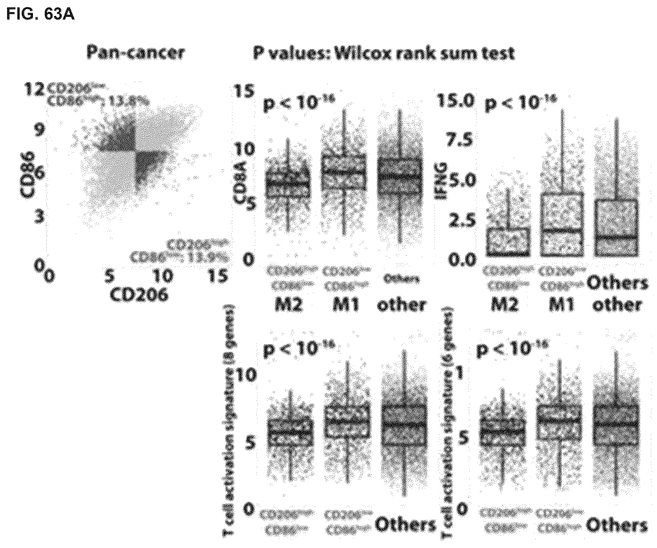

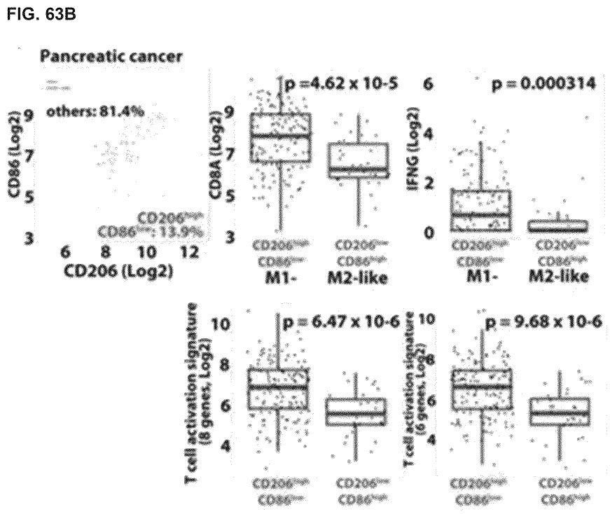

To examine whether the adverse disease outcome in CD206high cases is supported by immune subpopulation correlations in human pancreatic cancers, or across solid organ cancers, correlations of intratumoral macrophage subsets and surrogates of intratumoral CD8+ T cell function were investigated in TCGA pan-cancer and pancreatic adenocarcinoma data sets. After selecting tumors with M2-high fractions and low abundance of M1-like macrophages, there was a negative correlation with CD8 transcripts as well as measures of CD8+ T cell function including low expression of two previously described T cell activation response signatures (FIGS. 63A-B). To study the association of CD206 and clinical outcome further, we generated murine pancreatic cancers in CD206-deficient B6.129P2-Mrc1tm1Mnz/J mice.

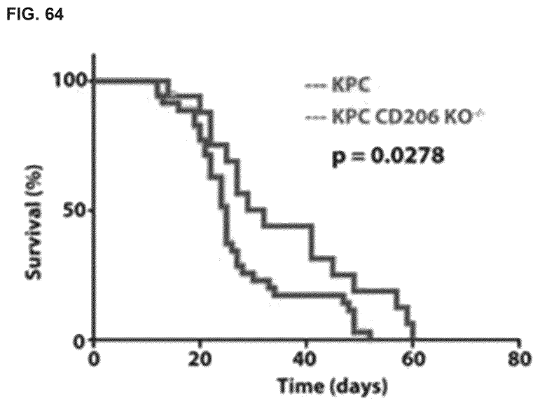

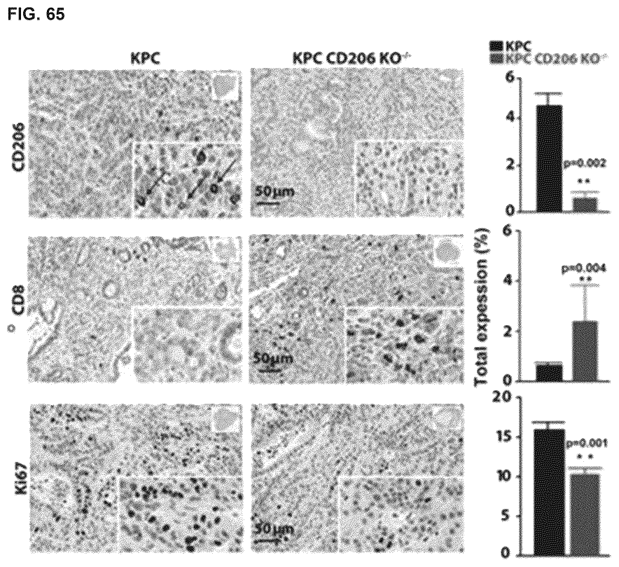

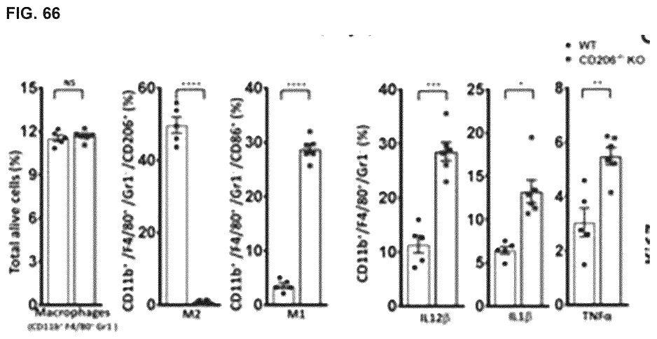

There was a discernable difference in survival of KPC CD206−/− vs CD206 wild type allografts with CD206-deficient tumors showing prolonged overall survival (median OS of KPC CD206−/− vs CD206 wild type, 32 vs 25 days; p=0.0278; FIG. 64). KPC tumors in CD206−/− mice showed absent CD206 expression and, in line with the negative correlation of CD206high and CD8 T cell function in the human cancer specimens, significantly increased intratumoral CD8+ T cell numbers compared to KPC tumors generated in CD206-proficient C57B/L6 wild type mice (FIG. 65). In summary, CD206-positive M2-like TAMs are negatively correlated with intratumoral T cell function. Pancreatic cancers allografted into CD206-deficient B6.129P2-Mrc1tm1Mnz/J mice have attenuated cancer progression compared to tumors generated in CD206-wildtype mice and share immunogenic traits including increased intratumoral CD8+ T cells observed in human CD206low tumors. KPC tumors in CD206−/− mice attracted an equal number of TAMs compared to KPC tumors grown in wild type mice, of note there was a significant shift towards a M1-like phenotype in the TAM population of CD206−/− mice (FIG. 66).

### Example 6: Effect of RP-182 on Tumors and Tumor Microenvironments

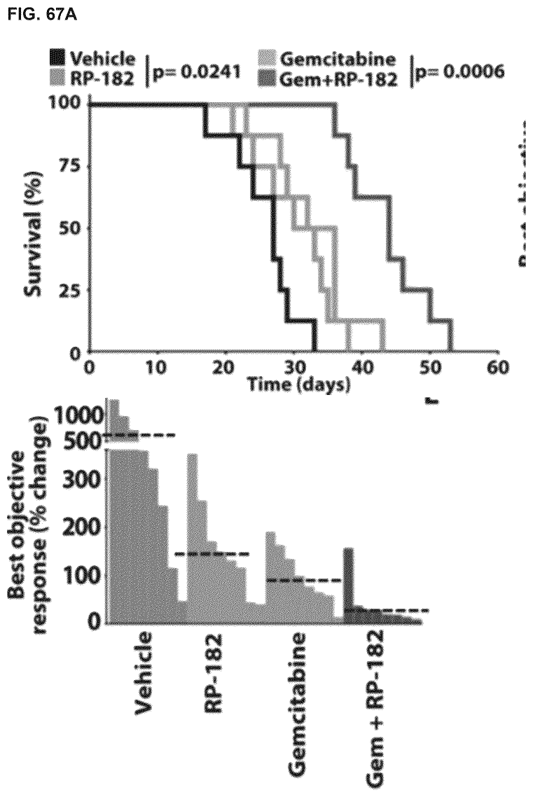

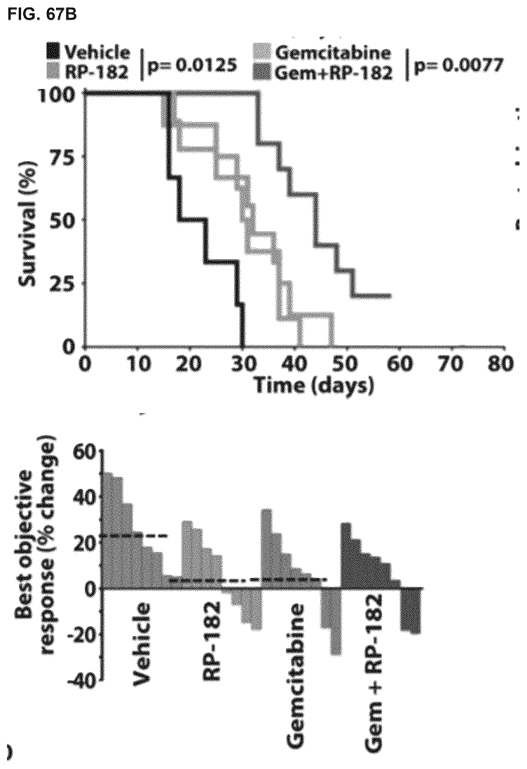

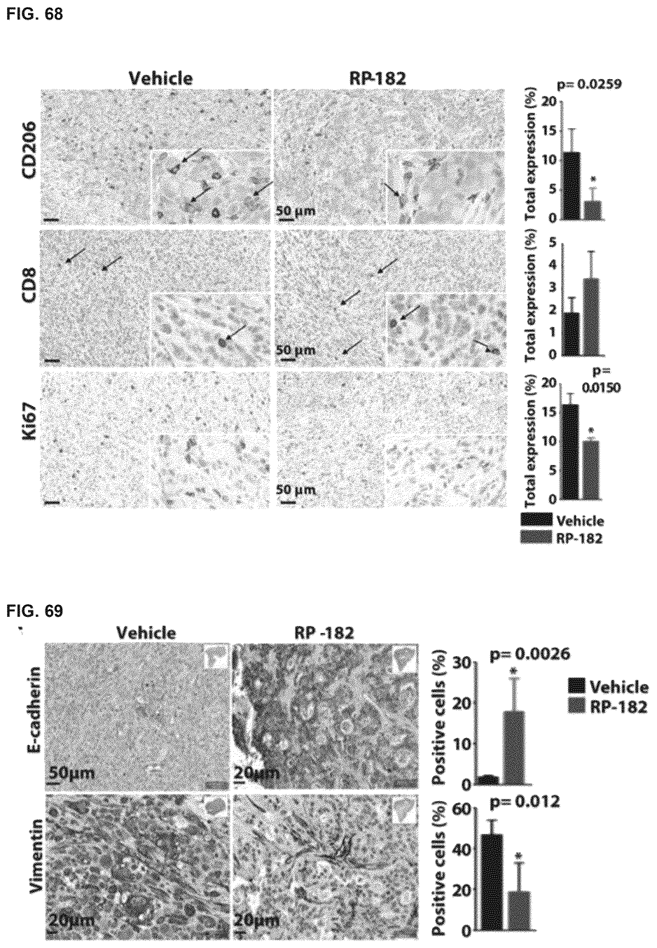

The effect of RP-182 on tumors was studied, and it was found that RP-182 mediates anti-tumor activity and reprograms the tumor microenvironment. RP-182 was tested in the autochthonous genetically engineered Ras-driven KP16 and KPC models of pancreas cancer. Kaplan-Meier analysis and tumor growth measurements showed extension of survival and anti-tumor activity of RP-182 monotherapy yielding similar gains in survival and tumor suppression as gemcitabine (median overall survival (OS) of 20.5 vs 32 days in vehicle vs RP-182-treated KPC animals; p=0.0125, and 27 vs 31.5 days in KP16 animals; p=0.0241) (FIGS. 67A-B). Animals treated with the combination of RP-182 and gemcitabine were afforded in both models the greatest extension of survival with outcome in the combination cohort improved compared to single-agent treatment (34 vs 44 days in gemcitabine vs combination group in KP16 mice; p=0.0006 and 24.5 vs 42.6 days in KPC; p=0.0002, respectively) (FIG. 67A-B). Tumor tissues harvested at study endpoint showed reduced stromal CD206-positive macrophages and decreased nuclear Ki67 expression (FIG. 68). RP-182 induced E-cadherin expression and reduced expression of the epithelial-to-mesenchymal transition (EMT) marker vimentin (FIG. 69).

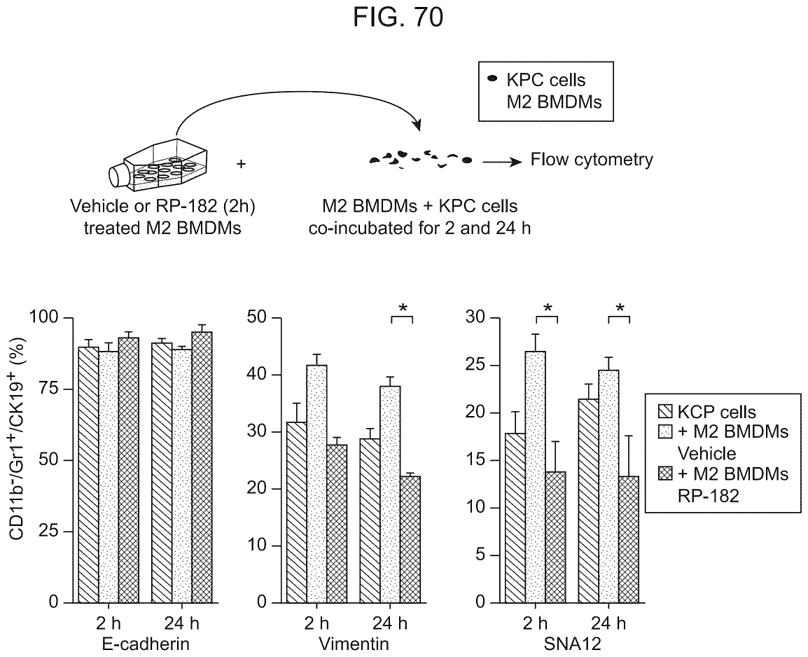

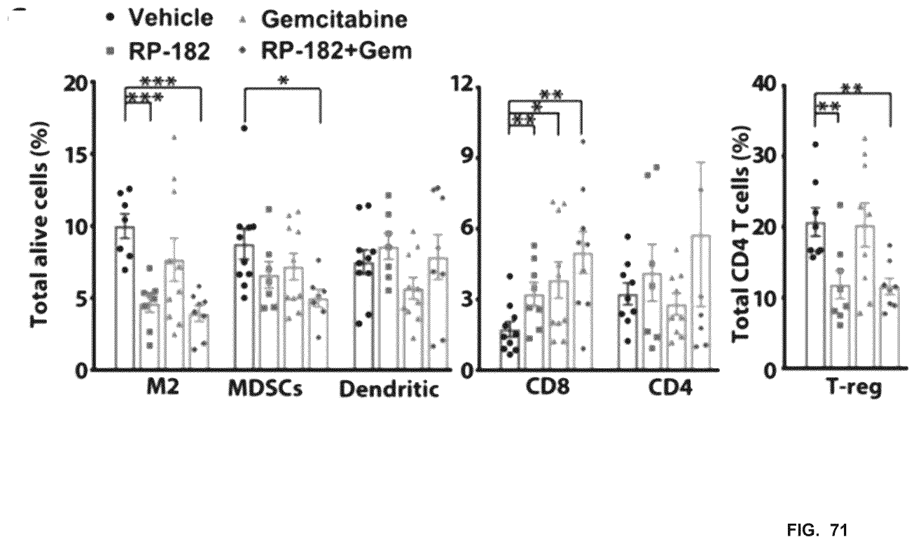

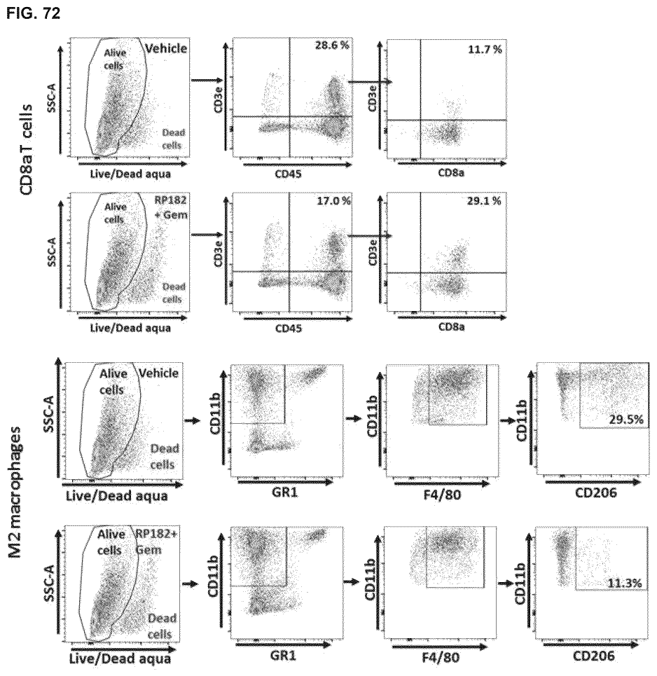

In vitro, the expression of the EMT markers vimentin and SNAIL in murine pancreatic cancer cells induced upon co-culture with M2 BMDMs was reduced when macrophages were pretreated with RP-182 compared to vehicle control (FIG. 70). Flow cytometry studies of tumor digests from KP16 mice treated for 7 days with RP-182 alone, and RP-182 in combination with gemcitabine confirmed reduced M2-like TAM fractions in RP-182 and RP-182 in combination with gemcitabine-treated mice (10.3% vs 4.61%, p=0.001 and 10.3 vs 3.91%, p=0.0003 respectively) (FIG. 71). RP-182 also decreased immune suppressive CD4 positive T regulatory (Tregs), and in combination with gemcitabine myeloid-derived suppressor cells (MDSCs) (8.75 vs 4.99%, p=0.015). Either alone or in combination with gemcitabine, RP_182 increased intratumoral CD8+ T cells (1.74 vs 3.40%, p=0.032 and 1.74 vs 4.99%, p=0.020, respectively) (FIG. 71 and FIG. 72). The reduction in the MDSC population occurred nearly exclusively in the CD206high monocytic MDSC subset whereas CD206low polymorphonuclear MDSCs did not show any change (FIG. 73).

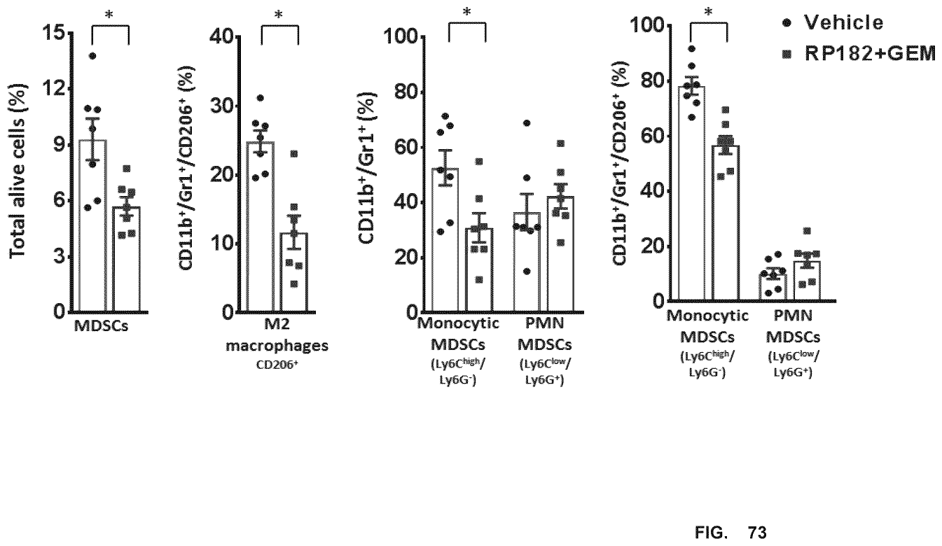

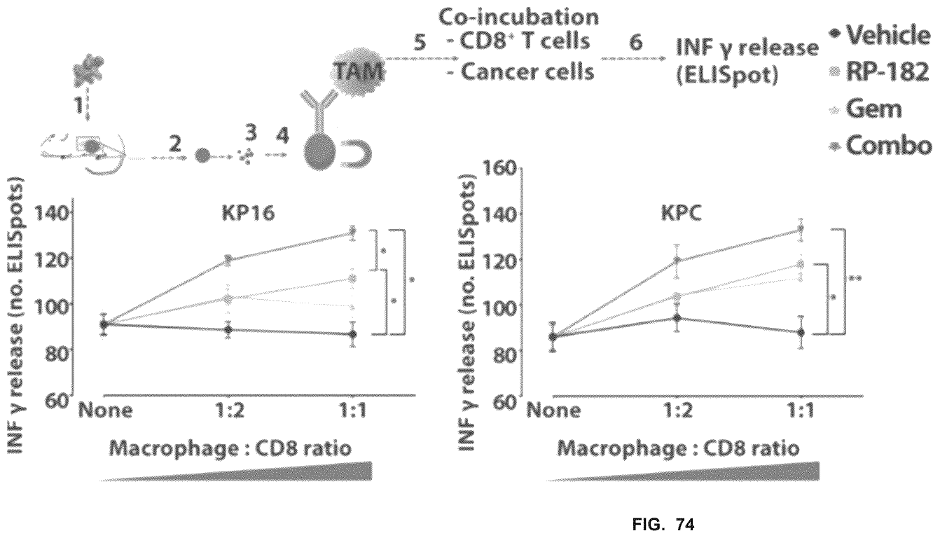

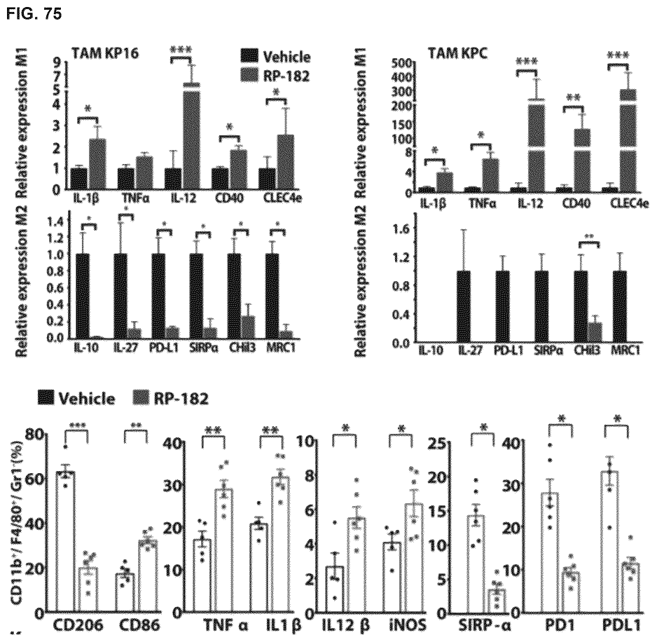

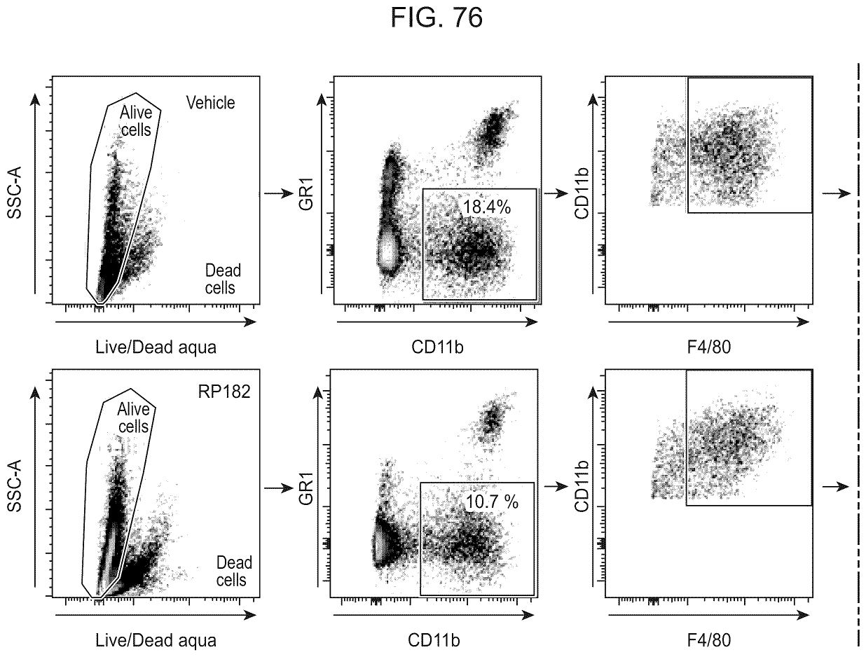

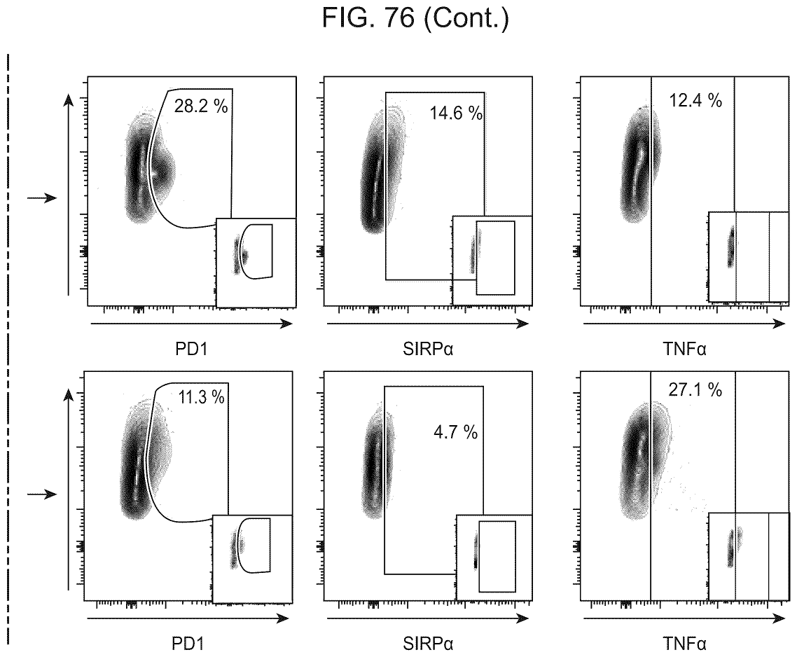

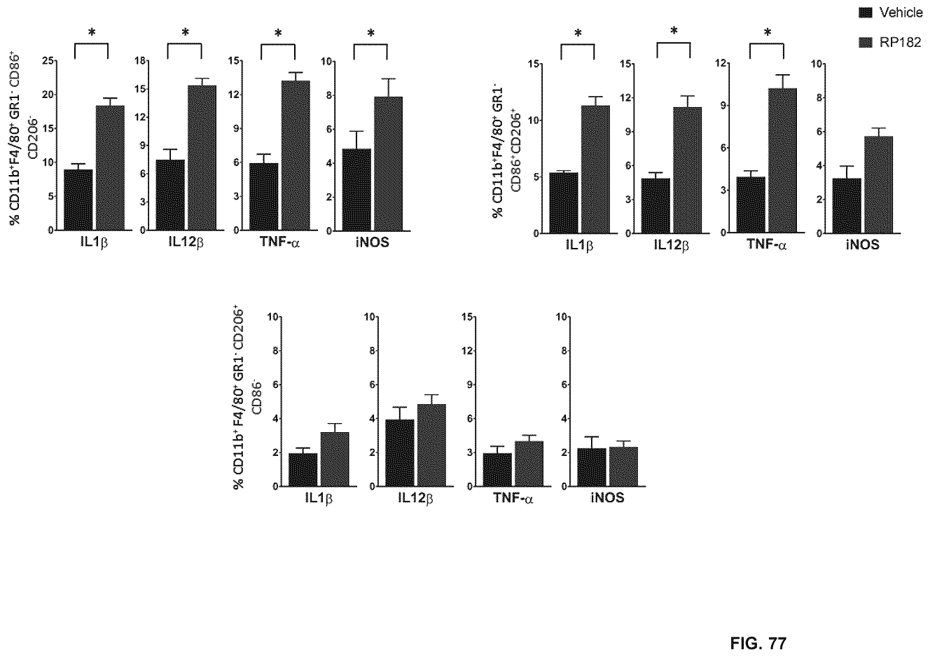

Next, equal numbers of TAMs were isolated from treated murine KPC and KP16 pancreatic tumors and evaluated their impact on T cell function. Whereas TAMs isolated from vehicle-treated animals did not induce increments of interferon gamma (INFγ) release, TAMs isolated from animals treated with RP-182, or RP-182 in combination with gemcitabine, showed activating T cell function (FIG. 74), suggesting a switch of the TAM population towards an anti-tumor, pro-inflammatory M1-like phenotype. Indeed, gene expression analysis of TAMs isolated from tumors of RP-182-treated animals and flow cytometry analysis of the TAM population confirmed a switch from decreased M2- to an increased M1-fraction in RP-182-administered animals (FIG. 75 and FIG. 76). Increased fractions of macrophages staining positive for the M1 cytokines IL-1β, IL-12β, TNFα and M1 marker iNOS were observed in RP-182-induced double positive CD86+CD206+ and the CD86+CD206− M1-like cells but not in the CD86−CD206+ M2-like TAMs (FIG. 77), findings in line with the reprogramming effect of RP-182 observed on M2 BMDMs in vitro.

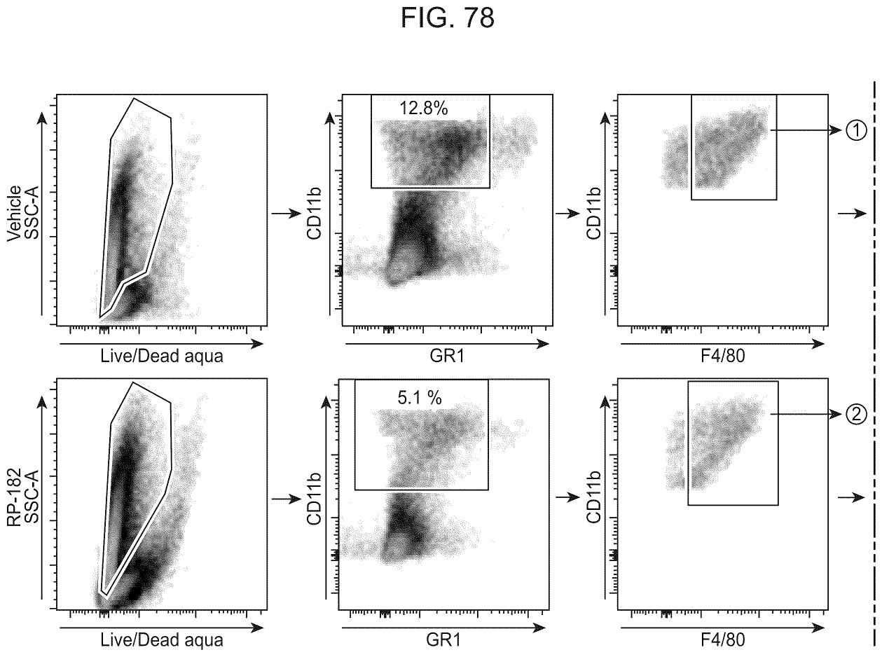

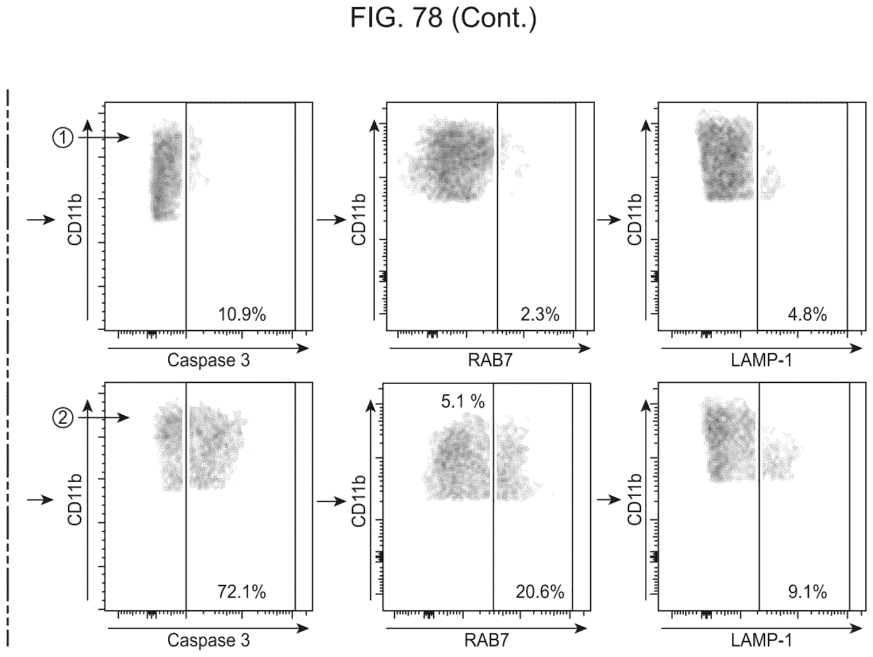

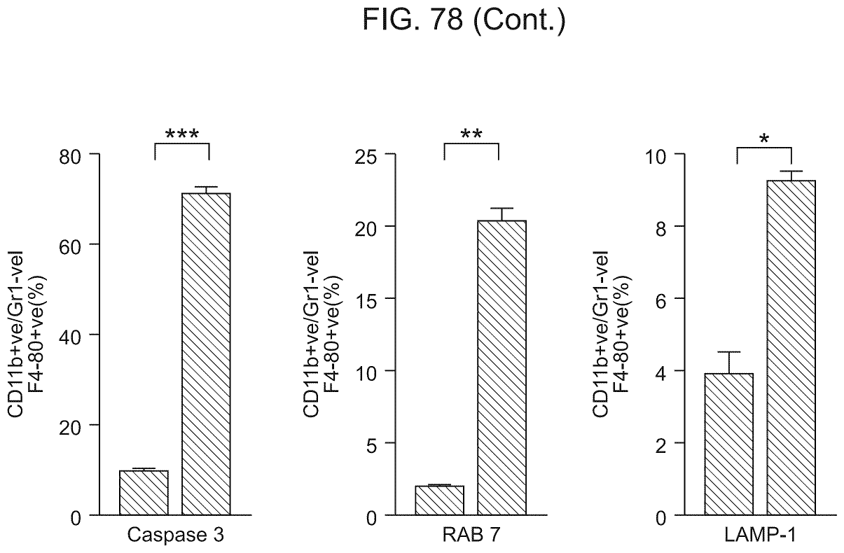

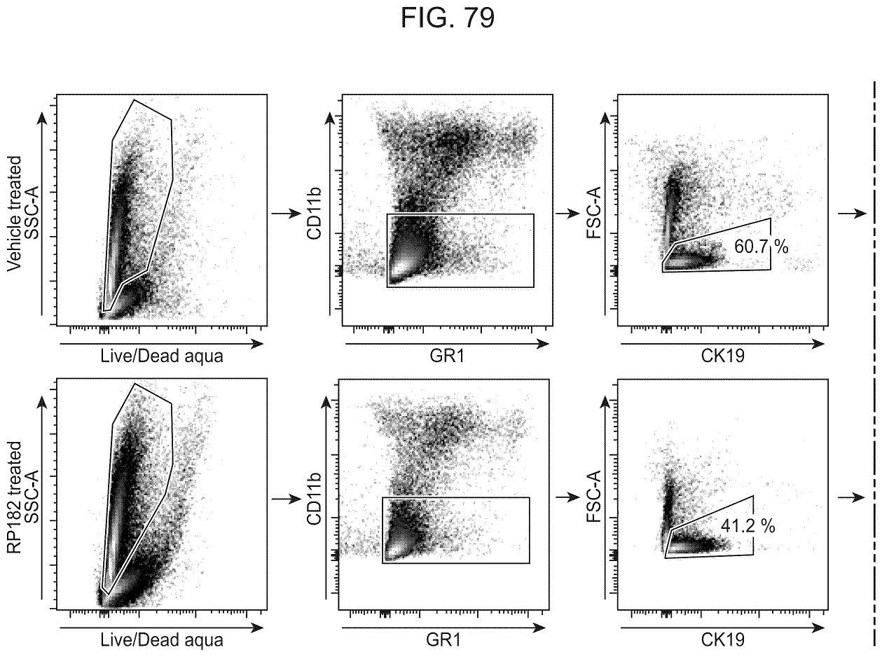

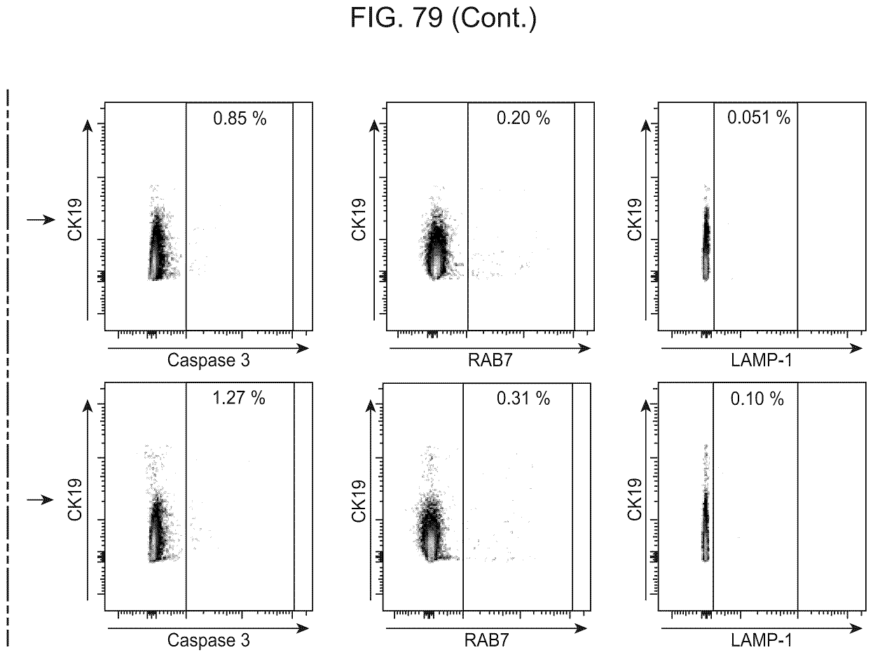

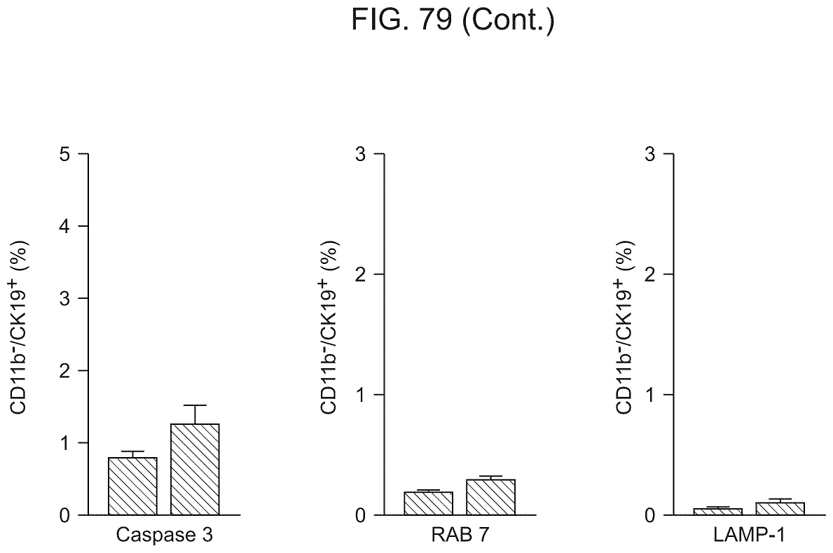

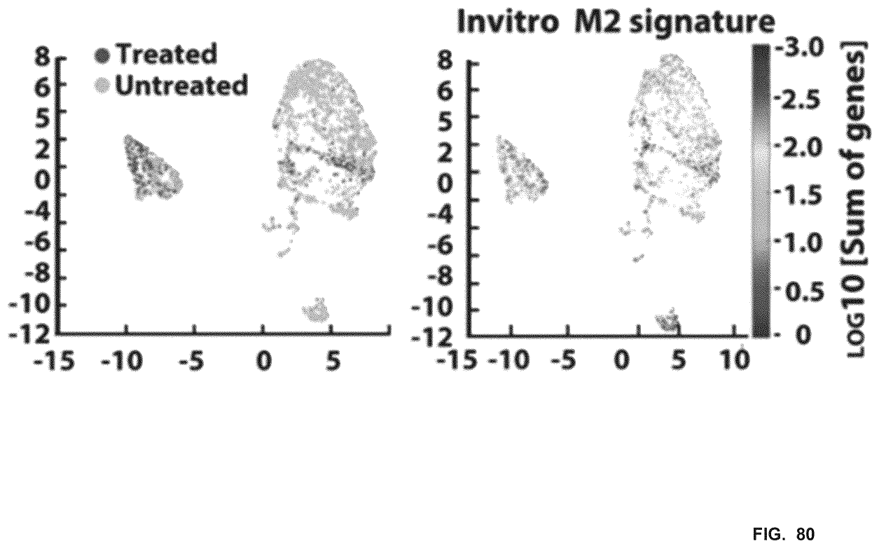

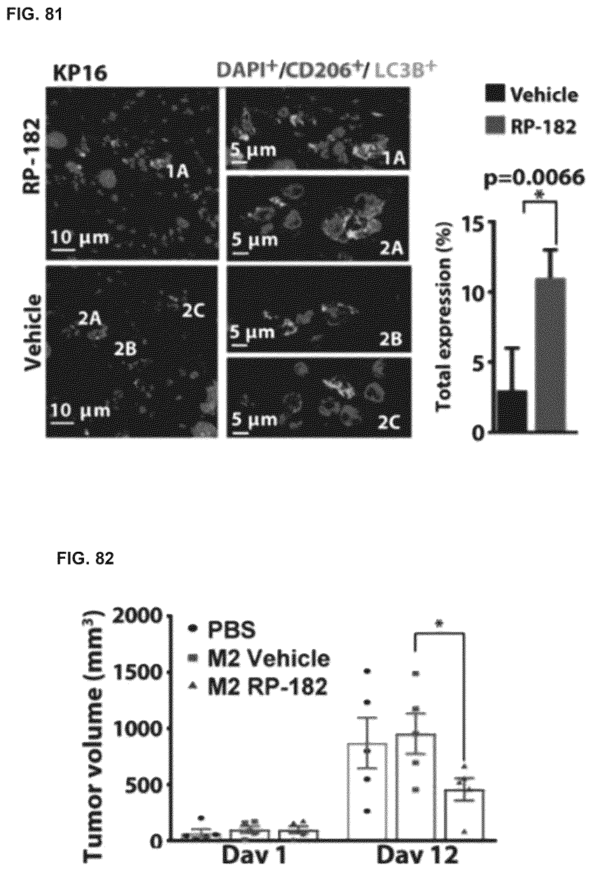

In line with RP-182's mechanism of action in vitro, cleaved caspase 3, Rab7, and LAMP-1 positive TAM fractions were significantly higher in RP-182 vs vehicle-treated tumors (10.9 vs 72.1%, 2.7 vs 19.8%, and 3.9 vs 9.2%, respectively) (FIG. 78). Induction of apoptosis and phagocytosis was selective for CDIIb+F4/80+Gr-1− macrophages as CD11b−CK19-9+ cancer cells showed only minimal or no changes (FIG. 79). Furthermore, when applying the previously differentially expressed gene (DEG) set obtained from RP-182-treated vs untreated M2 BMDMs onto whole transcriptome analysis of single cells from KPC tumor digests, there was a significant enrichment of genes altered by RP-182 in vitro in the TAM cell cluster formed by the treated cohort (FIG. 80). Dual staining of RP-182-treated tumors with the markers LC3 and CD206 showed that RP-182 induced autophagosome formation in CD206-positive TAMs phenocopying the induced LC3 expression in human and murine M2-like macrophages in vitro (FIG. 81). RP-182-induced changes in M2 macrophages were associated with a tumor growth restricting impact upon intratumoral in vivo transfer of M2 BMDMs pretreated with RP-182 (FIG. 82).

### Example 7: RP-182 Cooperation with Immune Checkpoint Inhibition and Effect on Antitumor Immunogenicity and Disease Outcomes

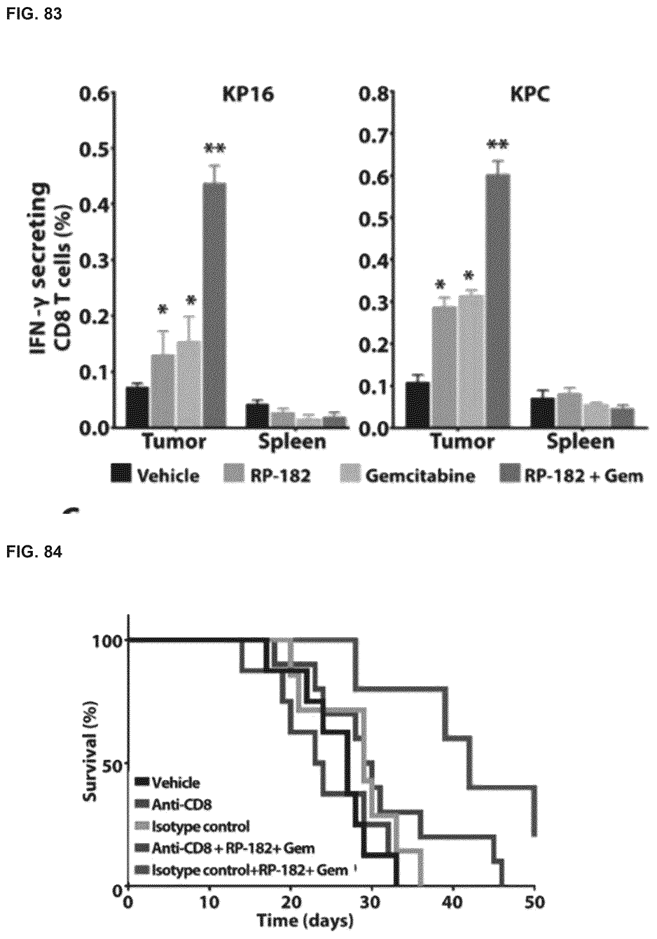

The effects of RP-182 on intratumoral T cell function were examined by measuring antigen recognition and T cell activation via interferon gamma (INFγ) release upon co-culture with KPC and KP16 cancer cells (EliSpot assay). Intratumoral T cells from animals treated with RP-182, and RP-182 in combination with gemcitabine, showed significantly stronger activation upon co-culture with cancer cells compared to T cells isolated from animals treated with vehicle control suggesting improved tumor antigen recognition following RP-182 treatment (FIG. 83). Improved T cell function was selective for T cells isolated from tumors and not observed in T cells isolated from spleens.

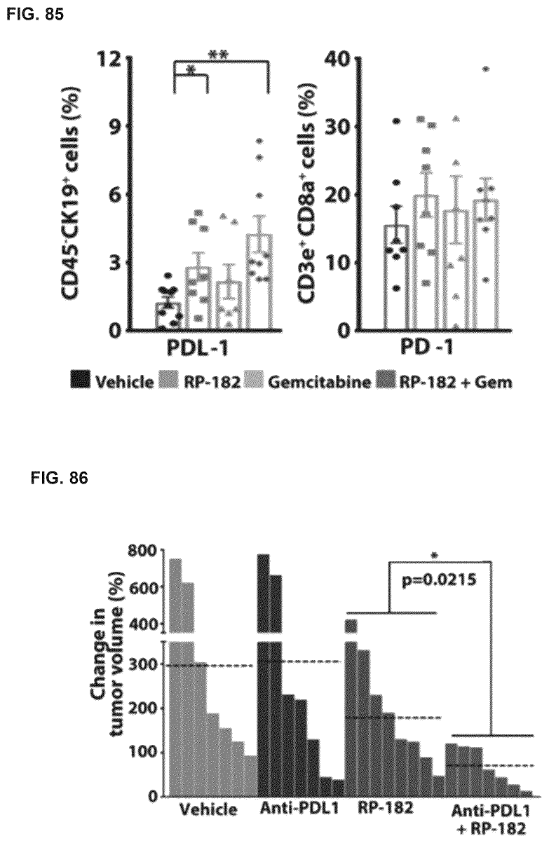

To link the above increased tumor cell recognition to the observed anti-tumor activity in vivo, the efficacy studies were repeated with RP-182 and gemcitabine treatment in mice depleted of CD8+ T cells. Mice void of CD8+ T cells and treated with RP-182 and gemcitabine displayed a reduction in extension of survival when compared to mice treated with isotype control, indicating the involvement of CD8+ T cells in RP-182's mechanism of action (FIG. 84). Murine pancreatic cancers showed increased PD-L1 expression on CK19-positive cancer cells upon treatment with RP-182 (FIG. 85). To test whether these elevated levels of checkpoint expression can be exploited for combination therapies, and whether anti-TAM therapy via RP-182 might cooperate with PD-L1 immune checkpoint inhibition in pancreas cancer not known to respond to single agent anti-PD-1/PD-L1 therapy, RP-182 was combined with anti-PD-L1 treatment. Anti-tumor activity of the combination was enhanced compared to single agent therapy (p=0.0215) (FIG. 86).

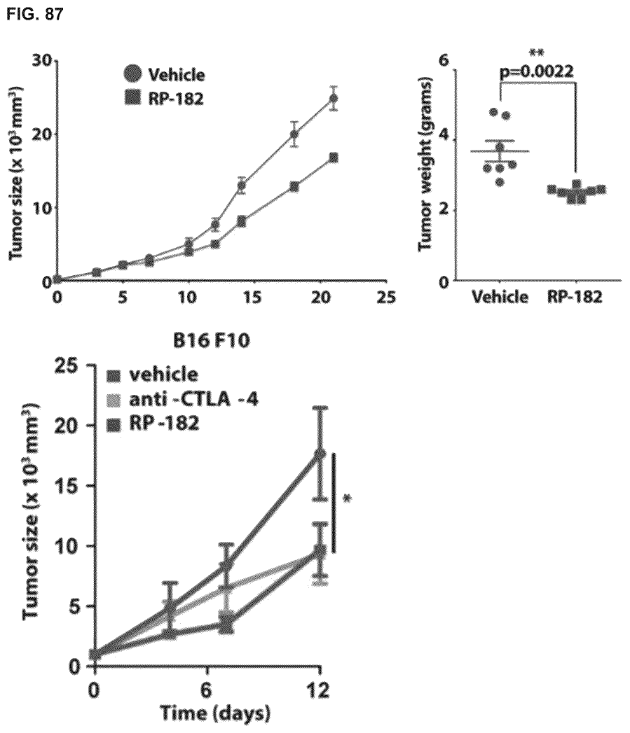

Next, it was examined whether the above anti-tumor activity extends onto additional cancer models including patient-derived xenotransplantation models. RP-182 reduced growth of CT-26 colon tumors and murine B16 melanomas, where it showed equal efficacy as standard anti-CTLA4 checkpoint therapy (FIG. 87). Using previously genotyped human pancreatic cancer tissues from NCI's Patient-Derived Models Repository (PDMR; https://pdmr.cancer.gov/), patient-derived xenotransplants (PDX) with CD206high and CD206low expression levels were generated and treated with vehicle, control peptide RP-426, or RP-182. While RP-182 reduced tumor growth in the CD206high PDX models compared to vehicle and RP-426 control, there was no effect in the CD206low models.

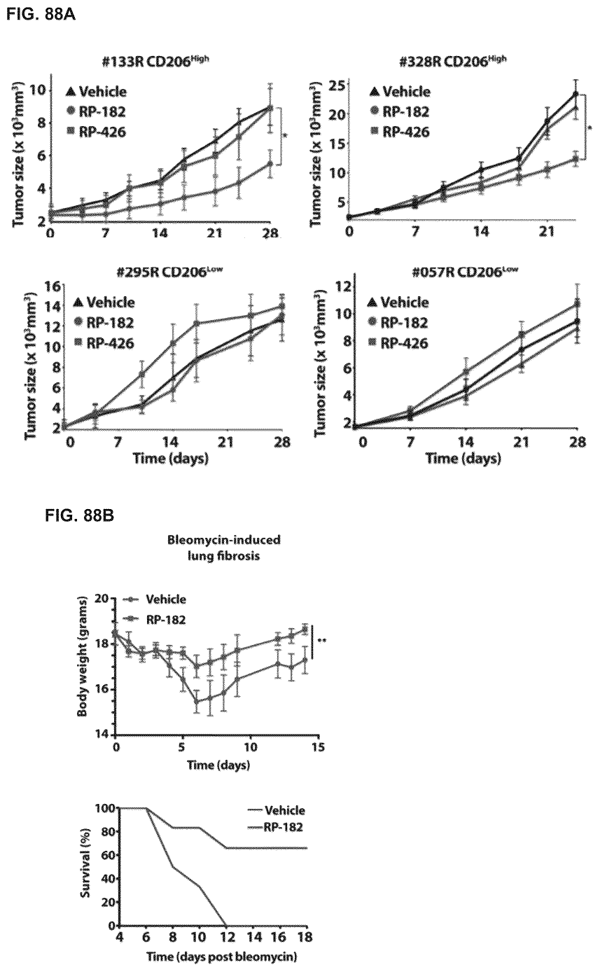

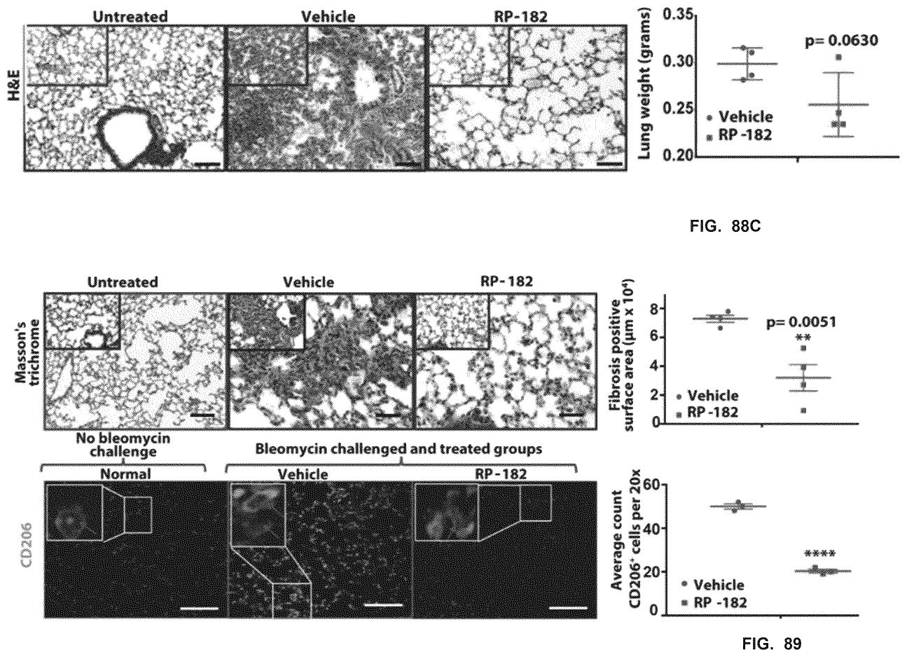

Considering that CD206-positive, alternatively activated macrophages are involved in other disease processes, RP-182 was tested in a bleomycin lung fibrosis model next. Treatment with RP-182 resulted in increased animal weight and improved overall survival and diminished pulmonary fibrosis (FIGS. 88A-C). Correlative lung tissue studies showed reduction of M2-like macrophages measured by expression levels of CD206 (FIG. 89). These findings suggest that RP-182 modulates macrophage activity across several murine and human cancer models and possibly includes non-cancerous disease models driven by CD206-positive macrophages overall suggesting a wide applicability. CD206 expression status may aid future selection of tumors most likely to respond.

### Example 8: Effect of RP-182 on Cancer Cell Phagocytosis by M1-Like Macrophages

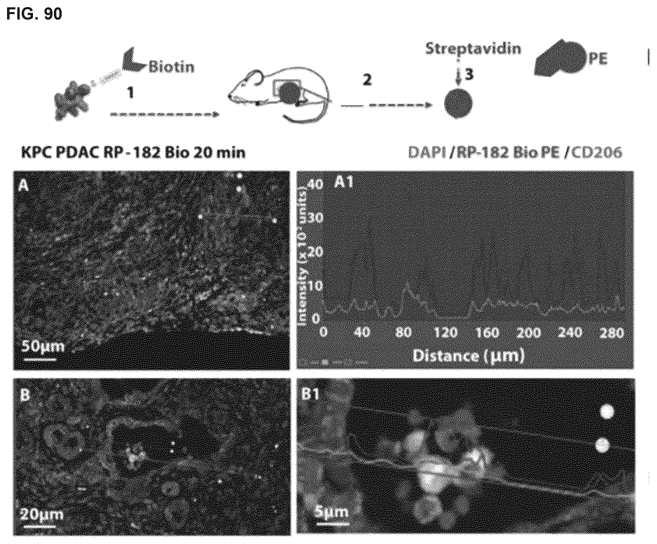

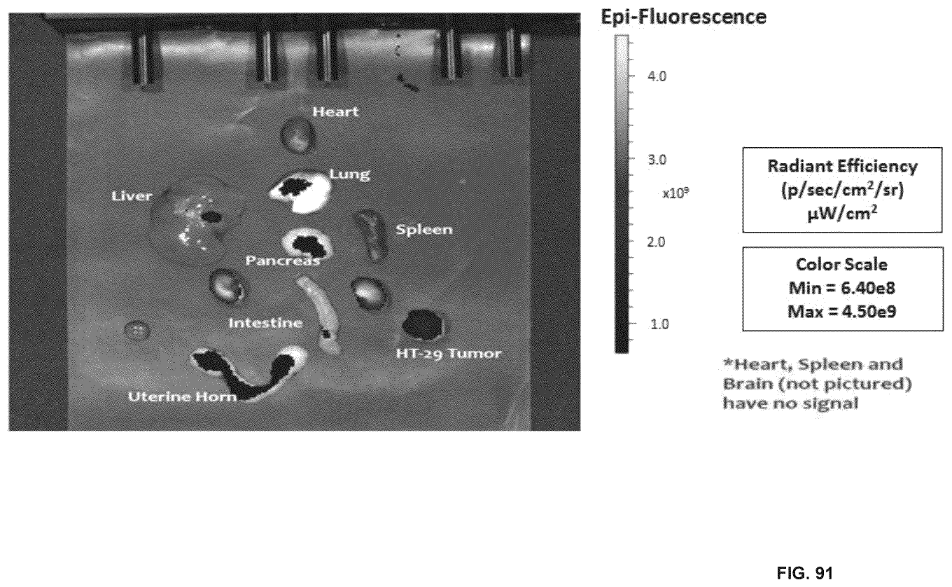

To confirm that RP-182 is able to efficiently engage CD206 positive target cells in pancreatic tumors after systemic administration, KPC mice were dosed with 20 mg/kg of RP-182 carrying a biotin. Tumors were harvested, embedded, and co-stained with anti-CD206 antibody probes and AlexaFluor-streptavidin to detect intratumoral RP-182 (NCGC-00510434; FIG. 15B). Multicolor confocal microscopy measuring staining intensities across linear sectional distances (in μm) showed remarkable co-localization of RP-182 with CD206-positive cells in the microenvironment of pancreatic KPC tumors suggesting RP-182 is binding its target (FIG. 90). Photon xenogene quantification of AlexaFluor 480-RP-182 in organs showed significant enrichment in tumor and kidney compared to other organs (FIG. 91). Fourteen-day toxicity studies of continuous dosing of up to 30 mg/kg daily dosing of RP-182 did not show hematological changes in total blood of dosed animals or any change in total body or selective organ weights.

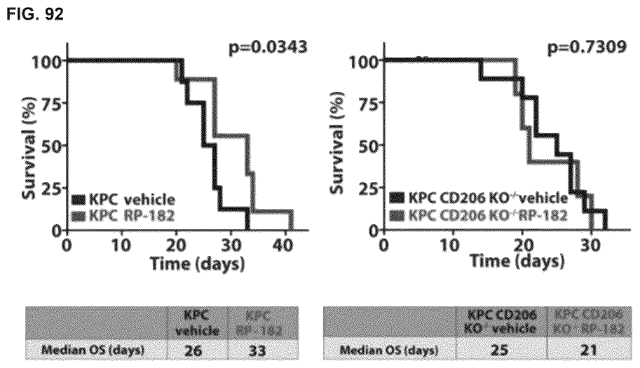

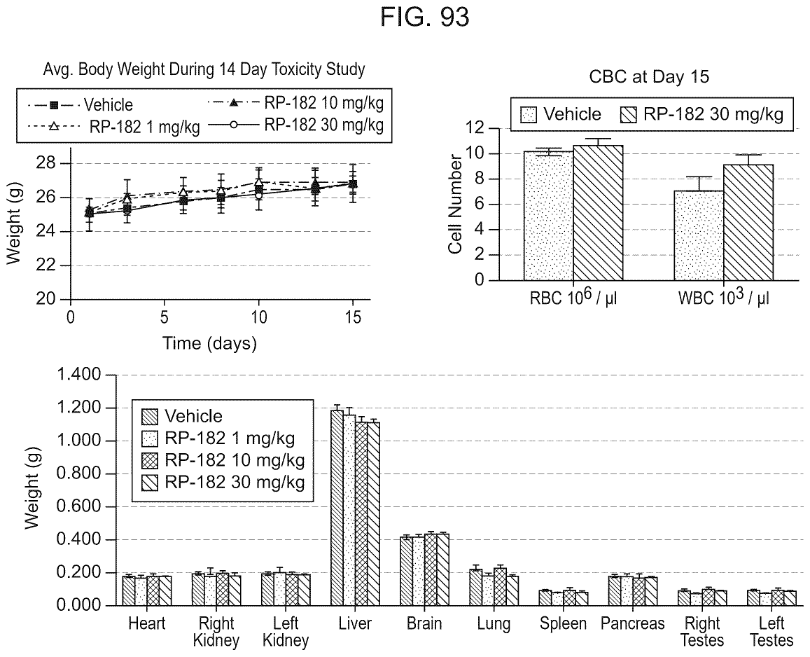

In line with its selectivity for its target CD206 and CD206 expressing M2 macrophages, treatment with RP-182 mediated a survival gain C57BL/6 wild type mice allografted with KPC tumors but not in CD206-deficient B6.129P2-Mrc1tm1Mnz/J mice with KPC tumors lacking the target receptor of RP-182 (FIG. 92). The smaller impact of RP-182 on overall survival compared to autochthonous KPC tumors shown in FIG. 67B might be due to differences between spontaneous KPC tumors and KPC tumors generated from allografted cells. RP-182 did not produce discernible hematological changes in total blood of dosed animals or any change in total body or selective organ weights upon preliminary toxicity testing (FIG. 93).

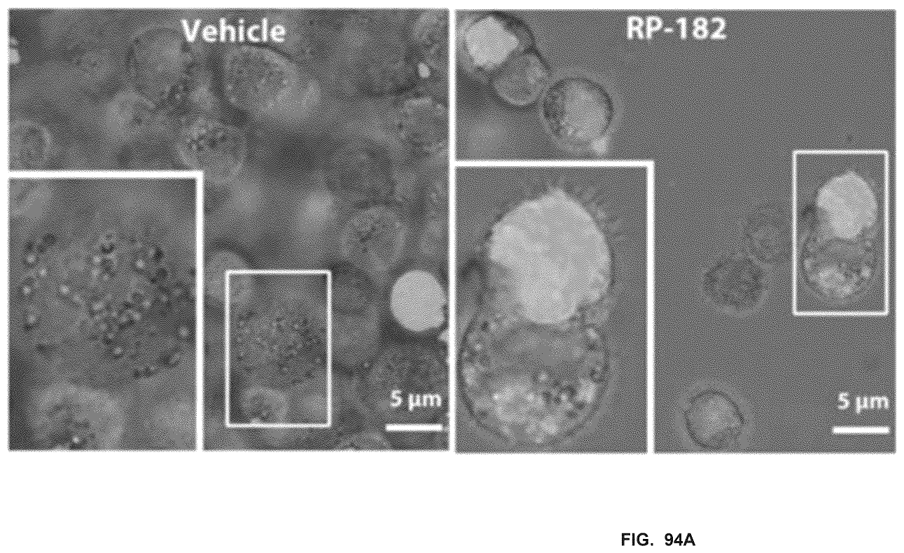

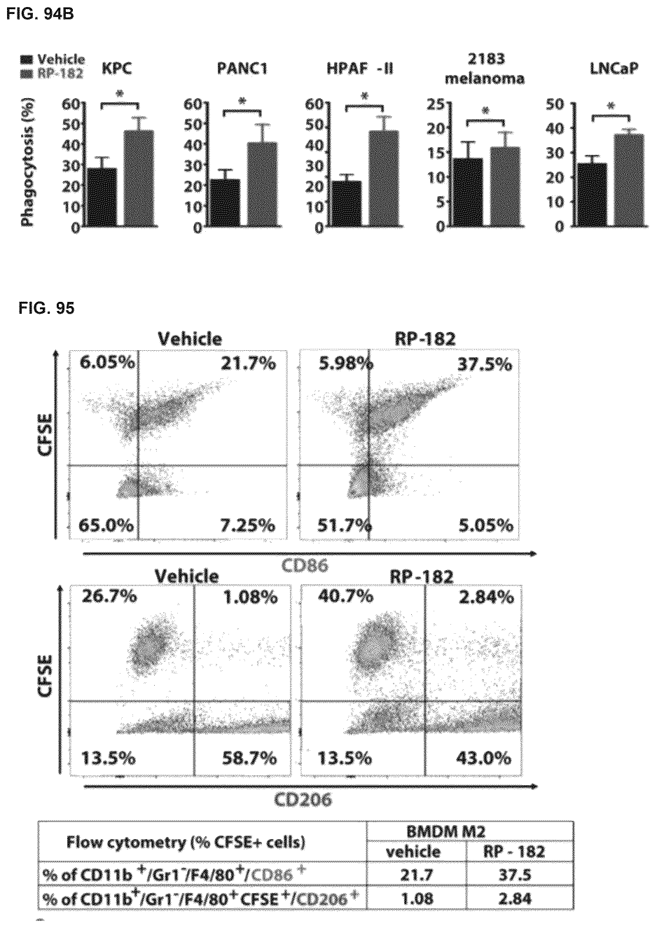

The loss of the SIRPα receptor involved in the ‘do-not-eat me’ signaling of innate immune cells on macrophages isolated from murine pancreatic tumors treated with RP-182 (FIG. 75) prompted us to explore whether T cell-independent innate mechanisms of RP-182 such as cancer cell phagocytosis might contribute to the anti-tumor activity of RP-182. Thus, phagocytosis was measured of several different human and murine cancer cell lines labelled with the green fluorescent dye carboxyfluorescein succinimidyl ester (CFSE) in M2 BMDMs treated with RP-182 nextRP-182 increased cancer cells phagocytosis (measured by engulfed CSFE-positive cells; phagocytotic index after 2 hours treatment with RP-182) of several murine and human cancer cell lines by 28.2 to 46.6% (FIGS. 94A-B). Cancer cell phagocytosis was exclusively observed at baseline in the CD86-positive M1 population, and increased after exposure to RP-182 (FIG. 95). While the increase in cancer cell phagocytosis by M1 macrophages is similar to the fraction of reprogrammed CD86 positive M1 cells, it cannot be ruled out that the reduction of suppressive M2 cues releasing inhibition of M1 function is, in part, responsible for the observed increased cancer cell phagocytosis effect of RP-182.

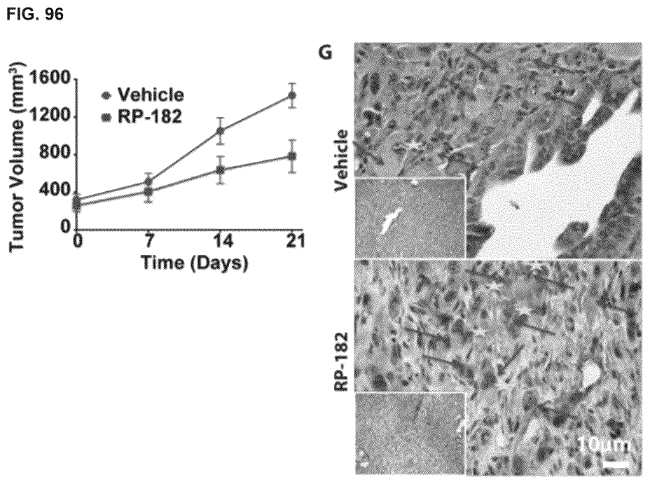

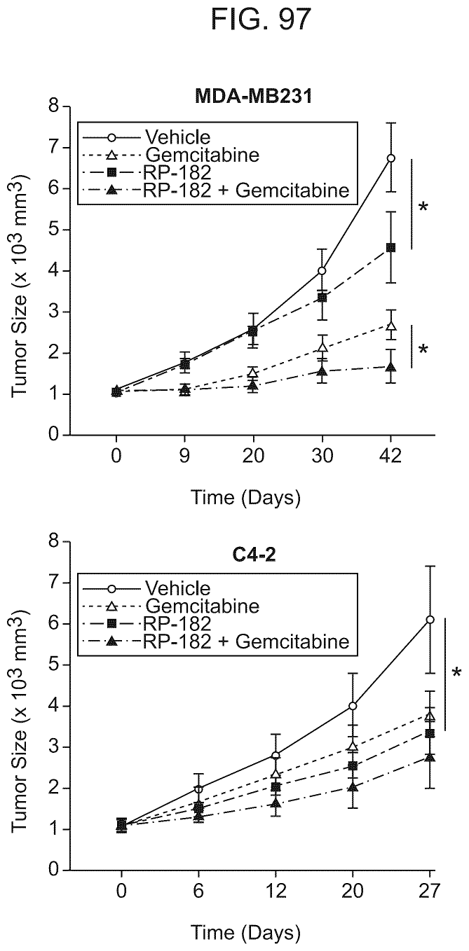

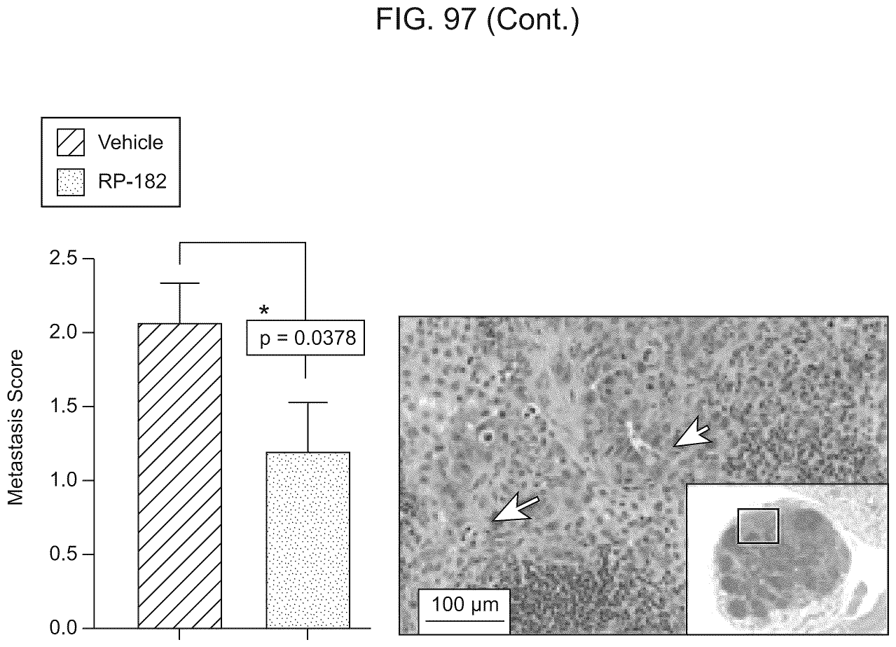

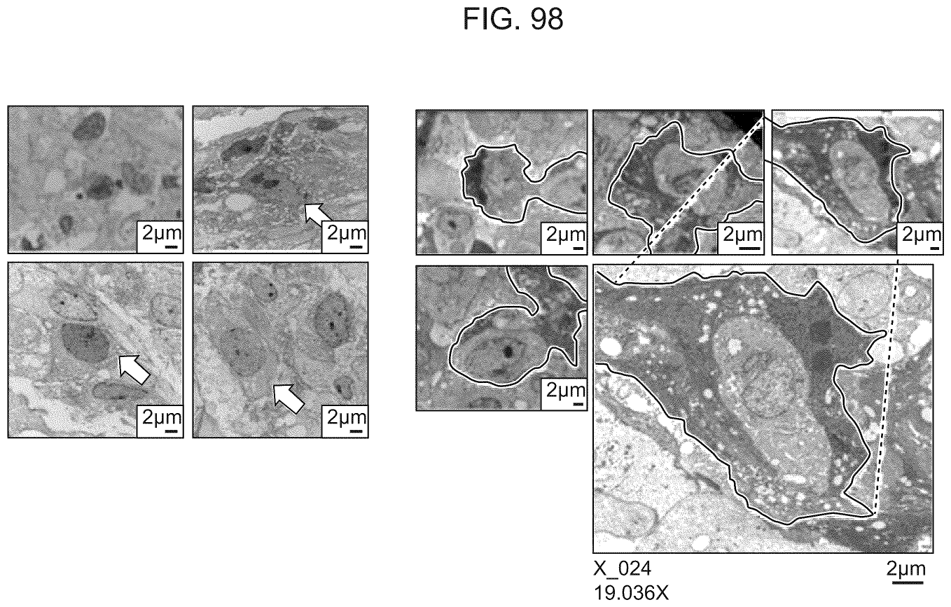

To show that the induced cancer cell phagocytosis as an innate mechanism of action of RP-182 is involved in RP-182's anti-tumor activity, KPC, MDA-MB231 breast and C4-2 prostate tumors were established in homozygous nu/J mice which are deficient of mature T lymphocytes and unable to mount cell-mediated anti-tumor immune responses but retain B cell, natural killer (NK), and myeloid cell function. RP-182 monotherapy reduced tumor growth across these tumor models, and improved anti-tumor activity of the standard gemcitabine model and reduced metastatic dissemination in the MDA-MB231 model (FIG. 96; FIG. 97). H&E review of RP-182-treated tumors revealed features of macrophage activation and cancer cell phagocytosis. Upon treatment with RP-182 TAMs lost their ballooned, eosinophilic cytoplasm displaying increased hematoxylin uptake with numerous intracellular inclusions of nuclear material or cellular debris. Tissue sections interrogated by electron microscopy showed multiple complete inclusions of cancer cells in TAMs, partial phagocytosis of cancer cells, or clasping of activated macrophages onto cancer cells compared to vehicle-treated tumors (FIG. 98).

Thus, RP-182 enhances via CD206 adaptive and innate immune cell functions in tumors to-date not known to be responsive to immune checkpoint blockade.

### Discussion

Tumor-associated macrophages, which successfully recognize and infiltrate affected tissues, are positioned to be able to initiate a profound, anti-tumor immune response. Unfortunately, many tumors are able to alter the behavior of these cells and coax them into supporting vascularization, tumor growth, invasion, and metastasis.

RP-182 was selectively killing these problematic macrophages, initiating an apoptotic process that then depletes them at tumor sites. Evaluation of RP-182's biological actions within M2-macrophages and other immune cells showed that this peptide also alters the function of these macrophages shifting them from an immune-suppressive state into a pro-inflammatory, phagocytosing phenotype able to mediate immune anti-tumor activity. The activation of phagocytosis, autophagy, and NF-kB signaling within RP-182-responding macrophages rapidly leads to upregulation of M1 markers followed by downregulation of M2 markers. These phenotypic changes are accompanied by improved phagocytic function, and notably by a reduction of their overall immunosuppressive character. Both, the reprogramming towards a M1 phenotype of cells which lose MRC1/CD206 expression and escape apoptosis together with the induction of cell death in MRC1/CD206-carrying macrophages upon longer exposure with RP-182 robustly shifts the TAM population toward a M1 phenotype which restores immune surveillance in the tumor microenvironment. The mannose receptor MRC1/CD206 is expressed on the cell surface and intimately connected to its main functions of phagocytosis of pathogenic micro-organisms through recognition of their surface, mannose-containing glycoproteins (MGPs) as well as its scavenger function, in particular collagen, at sites of tissue injury.

RP-182 activates via the mannose receptor MRC1/CD206 phagocytosis and autophagy in M2-like macrophages, which reverts these cells into an anti-tumor M1-like phenotype with elevated M1 cytokine production and the ability to phagocytose cancer cells. In addition, RP-182 induces via an autocrine positive feedforward loop involving TNFα signaling induction of apoptosis via cleaved caspase 8 promoting the depletion of this population and further shifting the balance towards the pro-inflammatory, anti-tumor M1 phenotype. RP-182 was tested in a lung fibrosis model characterized by the extravasation of CD206-positive alveolar macrophages. The observed decreased deposition of collagen and reduced fibrosis associated with the therapeutic merit of RP-182 in this inflammatory model appears consistent with the known anti-fibrotic activity of activated M2 macrophages.

Kinetically, induction of phagocytosis and autophagy by RP-182 in CD206-positive M2-like macrophages was followed by induction of apoptosis, reduction of M2-like macrophages in the TAM population, and increased CD8 cytotoxic T cell infiltration and function. The altered TAM phenotype was also associated with a less-EMT-like cancer phenotype in RP-182-treated tumors. Of note, the increased M1 population transformed from M2-like cells improved innate anti-tumor immunity via increased cancer cell phagocytosis both in vitro and in vivo. Results from the studies in human CD206high vs CD206low PDX models and in CD206−/− knockout allografts suggest that CD206 levels might be used as a future biomarker for this approach.

In summary, the results presented herein show that biophysical similarities beyond primary amino acid sequence alignments can detect previously unknown homologies between HDPs and regulators of the innate immune system and that these motifs can be used to design effective therapeutics. RP-182 is a 10mer synthetic HDP derived from screening for biophysical homologies across HDPs and mediators involving innate immune processes. A conformational switch of the mannose receptor MRC1/CD206 by RP-182 reprograms M2-like TAM in the tumor stroma and improves intratumoral innate and adaptive anti-tumor immunity and tumor control.

### Materials and Methods

### Peptides, Cell Lines, and Chemical Sources

Peptides were synthesized by Poly Peptide Laboratories, San Diego, CA. Peptides included RP-182, KFRKAFKRFF; RP-832C, RWKFGGFKWR; RP-185, FFKKFFKKFK; AVP, EKLSAFRNFF; LL37F1, FFRKSKEKIG; and RP-426 KARKAAKRAF. PANC-1 (CRL-1469), HPAF-II (CRL-1997), and LNCaP cells (CRL-1740) were purchased from the American Type Culture Collection (ATCC, Manassas, VA), primary murine KP16 and KPC pancreas cancer cell lines were derived from fresh tumor tissue and the primary patient-derived low-passage melanoma line 2183 was provided. In accordance with AACR practices, cells were authenticated by SNP genotyping using Illumina MiSeq sequencing and confirmed to be Mycoplasma-free. Mesenchymal Stem Cells, human fibroblasts, and endothelial cells were purchased from Cellular Dynamics International Inc., and DC2.4 mouse dendritic cells were purchased from Millipore Sigma. The cells were maintained according to instructions from supplier or in RPMI 1640 medium with 10% (v/v) FBS and incubated at 37° C. in a 5.0% CO2 atmosphere. Small molecule inhibitors were purchased from Selleckchem Inc. (Houston, TX) and included nuclear factor ‘kappa-light-chain-enhancer’ of activated B-cells (NF-kB) inhibitors JSH-23 (Cat #S7351), QNZ (EVP4593) (Cat #S4902), mitogen-activated protein kinase (MAPK) inhibitor selumetinib (AZD6244) (Cat #S1008), Ras-related C3 botulinum toxin substrate 1 (Rac1) GTPase inhibitor NSC 23766 (Cat #S8031), and cell division control protein 42 homolog (CDC42) inhibitor ZCL278 (Cat #S7293).

### Synthesis of Peptide Analogs

The diazirine containing biotinylated RP-182 analog (NCGC-00510434) and biotinylated RP-426 were synthesized and purified to >95% purity commercially by RS Synthesis LLC (Louisville, KY). The Fmoc-diazirine-containing phenylalanine analog was prepared as shown in FIGS. 15A-B. Separation of the enantiomers was resolved on a chiral column (Chiralpac IB 4.6×250 mm, 100% EOH; 1 ml/min). The biotin was introduced on the side chain of a lysine residue that was coupled to either polyethylene glycol (PEG) or hydrocarbon polyether linkers (see FIGS. 15A-B).

### Recombinant Human and Mouse MRC1/CD206

Recombinant human CD206 was purchased from R&D Systems(Cat #2534-MR/CF). Recombinant mouse CD206 was produced by Protein Expression Laboratory (FNLCR, Frederick, MD). Briefly, mouse CD206 (NM_008625.2) cDNA fragment encoding 23-1387 sequence was optimized for human codon usage and generated in pDEST vector with N-terminal honeybee melittin signal peptide and C-terminal 6×His tag. Protein was transiently expressed in Expi293E cells and purified from the supernatant, collected 72 h post transfection, using nickel affinity chromatography. CD206 was eluted in 20 mM HEPES, pH 7.2, 300 mM NaCl, 250 mM imidazole and dialyzed into PBS, pH 7.4. For electron microscopy experiments, the mouse CD206 was further purified by size exclusion chromatography and eluted in PBS, pH 7.4.

### Biophysical Homology Screening

To screen for phylogenetically conserved homologies beyond primary amino acid structures the design heuristic Molly font was used (FIG. 1). Chemical properties of amino acids captured by Molly font include amino acid volume encoded by circle size (measured in cubic Angstroms (radius of gyration) in H2O) and the hydrophobicity/hydrophilicity of amino acids converted to a color scale. The most hydrophobic amino acids are depicted in the most intense cyan color, while those that are less hydrophobic are proportionally less concentrated cyan (FIG. 1). Amino acids that are most hydrophilic possess the deepest magenta color and a graduated scale of less intense magenta color is used for amino acids of lower hydrophilic character. Implicit in this scheme is that, within a particular cue, i.e., among hydrophobic or hydrophilic amino acids of very similar properties, exchanges would be more likely to occur (generating the variability one observes in proteins of similar function from evolutionarily distant organisms) subject to the specific structural constraints imposed on each particular protein for it to retain its functionality. Representative glyphs included as mnemonic symbols characterize additional chemical properties of amino acids, including charge of amino acids (“+” or a “−” sign) incorporated within their glyph, and dissociation constant of their ionizable protons encoded by the thickness of the glyph. Numerical values are the energies in kcal/mol required to move the amino acid side chain from the interior of lipid bilayer to the outside aqueous environment (32).

### In Silico Docking

Protein-protein interactions of α-helices derived from identified biophysical homology sequences with putative target receptors were evaluated by in silico docking utilizing ClusPro® server (Boston University, MA), which performs direct docking in three computational steps: (1) rigid body docking by sampling billions of conformations, (2) root-mean-square deviation (RMSD) based clustering of the 1,000 lowest energy structures generated to find the largest clusters, that will represent the most likely models of the complex, and (3) refinement of selected structures using energy minimization. Docking with each energy parameter set results in ten models defined by centers of highly populated clusters of the lowest energy docked structures. Consideration of the centers of the largest clusters of these low energy structures, rather than simply low energy structures, is unique to ClusPro® and implicitly accounts for some of the entropic effects aligning cluster populations to cluster probabilities under natural assumptions. Structures were compared by ranking plotted binding coefficients that combines cluster probabilities with binding energies.

### MRC1/CD206 Modeling

The protein sequence (1,456 aa) for human MRC1/CD206 was obtained from UniProt (UniProt ID P22897-1 NCBI ID: NP_002429.1) and includes two N-terminus domains (Ricin-B-type Lectin and a Fibronectin type-II) followed by eight C-type lectin domains (numbered 1 to 8), a transmembrane domain (TM), and a cytoplasmic domain. The Iterative Threading and ASSEmbly Refinement software, I-TASSER (https://zhanglab.ccmb.med.umich.edu/I-TASSER/), was used to generate 3D models of CD206. I-TASSER utilizes a hierarchical approach that identifies 3D templates from the RCSB-PDB (http://www.rcsb.org) using a multiple threading approach. Full-length models will eventually be constructed by iterative template fragment assembly simulations.

To assist modeling, we extracted from UniProt (www.uniprot.org) the cysteine residue positions that participate in the disulfide bonds and supplied this list as distance restraints during I-TASSER modeling. The top threading RCSB-PDB template IDs identified by I-TASSER were 5ao5, 3jav, 5ao6 and 4igl. The normalized B-factor values for the models fluctuated around zero, indicating acceptable local accuracy of the model(s). Model confidence was measured by C-score, and the C-score for models ranged between −5 and 2, where higher values indicates higher confidence. The top 4 models had the following C-scores, −0.35, −1.93, −2.89 and −2.97. The top 4 models were analyzed and based on the comparison of Small Angle X-ray Scattering model (SAXS) and the I-TASSER predicted secondary structure confidence and C-scores, the top-ranked I-TASSER model was identified as a possible structural fold for CD206.

### Small-Angle X-Ray Scattering (SAXS) Data Collection and Analysis

SAXS data was collected at the 121D-B beamline of the Advanced Photon Source (APS), Argonne National Laboratory, Lemont, IL. Photon energy was 13.3-KeV and sample-to-detector distance was 2 m to achieve a q range of 0.005<q<0.90 Å-1, where q=(4π/λ) sin θ, and 2θ is the scattering angle. Concentration series measurements for CD206 in buffer containing 50 mM Hepes, 100 mM NaCl, 1 mM DTT were carried out to extrapolate the data to infinite dilution for removing the scattering contribution due to interparticle interactions (concentration effect). Thirty 2D-image frames were recorded for sample solution and their matching buffer using a flow cell, with the exposure time of 0.75-1 sec to minimize radiation damage and to yield optimal signal-to-noise ratio. The 2D images were reduced to 1D scattering profiles and averaged using the Matlab software package at the beamlines.

The buffer background subtraction and intensity extrapolation to infinite dilution were carried out using MatLab script developed by 12-ID-B beamline. The radius of gyration (Rg) was generated from Guinier plot in the range of qRg<1.3. For comparison, Rg was also calculated in real and reciprocal spaces using program GNOM (https://www.embl-hamburg.de/biosaxs/manuals/gnom.html). The pair-distance distribution function P(r) and maximum dimension (Dmax) were also calculated using GNOM. The molecular weights were estimated using two methods based on Porod volume, Vporod, and correlation volume, Vc. Based on the silico models of CD206 monomer derived from iTASSER, the fit of dimer of CD206 to SAXS experimental data were calculated using the program CORAL.

### Electron Microscopy

Purified recombinant mouse CD206 full length protein as well as the complexes with the peptides RP-182, RP-185, RP-832C, AVP1, LL37F1 and RP-426 were analyzed by negative stain electron microscopy. A 3 μL aliquot containing ˜0.01 mg/mL of the sample was applied for 20 seconds onto a carbon-coated 200 Cu mesh grid that had been glow discharged at 30 mA for 30 sec, then negatively stained with 0.7% (w/v) uranyl formate for 40 sec. Data for the CD206 unbound and the complexes with RP-182, RP-426 and RP-832C were collected using a FEI T20 electron microscope operating at 200 kV, with an electron dose of ˜40 e-/Å2 and a magnification of 100,000× that resulted in a pixel size of 2.19 Å at the specimen plane. Images were acquired with an Eagle 2 k×2 k CCD camera (http://FEI.com) using a nominal defocus of 1500 nm and the SerialEM software (54). Data for the complexes with RP-185, AVP1 and LL37F1 were collected using a FEI Talos electron microscope operating at 200 KV, with an electron dose of ˜40 e-/Å2 and a magnification of 73,000× that resulted in a pixel size of 1.98 Å at the specimen plane. Images were acquired with a Ceta 4 k×4 k CCD camera (http://FEI.com) using a nominal defocus of 1200 nm and the EPU software. For electron microscopy data processing, particles were selected from the micrographs, extracted, and a reference-free 2D class averages were obtained using RELION 2.1.0. Microscale thermophoresis and cellular thermal shift assays.

The binding of RP-182 and RP-426 peptides to purified recombinant MRC1/CD206 was evaluated by microscale thermophoresis (MST) using a label-free approach. Specifically, two-fold serial dilutions of the peptides were prepared in PBS and incubated with the same volume of 250 nM recombinant human and mouse CD206 in PBS. After a 5 min incubation at room temperature (RT), measurements were carried out in standard capillaries using a Monolith NT. Labelfree instrument (Nanotemper Technologies) with 40% LED excitation power, 40% IR-laser power, and laser on and off times of 30 s and 5 s, respectively. KD values were calculated by fitting the T-Jump signal of the thermograph using MOAffinity analysis software (Nanotemper Technologies).

The target engagement of the peptides in macrophages was assessed using the cellular thermal shift assay (CETSA) following the Jafari et al. protocol with minor modifications. Briefly, a suspension of M2-polarized macrophages was prepared using Cell dissociation buffer (Gibco BRL) for 5 minutes at RT followed by one wash step in DMEM (Gibco, Cat. #11965118). Aliquots of 6×105 cells were incubated with 100 μM RP-182, 100 μM RP-426, or the equivalent volume of PBS, for 45 min at 37° C. After treatment, cells were collected by centrifugation for 5 min at 300×g and re-suspended in 600 μL DMEM. Fifty μL aliquots of the cell suspension were heated for 3 min in a temperature range of 37 to 64° C. with 3° C. steps, cooled at RT for 3 min, and lysed in 10 μL DMEM containing NP-40 (1% (v/v) final concentration) and Halt protease inhibitor cocktail (ThermoFisher, Cat. #78430) supported by three freeze-thaw cycles. Samples were centrifuged at 20,000×g for 20 min at 4° C., and the supernatant was subsequently analyzed by western blot using 12-230 kDa Peggy Sue separation module (ProteinSimple, Cat #, SM-S001) and Peggy Sue instrument (Protein Simple) with following settings: electrophoresis 250 volts for 45 min; blocking, 23 min; primary antibody, 30 min; secondary antibody, 30 min. Quantitative analysis of CD206 level was performed using anti-CD206 antibody at 1:70 concentration multiplexed with anti-SOD1 antibody at 1:300 dilution, as internal control for normalization, and Compass software (ProteinSimple, San Jose, CA).

### MRC1/CD206 Fragment Analysis by LC-MS/MS

To identify the binding domain of MRC1/CD206 to RP-182, we utilized two different approaches. First, 5 μg of trypsin (Thermo Scientific, Cat #90057) digested fragments of recombinant human CD206 was incubated with biotinylated RP-182 (NCGC-00510434) immobilized to magnetic beads (Thermo Scientific, Cat #65001) or bead alone for 4 h at RT. Samples on the beads were eluted after washing the beads three times in PBS containing 0.05% Tween-20 PBS-T), desalted using C18-ziptip (Millipore, ZTC18S960), and analyzed by LC-MS/MS. Second, RP-182 analog with diazirine and biotin (NCGC-00510434) was cross-linked by photo-labeling to full length recombinant CD206 and then digested with trypsin. In detail, 50 μg MRC1/CD206 protein was incubated with 100 μM NCGC-00510434 or PBS, incubated 5 min at RT, photo-labeled for 30 min on ice and digested with trypsin. Samples were incubated with streptavidin magnetic beads for 4 h at RT. Beads were collected and washed three times with PBS-T. Samples on the beads were processed as described above and analyzed by LC-MS/MS.

The LC-MS/MS analysis of samples were carried out using a Thermo Scientific Q-Exactive hybrid Quadrupole-Orbitrap Mass Spectrometer and a Thermo Dionex UltiMate 3000 RSLCnano System. Peptide mixture from each sample was loaded onto a peptide trap cartridge, eluted onto a reversed-phase PicoFrit column (New Objective, Woburn, MA) using a linear gradient of acetonitrile (3-36%) in 0.1% (v/v) formic acid, ionized and sprayed into the mass spectrometer, using a Nanospray Flex Ion Source ES071 (Thermo Scientific) under the following settings: spray voltage 1.8 kV, capillary temperature 250° C. For peptide identification and protein assembly, data were analyzed using the Thermo Proteome Discoverer 1.4.1 platform (Thermo Scientific, Bremen, Germany). Database search against CD206 sequence was performed based on the SEQUEST algorithms through the Proteome Discoverer 1.4.1 platform. Carbamidomethylation of cysteines was set as a fixed modification, and Oxidation and Deamidation Q/N-deamidated (+0.98402 Da) were set as dynamic modifications. The minimum peptide length was specified to be five amino acids with maximum false peptide discovery rate of 0.01. The precursor and fragment mass tolerance was set to 15 ppm and 0.05 Da, respectively.

### Proteomic Analysis of CD206 Complex by LC-MS/MS

To identify proteins involved in downstream signaling induced by RP-182 via CD206, 5×106 M2 cells per treatment were re-suspended into 3 mL RPMI media and incubated with 100 μM biotinylated RP-182 or PBS for 30 min at 37° C. Cells were pelleted and lysed with 500 μL Pierce IP Lysis buffer (Thermo Scientific, Cat #87787) with protease and phosphatase inhibitors (Thermo Scientific, Cat #78440) for 15 min at 4° C., then cleared by centrifugation at 15,000×g for 15 minutes at 4° C. Supernatants were transferred to new tubes and incubated with 20 μL streptavidin magnetic beads (Thermo Scientific, Cat #65001) for 30 minutes at 4° C. Beads were collected and washed four times with PBS-T. Samples on the beads were separated by SDS-PAGE, reduced with DTT, alkylated with iodoacetamide and digested with MS grade trypsin. Digested peptide mixture were concentrated and desalted using C18 Zip-Tip, reconstituted in 20 μL of 0.1% formic acid and analyzed by LC-MS/MS as described above.

Raw data files were screened against a mouse protein sequence database using the Proteome Discoverer 1.4 software (Thermo Scientific, San Jose, CA) based on the SEQUEST algorithm. Carbamidomethylation (+57.021 Da) of cysteines was a fixed modification, and Oxidation/+15.995 Da (M), Deamidated/+0.984 Da (N, Q), Methyl/+14.016 Da (K, R), Acetyl/+42.011 Da (K), Phospho/+79.966 Da (S, T, Y), Dimethyl/+28.031 Da (K, R) were set as dynamic modifications. The minimum peptide length was specified to be five amino acids. The precursor mass tolerance was set to 15 ppm, whereas fragment mass tolerance was set to 0.05 Da. The maximum false peptide discovery rate was specified as FDR<0.01.

### Murine and Human Macrophages

Murine monocyte precursor cells were obtained by flushing out the bone marrow from the femur bones of 6-8 week old healthy C57B/L mice. Experiments were conducted according to protocols and policies approved by the Institutional Animal Care and Use Committee (ACUC) of the National Institutes of Health (ACUC protocol SB-210-3) and NIHs policy on humane care and use of laboratory animals (https://olaw.nih.gov/home.htm). After 1 week of incubation at 37° C. with 5% CO2, bone marrow progenitors were polarized into M1 and M2 macrophages with the use of respective cytokines as shown in FIG. 14A. Human macrophages were obtained from peripheral blood mononuclear cells (PBMCs) acquired from de-identified healthy human donors from the NIH, Department of Transfusion Medicine (DTM) (under Institutional Review Board (IRB)-approved NIH protocol 99-C-0168) using Classical Monocyte Isolation Kit, human (Cat #130-117-337 MACS Miltenyi Biotec, San Diego, CA) and were depleted of CD14 positive cells and polarized into M1 and M2 macrophages with the use of cytokines, as described in FIG. 21B-E (57). Recombinant mouse M-CSF (Cat #PMC2044) and IFN-γ Recombinant Human Protein (Cat #PHC4033) was purchased from ThermoFisher Scientific, recombinant mouse INFγ (Cat #485-MI-100), mouse IL-4 (Cat #404-ML-050), Recombinant Human IL-4 Protein (Cat #204-IL-050), Recombinant Human IL-13 Protein (Cat #213-ILB-025), and Recombinant Human IL-6 Protein (Cat #206-IL-050) from R&D Systems (Minneapolis, MN.). Human Granulocyte Macrophage Colony Stimulating Factor (hGM-CSF) (Cat #8922SC), human Macrophage Colony Stimulating Factor (hM-CSF) (Cat #8929SC) purchased from Cell Signaling and Lipopolysaccharides from Escherichia coli 0111:B4 (Cat #L3012-5MG) was purchased from Sigma Aldrich.

### RNA Sequencing (RNASeq) Experiments and Data Analysis

Total RNA was harvested from M1- and M2-polarized macrophages treated for 2 h with 20 μM RP-182 or vehicle and subject to global RNASeq analysis on the Illumina NextSeq500 sequencer. Reads were trimmed to remove adapter sequences as well as reads less than 25 base pairs (bp) using the Trimmomatic ver 0.32 tool. Trimmed reads were mapped to mouse genome mm10 using STAR aligner. Transcriptome barn along with genome barn was created to use for RSEM quantification (accurate transcript quantification from RNASeq data with or without a reference genome).

EdgeR (Empirical Analysis of Digital Gene Expression Data; v3.30.09) analysis was conducted in R which is based on a negative binomial model and count data. In the edgeR analysis, low count transcripts were excluded manually and only genes with at least 1 count per million were used for further analysis. A normalization factor was calculated using the trimmed mean of M values (TMM) method, and the dispersion parameter for each gene was estimated as the Cox-Reid common dispersion method. The GLM (Generalized Linear M) likelihood ratio test is based on fitting negative binomial GLMs with Cox-Reid dispersion estimates in order to take known sources of variation into account. Significant DEGs were detected with a cut-off value of false discovery rate (FDR)<0.05 and log 2 fold change >1.

Functional GO Enrichment and Network Analyses: Differentially expressed genomic features (p<0.05; q≤0.05) in M2 macrophages after RP-182 treatment were imported into Cytoscape (v. 3.7.1) to assess functional gene ontology enrichment and visualize GO term interaction network. After import, setsApp (v. 2.2.0) plug-in was used to separate features by up- or down-regulation. Once separated, functional analysis and network construction were completed using the ClueGO plug-in (v. 2.5.4). A two-sided (enrichment/depletion) hypergeometric test with Bonferroni step down was used to determine KEGG pathways (v. 27.02.2019) enrichment. Analysis thresholds included: enrichment significance of p≤0.05, a minimum of 5% gene inclusion and a kappa score threshold of ≥0.4. ClueGO uses kappa scores to determine likelihood of GO term interactions and groupings.

For Pathway Studio® analysis, differentially expressed gene sets of RP-182- vs vehicle-treated M2 BMDMs were calculated. DESeq2 Analysis (version 1.22.2; in R) yielded 1,224 differentially expressed genes (DEGs) at p≤0.05 and 382 DEGS at FDR adjusted q≤0.05 and were used for Gene Ontology analysis at Mouse Genome Informatics (http://www.informatics.jax.org/). Gene set enrichment analysis of top 25

DEGs generated p-values for enrichment and enrichment score which were both incorporated into ranking metric. Leading Edge Analysis was used to identify most common genes across enriched gene sets that remained consistent despite ranking metric change which were imputed into Pathway Studio® finder (https://www.pathwaystudio.com/).

### Single-Cell RNA-Sequencing

Normal pancreas and pancreatic tumors from treated and untreated KPC mice were harvested, and single cell suspensions were prepared using mouse tumor dissociation kit (#130-096-730, MACS Miltenyi Biotec, San Diego, CA) and a Gentle Macs Agitator (Miltenyi Biotec, San Diego, CA) as per company protocol. 9,000-12,000 cells were used to generate single-cell barcoded cDNA libraries using the 10× Genomics Chromium Controller. Single Cell 5′ Library and Gel Bead Kit (10× Genomics, Cat #1000006) was used to generate gene expression libraries. Manufacturer's instructions were followed for generating the libraries, which were then sequenced on multiple runs of Illumina NextSeq500 with V2 chemistry. Sequencing runs for the gene expression libraries were setup as 26 cycles+8 cycles+57cycles non-symmetric runs.

### Single Cell Transcriptome

For single cell gene expression libraries, de-multiplexing, alignment to the mm10 transcriptome, gene-barcode matrices generation were using the 10× Genomics Cellranger toolkit (v2.2.0) for each data set. Library batches were normalized using the CellRanger Aggregate function to aggregate all treated and untreated tumor samples datasets together, and the resulting gene-barcode matrices were fed into Seurat(v2.3.4). Each sample in the sample set was preprocessed in Seurat (v2.3.4) by removing genes that were detected in fewer than 3 cells and removing cells that had fewer than 100 genes detected. The samples were then further processed by using the global-scaling normalization method LogNormalize, that normalizes the gene expression measurements for each cell by the total expression, multiplies this number by a scale factor (10,000 by default), and log-transforms the result. The number of principal components to use for analysis was estimated on each sample set by using the calcPCA function in URD while setting the mp.factor parameter to 2. The total number of significant principal components identified by this algorithm was taken as the estimated number of principal components to use in Seurat. Dimensionality reduction was carried out in Seurat via principal component analysis followed by clustering t-SNE visualization using the top significant components. Major clusters were denoted by differentially expressed canonical marker genes, and these were subjected to additional rounds of cluster refinement. For the CD11b+ and Krt19, CD11c, and Ly6G negative cells, the marker gene ITGAM was used to identify CD11b+ cells, the cells co-expressing the Krt19 gene (cytokeratin-19), ITGAX (CD11c), and LY6G were removed. The filtered gene-barcode matrix for CD11b+ and Krt19, ITGAX, and LY6G negative cells were used for cluster refinement and analysis. Differential expression analysis was performed using EdgeR program. Differentially expressed genes from RP-182 treated M2 BMDMs (adjusted p-values <0.05) were ranked and a fold-change cut-off (−1<Log(FC)>1) was applied. All genes above the fold-change p-value threshold reported by EdgeR were submitted to GSEA program, compared to their counterparts from the bulk RNASeq dataset, and marker genes discriminating a specific subpopulations were identified using the FindMarkers function. The top markers ranked by Bonferroni adjusted p-values are displayed on a log (10) fold-change color scale, normalized across all cells.

### Co-Immunoprecipitation Experiments

M2 macrophages were re-suspended in RPMI media and treated with 100 μM peptide or PBS for 10 min at 37° C. Cells were pelleted and lysed with 500 μL Pierce IP Lysis buffer (Thermo Scientific, Cat #87787) containing protease and phosphatase inhibitors (Thermo Scientific, Cat #78440). To pull-down CD206 associated proteins via biotinylated RP-182 or RP-426, 50 μL of streptavidin beads (Thermo Scientific, Cat #65001) were added to cleared cell lysates and incubated for 30 min with gentle mixing at RT. Supernatants from the beads were collected after washing the beads four times with PBS containing 0.05% Tween-20, incubating in PBS with 0.1% SDS and boiling for 5 min at 95° C. To validate GRB2 interacting proteins, 25 μg anti-GRB2 antibody was immobilized onto agarose resin using the Pierce Classic IP Kit (Thermo Scientific, Cat #26146) and co-immunoprecipitation was performed following the manufacturer's protocol. Specifically, lysates were incubated with the immobilized antibody overnight at 4° C. with gentle rotation. The columns were washed three times with PBS containing 0.25% Triton X100, and eluted using supplied elution buffer (Thermo Scientific, Cat #21027). Samples were visualized by western blotting using indicated antibodies. Activated forms of the RhoGTPases Rac1 and CDC42 (GTP-Rac1 and GTP-CDC42) were measured with the RhoA/Rac1/Cdc42 combo activation assay kit following the manufacturer's instruction (Abcam, Cambridge, MA).

### Immunofluorescence Assays

Immunocytochemistry analysis was carried out using Zeiss LSM 880 confocal microscope. 50,000 myeloid progenitors were seeded onto 8-well chamber slides, polarized into M1 and M2 macrophages. Cells were treated with 20 μM RP-182 for 2 h at 37° C. followed by fixation with 4% paraformaldehyde for 15 min, permeabilization with 0.3% Triton for 5 min, and blocking with 3% BSA in PBS for 1 h. After blocking, cells were incubated with respective primary antibody (Tables 6A-D) for 1 h at RT. Staining with secondary antibodies was carried out for 1 h at RT, followed by washing and addition of DAPI with mounting media (H-1200 Vectashield, Burlingame, CA). Images were taken at 63× magnification, and three separate images for each treatment group containing about 200 cells were analyzed using ImagePro software (Media Cybernetics, Rockville, MD). The number of automatically counted bright objects (fluorescence of secondary antibodies for specific proteins) was normalized to the number of DAPI stained nuclei. For relative comparison, the fluorescence ratio for vehicle treated cells was set to 1.

### Cell Viability Assay

Dose-response curves in terms of cell viability were determined using the Live/Dead Viability Cytotoxicity Kit (#L3224, ThermoFisher Scientific, Grand Island, NY). Macrophages were seeded onto glass bottom 96 well plates and polarized into M1 and M2. Cells were treated with different concentrations of RP-182 and control peptide RP-426 ranging from 0.01 μM to 100 μM for 48 hours. After drug treatment, 100 μL mixture of 2 μM calcein-AM and 4 μM ethidium homodimer was added and incubated for 1 h. The images were taken in a similar fashion explained in the immunofluorescence assay. 200 cells were counted manually from 3 different random regions across the three technical replicates and percentage of alive cells was calculated using GraphPad Prism version 7.0.

### Animal Models

Colonies of transgenic mice were established at the National Cancer Institute (NCI) in Bethesda, MD, and all animal experiments were conducted according to protocols and policies approved by the Institutional Animal Care and Use Committees (ACUC) of the National Institutes of Health. All animal studies were conducted under ACUC-approved protocols SB-210 and SB-211. Mice with individual genes for Pdx-1-cre, LSL-KrasG12D/+, Trp53R172H/+, and Ink4a(p16)/Arf(p19) flox/flox were obtained from NCI's Mouse Repository, Frederick National Laboratory of Cancer Research. https://frederick.cancer.gov/science/technology/mouserepository) and crossed to create animals with the triple genotype of Pdx-1-cre; LSL-KrasG12D/+; Ink4a(p16)/Arf(p19)flox/flox (KP16) or Pdx-1-cre; LSL-Kras G12D/+; LSL-Trp53R172H/+(KPC) (49, 60). B6.129P2-Mrc1tm1Mnz/J mice were obtained from The Jackson Laboratory (JAX stock #007620) (61). Genotypes were verified using PCR methods performed by Transnetyx, Inc. (Cordova, TN).

Human pancreatic cancer tissues for xenotransplantation were obtained from the NCI Patient-Derived Models Repository (PDMR; https://pdmr.cancer.gov/) initiative and subcutaneously implanted into NOD-scid IL2Rgammanull (NSG) immuno-deficient mice (F0 generation). After tumors reached 2 cm, tumors were explanted, cut into equal pieces, and re-generated in another generation (F1 generation). Treatment experiments were carried out in F2 mice.

Syngeneic murine models of cancer included the murine CT-26 colon cancer and B16 melanoma models. Approximately 1×106 CT-26 cells/100 μL media were implanted subcutaneously into 6 to 8-week old BALB/c mice. When tumors reach ˜50 mm3 volume, mice were started on treatment as outlined below. Tumor volume (mm3) was calculated as (L×W2)/2, with L=length (mm) and W=width (mm) during two-dimensional caliper measurements and total body weights were recorded twice per week. Two hours after the last injections, mice were sacrificed, tumors excised, weighed, and fixed in formalin. Similarly, 0.5×106 murine B16 melanoma cells were subcutaneously injected into the flank of BALB/c animals and treatment was started upon tumors reaching ˜50-100 mm3 volume. 0.5×106 human breast MDA-MB23, prostate C₄₋₂, or KPC cells were subcutaneously injected into the flank of homozygous female athymic (nu/J) nude mice. KPC tumors were treated for 3 weeks after tumors reached 250 mm3, C4-2 tumors were treated for 4 weeks and MDA-MB231 tumors for 6 weeks after tumors had reached 100 mm3 volume, at which point the draining lymph node basin was removed for H&E staining determination of locoregional metastatic index (number lymph nodes involved by cancer per total number of excised and examined lymph nodes in draining basin).

### Animal Imaging

Mice with the KP16 and KPC genotypes were imaged with ultrasound weekly, starting at six weeks of life. Ultrasound imaging was performed using a 40 mHz transducer and a Vevo700 ultrasound machine (Visualsonics, Toronto, Canada). Mice were anesthetized with Isoflurane (Baxter, Deerfield, IL), shaved, and injected intraperitoneally with 1.5 ml of normal saline (eBioscience, San Jose, CA). B-mode images were recorded to obtain tumor measurements.

### Treatment Protocols

KP16 and KPC mice were treated for 7 days prior to harvest of tumors used in flow cytometry, pull down of immune cells, or immune assays, or until a predefined study endpoint. All animal treatments started after ultrasound confirmed a pancreatic tumor measuring ≥4-5 mm and randomization of individual animals to treatment groups. Animal survival was measured from the first day of treatment until death. Animals in control and treatment cohorts were allowed to progress under continuous treatment administration until they reached study end-point (determined as 20% weight loss, recognizable signs of morbidity, general lack of reflexes, abnormal posture, loss of ability to ambulate, labored respiration, inability to drink or feed) where, in order to avoid animal suffering, animals were euthanized in accordance with ACUC animal care guidelines. For experiments in KP16 and KPC mice, normal saline as vehicle, 20 mg/kg RP-182 (PolyPeptide Group, San Diego, CA), 50 mg/kg Gemcitabine (Fresenius Kabi, Lake Zurich, IL), or RP-182 in combination with gemcitabine was injected intraperitoneally (IP) with final volume of 200 μL was administered. RP-182 was injected every other day and gemcitabine was injected 2 times a week. Anti-PD-L1 (Biolegend, Cat. #124329) was administered three times weekly at 150 μg per mouse via intraperitoneal injection. Mice were treated IP with 100 μg anti-CTLA-4 antibody (Bioxcell; 9D9) twice a week. For CD8 depletion, two doses of 100 μg anti-mCD8 (Bioxcell, Cat. #BE0061) per mouse on day 1 and 5 were administered. Rat isotype control IgG1 (Bioxell, Cat. #BE0090) was given at equivalent doses at the same schedule. Mice with CT-26, MDA-MB231, C4-2, and B16 tumors received 10 mg/kg RP-182 via IP injection daily for tumor growth studies, gemcitabine dosing was unchanged, the docetaxol dose delivered to the C4-2 model was docetaxel dosed 2.5 mg/kg daily for 7 days and then discontinued. For intratumoral injections, 50,000 BMDMs pretreated for 2 hours with vehicle or 20 μM RP-182 were injected on days 2, 5, 7, and 9 into KPC tumors ≥500 mm3 grown in C57B/L wild type mice. Prior to injection, M2 BMDMs grown and polarized on T75 flasks were washed ×2, lifted and counted, and resuspended in HBSS for injection volume of <50 μL.

### Bleomycin Lung Fibrosis Model

To facilitate intratracheal bleomycin installation, animals were anesthetized for a short period of time. A single dose of 0.5 mg/kg (1-4 U/mg) bleomycin in sterile isotonic saline (total volume 50 L) was intratracheally administered via a 22 gauge plastic cannula to a total of n=12 BALB/c mice, and the same volume of sterile saline was administered to control group of mice (N=6 mice). Mice instilled with bleomycin were randomized on day 1 to receive 20 mg/kg RP via daily IP injection or vehicle control. Mice underwent daily weight measures, and animal survival was measured from the first day of treatment until death. Animals were allowed in control and RP-182-treatment cohorts to progress under continuous treatment conditions until they reached study end-point (determined as 20% weight loss, recognizable signs of morbidity, general lack of reflexes, abnormal posture, loss of ability to ambulate, labored respiration, inability to drink or feed, determined as moribund with poor survival as per study veterinarian) where, in order to avoid animal suffering, animals were euthanized in accordance with ACUC animal care guidelines. Only ‘warm’ necropsy specimens (lungs) were used for tissue analysis. Lungs were weighed prior to fixation in formalin and embedding in paraffin, and stained with H&E, Masson's trichrome, and anti-CD206. ImageJ was used to quantify the level of fibrosis between the vehicle and RP-182-treated group.

### Flow Cytometry Analysis

Multicolor flow cytometry analysis was performed after 7 days of treatment with RP-182, gemcitabine, the combination, or vehicle. After animal euthanasia, pancreatic tumors were harvested, washed with PBS, and minced with a scalpel. Tumors were

digested using mouse tumor dissociation kit (#130-096-730, MACS Miltenyi Biotec, San Diego, CA) and a Gentle Macs Agitator (Miltenyi Biotec, San Diego, CA) as per company protocol. Tumor lysates were passed through a 70 μm filter, washed in PBS, and stained for flow cytometry analysis. BMDMs subject to flow cytometry were treated with RP-182 and control peptides for 2 or 24 hours at 37° C. prior to staining. Cells were stained with the Live/Dead Fixable Blue Dead Cell Stain Kit (ThermoFisher Scientific), and antibodies coupled to fluorophores as listed in Tables 6A-D (antibodies for flow cytometry). Stained cells were washed with FACS buffer prior to sample acquisition by the BD LSRFortessa SORP I flow cytometer (BD Bioscience). Flow cytometry data was analyzed using FlowJo software (TreeStar, Ashland, OR).

### Histology

Harvested tumors were prepared for histological analysis using standard protocols and 4% paraformaldehyde. In addition to H&E staining, tumors were interrogated by immunocytochemistry and immunohistochemistry using antibodies listed in Tables 6A-D (antibodies for tissue staining). A commercially available pancreatic carcinoma tissue microarray (TMA) with 80 single cores per case (70 from adenocarcinoma pancreas, 10 normal pancreas) was purchased from US Biomax, Inc., Derwood, MD (Cat. No. PA801) for anti-CD206 staining. Brightfield images (immunostaining) were acquired using an Aperio ScanScope XT (Aperio, Vista, CA, USA) for whole slide scanning at 40× magnification and analyzed using ImageScope Analysis. Quantitative analysis was performed using the Aperio membrane algorithm. For immunofluorescence analysis, slides were de-paraffinized as follows: 5 min Xylene immersion twice, 5 min 100% ethanol twice, 5 min 95% ethanol, 5 min 80% ethanol, 5 min 70% ethanol, 5 min rehydration in water. Following antigen retrieval after 60 min incubation at 60° C., slides were blocked with H2O2 peroxidase, washed twice in PBS Blocking solution with FBS or NGS for 30 min, and incubated with the primary antibodies overnight at 4° C. Alexa 488 anti-mouse or Alexa 594 anti-rabbit secondary antibodies were incubated for 30 min at 27° C. Following DAPI counterstaining for 1 min, slides were then cover slipped with glycerol mounting media. Images were collected and analyzed using Zeiss AxioScan imaging.

### TCGA Gene Expression Data Analysis

Genomic data from TCGA project are available from the National Cancer Institute's Genomic Data Commons (https://gdc.cancer.gov/). Gene-level gene expression data from RNA-seq experiments of all tumors (N=9,452) and adenocarcinoma of the pancreas (N=125) were included in analyses and correlated with individual gene expression levels and immune signature scores as previously shown (62, 63).

Isolation of Tumor-Associated Macrophages and Bone Marrow Derived Macrophages (BMDM) and qRT-PCR

TAM's were isolated from tumor digests derived from KP16 and KPC tumors using the EasySep™ Mouse PE positive selection kit (Cat. #18554, StemCell), EasySep™ Mouse custom enrichment kit (Cat. #19709, StemCell) and CD11b (clone M1/70, BD Biosciences), Gr1 (clone RB6-8C5, Biolegend) antibodies. Following tumor harvest and digestion, Gr-1 positive cells were first removed prior to isolation of CD11b− positive macrophages via magnetic cell isolation. Total RNA from TAM isolates and bone-marrow derived macrophage (BMDM) was extracted using RNeasy Mini Kit (Ref: #74104, Qiagen). Bone marrow derived macrophages were sorted for CD11b+GR1−F4/80+CD206+ population using fluorescence activated cell sorting (FACS) FACS sorter and lysates from the same cells used for RT-PCR. Superscript III First-Strand (Ref: #18080-051, Invitrogen) synthesis system for RT-PCR was used to generate cDNA. Following first strand cDNA synthesis, individual primer master mix (IL1b-Mm00434228 m1, TNFα-Mm00443258_m1, IL12-Mm01288989_m1, CD40-Mm00441891 m1, CLEC4e-Mm01183703 m1, CD86-Mm00444540, IL10-Mm01288386_m1, IL27-Mm00461162, PDL1-Mm00452054 m1, SIRPa-Mm00455928, Chil3-Mm00657889_m1, MRC1-Mm01339362_m1, actb-Mm02619580_g1; gapdh-Mm99999915_g1; TaqMan Assays, ThermoFisher Scientific) was added and qRT-PCR reactions were carried out and read in a BioRad CFX96 cycler. Target gene expression was calculated using ‘relative gene expression=2−(ΔCt)’, where ΔCt is the cycle number of target genes (Cttarget) normalized to a reference/housekeeping gene (Ctreference). Individual qRT-PCR reactions were run in triplicate, and graphs were generated by GraphPad Prism.

### ELISpot Assays

T-cell reactivity to cancer cells was assessed in 20-hour co-culture assays in flat-bottom 96-well PVDF-membrane microtiter plates (Cat. #MAIPSWU10, EMD Millipore). CD8a+ T cells were isolated with the EasySep™ Mouse CD8a Positive Selection Kit II (Cat. #18953, StemCell) from single-cell suspensions of whole digested tumors or spleens. 1×105 KP16 cancer cells were co-cultured with 4×104 isolated CD8a+ T cells, positive controls included CD8a+ T cells with PMA/ionomycin, while negative controls contained CD8a+ T cells only. Visualization of immobilized cytokine as ‘ImmunoSpots’ was carried out according to the manufacturer's instructions (Cat. #3321-2A, Mabtech), ELISpots were read and quantified in an ImmunoSpot S6 universal analyzer (C.T.L.). 4×104 CD8+ T cells after pulldown were added to 1×105 KP16 cancer cells and Elispots were then analyzed. In the triple co-culture experiment, 4×104 TAMs, isolated from tumor and spleen from tumor-bearing animals via CD11b pulldown following negative selection for Gr-1, were added to 1×105 KP16 cancer cells and 4×104 CD8+ T cells isolated from spleen of tumor-bearing mice. T-cell reactivity to cancer cells was assessed after 20-hour co-culture as above.

### Phagocytosis Assay

The ability of BMDM's and TAM's to phagocytose cancer cells was analyzed by confocal microscopy and flow cytometry. Cancer cell lines including KPC, PANC1, HPAF-II (pancreatic cancer), the primary melanoma tissue culture line 2183, and LNCaP (prostate cancer) were labelled with CFSE (5-(and-6)-carboxyfluorescein diacetate, succinimidyl ester) dye (#C1157, ThermoFisher Scientific, Grand Island, NY) for 1 hour at 37° C. and 5% CO2 according to manufacturer's instruction. CFSE labeled cancer cells were added onto RP-182 or vehicle (2 hours) pretreated BMDMs and incubated for 6 hours prior to twice washing to remove excess cancer cells. Images were taken using Zeiss LSM 880 confocal microscope at 63× using green and phase contrast channels. For flow cytometry, CFSE-labelled cancer cells were incubated with RP-182 and vehicle-pretreated macrophages (2 hours) cultured on T75 flasks for 4 hours. Excess CFSE-labelled cells were washed, macrophages were harvested from 175 flasks and analyzed on BD LSRFortessa SORP I flow cytometer (BD Bioscience). For the phagocytosis functional assay involving beads, the pHrodo™ Red E. coli BioParticles™ Phagocytosis Kit was used (#A10025, ThermoFisher Scientific, Grand Island, NY). Macrophage polarization, treatments, and analysis for flow and immunofluorescence assays were done in similar fashion explained in the previous experiment.

### Statistical Analysis

Data was statistically analyzed using SPSS software version 16 (IBM, Armonk, NY). Tumor volumes were compared between all four groups using best objective response (BOR), best response recorded from the start of the study treatment compared to any of the follow-up measurements, or absolute measurements (in mm3). Continuous data, including tumor volumes, gene expression levels, or immune cell population percentages, was compared using student's t-test in GraphPad Prism. The log-rank test was used to compare Kaplan-Meier curves. Error bars indicate standard error of the means (SEM) unless otherwise indicated. Calculated p values were given by number and asterisk(s) with * indicating p<0.05, ** p<0.01, and *** p<0.001.

Notwithstanding the appended claims, the disclosure is also defined by the following clauses:

1. A method of modulating macrophage activity, the method comprising:
contacting a macrophage with a CD206-binding agent to modulate activity of the macrophage.

2. The method of clause 1, wherein the CD206-binding agent binds to a site selected from fibronectin II domain of CD206, C-type lectin carbohydrate recognition domain 3 (CRD3) of CD206, C-type lectin carbohydrate recognition domain 4 (CRD4) of CD206 and C-type lectin carbohydrate recognition domain 5 (CRD5) of CD206.

3. The method of clause 1, wherein the CD206-binding agent binds to CD206 with a binding energy of at least −650 kcal/mol.

4. The method of clause 1, wherein the macrophage activity that is modulated is macrophage polarization.

5. The method of clause 1, wherein viability of the macrophage is reduced.

6. The method of clause 1, wherein the macrophage is a M2 macrophage or a tumor associated macrophage (TAM).

7. The method of clause 1, wherein the CD206-binding agent inhibits macrophage activity.

8. The method of clause 1, wherein the CD206-binding agent induces apoptosis of the macrophage.

9. The method of clause 1, wherein the CD206-binding agent stimulates phagocytosis.

10. The method according to any one of clauses 1-9, wherein the macrophage is in vitro.

11. The method according to any one of clauses 1-9, wherein the macrophage is in vivo.

12. The method of any one of clauses 1-11, wherein the CD206-binding agent is an immunomodulatory peptide.

13. The method of clause 12, wherein the immunomodulatory peptide is of 5 to 18 amino acid residues in length, the peptide comprising: a striapathic region of alternating hydrophilic and hydrophobic modules that adopts an amphipathic conformation under physiological conditions.

14. The method of clause 13, wherein the striapathic region comprises:
3 or more hydrophobic modules; and
2 or more hydrophilic modules each comprising at least one cationic residue;
wherein the immunomodulatory peptide specifically binds CD206.

15. The method of any one of clauses 12-14, wherein the immunomodulatory peptide comprises a sequence defined by one of the formulae:

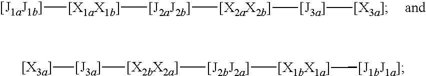

wherein:
J₁ₐ, J_{1b}, J₂ₐ, J_{2b} and J₃ₐ are each independently selected from phenylalanine, tryptophan, alanine, valine, and glycine; and
X₁ₐ, X_{1b}, X₂ₐ, X_{2b} and X₃ₐ are each independently selected from lysine, arginine, histidine, aspartic acid, glutamic acid, asparagine and glutamine.

16. The method of clause 15, wherein:
J₁ₐ, J_{1b}, J₂ₐ, J_{2b} and J₃ₐ are each phenylalanine; and
X₁ₐ, X_{1b}, X₂ₐ, X_{2b} and X₃ₐ are each independently selected from lysine and arginine.

17. The method of any one of clauses 12-16, wherein the immunomodulatory peptide comprises
a) a sequence selected from:

KFRKAFKRFF (RP182);
FFRKFAKRFK (RP183);
FFKKFFKKFK (RP185);
FFKKFFKKFK (RP186);
and
FFKKFFKKFK (RP233);

or
b) a sequence having one or two amino acid substitutions relative to the sequence defined in a).

18. The method of clause 17, wherein the immunomodulatory peptide comprises the amino acid sequence KFRKAFKRFF (RP182)

19. The method of clause 17, wherein the immunomodulatory peptide comprises the amino acid sequence FFRKFAKRFK (RP183).

20. The method of clause 17, wherein the immunomodulatory peptide comprises the amino acid sequence FFKKFFKKFK (RP185).

21. The method of any one of clauses 12-16, wherein the immunomodulatory peptide comprises:
a) a peptide sequence selected from:

RWKFGGFKWR (RP832C);
FKWRGGRWKF (RP837C);
FWKRGGRKWF (RP837A);
FWKRFV (RP837N);
FVRKWR (RP837C1);
FAOOFAOOFO (RP850);
FWKRFVRKWR (RP837);
FWKKFVKKWK (RP841);
WWHHWWHHWH (RP847);
WWRHWWHRWR (RP848);
WWKHWWHKWK (RP849);
GDRGIKGHRGF (RP842);
LYKKIIKKLL (RP846);
FYPDFFKKFF (RP844);
FFRKSKEKIG (RP853);
FFRHFATHLD (RP845);
and
EKLSAFRNFF (RP843);

or
b) a sequence having one or two amino acid substitutions relative to the sequence defined in a).

22. The method of clause 21, wherein the one or two amino acid substitutions defined in b) consist of a highly conservative substitution of a cationic amino acid of the sequence.

23. The method of clause 21, comprising the peptide sequence selected from: RWKFGGFKWR (RP832C), FKWRGGRWKF (RP837C) and FWKRGGRKWF (RP837A).

24. The method of clause 21, comprising the peptide sequence selected from FWKRFV (RP837N) and FVRKWR (RP837C1).

25. The method of clause 21, comprising a peptide sequence selected from FAOOFAOOFO (RP850), FWKRFVRKWR (RP837) and FWKKFVKKWK (RP841).

26. The method of clause 21, comprising a peptide sequence selected from WWHHWWHHWH, WWRHWWHRWR and WWKHWWHKWK (RP847-849).

27. The method of clause 21, comprising the peptide sequence GDRGIKGHRGF (RP842).

28. The method of clause 21, comprising the peptide sequence LYKKIIKKLL (RP846).

29. The method of clause 21, comprising the peptide sequence FYPDFFKKFF (RP844).

30. The method of clause 21, comprising the peptide sequence FFRKSKEKIG (RP853).

31. The method of clause 21, comprising the peptide sequence FFRHFATHLD (RP845).

32. The method of clause 21, comprising the peptide sequence EKLSAFRNFF (RP843).

33. A method of inhibiting growth of a CD206-expressing cell, the method comprising contacting a target CD206-expressing cell with a CD206-binding agent to inhibit growth of the cell.

34. The method of clause 33, wherein the target CD206-expressing cell is a cancer cell.

35. The method of clause 34, wherein the cancer cell is a pancreatic cancer cell, a prostate cancer cell, a colon cancer cell, a skin cancer cell or breast cancer cell.

36. The method of any one of clauses 33-35, wherein the contacting the target CD206-expressing cell comprises administering to a subject in need thereof a therapeutically effective amount of the CD206-binding agent to treat the subject for cancer.

37. The method of clause 36, wherein the cancer is a solid tumor cancer.

38. The method of clause 37, wherein the cancer is a cholangiocarcinoma, pancreatic, prostate, colon, breast, bladder or skin.

39. The method of any one of clauses 36-38, wherein the CD206-binding agent is an immunomodulatory peptide.

40. The method of clause 39, wherein the immunomodulatory peptide is of 5 to 18 amino acid residues in length, the peptide comprising: a striapathic region of alternating hydrophilic and hydrophobic modules that adopts an amphipathic conformation under physiological conditions.

41. The method of clause 40, wherein the striapathic region comprises:
3 or more hydrophobic modules; and
2 or more hydrophilic modules each comprising at least one cationic residue;
wherein the immunomodulatory peptide specifically binds CD206.

42. The method of any one of clauses 39-41, wherein the immunomodulatory peptide comprises a sequence defined by one of the formulae:

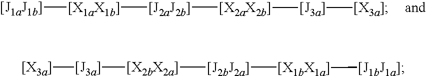

wherein:
J₁ₐ, J_{1b}, J₂ₐ, J_{2b} and J₃ₐ are each independently selected from phenylalanine, tryptophan, alanine, valine, and glycine; and
X₁ₐ, X_{1b}, X₂ₐ, X_{2b} and X₃ₐ are each independently selected from lysine, arginine, histidine, aspartic acid, glutamic acid, asparagine and glutamine.

43. The method of clause 42, wherein:
J₁ₐ, J_{1b}, J₂ₐ, J_{2b} and J₃ₐ are each phenylalanine; and
X₁ₐ, X_{1b}, X₂ₐ, X_{2b} and X₃ₐ are each independently selected from lysine and arginine.

44. The method of any one of clauses 39-43, wherein the immunomodulatory peptide comprises
a) a sequence selected from:

KFRKAFKRFF (RP182);
FFRKFAKRFK (RP183);
FFKKFFKKFK (RP233);
FFKKFFKKFK (RP186);
and
FFKKFFKKFK (RP185);

or
b) a sequence having one or two amino acid substitutions relative to the sequence defined in a).

45. The method of clause 44, wherein the immunomodulatory peptide comprises the amino acid sequence KFRKAFKRFF (RP182)

46. The method of clause 44, wherein the immunomodulatory peptide comprises the amino acid sequence FFRKFAKRFK (RP183).

47. The method of clause 44, wherein the immunomodulatory peptide comprises the amino acid sequence FFKKFFKKFK (RP185).

48. The method of any one of clauses 39-43, wherein the immunomodulatory peptide comprises:
a) a peptide sequence selected from:

RWKFGGFKWR (RP832C);
FKWRGGRWKF (RP837C);
FWKRGGRKWF (RP837A);
FWKRFV (RP837N);
FVRKWR (RP837C1);
FAOOFAOOFO (RP850);
FWKRFVRKWR (RP837);
FWKKFVKKWK (RP841);
WWHHWWHHWH (RP847);
WWRHWWHRWR (RP848);
WWKHWWHKWK (RP849);
GDRGIKGHRGF (RP842);
LYKKIIKKLL (RP846);
FYPDFFKKFF (RP844);
FFRKSKEKIG (RP853);
FFRHFATHLD (RP845);
and
EKLSAFRNFF (RP843);

or
b) a sequence having one or two amino acid substitutions relative to the sequence defined in a).

49. The method of clause 48, wherein the one or two amino acid substitutions defined in b) consist of a highly conservative substitution of a cationic amino acid of the sequence.

50. The method of clause 48, comprising the peptide sequence selected from: RWKFGGFKWR (RP832C), FKWRGGRWKF (RP837C) and FWKRGGRKWF (RP837A).

51. The method of clause 48, comprising the peptide sequence selected from FWKRFV (RP837N) and FVRKWR (RP837C1).

52. The method of clause 48, comprising a peptide sequence selected from FAOOFAOOFO (RP850), FWKRFVRKWR (RP837) and FWKKFVKKWK (RP841).

53. The method of clause 48, comprising a peptide sequence selected from WWHHWWHHWH, WWRHWWHRWR and WWKHWWHKWK (RP847-849).

54. The method of clause 48, comprising the peptide sequence GDRGIKGHRGF (RP842).

55. The method of clause 48, comprising the peptide sequence LYKKIIKKLL (RP846).

56. The method of clause 48, comprising the peptide sequence FYPDFFKKFF (RP844).

57. The method of clause 48, comprising the peptide sequence FFRKSKEKIG (RP853).

58. The method of clause 48, comprising the peptide sequence FFRHFATHLD (RP845).

59. The method of clause 48, comprising the peptide sequence EKLSAFRNFF (RP843).

60. A method of treating a subject for a condition associated with chronic inflammation, the method comprising:
administering a therapeutically effective amount of a CD206-binding agent to a subject to treat the subject for the condition associated with chronic inflammation.

61. The method of clause 60, wherein the condition associated with chronic inflammation is selected from scleroderma or multiple sclerosis, irritable bowel disease, ulcerative colitis, colitis, Crohn's disease, idiopathic pulmonary fibrosis, asthma, keratitis, arthritis, osteoarthritis, rheumatoid arthritis, auto-immune diseases, a feline or human immunodeficiency virus (FIV or HIV) infection, cancer, age-related inflammation and/or stem cell dysfunction, graft-versus-host disease (GVHD), keloids, obesity, diabetes, diabetic wounds, other chronic wounds, atherosclerosis, Parkinson's disease, Alzheimer's disease, macular degeneration, gout, gastric ulcers, gastritis, mucositis, toxoplasmosis, and chronic viral or microbial infections.

62. The method of any one of clauses 60-61, wherein the CD206-binding agent is administered in conjunction with another drug known to be effective in treating the condition.

63. The method of clause 62, wherein the condition is cancer.

64. The method of clause 63, wherein the cancer is pancreatic, prostate, colon, breast or skin.

65. The method of clause 64, further comprising administering an effective amount of a chemotherapeutic agent, antibody agent or cell therapy to the subject.

66. The method of clause 65, wherein the chemotherapeutic agent, antibody agent or cell therapy is selected from steroids, anthracyclines, thyroid hormone replacement drugs, thymidylate-targeted drugs, antibodies, checkpoint inhibitor drugs, Chimeric Antigen Receptor/T cell therapies, and other cell therapies.

67. The method of clause 60, wherein the condition associated with chronic inflammation is a fibrosis or scleroderma.

68. The method of any one of clauses 60-67, wherein the CD206-binding agent is an immunomodulatory peptide.

69. The method of clause 68, wherein the immunomodulatory peptide is of 5 to 18 amino acid residues in length, the peptide comprising: a striapathic region of alternating hydrophilic and hydrophobic modules that adopts an amphipathic conformation under physiological conditions.

70. The method of clause 69, wherein the striapathic region comprises:
3 or more hydrophobic modules; and
2 or more hydrophilic modules each comprising at least one cationic residue;
wherein the immunomodulatory peptide specifically binds CD206.

71. The method of any one of clauses 68-70, wherein the immunomodulatory peptide comprises a sequence defined by one of the formulae:

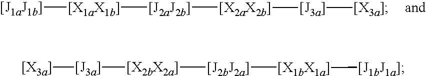

wherein:
J₁ₐ, J_{1b}, J₂ₐ, J_{2b} and J₃ₐ are each independently selected from phenylalanine, tryptophan, alanine, valine, and glycine; and
X₁ₐ, X_{1b}, X₂ₐ, X_{2b} and X₃ₐ are each independently selected from lysine, arginine, histidine, aspartic acid, glutamic acid, asparagine and glutamine.

72. The method of clause 71, wherein:
J₁ₐ, J_{1b}, J₂ₐ, J_{2b} and J₃ₐ are each phenylalanine; and
X₁ₐ, X_{1b}, X₂ₐ, X_{2b} and X₃ₐ are each independently selected from lysine and arginine.

73. The method of any one of clauses 68-72, wherein the immunomodulatory peptide comprises
a) a sequence selected from:

KFRKAFKRFF (RP182);
FFRKFAKRFK (RP183);
FFKKFFKKFK (RP185);
FFKKFFKKFK (RP186);
and
FFKKFFKKFK (RP233);

or
b) a sequence having one or two amino acid substitutions relative to the sequence defined in a).

74. The method of clause 73, wherein the immunomodulatory peptide comprises the amino acid sequence KFRKAFKRFF (RP182)

75. The method of clause 73, wherein the immunomodulatory peptide comprises the amino acid sequence FFRKFAKRFK (RP183).

76. The method of clause 73, wherein the immunomodulatory peptide comprises the amino acid sequence FFKKFFKKFK (RP185).

77. The method of any one of clauses 68-72, wherein the immunomodulatory peptide comprises:
a) a peptide sequence selected from:

RWKFGGFKWR (RP832C);
FKWRGGRWKF (RP837C);
FWKRGGRKWF (RP837A);
FWKRFV (RP837N);
FVRKWR (RP837C1);
FAOOFAOOFO (RP850);
FWKRFVRKWR (RP837);
FWKKFVKKWK (RP841);
WWHHWWHHWH (RP847);
WWRHWWHRWR (RP848);
WWKHWWHKWK (RP849);
GDRGIKGHRGF (RP842);
LYKKIIKKLL (RP846);
FYPDFFKKFF (RP844);
FFRKSKEKIG (RP853);
FFRHFATHLD (RP845);
and
EKLSAFRNFF (RP843);

or
b) a sequence having one or two amino acid substitutions relative to the sequence defined in a).

78. The method of clause 77, wherein the one or two amino acid substitutions defined in b) consist of a highly conservative substitution of a cationic amino acid of the sequence.

79. The method of clause 77, comprising the peptide sequence selected from: RWKFGGFKWR (RP832C), FKWRGGRWKF (RP837C) and FWKRGGRKWF (RP837A).

80. The method of clause 77, comprising the peptide sequence selected from FWKRFV (RP837N) and FVRKWR (RP837C1).

81. The method of clause 77, comprising a peptide sequence selected from FAOOFAOOFO (RP850), FWKRFVRKWR (RP837) and FWKKFVKKWK (RP841).

82. The method of clause 77, comprising a peptide sequence selected from WWHHWWHHWH, WWRHWWHRWR and WWKHWWHKWK (RP847-849).

83. The method of clause 77, comprising the peptide sequence GDRGIKGHRGF (RP842).

84. The method of clause 77, comprising the peptide sequence LYKKIIKKLL (RP846).

85. The method of clause 77, comprising the peptide sequence FYPDFFKKFF (RP844).

86. The method of clause 77, comprising the peptide sequence FFRKSKEKIG (RP853).

87. The method of clause 77, comprising the peptide sequence FFRHFATHLD (RP845).

88. The method of clause 77, comprising the peptide sequence EKLSAFRNFF (RP843).

89. A method of converting a phenotype of a macrophage from an M2 phenotype to an M1 phenotype, the method comprising contacting a macrophage having an M2 phenotype with a CD206-binding agent in a manner sufficient to convert the phenotype of the macrophage to an M1 phenotype.

90. The method of clause 89, wherein contacting the CD206-binding agent induces a conformational change in a CD206 receptor of the macrophage sufficient to convert the phenotype of the macrophage to an M1 phenotype.

91. The method of clause 89-90, wherein converting the phenotype of the macrophage comprises inducing expression of CD86 by the macrophage.

92. The method of any one of clauses 89-91, wherein converting the phenotype of the macrophage comprises reducing expression of CD206 or CD163 by the macrophage.

93. The method of any one of clauses 89-92, wherein converting the phenotype of the macrophage comprises converting the macrophage to a phenotype that exhibits upregulation of M1 cytokines and markers.

94. The method of clause 93, wherein M1 cytokine and marker is selected from the group consisting of IL-1β, IL-12, TNFα and nitric oxide synthetase.

95. The method of any one of clauses 89-94, wherein converting the phenotype of the macrophage comprises converting the macrophage to a phenotype that exhibits decreased expression of signal regulatory protein α(SIRPα)

96. The method of any one of clauses 89-95, wherein the CD206-binding agent binds to a site selected from fibronectin II domain of CD206, C-type lectin carbohydrate recognition domain 3 (CRD3) of CD206, C-type lectin carbohydrate recognition domain 4 (CRD4) of CD206 and C-type lectin carbohydrate recognition domain 5 (CRD5) of CD206.

97. The method according to any one of clauses 89-96, wherein the macrophage is contacted with the CD206-binding agent in vivo.

98. The method according to any one of clauses 89-97, wherein the macrophage is contacted with the CD206-binding agent in vitro.

99. The method of any one of clauses 89-98, wherein the CD206-binding agent is an immunomodulatory peptide.

100. The method of clause 99, wherein the immunomodulatory peptide is of 5 to 18 amino acid residues in length, the peptide comprising: a striapathic region of alternating hydrophilic and hydrophobic modules that adopts an amphipathic conformation under physiological conditions.

101. The method of clause 100, wherein the striapathic region comprises:
3 or more hydrophobic modules; and
2 or more hydrophilic modules each comprising at least one cationic residue;
wherein the immunomodulatory peptide specifically binds CD206.

102. The method of any one of clauses 99-101, wherein the immunomodulatory peptide comprises a sequence defined by one of the formulae:

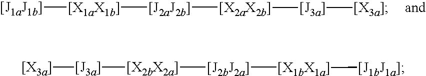

wherein:
J₁ₐ, J_{1b}, J₂ₐ, J_{2b} and J₃ₐ are each independently selected from phenylalanine, tryptophan, alanine, valine, and glycine; and
X₁ₐ, X_{1b}, X₂ₐ, X_{2b} and X₃ₐ are each independently selected from lysine, arginine, histidine, aspartic acid, glutamic acid, asparagine and glutamine.

103. The method of clause 102, wherein:
J₁ₐ, J_{1b}, J₂ₐ, J_{2b} and J₃ₐ are each phenylalanine; and
X₁ₐ, X_{1b}, X₂ₐ, X_{2b} and X₃ₐ are each independently selected from lysine and arginine.

104. The method of any one of clauses 99-103, wherein the immunomodulatory peptide comprises
a) a sequence selected from:

KFRKAFKRFF (RP182);
FFRKFAKRFK (RP183);
FFKKFFKKFK (RP185);
FFKKFFKKFK (RP186);
and
FFKKFFKKFK (RP233);

or
b) a sequence having one or two amino acid substitutions relative to the sequence defined in a).

105. The method of clause 104, wherein the immunomodulatory peptide comprises the amino acid sequence KFRKAFKRFF (RP182)

106. The method of clause 104, wherein the immunomodulatory peptide comprises the amino acid sequence FFRKFAKRFK (RP183).

107. The method of clause 104, wherein the immunomodulatory peptide comprises the amino acid sequence FFKKFFKKFK (RP185).

108. The method of any one of clauses 99-103, wherein the immunomodulatory peptide comprises:
a) a peptide sequence selected from:

RWKFGGFKWR (RP832C);
FKWRGGRWKF (RP837C);
FWKRGGRKWF (RP837A);
FWKRFV (RP837N);
FVRKWR (RP837C1);
FAOOFAOOFO (RP850);
FWKRFVRKWR (RP837);
FWKKFVKKWK (RP841);
WWHHWWHHWH (RP847);
WWRHWWHRWR (RP848);
WWKHWWHKWK (RP849);
GDRGIKGHRGF (RP842);
LYKKIIKKLL (RP846);
FYPDFFKKFF (RP844);
FFRKSKEKIG (RP853);
FFRHFATHLD (RP845);
and
EKLSAFRNFF (RP843);

or
b) a sequence having one or two amino acid substitutions relative to the sequence defined in a).

109. The method of clause 108, wherein the one or two amino acid substitutions defined in b) consist of a highly conservative substitution of a cationic amino acid of the sequence.

110. The method of clause 108, comprising the peptide sequence selected from: RWKFGGFKWR (RP832C), FKWRGGRWKF (RP837C) and FWKRGGRKWF (RP837A).

111. The method of clause 108, comprising the peptide sequence selected from FWKRFV (RP837N) and FVRKWR (RP837C1).

112. The method of clause 108, comprising a peptide sequence selected from FAOOFAOOFO (RP850), FWKRFVRKWR (RP837) and FWKKFVKKWK (RP841).

113. The method of clause 108, comprising a peptide sequence selected from WWHHWWHHWH, WWRHWWHRWR and WWKHWWHKWK (RP847-849).

114. The method of clause 108, comprising the peptide sequence GDRGIKGHRGF (RP842).

115. The method of clause 108, comprising the peptide sequence LYKKIIKKLL (RP846).

116. The method of clause 108, comprising the peptide sequence FYPDFFKKFF (RP844).

117. The method of clause 108, comprising the peptide sequence FFRKSKEKIG (RP853).

118. The method of clause 108, comprising the peptide sequence FFRHFATHLD (RP845).

119. The method of clause 108, comprising the peptide sequence EKLSAFRNFF (RP843).

120. A method of treating a subject for a neoplastic condition, the method comprising administering to a therapeutically effective amount of a CD206-binding agent to a subject diagnosed as having a neoplastic condition to treat the neoplastic condition in the subject.

121. The method of clause 120, wherein the neoplastic condition is a solid-tumor cancer.

122. The method of any one of clauses 120-121, wherein the neoplastic condition is a cancer selected from the group consisting of pancreatic cancer, prostate cancer, colon cancer, breast cancer and skin cancer.

123. The method of any one of clauses 120-122, further comprising administering an effective amount of a chemotherapeutic agent, antibody agent or cell therapy to the subject.

124. The method of clause 123, wherein the chemotherapeutic agent, antibody agent or cell therapy is selected from steroids, anthracyclines, thyroid hormone replacement drugs, thymidylate-targeted drugs, antibodies, checkpoint inhibitor drugs, Chimeric Antigen Receptor/T cell therapies, and other cell therapies.

125. The method of clause 124, wherein the chemotherapeutic agent is a non-peptidic compound that reduces proliferation of cancer cells.

126. The method of any one of clauses 124-125, wherein the chemotherapeutic agent is a compound selected from the group consisting of alkylating agents, metal complexes, nitrosoureas, antimetabolites, antitumor antibiotics, plant (vinca) alkaloids, hormone modulators, steroid hormones.

127. The method of clause 126, wherein the antibody agent is a chemotherapeutic antibody agent.

128. The method of clause 127, wherein the antibody agent is an antibody raised against a tumor-associated antigen selected from the group consisting of CD20, CD30, CD33, CD52, EpCAM, CEA, gpA33, Mucins, TAG-72, CAIX, PSMA, Folate-binding protein, Gangliosides (e.g., GD2, GD3, GM2, etc.), Le y, VEGF, VEGFR, Integrin alpha-V-beta-3, Integrin alpha-5-beta-1, EGFR, ERBB2, ERBB3, MET, IGF1R, EPHA3, TRAILR1, TRAILR2, RANKL, FAP and Tenascin.

129. The method of 124, wherein the immune checkpoint inhibitor is an inhibitory compound that targets one or more of PD1, PD-L1, CTLA4, TIM3, LAG3, VISTA, BTLA, TIGIT, LAIR1, CD160, 2B4 and TGFRβ.

130. The method of any one of clauses 120-129, wherein the CD206-binding agent is an immunomodulatory peptide.

131. The method of clause 130, wherein the immunomodulatory peptide is of 5 to 18 amino acid residues in length, the peptide comprising: a striapathic region of alternating hydrophilic and hydrophobic modules that adopts an amphipathic conformation under physiological conditions.

132. The method of clause 131, wherein the striapathic region comprises:
3 or more hydrophobic modules; and
2 or more hydrophilic modules each comprising at least one cationic residue;
wherein the immunomodulatory peptide specifically binds CD206.

133. The method of any one of clauses 130-132, wherein the immunomodulatory peptide comprises a sequence defined by one of the formulae:

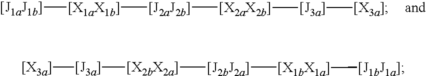

wherein:
J₁ₐ, J_{1b}, J₂ₐ, J_{2b} and J₃ₐ are each independently selected from phenylalanine, tryptophan, alanine, valine, and glycine; and
X₁ₐ, X_{1b}, X₂ₐ, X_{2b} and X₃ₐ are each independently selected from lysine, arginine, histidine, aspartic acid, glutamic acid, asparagine and glutamine.

134. The method of clause 133, wherein:
J₁ₐ, J_{1b}, J₂ₐ, J_{2b} and J₃ₐ are each phenylalanine; and
X₁ₐ, X_{1b}, X₂ₐ, X_{2b} and X₃ₐ are each independently selected from lysine and arginine.

135. The method of any one of clauses 130-134, wherein the immunomodulatory peptide comprises
a) a sequence selected from:

KFRKAFKRFF (RP182);
FFRKFAKRFK (RP183);
FFKKFFKKFK (RP185);
FFKKFFKKFK (RP186);
and
FFKKFFKKFK (RP233);

or
b) a sequence having one or two amino acid substitutions relative to the sequence defined in a).

136. The method of clause 135, wherein the immunomodulatory peptide comprises the amino acid sequence KFRKAFKRFF (RP182)

137. The method of clause 135, wherein the immunomodulatory peptide comprises the amino acid sequence FFRKFAKRFK (RP183).

138. The method of clause 135, wherein the immunomodulatory peptide comprises the amino acid sequence FFKKFFKKFK (RP185).

139. The method of any one of clauses 130-134, wherein the immunomodulatory peptide comprises:
a) a peptide sequence selected from:

RWKFGGFKWR (RP832C);
FKWRGGRWKF (RP837C);
FWKRGGRKWF (RP837A);
FWKRFV (RP837N);
FVRKWR (RP837C1);
FAOOFAOOFO (RP850);
FWKRFVRKWR (RP837);
FWKKFVKKWK (RP841);
WWHHWWHHWH (RP847);
WWRHWWHRWR (RP848);
WWKHWWHKWK (RP849);
GDRGIKGHRGF (RP842);
LYKKIIKKLL (RP846);
FYPDFFKKFF (RP844);
FFRKSKEKIG (RP853);
FFRHFATHLD (RP845);
and
EKLSAFRNFF (RP843);

or
b) a sequence having one or two amino acid substitutions relative to the sequence defined in a).

140. The method of clause 139, wherein the one or two amino acid substitutions defined in b) consist of a highly conservative substitution of a cationic amino acid of the sequence.

141. The method of clause 139, comprising the peptide sequence selected from: RWKFGGFKWR (RP832C), FKWRGGRWKF (RP837C) and FWKRGGRKWF (RP837A).

142. The method of clause 139, comprising the peptide sequence selected from FWKRFV (RP837N) and FVRKWR (RP837C1).

143. The method of clause 139, comprising a peptide sequence selected from FAOOFAOOFO (RP850), FWKRFVRKWR (RP837) and FWKKFVKKWK (RP841).

144. The method of clause 139, comprising a peptide sequence selected from WWHHWWHHWH, WWRHWWHRWR and WWKHWWHKWK (RP847-849).

145. The method of clause 139, comprising the peptide sequence GDRGIKGHRGF (RP842).

146. The method of clause 139, comprising the peptide sequence LYKKIIKKLL (RP846).

147. The method of clause 139, comprising the peptide sequence FYPDFFKKFF (RP844).

148. The method of clause 139, comprising the peptide sequence FFRKSKEKIG (RP853).

149. The method of clause 139, comprising the peptide sequence FFRHFATHLD (RP845).

150. The method of clause 139, comprising the peptide sequence EKLSAFRNFF (RP843).

151. A method of combination therapy, the method comprising administering to a subject in need thereof:
a CD206-binding agent; and
one or more of a chemotherapeutic agent, antibody agent, an immune checkpoint inhibitor drug or cell therapy.

152. The method of clause 151, wherein the chemotherapeutic agent is a non-peptidic compound that reduces proliferation of cancer cells.

153. The method of clause 151, wherein the chemotherapeutic agent is a compound selected from the group consisting of alkylating agents, metal complexes, nitrosoureas, antimetabolites, antitumor antibiotics, plant (vinca) alkaloids, hormone modulators, steroid hormones.

154. The method of clause 151, wherein the antibody agent is a chemotherapeutic antibody agent.

155. The method of clause 154, wherein the antibody agent is an antibody raised against a tumor-associated antigen selected from the group consisting of CD20, CD30, CD33, CD52, EpCAM, CEA, gpA33, Mucins, TAG-72, CAIX, PSMA, Folate-binding protein, Gangliosides (e.g., GD2, GD3, GM2, etc.), Le y, VEGF, VEGFR, Integrin alpha-V-beta-3, Integrin alpha-5-beta-1, EGFR, ERBB2, ERBB3, MET, IGF1R, EPHA3, TRAILR1, TRAILR2, RANKL, FAP and Tenascin.

156. The method of clause 151, wherein the immune checkpoint inhibitor is an inhibitory compound that targets one or more of PD1, PD-L1, CTLA4, TIM3, LAG3, VISTA, BTLA, TIGIT, LAIR1, CD160, 2B4 and TGFR.

157. The method of any one of clauses 151-156, wherein the CD206-binding agent is an immunomodulatory peptide.

158. The method of clause 157, wherein the immunomodulatory peptide is of 5 to 18 amino acid residues in length, the peptide comprising: a striapathic region of alternating hydrophilic and hydrophobic modules that adopts an amphipathic conformation under physiological conditions.

159. The method of clause 158, wherein the striapathic region comprises:
3 or more hydrophobic modules; and
2 or more hydrophilic modules each comprising at least one cationic residue;
wherein the immunomodulatory peptide specifically binds CD206.

160. The method of any one of clauses 157-159, wherein the immunomodulatory peptide comprises a sequence defined by one of the formulae:

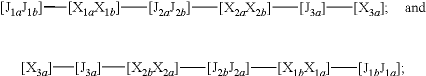

wherein:
J₁ₐ, J_{1b}, J₂ₐ, J_{2b} and J₃ₐ are each independently selected from phenylalanine, tryptophan, alanine, valine, and glycine; and
X₁ₐ, X_{1b}, X₂ₐ, X_{2b} and X₃ₐ are each independently selected from lysine, arginine, histidine, aspartic acid, glutamic acid, asparagine and glutamine.

161. The method of clause 160, wherein:
J₁ₐ, J_{1b}, J₂ₐ, J_{2b} and J₃ₐ are each phenylalanine; and
X₁ₐ, X_{1b}, X₂ₐ, X_{2b} and X₃ₐ are each independently selected from lysine and arginine.

162. The method of any one of clauses 157-161, wherein the immunomodulatory peptide comprises
a) a sequence selected from:

KFRKAFKRFF (RP182);
FFRKFAKRFK (RP183);
FFKKFFKKFK (RP185);
FFKKFFKKFK (RP186);
and
FFKKFFKKFK (RP233);

or
b) a sequence having one or two amino acid substitutions relative to the sequence defined in a).

163. The method of clause 162, wherein the immunomodulatory peptide comprises the amino acid sequence KFRKAFKRFF (RP182)

164. The method of clause 162, wherein the immunomodulatory peptide comprises the amino acid sequence FFRKFAKRFK (RP183).

165. The method of clause 162, wherein the immunomodulatory peptide comprises the amino acid sequence FFKKFFKKFK (RP185).

166. The method of any one of clauses 157-161, wherein the immunomodulatory peptide comprises:
a) a peptide sequence selected from:

RWKFGGFKWR (RP832C);
FKWRGGRWKF (RP837C);
FWKRGGRKWF (RP837A);
FWKRFV (RP837N);
FVRKWR (RP837C1);
FAOOFAOOFO (RP850);
FWKRFVRKWR (RP837);
FWKKFVKKWK (RP841);
WWHHWWHHWH (RP847);
WWRHWWHRWR (RP848);
WWKHWWHKWK (RP849);
GDRGIKGHRGF (RP842);
LYKKIIKKLL (RP846);
FYPDFFKKFF (RP844);
FFRKSKEKIG (RP853);
FFRHFATHLD (RP845);
and
EKLSAFRNFF (RP843);

or
b) a sequence having one or two amino acid substitutions relative to the sequence defined in a).

167. The method of clause 166, wherein the one or two amino acid substitutions defined in b) consist of a highly conservative substitution of a cationic amino acid of the sequence.

168. The method of clause 166, comprising the peptide sequence selected from: RWKFGGFKWR (RP832C), FKWRGGRWKF (RP837C) and FWKRGGRKWF (RP837A).

169. The method of clause 166, comprising the peptide sequence selected from FWKRFV (RP837N) and FVRKWR (RP837C1).

170. The method of clause 166, comprising a peptide sequence selected from FAOOFAOOFO (RP850), FWKRFVRKWR (RP837) and FWKKFVKKWK (RP841).

171. The method of clause 166, comprising a peptide sequence selected from WWHHWWHHWH, WWRHWWHRWR and WWKHWWHKWK (RP847-849).

172. The method of clause 166, comprising the peptide sequence GDRGIKGHRGF (RP842) or LYKKIIKKLL (RP846).

173. The method of clause 166, comprising the peptide sequence FYPDFFKKFF (RP844).

174. The method of clause 166, comprising the peptide sequence FFRKSKEKIG (RP853).

175. The method of clause 166, comprising the peptide sequence FFRHFATHLD (RP845).

176. The method of clause 166, comprising the peptide sequence EKLSAFRNFF (RP843).

177. An active agent that binds to an activity modulating domain of CD206.

178. The active agent according to clause 177, wherein the active agent binds to an activity modulating domain of CD206 selected from fibronectin II domain of CD206, C-type lectin carbohydrate recognition domain 3 (CRD3) of CD206, C-type lectin carbohydrate recognition domain 4 (CRD4) of CD206 and C-type lectin carbohydrate recognition domain 5 (CRD5) of CD206.

179. The active agent of clause 178, wherein the active agent binds to CRD5 domain of CD206.

180. The active agent of clause 178, wherein the active agent binds to fibronectin II domain of CD206.

181. The active agent of clause 178, wherein the active agent binds to CRD3 domain of CD206.

182. The active agent of any one of clauses 177 to 181, wherein the active agent is an immunomodulatory peptide.

183. The active agent of clause 182, wherein the immunomodulatory peptide is of 5 to 18 amino acid residues in length, the peptide comprising: a striapathic region of alternating hydrophilic and hydrophobic modules that adopts an amphipathic conformation under physiological conditions.

184. The active agent of clause 183, wherein the striapathic region comprises:
3 or more hydrophobic modules; and
2 or more hydrophilic modules each comprising at least one cationic residue;
wherein the immunomodulatory peptide binds to the activity modulating domain of CD206.

185. The active agent of any one of clauses 182-184, wherein the immunomodulatory peptide comprises a sequence defined by one of the formulae:

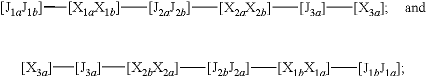

wherein:
J₁ₐ, J_{1b}, J₂ₐ, J_{2b} and J₃ₐ are each independently selected from phenylalanine, tryptophan, alanine, valine, and glycine; and
X₁ₐ, X_{1b}, X₂ₐ, X_{2b} and X₃ₐ are each independently selected from lysine, arginine, histidine, aspartic acid, glutamic acid, asparagine and glutamine.

186. The active agent of clause 182 or 183, wherein the immunomodulatory peptide comprises a sequence defined by one of the formulae:

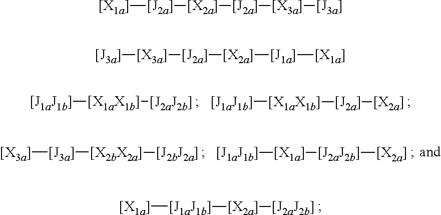

wherein:
J₁ₐ, J_{1b}, J₂ₐ, J_{2b} and J₃ₐ are each independently selected from phenylalanine, tryptophan, alanine, valine, leucine, isoleucine, methionine, tyrosine, threonine, serine, cysteine, proline, and glycine; and
X₁ₐ, X_{1b}, X₂ₐ, X_{2b} and X₃ₐ are each independently selected from lysine, arginine, histidine, aspartic acid, glutamic acid, asparagine and glutamine.

187. The active agent of clause 185 or 186, wherein:
J₁ₐ, J_{1b}, J₂ₐ, J_{2b} and J₃ₐ are each phenylalanine; and
X₁ₐ, X_{1b}, X₂ₐ, X_{2b} and Xu are each independently selected from lysine and arginine.

188. The active agent of clause 185, wherein the immunomodulatory peptide comprises
a) a sequence selected from:

KFRKAFKRFF (RP182);
FFRKFAKRFK (RP183);
FFKKFFKKFK (RP185);
FFKKFFKKFK (RP186);
and
FFKKFFKKFK (RP233);

or
b) a sequence having one or two amino acid substitutions relative to the sequence defined in a).

189. The active agent of clause 188, wherein the immunomodulatory peptide comprises the amino acid sequence KFRKAFKRFF (RP182)

190. The active agent of clause 188, wherein the immunomodulatory peptide comprises the amino acid sequence FFRKFAKRFK (RP183).

191. The active agent of clause 189, wherein the immunomodulatory peptide comprises the amino acid sequence FFKKFFKKFK (RP185).

192. The active agent of clause 183, wherein the immunomodulatory peptide comprises:
a) a peptide sequence selected from:

RWKFGGFKWR (RP832C);
FKWRGGRWKF (RP837C);
FWKRGGRKWF (RP837A);
FWKRFV (RP837N);
FVRKWR (RP837C1);
FAOOFAOOFO (RP850);
FWKRFVRKWR (RP837);
FWKKFVKKWK (RP841);
WWHHWWHHWH (RP847);
WWRHWWHRWR (RP848);
WWKHWWHKWK (RP849);
GDRGIKGHRGF (RP842);
LYKKIIKKLL (RP846);
FYPDFFKKFF (RP844);
FFRKSKEKIG (RP853);
FFRHFATHLD (RP845);
and
EKLSAFRNFF (RP843);

or
b) a sequence having one or two amino acid substitutions relative to the sequence defined in a).

193. The active agent of clause 192, wherein the one or two amino acid substitutions defined in b) consist of a highly conservative substitution of a cationic amino acid of the sequence.

194. The active agent of clause 192, comprising the peptide sequence selected from: RWKFGGFKWR (RP832C), FKWRGGRWKF (RP837C) and FWKRGGRKWF (RP837A).

195. The active agent of clause 192, comprising the peptide sequence selected from FWKRFV (RP837N) and FVRKWR (RP837C1).

196. The active agent of clause 192, comprising a peptide sequence selected from FAOOFAOOFO (RP850), FWKRFVRKWR (RP837) and FWKKFVKKWK (RP841).

197. The active agent of clause 192, comprising a peptide sequence selected from WWHHWWHHWH, WWRHWWHRWR and WWKHWWHKWK (RP847-849).

198. The active agent of clause 183, comprising a peptide sequence selected from MVFRDVGNRN, LFWKRFVEFF, AIRRIPRRIR, LAERAFHRFF, DVRMRLRSEV, FFNRFANERH, GFRELFRQLD, SQLPAFKRFF, RRAELGFKWR, MFEREVKNAM, REVKNAMRRW, IENAAFKRFF, FYPDFFKKFF, and KKIRVRSLA

199. The active agent of clause 192, comprising the peptide sequence GDRGIKGHRGF (RP842).

200. The active agent of clause 192, comprising the peptide sequence LYKKIIKKLL (RP846).

201. The active agent of clause 192, comprising the peptide sequence FYPDFFKKFF (RP844).

202. The active agent of clause 192, comprising the peptide sequence FFRKSKEKIG (RP853).

203. The active agent of clause 192, comprising the peptide sequence FFRHFATHLD (RP845).

204. The active agent of clause 192, comprising the peptide sequence EKLSAFRNFF (RP843).

205. The active agent of clause 186, wherein the immunomodulatory peptide comprises:
a) a peptide sequence selected from:

AFKRFF (182-FN6);
FFKKFF (185-FN6);
FWKRFV (837-FN6);
WVRRVV (WLUB-F1-N6);
IFKKIE (CEC-F1-N6)
FLRNLV (LL37F-3-N6);
FLHSAK (MAG-F1-N6);
FFHHIF (PISC-F-N6);
FFKKAA (PLEU-F-N6);
ALKKVF (PSEU-F-N6);
LYKKII (CXCL4-F-N6);
LFRRAF (IL24-FN6);
FLKRLL (IL7-FN6);
FFRRFA (ABCP-FN6);
FFRHFA (EIP-FN6);
AIRRIP (gP120-FN6);
AFHRFF (GP2B-FN6);
FFNRFA (MCPH-FN6);
AFKRFF (SPRA-FN6);
AFKRFF (TPRO-FN6);
IVRRAD (COL18-FN6);
FWRWFK (HX5/CPAP);
KFWRWF (HX6/YJPA);
WFRFWK (HX7/CLPB)
KWFRFW (HX8/ATG1);
AFHHFF (HEX16F/STPK);
FFRNFA (HEXF13/SIF1);
AFHRFF (HEX9F/THIF);
FFRQFA (HEXF1/ATPB);
AFNRFF (HEX2F/AATF);
WIQRMM (CXCL13-FN6);
WVQRVV (CXCL8-FN6);
AFRNFF (HEX3F/FBNA);
and
TLRRFM (HEX18/HSHK);
or

b) a sequence having one or two amino acid substitutions relative to the sequence defined in a).

206. The active agent of clause 186, wherein the immunomodulatory peptide comprises:
a) a peptide sequence selected from:

DVRMRL (MCMV-FN6);
and
RRAELG (TONB-FN6)

or
b) a sequence having one or two amino acid substitutions relative to the sequence defined in a).

207. The active agent of clause 182, wherein the immunomodulatory peptide comprises:
a) a peptide sequence selected from:

AFFRFF (HEX13F/TGME);
FWRWFA (HX1/MMPL);
AFWRWF (HX2/ABCT);
WFRFWA (HX3/GTRF);
AWFRFW (HX4/AXES);
VAVRIW (HX9/IDRF/AMIA);
FFRFFA (HEXF2/AMT1);
and

or
b) a sequence having one or two amino acid substitutions relative to the sequence defined in a).

208. The active agent of clause 186, wherein the immunomodulatory peptide comprises:
a) a peptide sequence selected from:

FFKKFF;
WWKKFF;
FWKKWF;
FFKKWW;
WWKKWW;
YYKKYY;
IIKKYY;
YIKKIY;
YYKKII;
IIKKII;
MMKKMM;
LLKKMM;
MLKKLM;
MMKKLL;
LLKKLL;
VVKKVV;
AAKKVV;
VAKKAV;
VVKKAA;
AAKKAA;
GGKKGG;
TTKKGG;
GTKKTG;
GGKKTT;
TTKKTT;
SSKKSS;
CCKKSS;
SCKKCS;
SSKKCC;
and
CCKKCC;

or
b) a sequence having one or two amino acid substitutions relative to the sequence defined in a).

209. The active agent of clause 186, wherein the immunomodulatory peptide comprises:
a) a peptide sequence selected from:

FKFKFK;
WKWKWK;
YKYKYK:
IKIKIK;
MKMKMK;
LKLKLK;
VKVKVK;
AKAKAK;
GKGKGK;
TKTKTK;
SKSKSK;
CKCKCK;
KFKFKF;
KWKWKW;
KYKYKY;
KIKIKI;
KMKMKM;
KLKLKL;
KVKVKV;
KAKAKA;
KGKGKG;
KTKTKT;
KSKSKS;
and
KCKCKC;

or
b) a sequence having one or two amino acid substitutions relative to the sequence defined in a).

210. The active agent of clause 182, wherein the immunomodulatory peptide comprises a peptide from table 1.

211. The active agent of clause 210, wherein the immunomodulatory peptide comprises a peptide of table 1 truncated by 1 or 2 amino acids at the N-terminus.

212. The active agent of clause 210, wherein the immunomodulatory peptide comprises a peptide of table 1 truncated by 1 or 2 amino acids at the C-terminus.

213. The active agent of clause 182 or 183, wherein the immunomodulatory peptide comprises a sequence defined by one of the formulae:

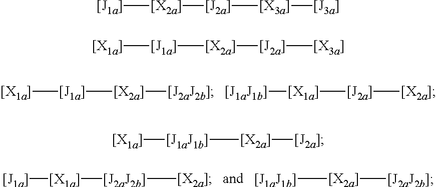

wherein:
J₁ₐ, J_{1b}, J₂ₐ, J_{2b} and J₃ₐ are each independently selected from phenylalanine, tryptophan, alanine, valine, leucine, isoleucine, methionine, tyrosine, threonine, serine, cysteine, proline, and glycine; and
X₁ₐ, X_{1b}, X₂ₐ, X_{2b} and X₃ₐ are each independently selected from lysine, arginine, histidine, aspartic acid, glutamic acid, asparagine and glutamine.

214. The active agent of clause 213, wherein the immunomodulatory peptide comprises:
a) a peptide sequence selected from:

AFKRF;
FFKKF;
FWKRF;
WVRRV;
IFKKI;
FLRNL;
FLHSA;
FFHHI;
FFKKA;
ALKKV;
LYKKI;
LFRRA;
FLKRL;
FFRRF;
FFRHF;
AIRRI;
AFHRF;
FFNRF;
IVRRA;
FWRWF;
KFWRW;
WFRFW;
KWFRF;
AFHHF;
FFRNF;
FFRQF;
AFNRF;
WIQRM;
WVQRV;
AFRNF;
TLRRF;
FKRFF;
FKKFF;
WKRFV;
VRRVV;
FKKIE;
LRNLV;
LHSAK;
FHHIF;
FKKAA;
LKKVF;
YKKII;
FRRAF;
LKRLL;
FRRFA;
FRHFA;
IRRIP;
FHRFF;
FNRFA;
VRRAD;
WRWFK;
FRFWK;
FHHFF;
FRNFA;
FRQFA;
FNRFF;
IQRMM;
VQRVV;
FRNFF;
LRRFM;
DVRMR;
VRMRL;
RRAEL;
RAELG;
and
RWKFG;

or
b) a sequence having one or two amino acid substitutions relative to the sequence defined in a).

215. The active agent of clause 182, wherein the immunomodulatory peptide comprises:
a)
i) a peptide sequence selected from:

AFWRW;
AWFRF;
VAVRI;
FFRFF;
AFFRF;
WRWFA;
FRFWA;
AVRIW;
and
FRFFA;

or
ii) a sequence having one or two amino acid substitutions relative to the sequence defined in i); or
b)
i) a peptide sequence selected from:

FFKKF;
WWKKF;
FWKKW;
FFKKW;
WWKKW;
YYKKY;
IIKKY;
YIKKI;
YYKKI;
IIKKI;
MMKKM;
LLKKM;
MLKKL;
MMKKL;
LLKKL;
VVKKV;
AAKKV;
VAKKA;
VVKKA;
AAKKA;
GGKKG;
TTKKG;
GTKKT;
GGKKT;
TTKKT;
SSKKS;
CCKKS;
SCKKC;
SSKKC;
and
CCKKC;
FKKFF;
WKKFF;
WKKWF;
FKKWW;
WKKWW;
YKKYY;
IKKYY;
IKKIY;
YKKII;
IKKII;
MKKMM;
LKKMM;
LKKLM;
MKKLL;
LKKLL;
VKKVV;
AKKVV;
AKKAV;
VKKAA;
AKKAA;
GKKGG;
TKKGG;
TKKTG;
GKKTT;
TKKTT;
SKKSS;
CKKSS;
CKKCS;
SKKCC;
and
CKKCC;

or
ii) a sequence having one or two amino acid substitutions relative to the sequence defined in i).

216. The active agent of clause 213, wherein the immunomodulatory peptide comprises:
a) a peptide sequence selected from:

FKFKF;
WKWKW;
YKYKY:
IKIKI;
MKMKM;
LKLKL;
VKVKV;
AKAKA;
GKGKG;
TKTKT;
SKSKS;
CKCKC;
KFKFK;
KWKWK;
KYKYK;
KIKIK;
KMKMK;
KLKLK;
KVKVK;
KAKAK;
KGKGK;
KTKTK;
KSKSK;
and
KCKCK;

or
b) a sequence having one or two amino acid substitutions relative to the sequence defined in a).

217. The active agent of clause 182 or 183, wherein the immunomodulatory peptide comprises a sequence defined by one of the formulae:

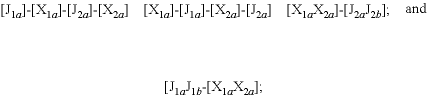

wherein:
J₁ₐ, J_{1b}, J₂ₐ, and J_{2b} are each independently selected from phenylalanine, tryptophan, alanine, valine, leucine, isoleucine, methionine, tyrosine, threonine, serine, cysteine, proline, and glycine; and
X₁ₐ, X_{1b}, X₂ₐ, and X_{2b} are each independently selected from lysine, arginine, histidine, aspartic acid, glutamic acid, asparagine and glutamine.

218. The active agent of clause 217, wherein the immunomodulatory peptide comprises:
a) a peptide sequence selected from:

AFKR;
FFKK;
FWKR;
WVRR;
IFKK;
FLRN;
FLHS;
FFHH;
ALKK;
LYKK;
LFRR;
FLKR;
FFRR;
FFRH;
AIRR;
AFHR;
FFNR;
IVRR;
FWRW;
KFWR;
WFRF;
KWFR;
AFHH;
FFRN;
FFRQ;
AFNR;
WIQR;
WVQR;
AFRN;
TLRR;
KRFF;
KKFF;
KRFV;
RRVV;
KKIE;
RNLV;
HSAK;
HHIF;
KKAA;
KKVF;
KKII;
RRAF;
KRLL;
RRFA;
RHFA;
RRIP;
HRFF;
NRFA;
RRAD;
RWFK;
RFWK;
HHFF;
RNFA;
RQFA;
NRFF;
QRMM;
QRVV;
RNFF;
RRFM;
VRMR;
RMRL;
RAEL;
AELG;
and
WKFG;

or
b) a sequence having one or two amino acid substitutions relative to the sequence defined in a).

219. The active agent of clause 182, wherein the immunomodulatory peptide comprises:
a) a peptide sequence selected from:

FWRW;
AFWR;
WFRF;
AWFR;
VAVR;
FFRF;
AFFR;
RWFA;
WRWF;
RFWA;
FRFW;
VRIW;
RFFA;
and
FRFF;

or
b) a sequence having one or two amino acid substitutions relative to the sequence defined in a).

220. The active agent of clause 217, wherein the immunomodulatory peptide comprises:
a) a peptide sequence selected from:

FFKK;
WWKK;
FWKK;
YYKK;
IIKK;
YIKK;
MMKK;
LLKK;
MLKK;
VVKK;
AAKK;
VAKK;
GGKK;
TTKK;
GTKK;
SSKK;
CCKK;
SCKK;
KKFF;
KKWF;
KKWW;
KKYY;
KKIY;
KKII;
KKMM;
KKLM;
KKLL;
KKVV;
KKAV;
KKAA;
KKGG;
KKTG;
KKTT;
KKSS;
KKCS;
and
KKCC;

or
b) a sequence having one or two amino acid substitutions relative to the sequence defined in a).

221. The active agent of clause 217, wherein the immunomodulatory peptide comprises:
a) a peptide sequence selected from:

FKFK;
WKWK;
YKYK:
IKIK;
MKMK;
LKLK;
VKVK;
AKAK;
GKGK;
TKTK;
SKSK;
CKCK;
KFKF;
KWKW;
KYKY;
KIKI;
KMKM;
KLKL;
KVKV;
KAKA;
KGKG;
KTKT;
KSKS;
and
KCKC;

or
b) a sequence having one or two amino acid substitutions relative to the sequence defined in a).

222. The active agent of any one of clauses 205 to 221, wherein the one or two amino acid substitutions defined in b) consist of a highly conservative substitution of a cationic amino acid of the sequence.

223. The active agent of any one of clauses 177 to 181, wherein the active agent is a small molecule active agent.

224. The active agent of clause 223, wherein the small molecule active agent is described by formula (I):

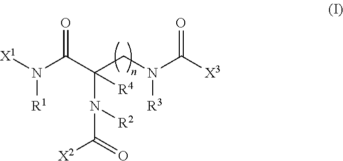

wherein:
R¹-R⁴ are each independently selected from hydrogen, alkyl and substituted alkyl;
X¹ is selected from alkyl, substituted alkyl, aryl, substituted aryl, aralkyl, substituted aralkyl, heterocycle, substituted heterocycle, heteroaryl, substituted heteroaryl;
X² is selected from alkyl, substituted alkyl, aryl, substituted aryl, amino, substituted amino, heteroaryl, substituted heteroaryl, heterocycle, substituted heterocycle;
X³ is selected from alkyl, substituted alkyl, aryl, substituted aryl, naphthyl, substituted naphthyl, heterocycle, substituted heterocycle, heteroaryl, substituted heteroaryl, aryl heterocycle, substituted aryl heterocycle; and
n is an integer from 1 to 10,
or a pharmaceutically acceptable salt or solvate thereof.

225. The active agent of clause 224, wherein the small molecule active agent is described by formula (Ia):

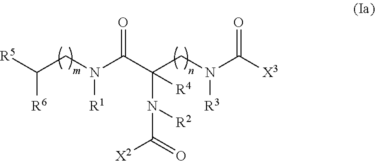

wherein:
R¹-R⁴ are each independently selected from hydrogen, and alkyl;
R⁵-R⁶ are each independently selected from aryl and substituted aryl;
X² is selected from alkyl, and NR^{2a}R^{2b}, where R2a and R2b are independently selected from hydrogen, aryl, and substituted aryl;
X³ is selected from aryl, substituted aryl, naphthyl, substituted naphthyl, carbazole, and substituted carbazole;
n is an integer from 1 to 6; and
m is an integer from 1 to 6.

226. The active agent of clause 225, wherein the small molecule active agent is a compound selected from the group consisting of:

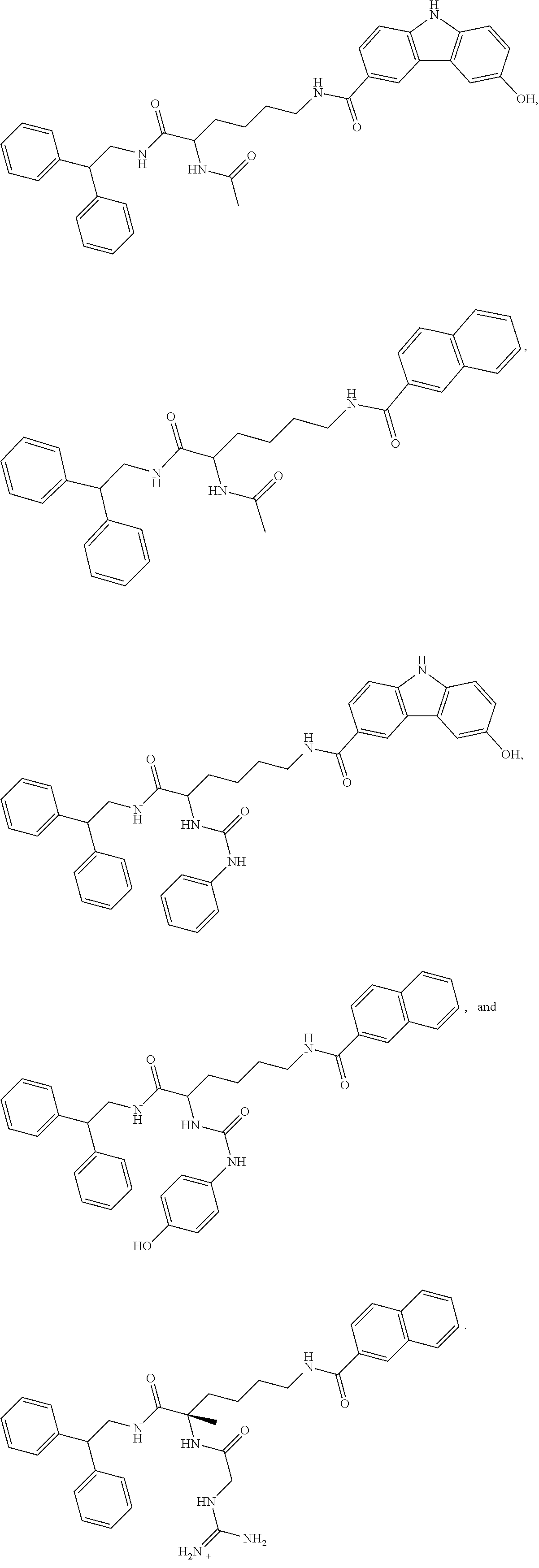 [SMILES: CC(=O)NC(CCCCNC(=O)c1ccc2[nH]c3ccc(O)cc3c2c1)C(=O)NCC(c1ccccc1)c1ccccc1 ; CC(=O)NC(CCCCNC(=O)C1=Cc2ccccc2CC=C1)CC(=O)NCC(c1ccccc1)c1ccccc1 ; O=C(Nc1ccccc1)NC(CCCCNC(=O)c1ccc2[nH]c3ccc(O)cc3c2c1)C(=O)NCC(c1ccccc1)c1ccccc1 ; O=C(Nc1ccc(O)cc1)NC(CCCCNC(=O)c1ccc2ccccc2c1)C(=O)NCC(c1ccccc1)c1ccccc1 ; C[C@@](CCCCNC(=O)c1ccc2ccccc2c1)(NC(=O)CNC(=N)N)C(=O)NCC(c1ccccc1)c1ccccc1]

227. The active agent of clause 223, wherein the small molecule active agent is described by the formula (II):

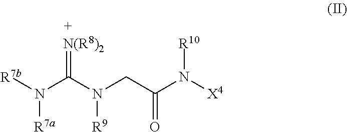

wherein:
R^{7a}, R^{7b}, R⁸, R⁹ and R¹⁰ are each independently selected from hydrogen, alkyl and substituted alkyl; and
X⁴ is selected from alkyl, aryl, aralkyl, heterocycle, and heteroaryl, acyl, wherein X⁴ is optionally further substituted with one or more groups selected from, alkyl, substituted alkyl, aryl, substituted aryl, amino, substituted amino, carboxamide, substituted carboxamide, heterocycle, substituted heterocycle, and a second compound of formula (II)
or a pharmaceutically acceptable salt or solvate thereof.

228. The active agent of clause 227, wherein the small molecule active agent is described by the formula (IIa):

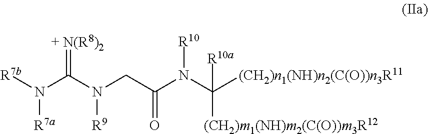

wherein:
R^{7a}, R^{7b}, R⁸, R⁹, R¹⁰ and R^{10a} are each independently selected from hydrogen, and alkyl;
R¹¹ and R¹² are each independently selected from aryl, substituted aryl, heteroaryl, substituted heteroaryl, naphthyl, substituted naphthyl, carbazole, and substituted carbazole;
n₁ and m₁ are each independently an integer from 1 to 10;
n₂ and m₂ are each independently 0 or 1; and
n₃ and m₃ are each independently 0 or 1.

229. The active agent of clause 228, wherein the small molecule active agent is a compound selected from the group consisting of:

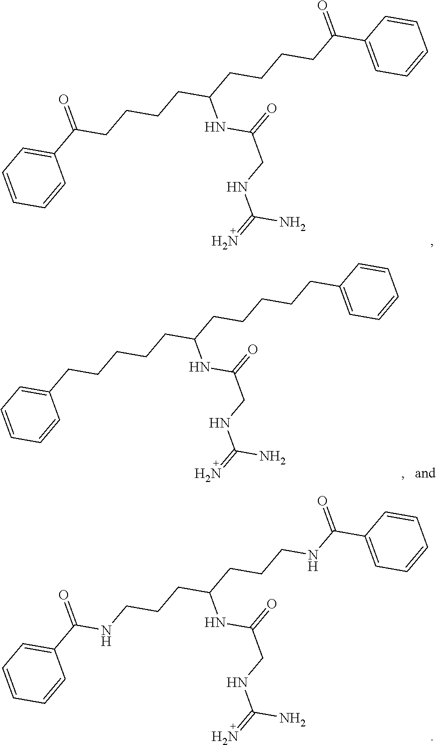 [SMILES: N=C(N)NCC(=O)NC(CCCCC(=O)c1ccccc1)CCCCC(=O)c1ccccc1 ; N=C(N)NCC(=O)NC(CCCCCc1ccccc1)CCCCCc1ccccc1 ; N=C(N)NCC(=O)NC(CCCNC(=O)c1ccccc1)CCCNC(=O)c1ccccc1]

230. The active agent of clause 227, wherein the small molecule active agent is a compound selected from the group consisting of:

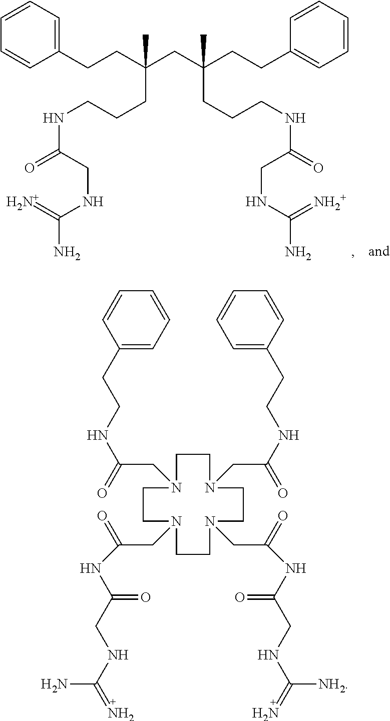 [SMILES: C[C@](CCCNC(=O)CNC(N)=[NH2+])(CCc1ccccc1)C[C@](C)(CCCNC(=O)CNC(N)=[NH2+])CCc1ccccc1 ; N=C(N)NCC(=O)NC(=O)CN1CCN(CC(=O)NCCc2ccccc2)CCN(CC(=O)NCCc2ccccc2)CCN(CC(=O)NC(=O)CNC(=N)N)CC1]

231. The active agent of clause 223, wherein the small molecule active agent is described by the formula (III):

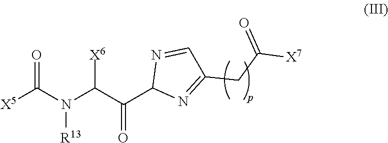

wherein:
R¹³ is selected from hydrogen, alkyl and substituted alkyl;
X⁵ is selected from alkyl, substituted alkyl, aryl, substituted aryl, amino, substituted amino, heteroaryl, substituted heteroaryl, heterocycle, substituted heterocycle;
X⁶ is selected from alkyl, substituted alkyl, aryl, substituted aryl, aralkyl, substituted aralkyl, heterocycle, substituted heterocycle, heteroaryl, substituted heteroaryl;
X⁷ is selected from alkyl, substituted alkyl, aryl, substituted aryl, naphthyl, substituted naphthyl, heterocycle, substituted heterocycle, heteroaryl, substituted heteroaryl, aryl heterocycle, substituted aryl heterocycle; and
p is an integer from 1 to 10,
or a pharmaceutically acceptable salt or solvate thereof.

232. The active agent of clause 231, wherein the small molecule active agent is:

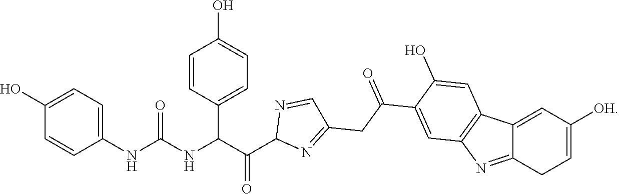 [SMILES: O=C(Nc1ccc(O)cc1)NC(C(=O)C1N=CC(CC(=O)c2cc3c(cc2O)C2=CC(O)=CCC2=N3)=N1)c1ccc(O)cc1]

233. The active agent of clause 177, wherein the active agent is a specific binding member.

234. The active agent of clause 233, wherein the specific binding member is an antibody, or a binding fragment thereof.

235. The active agent of clause 233 or 234, wherein the specific binding member targets a sequence of CD206 selected from the group consisting of NFGDLVSIQSESEKK, NDAQSAYFIGLLISL, SKEKETMDNARAF, and EDENCVTMYSNSGFWN.

236. The active agent of clause 235, wherein the specific binding member targets a NFGDLVSIQSESEKK sequence of CD206.

237. The active agent of clause 235, wherein the specific binding member targets a NDAQSAYFIGLLISL sequence of CD206.

238. The active agent of clause 235, wherein the specific binding member targets a SKEKETMDNARAF sequence of CD206.

239. The active agent of clause 235, wherein the specific binding member targets a EDENCVTMYSNSGFWN sequence of CD206.

240. A method comprising:
contacting a macrophage comprising CD206 with a compound; and
determining if the compound binds to an activity modulating domain of CD206.

241. The method according to clause 240, further comprising determining the activity modulating domain of CD206 that binds the compound.

242. The method according clause 240 or 241, wherein the macrophage is a macrophage comprising one or more mutations in the activity modulating domains of CD206.

243. The method according to any one of clauses 240-242, wherein the activity modulating domain of CD206 is selected from the group consisting of fibronectin II domain of CD206, C-type lectin carbohydrate recognition domain 3 (CRD3) of CD206, C-type lectin carbohydrate recognition domain 4 (CRD4) of CD206 and C-type lectin carbohydrate recognition domain 5 (CRD5) of CD206.

244. The method of clause 243, wherein the activity modulating domain of CD206 is the CRD5 domain.

245. The method of clause 243, wherein the activity modulating domain of CD206 is the fibronectin II domain.

246. The method of clause 243, wherein the activity modulating domain of CD206 is the CRD3 domain.

247. The method of any one of clauses 243 to 246, wherein the compound is an immunomodulatory peptide.

248. The method of clause 247, wherein the immunomodulatory peptide is of 5 to 18 amino acid residues in length, the peptide comprising: a striapathic region of alternating hydrophilic and hydrophobic modules that adopts an amphipathic conformation under physiological conditions.

249. The method of clause 248, wherein the striapathic region comprises:
3 or more hydrophobic modules; and
2 or more hydrophilic modules each comprising at least one cationic residue;
wherein the immunomodulatory peptide binds to the activity modulating domain of CD206.

250. The method of any one of clauses 247 to 249, wherein the immunomodulatory peptide comprises a sequence defined by one of the formulae:

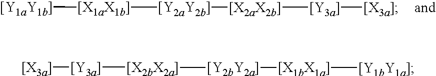

wherein:
Y₁ₐ, Y_{1b}, Y₂ₐ, Y_{2b} and Y₃ₐ are each independently selected from phenylalanine, tryptophan, alanine, valine, and glycine; and
X₁ₐ, X_{1b}, X₂ₐ, X_{2b} and X₃ₐ are each independently selected from lysine, arginine, histidine, aspartic acid, glutamic acid, asparagine and glutamine.

251. The method of clause 247 or 248, wherein the immunomodulatory peptide comprises a sequence defined by one of the formulae:

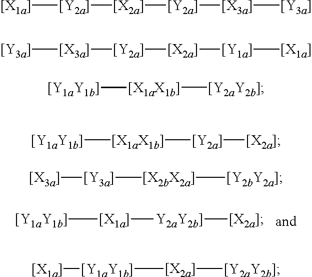

wherein:
Y₁ₐ, Y_{1b}, Y₂ₐ, Y_{2b} and Y₃ₐ are each independently selected from phenylalanine, tryptophan, alanine, valine, leucine, isoleucine, methionine, tyrosine, threonine, serine, cysteine, proline, and glycine; and
X₁ₐ, X_{1b}, X₂ₐ, X_{2b} and Xu are each independently selected from lysine, arginine, histidine, aspartic acid, glutamic acid, asparagine and glutamine.

252. The method of clause 250 or 251, wherein:
Y₁ₐ, Y_{1b}, Y₂ₐ, Y_{2b} and Y₃ₐ are each phenylalanine; and
X₁ₐ, X_{1b}, X₂ₐ, X_{2b} and X₃ₐ are each independently selected from lysine and arginine.

253. The method of clause 250, wherein the immunomodulatory peptide comprises
a) a sequence selected from:

KFRKAFKRFF (RP182);
FFRKFAKRFK (RP183);
FFKKFFKKFK (RP185);
FFKKFFKKFK (RP186);
and
FFKKFFKKFK (RP233);

or
b) a sequence having one or two amino acid substitutions relative to the sequence defined in a).

254. The method of clause 253, wherein the immunomodulatory peptide comprises the amino acid sequence KFRKAFKRFF (RP182)

255. The method of clause 253, wherein the immunomodulatory peptide comprises the amino acid sequence FFRKFAKRFK (RP183).

256. The method of clause 253, wherein the immunomodulatory peptide comprises the amino acid sequence FFKKFFKKFK (RP185).

257. The method of clause 248, wherein the immunomodulatory peptide comprises:
a) a peptide sequence selected from:

RWKFGGFKWR (RP832C);
FKWRGGRWKF (RP837C);
FWKRGGRKWF (RP837A);
FWKRFV (RP837N);
FVRKWR (RP837C1);
FAOOFAOOFO (RP850);
FWKRFVRKWR (RP837);
FWKKFVKKWK (RP841);
WWHHWWHHWH (RP847);
WWRHWWHRWR (RP848);
WWKHWWHKWK (RP849);
GDRGIKGHRGF (RP842);
LYKKIIKKLL (RP846);
FYPDFFKKFF (RP844);
FFRKSKEKIG (RP853);
FFRHFATHLD (RP845);
and
EKLSAFRNFF (RP843);

or
b) a sequence having one or two amino acid substitutions relative to the sequence defined in a).

258. The method of clause 257, wherein the one or two amino acid substitutions defined in b) consist of a highly conservative substitution of a cationic amino acid of the sequence.

259. The method of clause 257, comprising the peptide sequence selected from: RWKFGGFKWR (RP832C), FKWRGGRWKF (RP837C) and FWKRGGRKWF (RP837A).

260. The method of clause 257, comprising the peptide sequence selected from FWKRFV (RP837N) and FVRKWR (RP837C1).

261. The method of clause 257, comprising a peptide sequence selected from FAOOFAOOFO (RP850), FWKRFVRKWR (RP837) and FWKKFVKKWK (RP841).

262. The method of clause 257, comprising a peptide sequence selected from WWHHWWHHWH, WWRHWWHRWR and WWKHWWHKWK (RP847-849).

263. The method of clause 248, comprising a peptide sequence selected from MVFRDVGNRN, LFWKRFVEFF, AIRRIPRRIR, LAERAFHRFF, DVRMRLRSEV, FFNRFANERH, GFRELFRQLD, SQLPAFKRFF, RRAELGFKWR, MFEREVKNAM, REVKNAMRRW, IENAAFKRFF, FYPDFFKKFF, and KKIRVRSLA

264. The method of clause 257, comprising the peptide sequence GDRGIKGHRGF (RP842).

265. The method of clause 257, comprising the peptide sequence LYKKIIKKLL (RP846).

266. The method of clause 257, comprising the peptide sequence FYPDFFKKFF (RP844).

267. The method of clause 257, comprising the peptide sequence FFRKSKEKIG (RP853).

268. The method of clause 257, comprising the peptide sequence FFRHFATHLD (RP845).

269. The method of clause 257, comprising the peptide sequence EKLSAFRNFF (RP843).

270. The method of clause 251, wherein the immunomodulatory peptide comprises:
a) a peptide sequence selected from:

AFKRFF (182-FN6);
FFKKFF (185-FN6);
FWKRFV (837-FN6);
WVRRVV (WLUB-F1-N6);
IFKKIE (CEC-F1-N6)
FLRNLV (LL37F-3-N6);
FLHSAK (MAG-F1-N6);
FFHHIF (PISC-F-N6);
FFKKAA (PLEU-F-N6);
ALKKVF (PSEU-F-N6);
LYKKII (CXCL4-F-N6);
LFRRAF (IL24-FN6);
FLKRLL (IL7-FN6);
FFRRFA (ABCP-FN6);
FFRHFA (E1P-FN6);
AIRRIP (gP120-FN6);
AFHRFF (GP2B-FN6);
FFNRFA (MCPH-FN6);
AFKRFF (SPRA-FN6);
AFKRFF (TPRO-FN6);
IVRRAD (COL18-FN6);
FWRWFK (HX5/CPAP);
KFWRWF (HX6/YJPA);
WFRFWK (HX7/CLPB)
KWFRFW (HX8/ATG1);
AFHHFF (HEX16F/STPK);
FFRNFA (HEXF13/SIF1);
AFHRFF (HEX9F/THIF);
FFRQFA (HEXF1/ATPB);
AFNRFF (HEX2F/AATF);
WIQRMM (CXCL13-FN6);
WVQRVV (CXCL8-FN6);
AFRNFF (HEX3F/FBNA);
and
TLRRFM (HEX18/HSHK);

or
b) a sequence having one or two amino acid substitutions relative to the sequence defined in a).

271. The method of clause 251, wherein the immunomodulatory peptide comprises:
a) a peptide sequence selected from:

DVRMRL (MCMV-FN6);
and
RRAELG (TONB-FN6)

or
b) a sequence having one or two amino acid substitutions relative to the sequence defined in a).

272. The method of clause 247, wherein the immunomodulatory peptide comprises:
a) a peptide sequence selected from:

FWRWFA (HX1/MMPL);
AFWRWF (HX2/ABCT);
WFRFWA (HX3/GTRF);
AWFRFW (HX4/AXES);
VAVRIW (HX9/IDRF/AMIA);
FFRFFA (HEXF2/AMT1);
and
AFFRFF (HEX13F/TGME);

or
b) a sequence having one or two amino acid substitutions relative to the sequence defined in a).

273. The method of clause 251, wherein the immunomodulatory peptide comprises:
a) a peptide sequence selected from:

FFKKFF; WWKKFF; FWKKWF; FFKKWW; WWKKWW; YYKKYY;
IIKKYY; YIKKIY; YYKKII; IIKKII; MMKKMM; LLKKMM;
MLKKLM; MMKKLL; LLKKLL; VVKKVV; AAKKVV; VAKKAV;
VVKKAA; AAKKAA; GGKKGG; TTKKGG; GTKKTG; GGKKTT;
TTKKTT; SSKKSS; CCKKSS; SCKKCS; SSKKCC;
and
CCKKCC;

or
b) a sequence having one or two amino acid substitutions relative to the sequence defined in a).

274. The method of clause 251, wherein the immunomodulatory peptide comprises:
a) a peptide sequence selected from:

FKFKFK; WKWKWK; YKYKYK: IKIKIK; MKMKMK; LKLKLK;
VKVKVK; AKAKAK; GKGKGK; TKTKTK; SKSKSK; CKCKCK;
KFKFKF; KWKWKW; KYKYKY; KIKIKI; KMKMKM; KLKLKL;
KVKVKV; KAKAKA; KGKGKG; KTKTKT; KSKSKS;
and
KCKCKC;

or
b) a sequence having one or two amino acid substitutions relative to the sequence defined in a).

275. The method of clause 247, wherein the immunomodulatory peptide comprises a peptide from table 1.

276. The method of clause 275, wherein the immunomodulatory peptide comprises a peptide of table 1 truncated by 1 or 2 amino acids at the N-terminus.

277. The method of clause 275, wherein the immunomodulatory peptide comprises a peptide of table 1 truncated by 1 or 2 amino acids at the C-terminus.

278. The method of clause 247 or 248, wherein the immunomodulatory peptide comprises a sequence defined by one of the formulae:

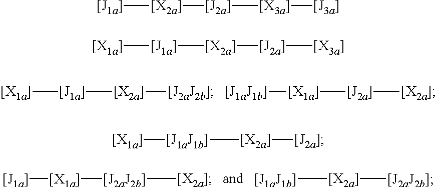

wherein:
J₁ₐ, J_{1b}, J₂ₐ, J_{2b} and J₃ₐ are each independently selected from phenylalanine, tryptophan, alanine, valine, leucine, isoleucine, methionine, tyrosine, threonine, serine, cysteine, proline, and glycine; and
X₁ₐ, X_{1b}, X₂ₐ, X_{2b} and X₃ₐ are each independently selected from lysine, arginine, histidine, aspartic acid, glutamic acid, asparagine and glutamine.

279. The method of clause 278, wherein the immunomodulatory peptide comprises:
a) a peptide sequence selected from:

AFKRF; FFKKF; FWKRF; WVRRV; IFKKI; FLRNL; FLHSA;
FFHHI; FFKKA; ALKKV; LYKKI; LFRRA; FLKRL; FFRRF;
FFRHF; AIRRI; AFHRF; FFNRF; IVRRA; FWRWF; KFWRW;
WFRFW; KWFRF; AFHHF; FFRNF; FFRQF; AFNRF; WIQRM;
WVQRV; AFRNF; TLRRF; FKRFF; FKKFF; WKRFV; VRRVV;
FKKIE; LRNLV; LHSAK; FHHIF; FKKAA; LKKVF; YKKII;
FRRAF; LKRLL; FRRFA; FRHFA; IRRIP; FHRFF; FNRFA;
VRRAD; WRWFK; FRFWK; FHHFF; FRNFA; FRQFA; FNRFF;
IQRMM; VQRVV; FRNFF; LRRFM; DVRMR; VRMRL; RRAEL;
RAELG;
and
RWKFG;

or
b) a sequence having one or two amino acid substitutions relative to the sequence defined in a).

280. The method of clause 247, wherein the immunomodulatory peptide comprises:
a) a peptide sequence selected from:

AFWRW; AWFRF; VAVRI; FFRFF; AFFRF; WRWFA; FRFWA;
AVRIW;
and
FRFFA;

or
b) a sequence having one or two amino acid substitutions relative to the sequence defined in a).

281. The method of clause 278, wherein the immunomodulatory peptide comprises:
a) a peptide sequence selected from:

FFKKF; WWKKF; FWKKW; FFKKW; WWKKW; YYKKY; IIKKY;
YIKKI; YYKKI; IIKKI; MMKKM; LLKKM; MLKKL; MMKKL;
LLKKL; VVKKV; AAKKV; VAKKA; VVKKA; AAKKA; GGKKG;
TTKKG; GTKKT; GGKKT; TTKKT; SSKKS; CCKKS; SCKKC;
SSKKC;
and
CCKKC; FKKFF; WKKFF; WKKWF; FKKWW; WKKWW; YKKYY;
IKKYY; IKKIY; YKKII; IKKII; MKKMM; LKKMM; LKKLM;
MKKLL; LKKLL; VKKVV; AKKVV; AKKAV; VKKAA; AKKAA;
GKKGG; TKKGG; TKKTG; GKKTT; TKKTT; SKKSS; CKKSS;
CKKCS; SKKCC;
and
CKKCC;

or
b) a sequence having one or two amino acid substitutions relative to the sequence defined in a).

282. The method of clause 278, wherein the immunomodulatory peptide comprises:
a) a peptide sequence selected from:

FKFKF; WKWKW; YKYKY: IKIKI; MKMKM; LKLKL; VKVKV;
AKAKA; GKGKG; TKTKT; SKSKS; CKCKC; KFKFK; KWKWK;
KYKYK; KIKIK; KMKMK; KLKLK; KVKVK; KAKAK; KGKGK;
KTKTK; KSKSK;
and
KCKCK;

or
b) a sequence having one or two amino acid substitutions relative to the sequence defined in a).

283. The method of clause 247 or 248, wherein the immunomodulatory peptide comprises a sequence defined by one of the formulae:

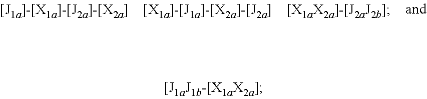

wherein:
J₁ₐ, J_{1b}, J₂ₐ, and J_{2b} are each independently selected from phenylalanine, tryptophan, alanine, valine, leucine, isoleucine, methionine, tyrosine, threonine, serine, cysteine, proline, and glycine; and
X₁ₐ, X_{1b}, X₂ₐ, and X_{2b} are each independently selected from lysine, arginine, histidine, aspartic acid, glutamic acid, asparagine and glutamine.

284. The method of clause 283, wherein the immunomodulatory peptide comprises:
a) a peptide sequence selected from:

AFKR; FFKK; FWKR; WVRR; IFKK; FLRN; FLHS; FFHH;
ALKK; LYKK; LFRR; FLKR; FFRR; FFRH; AIRR; AFHR;
FFNR; IVRR; FWRW; KFWR; WFRF; KWFR; AFHH; FFRN;
FFRQ; AFNR; WIQR; WVQR; AFRN; TLRR; KRFF; KKFF;
KRFV; RRVV; KKIE; RNLV; HSAK; HHIF; KKAA; KKVF;
KKII; RRAF; KRLL; RRFA; RHFA; RRIP; HRFF; NRFA;
RRAD; RWFK; RFWK; HHFF; RNFA; RQFA; NRFF; QRMM;
QRVV; RNFF; RRFM; VRMR; RMRL; RAEL; AELG;
and
WKFG;

or
b) a sequence having one or two amino acid substitutions relative to the sequence defined in a).

285. The method of clause 247, wherein the immunomodulatory peptide comprises:
a) a peptide sequence selected from:

FWRW; AFWR; WFRF; AWFR; VAVR; FFRF; AFFR; RWFA;
WRWF; RFWA; FRFW; VRIW; RFFA;
and
FRFF;

or
b) a sequence having one or two amino acid substitutions relative to the sequence defined in a).

286. The method of clause 283, wherein the immunomodulatory peptide comprises:
a) a peptide sequence selected from:

FFKK; WWKK; FWKK; YYKK; IIKK; YIKK; MMKK; LLKK;
MLKK; VVKK; AAKK; VAKK; GGKK; TTKK; GTKK; SSKK;
CCKK; SCKK; KKFF; KKWF; KKWW; KKYY; KKIY; KKII;
KKMM; KKLM; KKLL; KKVV; KKAV; KKAA; KKGG; KKTG;
KKTT; KKSS; KKCS;
and
KKCC;

or
b) a sequence having one or two amino acid substitutions relative to the sequence defined in a).

287. The method of clause 283, wherein the immunomodulatory peptide comprises:
a) a peptide sequence selected from:

FKFK; WKWK; YKYK: IKIK; MKMK; LKLK; VKVK; AKAK;
GKGK; TKTK; SKSK; CKCK; KFKF; KWKW; KYKY; KIKI;
KMKM; KLKL; KVKV; KAKA; KGKG; KTKT; KSKS;
and
KCKC;

or
b) a sequence having one or two amino acid substitutions relative to the sequence defined in a).

288. The method of any one of clauses 270 to 287, wherein the one or two amino acid substitutions defined in b) consist of a highly conservative substitution of a cationic amino acid of the sequence.

289. The method of any one of clauses 243 to 246, wherein the compound is a small molecule active agent.

290. The method of clause 289, wherein the small molecule active agent is described by formula (I):

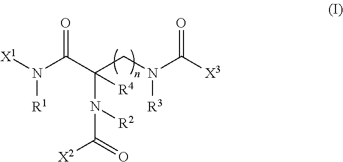

wherein:
R¹-R⁴ are each independently selected from hydrogen, alkyl and substituted alkyl;
X¹ is selected from alkyl, substituted alkyl, aryl, substituted aryl, aralkyl, substituted aralkyl, heterocycle, substituted heterocycle, heteroaryl, substituted heteroaryl;
X² is selected from alkyl, substituted alkyl, aryl, substituted aryl, amino, substituted amino, heteroaryl, substituted heteroaryl, heterocycle, substituted heteroaryl;
X³ is selected from alkyl, substituted alkyl, aryl, substituted aryl, naphthyl, substituted naphthyl, heterocycle, substituted heterocycle, heteroaryl, substituted heteroaryl, aryl heterocycle, substituted aryl heterocycle; and
n is an integer from 1 to 10,
or a pharmaceutically acceptable salt or solvate thereof.

291. The method of clause 290, wherein the small molecule active agent is described by formula (Ia):

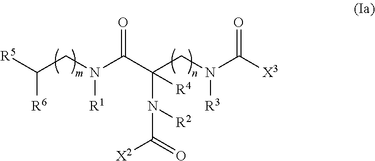

wherein:
R¹-R⁴ are each independently selected from hydrogen, and alkyl;
R⁵-R⁶ are each independently selected from aryl and substituted aryl;
X² is selected from alkyl, and NR^{2a}R^{2b}, where R2a and R2b are independently selected from hydrogen, aryl, and substituted aryl;
X³ is selected from aryl, substituted aryl, naphthyl, substituted naphthyl, carbazole, and substituted carbazole;
n is an integer from 1 to 6; and
m is an integer from 1 to 6;
wherein in some instances the small molecule active agent is a compound selected from the group consisting of:

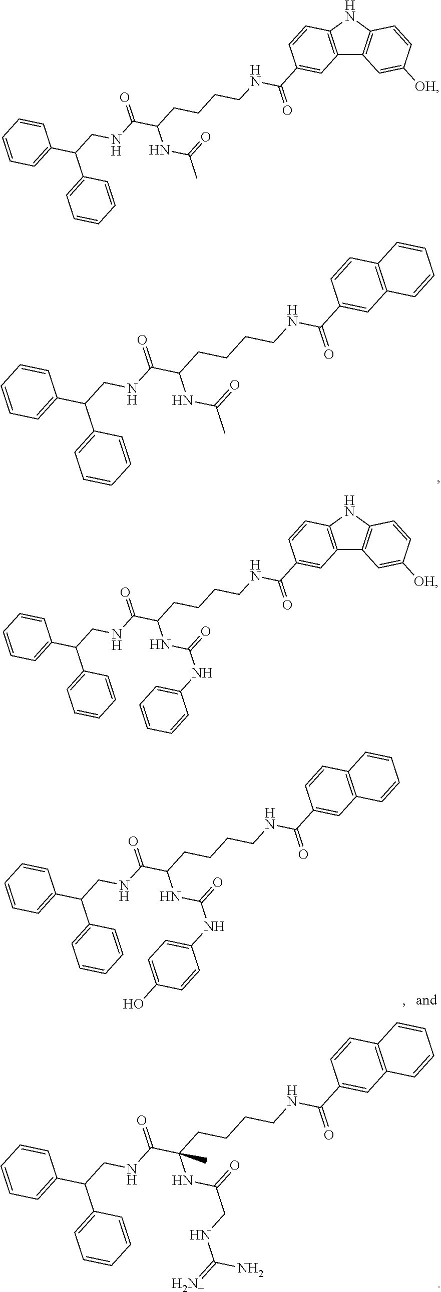 [SMILES: CC(=O)NC(CCCCNC(=O)c1ccc2[nH]c3ccc(O)cc3c2c1)C(=O)NCC(c1ccccc1)c1ccccc1 ; CC(=O)NC(CCCCNC(=O)c1ccc2ccccc2c1)C(=O)NCC(c1ccccc1)c1ccccc1 ; O=C(Nc1ccccc1)NC(CCCCNC(=O)c1ccc2[nH]c3ccc(O)cc3c2c1)C(=O)NCC(c1ccccc1)c1ccccc1 ; O=C(Nc1ccc(O)cc1)NC(CCCCNC(=O)c1ccc2ccccc2c1)C(=O)NCC(c1ccccc1)c1ccccc1 ; C[C@@](CCCCNC(=O)c1ccc2ccccc2c1)(NC(=O)CNC(N)=[NH2+])C(=O)NCC(c1ccccc1)c1ccccc1]

292. The method of clause 289, wherein the small molecule active agent is described by the formula (II):

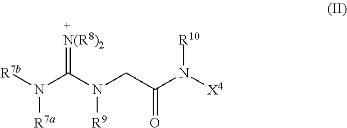

wherein:
R^{7a}, R^{7b}, R⁸, R⁹ and R¹⁰ are each independently selected from hydrogen, alkyl and substituted alkyl; and
X⁴ is selected from alkyl, aryl, aralkyl, heterocycle, and heteroaryl, acyl, wherein X⁴ is optionally further substituted with one or more groups selected from, alkyl, substituted alkyl, aryl, substituted aryl, amino, substituted amino, carboxamide, substituted carboxamide, heterocycle, substituted heterocycle, and a second compound of formula (II)
or a pharmaceutically acceptable salt or solvate thereof.

293. The method of clause 292, wherein the small molecule active agent is described by the formula (IIa):

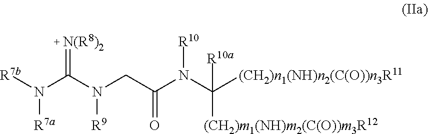

wherein:
R^{7a}, R^{7b}, R⁸, R⁹, R¹⁰ and R^{10a} are each independently selected from hydrogen, and alkyl;
R¹¹ and R¹² are each independently selected from aryl, substituted aryl, heteroaryl, substituted heteroaryl, naphthyl, substituted naphthyl, carbazole, and substituted carbazole;
n₁ and m₁ are each independently an integer from 1 to 10;
n₂ and m₂ are each independently 0 or 1; and
n₃ and m₃ are each independently 0 or 1.

294. The method of clause 293, wherein the small molecule active agent is a compound selected from the group consisting of:

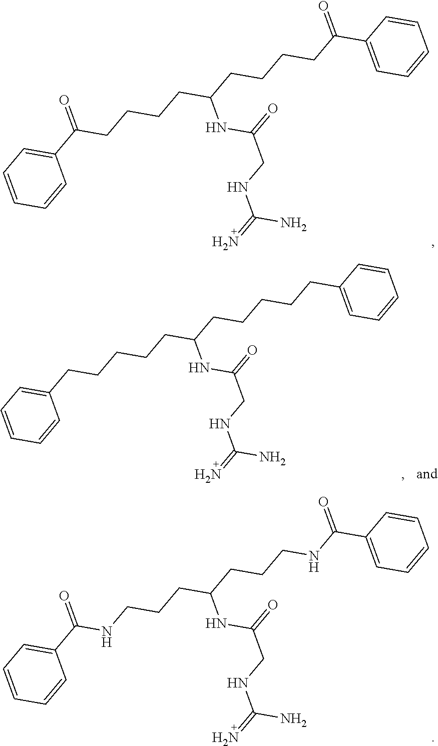 [SMILES: N=C(N)NCC(=O)NC(CCCCC(=O)c1ccccc1)CCCCC(=O)c1ccccc1 ; N=C(N)NCC(=O)NC(CCCCCc1ccccc1)CCCCCc1ccccc1 ; N=C(N)NCC(=O)NC(CCCNC(=O)c1ccccc1)CCCNC(=O)c1ccccc1]

295. The method of clause 292, wherein the small molecule active agent is a compound selected from the group consisting of:

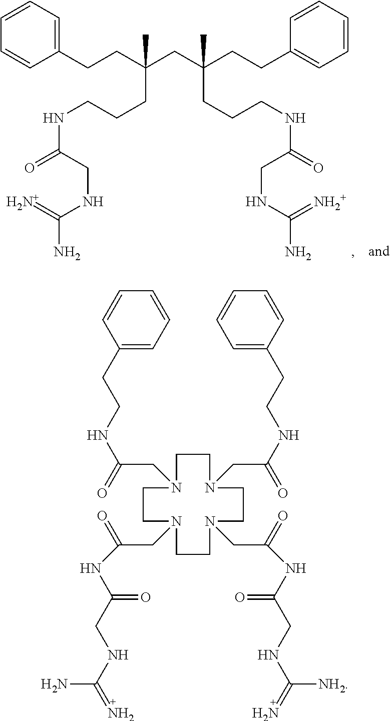 [SMILES: C[C@](CCCNC(=O)CNC(N)=[NH2+])(CCc1ccccc1)C[C@](C)(CCCNC(=O)CNC(N)=[NH2+])CCc1ccccc1 ; N=C(N)NCC(=O)NC(=O)CN1CCN(CC(=O)NCCc2ccccc2)CCN(CC(=O)NCCc2ccccc2)CCN(CC(=O)NC(=O)CNC(=N)N)CC1]

296. The method of clause 289, wherein the small molecule active agent is described by the formula (III):

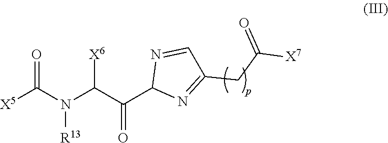

wherein:
R¹³ is selected from hydrogen, alkyl and substituted alkyl;
X⁵ is selected from alkyl, substituted alkyl, aryl, substituted aryl, amino, substituted amino, heteroaryl, substituted heteroaryl, heterocycle, substituted heteroaryl;
X⁶ is selected from alkyl, substituted alkyl, aryl, substituted aryl, aralkyl, substituted aralkyl, heterocycle, substituted heterocycle, heteroaryl, substituted heteroaryl;
X⁷ is selected from alkyl, substituted alkyl, aryl, substituted aryl, naphthyl, substituted naphthyl, heterocycle, substituted heterocycle, heteroaryl, substituted heteroaryl, aryl heterocycle, substituted aryl heterocycle; and
p is an integer from 1 to 10,
or a pharmaceutically acceptable salt or solvate thereof.

297. The method of clause 296, wherein the small molecule active agent is:

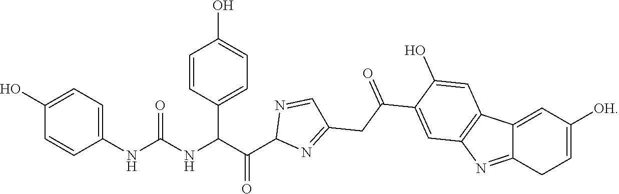 [SMILES: O=C(Nc1ccc(O)cc1)NC(C(=O)C1N=CC(CC(=O)c2cc3c(cc2O)C2=CC(O)=CCC2=N3)=N1)c1ccc(O)cc1]

298. The method of any one of clauses 243 to 246, wherein the compound is a specific binding member.

299. The method of any one of clauses 298, wherein the specific binding member is an antibody, or a binding fragment thereof.

300. The method of clause 298 or 299, wherein the specific binding member targets a sequence of CD206 selected from the group consisting of NFGDLVSIQSESEKK, NDAQSAYFIGLLISL, SKEKETMDNARAF, and EDENCVTMYSNSGFWN.

301. The method of clause 300, wherein the specific binding member targets a NFGDLVSIQSESEKK sequence of CD206.

302. The antibody of clause 300, wherein the specific binding member targets a NDAQSAYFIGLLISL sequence of CD206.

303. The antibody of clause 300, wherein the specific binding member targets a SKEKETMDNARAF sequence of CD206.

304. The antibody of clause 300, wherein the specific binding member targets a EDENCVTMYSNSGFWN sequence of CD206.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1-15. (canceled)

16. A compound described by formula (I): 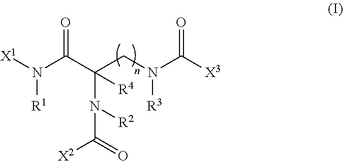
wherein:
R¹-R⁴ are each independently selected from hydrogen, alkyl and substituted alkyl;
X¹ is selected from alkyl, substituted alkyl, aryl, substituted aryl, aralkyl, substituted aralkyl, heterocycle, substituted heterocycle, heteroaryl, substituted heteroaryl;
X² is selected from alkyl, substituted alkyl, aryl, substituted aryl, amino, substituted amino, heteroaryl, substituted heteroaryl, heterocycle, substituted heterocycle;
X³ is selected from alkyl, substituted alkyl, aryl, substituted aryl, naphthyl, substituted naphthyl, heterocycle, substituted heterocycle, heteroaryl, substituted heteroaryl, aryl heterocycle, substituted aryl heterocycle; and
n is an integer from 1 to 10,
or a pharmaceutically acceptable salt or solvate thereof.

17. The compound of claim 16, described by formula (Ia): 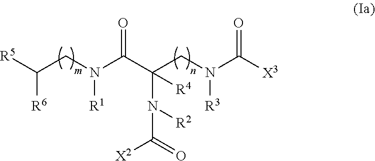
wherein:
R¹-R⁴ are each independently selected from hydrogen and alkyl;
R⁵-R⁶ are each independently selected from optionally substituted aryl;
X² is selected from optionally substituted alkyl, optionally substituted heteroaryl, and NR^{2a}R^{2b}, where R2a and R2b are independently selected from hydrogen, aryl, and substituted aryl;
X³ is selected from aryl, substituted aryl, naphthyl, substituted naphthyl, carbazole, and substituted carbazole;
n is an integer from 1 to 6; and
m is an integer from 1 to 6.

18. The compound of claim 17, wherein the compound selected from the group consisting of:

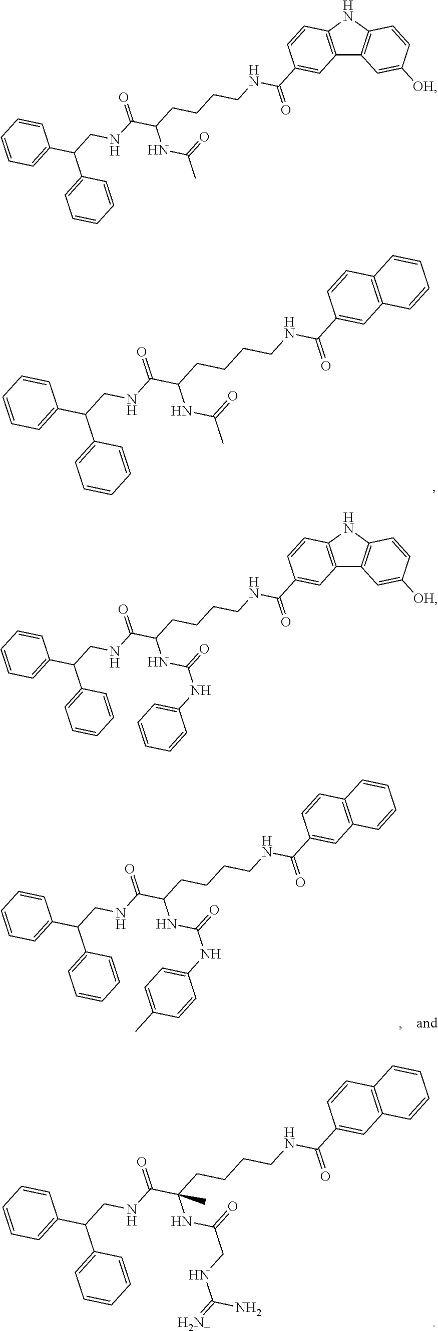 [SMILES: CC(=O)NC(CCCCNC(=O)c1ccc2[nH]c3ccc(O)cc3c2c1)C(=O)NCC(c1ccccc1)c1ccccc1 ; CC(=O)NC(CCCCNC(=O)c1ccc2ccccc2c1)C(=O)NCC(c1ccccc1)c1ccccc1 ; O=C(Nc1ccccc1)NC(CCCCNC(=O)c1ccc2[nH]c3ccc(O)cc3c2c1)C(=O)NCC(c1ccccc1)c1ccccc1 ; Cc1ccc(NC(=O)NC(CCCCNC(=O)c2ccc3ccccc3c2)C(=O)NCC(c2ccccc2)c2ccccc2)cc1 ; C[C@@](CCCCNC(=O)c1ccc2ccccc2c1)(NC(=O)CNC(N)=[NH2+])C(=O)NCC(c1ccccc1)c1ccccc1]

19. The compound of claim 17, wherein R¹-R⁴ are each hydrogen.

20. The compound of claim 17, wherein R⁴ is alkyl, and each of R¹-R³ are hydrogen.

21. The compound of claim 17, wherein R⁵ and R⁶ are each phenyl.

22. The compound of claim 17, wherein X² is C₁-C₆ alkyl.

23. The compound of claim 17, wherein X² is C₁-C₆ alkyl substituted with one or more groups selected from hydroxyl, amino, carboxamide, guanidine, acyl and halogen.

24. The compound of claim 17, wherein X² is optionally substituted heteroaryl.

25. The compound of claim 17, wherein X² is optionally substituted heteroaryl substituted with one or more groups selected from carbonyl and hydroxyl.

26. The compound of claim 17, wherein X² is NR^{2a}R^{2b}, where R^{2a} is an optionally substituted aryl group and R^{2b} is H.

27. The compound of claim 17, wherein X³ is an optionally substituted carbazole.

28. The compound of claim 17, wherein X³ is an optionally substituted napthyl.

29. The compound of claim 17, wherein m is 1 or 2.

30. A compound described by the formula (II): 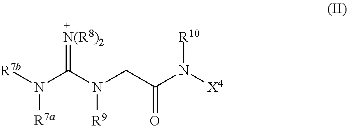
wherein:
R^{7a}, R^{7b}, R⁸, R⁹ and R¹⁰ are each independently selected from hydrogen, alkyl and substituted alkyl; and
X⁴ is selected from alkyl, aryl, aralkyl, heterocycle, and heteroaryl, acyl, wherein X⁴ is optionally further substituted with one or more groups selected from, alkyl, substituted alkyl, aryl, substituted aryl, amino, substituted amino, carboxamide, substituted carboxamide, heterocycle, substituted heterocycle, and a second compound of formula (II)
or a pharmaceutically acceptable salt or solvate thereof.

31. The compound of claim 30, wherein the compound is described by the formula (IIa): 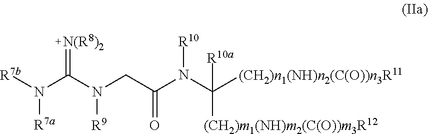
wherein:
R^{7a}, R^{7b}, R⁸, R⁹, R¹⁰ and R^{10a} are each independently selected from hydrogen, and alkyl;
R¹¹ and R¹² are each independently selected from aryl, substituted aryl, heteroaryl, substituted heteroaryl, naphthyl, substituted naphthyl, carbazole, and substituted carbazole;
n₁ and m₁ are each independently an integer from 1 to 10;
n₂ and m₂ are each independently 0 or 1; and
n₃ and m₃ are each independently 0 or 1.

32. The compound of claim 31, wherein the compound is selected from the group consisting of:

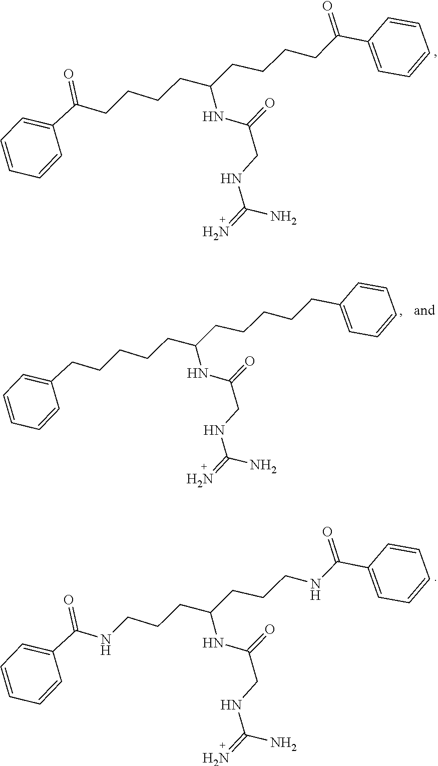 [SMILES: N=C(N)NCC(=O)NC(CCCCC(=O)c1ccccc1)CCCCC(=O)c1ccccc1 ; N=C(N)NCC(=O)NC(CCCCCc1ccccc1)CCCCCc1ccccc1 ; N=C(N)NCC(=O)NC(CCCNC(=O)c1ccccc1)CCCNC(=O)c1ccccc1]

33. The compound of claim 30, wherein the compound is selected from the group consisting of:

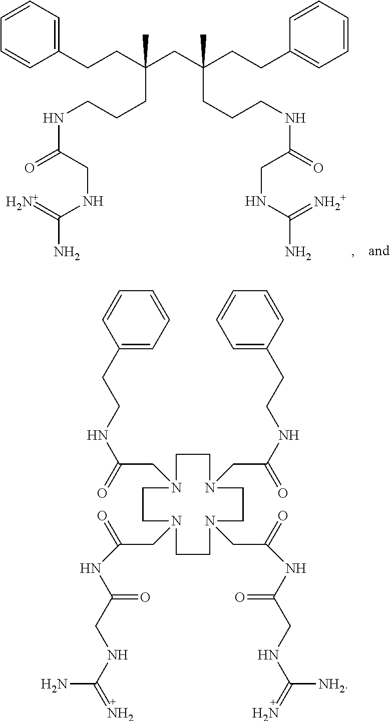 [SMILES: C[C@](CCCNC(=O)CNC(N)=[NH2+])(CCc1ccccc1)C[C@](C)(CCCNC(=O)CNC(N)=[NH2+])CCc1ccccc1 ; N=C(N)NCC(=O)NC(=O)CN1CCN(CC(=O)NCCc2ccccc2)CCN(CC(=O)NCCc2ccccc2)CCN(CC(=O)NC(=O)CNC(=N)N)CC1]

34. A compound described by the formula (III): 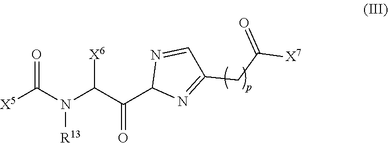
wherein:
R¹³ is selected from hydrogen, alkyl and substituted alkyl;
X⁵ is selected from alkyl, substituted alkyl, aryl, substituted aryl, amino, substituted amino, heteroaryl, substituted heteroaryl, heterocycle, substituted heterocycle;
X⁶ is selected from alkyl, substituted alkyl, aryl, substituted aryl, aralkyl, substituted aralkyl, heterocycle, substituted heterocycle, heteroaryl, substituted heteroaryl;
X⁷ is selected from alkyl, substituted alkyl, aryl, substituted aryl, naphthyl, substituted naphthyl, heterocycle, substituted heterocycle, heteroaryl, substituted heteroaryl, aryl heterocycle, substituted aryl heterocycle; and
p is an integer from 1 to 10,
or a pharmaceutically acceptable salt or solvate thereof.
